(19) United States
(12) Patent Application Publication
(10) Pub. No.: US 2013/0337485 A1
(43) Pub. Date: Dec. 19, 2013

(54) ENZYMATIC SYNTHESIS OF ACTIVE PHARMACEUTICAL INGREDIENT AND INTERMEDIATES THEREOF

(71) Applicants: Peter Mrak, Ljubljana (SI); Tadeja Zohar, Ljubljana (SI); Matej Oslaj, Ljubljana (SI); Gregor Kopitar, Ljubljana (SI)

(72) Inventors: Peter Mrak, Ljubljana (SI); Tadeja Zohar, Ljubljana (SI); Matej Oslaj, Ljubljana (SI); Gregor Kopitar, Ljubljana (SI)

(73) Assignee: LEK PHARMACEUTICALS D.D, Ljubljana (SI)

(21) Appl. No.: 13/995,420

(22) PCT Filed: Dec. 20, 2011
(86) PCT No.: PCT/EP2011/073412

§ 371 Date: Aug. 29, 2013

(30) Foreign Application Priority Data
Dec. 20, 2010 (EP) 10015774.2
May 31, 2011 (EP) 11168227.4

(51) Int. Cl. C12P 17/06; C12P 17/12; C12P 17/10

(52) U.S. Cl. CPC C12P 17/06; C12P 17/10; C12P 17/12
USPC 435/26; 435/121; 435/122; 435/125; 435/135; 435/189; 435/325; 435/348; 435/252.3; 435/254.2; 435/320.1

(57) ABSTRACT

The present invention discloses a process for preparing an active pharmaceutical ingredient (API) or intermediates thereof, notably particular step in the synthesis of an intermediate useful for example in the preparation of statins, by using an enzyme capable of catalyzing oxidation or dehydrogenation. The invention further provides an expression system effectively translating said enzyme. In addition, the invention relates to a specific use of such enzyme for preparing API or intermediate thereof, and in particular for preparing statin or intermediate thereof.

### FIELD OF THE INVENTION

The present invention relates in general to the field of chemical technology and in particular to a process for preparing an active pharmaceutical ingredient (API) or intermediates thereof by using an enzyme. Particularly, the present invention relates to a preparation of HMG-CoA reductase inhibitors, known also as statins, wherein a certain enzyme is provided for catalyzing a particular step in the synthesis. In certain embodiment, this invention relates to a process for preparing an intermediate by providing said enzyme. The invention further relates to an expression system effectively translating said enzyme. In addition, the invention relates to a specific use of such enzyme for preparing API or intermediate thereof, and in particular for preparing statin or intermediate thereof.

### BACKGROUND OF THE INVENTION

Synthetic routes are routinely performed by carrying out chemical reactions in vitro. The chemistry can become complex and can require expensive reagents, multiple long steps, possibly with low yields because of low stereoselectivity. In specific cases, it is possible to employ a microorganism or its part that is capable of using a starting material as a substrate in its biochemical pathways that convert the starting material to a desired compound. With the aid of microorganisms or their parts, like for example enzymes, optionally together with synthetic process steps, it may be possible to put together complete synthesis pathways for preparing an API or an intermediate thereof, however, it is a challenging task.

For example, Patel et al., Enzyme Microb. Technol., vol. 14, 778-784 (1992) describe a stereoselective microbial/enzymatic oxidation of 7-oxybicyclo[2.2.1]heptane-2,3-dimethanol to the corresponding chiral lactol and lactone, by using horse liver alcohol dehydrogenase or by microorganisms oxidizing the compound. Further, Moreno-Horn et al., Journal of Molecular Catalysis B:Enzymes, vol. 49, 24-27 (2007) describe the oxidation of 1,4-alkanediols into γ-lactones via γ-lactols using Rhodococcus erythropolis as biocatalyst.

A particular example of a complex synthesis route is the synthesis of inhibitors of the enzyme 3-hydroxy-3-methylglutaryl-coenzyme A reductase (HMG CoA reductase), which were found to be an excellent tool to reduce lipids and cholesterol blood levels in order to reduce mortality due to serious cardiovascular events in atherosclerosis triggered pathological processes and diseases. Among HMG CoA reductase inhibitors, a natural lovastatin is known, which has been superseded by semisynthetic pravastatin, simvastatin and later by completely synthetic atorvastatin, cerivastatin, rosuvastatin, fluvastatin, pitavastatin, bervastatin and dalvastatin. The clinically effective HMG CoA reductase inhibitors are also known as statins (characterised by INN name ending-statin).

All statins share a characteristic side chain consisting of respectively a heptenoic or heptanoic acid moiety (free acid, salt or lactone) connected to a statin backbone (Scheme 1). Biological activity of statins is linked to this structure and its stereochemistry. Normally, multiple chemical steps are required to prepare the heptenoic or heptanoic acid moiety. The construction of this side chain still represents a challenge for a chemist.

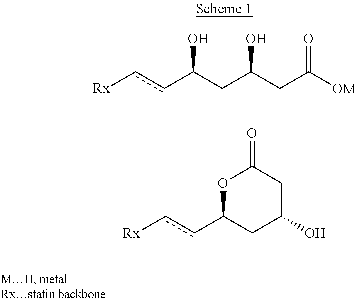

A first attempt to prepare a side chain and thus statin via lactol was disclosed in U.S. Pat. No. 7,414,119. WO 2006/134482 disclosed the application of aldolases (in particular 2-deoxyribose-5-phosphate aldolase; DERA; EC class EC 4.1.2.4) for the synthesis of lactols, which enabled direct coupling to obtain atorvastatin.

For further use and eventually yielding statin compounds lactol must be oxidised. In J. Am. Chem. Soc. 116 (1994), p. 8422-8423 and WO 2008/119810 the purified lactol was oxidised to lactone by Br₂/BaCO₃. WO 2006/134482 discloses a use of a catalytic dehydrogenation (e.g. Pt/C, Pd/C) for the same purpose.

It is an object of the present invention to provide a new process that generally allows oxidation or dehydrogenation in preparing API or intermediate thereof in reduced number of process steps, in shorter time, high yield and in an improved, more economic and simplified fashion. Specific aspects of the present invention can be applied for preparing statins or their intermediates.

### SUMMARY OF THE INVENTION

To solve the aforementioned object, the present invention provides a process according to claim 1. Preferred embodiments are set forth in the subclaims. The present invention further provides a reaction system according to claim 9, a process for preparing a pharmaceutical composition according to claim 13, expression systems according to claims 14 and 15 and uses according to claims 18 to 21.

Surprisingly it has been found that oxidation from certain lactol to lactone moieties typical for statin type compound synthesis is possible using particular enzymes. It is noted that oxidizing lactols with chemical reagents requires lactols to be first isolated, which is extremely burdensome and consumes large amounts of organic solvents. In addition, 4-hydroxy-lactols have to be first protected with the hydroxyl protection group, which adds to a number of reaction steps and to final impurity profile of an API. On the other hand, the oxidation or dehydrogenation of the compound of formula (II) performed with the enzyme catalyst as defined above according to the present invention is extremely favourable over the prior art chemical oxidation with Br₂/BaCO₃ or the chemical catalytic dehydrogenation (e.g. Pt/C, Pd/C). Chemical oxidants are not specific and thus need previous purification of lactol, otherwise too much reagent is consumed in oxidation of the side products or reagents, or even solvents. The purification of lactol is demanding and requires substantial amounts of a solvent for extraction, which is linked to the fact that lactol is normally hydrophilic and is hardly extracted to the organic solvent, such as for example ethylacetate. Also to note is that solvents used in the extraction need to be evaporated, which is not desired when working on the industrial scale. All aforementioned pitfalls of the chemical oxidation are solved by the process of the present invention. Circumventing the purification and evaporation steps by using the enzyme capable of catalyzing oxidation or dehydrogenation shortens significantly the process of preparing the compound of formula (I).

Although lactols such as compound (II) are non-natural type compounds, they have surprisingly been found to work as effective substrates for the enzymes disclosed herein. Based on this finding, use of the enzyme capable of catalyzing oxidation or dehydrogenation provides a valuable tool for generally preparing a synthetic API or synthetic intermediate thereof. Generally, the substrate within the use of the present invention for preparing a non-natural, synthetic API or intermediate thereof will therefore be different from naturally occurring ones of the enzyme capable of catalyzing oxidation or dehydrogenation, thereby excluding for example natural substrates selected from ethanol, methanol, acetaldehyde, acetic acid, naturally occurring sugars or amino acids, or sugar acids derived from sugars, including monosaccharides having a carboxylic group such as gluconic acid.

Aspects, advantageous features and preferred embodiments of the present invention summarized in the following items, respectively alone or in combination, contribute to solving the object of the invention.
(1) A process for preparing a compound of formula (I)

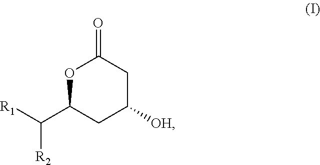

in which
R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, NR³CO(CH₂)ₙCH₃, CH₂—R⁵, optionally substituted mono- or bicyclic aryl, heterocyclic or alicyclic group; and
R₂ independently from R₁ denotes H, (CH₂)ₘ—CH₃, or aryl;
or both of R₁ and R₂ denote either X, OH or O((CH₂)ₙCH₃);
or R₁ and R₂ together denote ═O, ═CH—R⁵, or together form a ring —(CH₂)ₚ—, —(CH₂)ᵣ-(1,2-arylene)-(CH₂)ₛ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³; wherein
R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
R⁵ denotes optionally substituted mono- or bicyclic aryl, heterocyclic or alicyclic group,
X denotes F, Cl, Br or I;
n represents an integer from 0 to 10;
m represents an integer from 0 to 3;
p represents an integer from 2 to 6;
and at least one from r and s is 1;
or a pharmaceutically acceptable salt, or an ester, or a stereoisomer thereof, the process comprising bringing in contact a compound of formula (II),

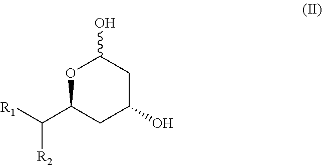

wherein R₁ and R₂ are defined as above, with an enzyme capable of catalyzing oxidation or dehydrogenation, and optionally salifying, esterifying or stereoselectively resolving the product.
(2) The process according to item 1, wherein
   R⁵ denotes a moiety selected from the formula (III), (IV), (V), (VI), (VII), (VIII) and (IX);

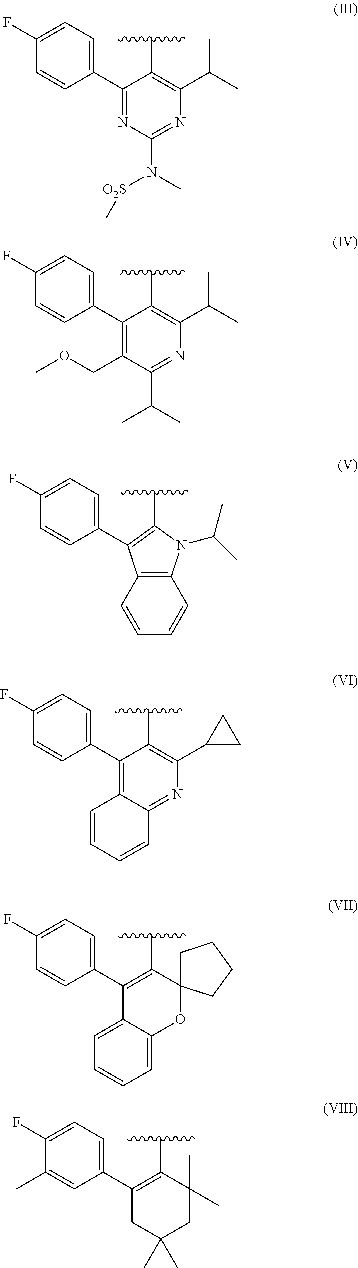 [SMILES: *c1c(-c2ccc(F)cc2)nc(N(C)S(C)(=O)=O)nc1C(C)C ; *c1c(C(C)C)nc(C(C)C)c(COC)c1-c1ccc(F)cc1 ; *c1c(-c2ccc(F)cc2)c2ccccc2n1C(C)C ; *c1c(C2CC2)nc2ccccc2c1-c1ccc(F)cc1 ; *C1=C(c2ccc(F)cc2)c2ccccc2OC12CCCC2 ; *C1=C(c2ccc(F)c(C)c2)CC(C)(C)CC1(C)C]

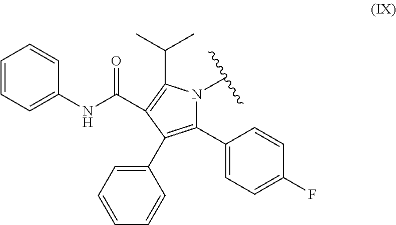 [SMILES: *n1c(-c2ccc(F)cc2)c(-c2ccccc2)c(C(=O)Nc2ccccc2)c1C(C)C]

(3) The process for preparing a compound of formula (I) according to items 1 or 2, in which R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, or NR³CO(CH₂)ₙCH₃, optionally substituted mono- or bicyclic aryl, heterocyclic or alicyclic group; and
   R₂ independently from R₁ denotes H, (CH₂)ₘ—CH₃, or aryl;
   or both of R₁ and R₂ denote X, OH or O(CH₂)ₙCH₃;
   or R₁ and R₂ together denote ═O, or together form a ring —(CH₂)ₚ—, —(CH₂)ᵣ-(1,2-arylene)-(CH₂)ₛ—, wherein
   any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
   each CH₂ linking carbon atoms can be replaced by O, S or NR³; wherein
   R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
   X denotes F, Cl, Br or I;
   n represents an integer from 0 to 10;
   m represents an integer from 0 to 3;
   p represents an integer from 2 to 6;
   and at least one from r and s is 1.
(4) The process for preparing a compound of formula (I) according to any one of the previous items, in which
   R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, or NR³CO(CH₂)ₙCH₃; and
   R₂ independently from R₁ denotes H or (CH₂)ₘ—CH₃;
   or both of R₁ and R₂ denote X, OH or O(CH₂)ₙCH₃;
   or R₁ and R₂ together denote ═O, —(CH₂)ₚ—, —(CH₂)ᵣ-(1,2-arylene)-(CH₂)ₛ—, wherein
   R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
   X denotes F, Cl, Br or I;
   n represents an integer from 0 to 10;
   m represents an integer from 0 to 3;
   p represents an integer from 2 to 6;
   and at least one from r and s is 1.
(5) The process according to any one of the previous items, wherein the enzyme capable of catalyzing oxidation or dehydrogenation is an oxidase or a dehydrogenase.
(6) The process according to any one of the previous items, wherein the enzyme capable of catalyzing oxidation or dehydrogenation is capable of catalyzing aldose dehydrogenation.
(7) The process according to any one of the previous items, wherein the enzyme capable of catalyzing oxidation or dehydrogenation is an aldose dehydrogenase.
(8) The process according to any one of the previous items, wherein the enzyme capable of catalyzing oxidation or dehydrogenation is specific for oxidation of lactol hydroxy group.
(9) The process according to any one of the previous items, wherein the enzyme capable of catalyzing oxidation or dehydrogenation is aldose 1-dehydrogenase (EC 1.1.5.2).
(10) The process according to any one of the previous items, wherein the enzyme capable of catalyzing oxidation or dehydrogenation is selected from a group of pyrroloquinoline quinine (PQQ) dependent dehydrogenases.
(11) The process according to any one of the previous items, wherein the enzyme is water soluble or membrane bound.
(12) The process according to any one of the previous items, wherein the enzyme is selected from the group consisting of dehydrogenases encoded by dehydrogenase-encoding genes comprised within, or constituted by, any one of nucleotide sequences of SEQ ID NOS. 01, 03, 05, 07, 09, 23, 25, 27, 29, 31, 33, 35, 37, 39, 41, 43, 45, 47, 49, 51, 53, 55, 57, 59 and 61; or dehydrogenases defined by any one of amino acid sequences comprised within, or constituted by, SEQ ID NOS. 02, 04, 06, 08, 10, 24, 26, 28, 30, 32, 34, 36, 38, 40, 42, 44, 46, 48, 50, 52, 54, 56, 58, 60 and 62; or any dehydrogenase having a nucleotide sequence identity or an amino acid sequence identity respectively of at least 50% to said sequences, preferably 70% to, more preferably 90% to said sequences, provided that the resulting sequence variants maintain dehydrogenase activity.
(13) The process according to any one of the previous items, wherein the enzyme is YliI aldose dehydrogenase or Gcd membrane bound glucose dehydrogenase.
(14) The process according to any one of the previous items, wherein 2-deoxyribose-5-phosphate aldolase (DERA, EC 4.1.2.4) enzyme is used for preparing the compound of formula (II).
(15) The process according to item 14, wherein 2-deoxyribose-5-phosphate aldolase (DERA, EC 4.1.2.4) enzyme is used for a synthetic step preceding, or alternatively simultaneously at least in an overlapping time period with, bringing in contact the compound of formula (II) with the enzyme capable of catalyzing oxidation or dehydrogenation.
(16) The process according to any one of the previous items, wherein the compound of formula (II) is brought in contact with the enzyme capable of catalyzing oxidation or dehydrogenation without prior isolation or purification of the compound of formula (II).
(17) The process according to any one of the previous items, wherein the enzyme capable of catalyzing oxidation or dehydrogenation, optionally also DERA enzyme independently, are comprised within living whole cell, inactivated whole cell, homogenized whole cell, or cell free extract; or are purified, immobilized and/or are in the form of an extracellularly expressed protein, preferably are within living whole cell, inactivated whole cell or homogenized whole cell, more preferably are within living whole cell or inactivated whole cell, particularly are comprised within living whole cell.
(18) The process according to any one of items 14 to 17, wherein the compound of formula (I) is prepared at least in part simultaneously with the preparation of the compound of formula (II).

In the preferred embodiments as defined in items 14 to 17, the arrangement of having the compound (II) prepared by using 2-deoxyribose-5-phosphate aldolase (DERA) enzyme and allowing the product to be used, preferably to be simultaneously used at least in an overlapping time period, with a subsequent oxidation reaction by the enzyme capable of catalyzing oxidation or dehydrogenation is especially advantageous in terms of process efficiency and reduced time required for the process. When the processes proceed at least partially simultaneously, the prepared compound of formula (II) can get immediately consumed as a substrate in a subsequent oxidation reaction, which shifts the steady state equilibrium of the first reaction in a direction of the product.
(19) The process according to any one of the previous items, wherein electron acceptor is comprised in the process or added to the enzyme capable of catalyzing oxidation or dehydrogenation, preferably oxygen is used or added; and/or wherein cofactor(s) of the enzymes in order to become functional is comprised in the process or added, such as cofactors selected from the group of FAD, NAD(P)⁺ and/or PQQ, and optionally further additives and auxiliary agents as disclosed herein.

When according to this further preferred embodiment oxygen is added to the enzyme capable of catalyzing oxidation or dehydrogenation, or the process is run in the presence of oxygen, like for example under aerated conditions, preferably when air is bubbled to the dehydrogenation or oxidation reaction catalysed by the dehydrogenation or oxidation enzyme, the reaction becomes irreversible, which secures the obtained product and further enhances shifting of the steady state equilibrium of the first reaction, when the compounds of formula (II) and (I) are prepared simultaneously. The presence of oxygen, particularly the presence of dissolved oxygen above 5%, wherein 100% dissolved oxygen is understood as saturated solution of oxygen at given process conditions, is again a favourable process parameter that increases yield and reduces reaction times. In addition, allowing oxygen to be present might in a specific case promote proliferation of a microorganism used. Similar explanations apply to the use of cofactor(s) and optional further additives and auxiliary agents useful for the respective enzyme.
(20) A reaction system, or a one-pot process for preparing a compound of formula (I)

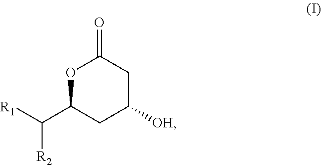

in which R₁ and R₂ are as defined in any one of items 1 to 4, or a pharmaceutically acceptable salt, ester or stereoisomer thereof,
the reaction system being capable of or arranged for, or the one-pot process comprising reacting a compound of formula (X),

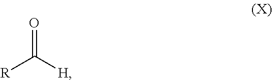

in which R denotes R₁—CH—R₂ moiety of formula (I), R₁ and R₂ being as defined in any one of terms 1 to 4,
with acetaldehyde in the presence of 2-deoxyribose-5-phosphate aldolase (DERA) enzyme and an enzyme capable of catalyzing oxidation or dehydrogenation, and
optionally salifying, esterifying or stereoselectively resolving the product.

Here, the enzyme capable of catalyzing oxidation or dehydrogenation may preferably be represented by an enzyme as defined in any one of items 5 to 13.

The term “reaction system” means a technical system, for example an in vitro-system, a reactor or a cultivation vessel or a fermentor.

The terms “capable of” or “arranged for” means that the reaction system is suitable, or conditions and configurations are set that the defined reaction can take place.
(21) The system or process according to item 20, wherein a compound of formula (II) as defined in item 1 is generated as intermediate, which is not isolated.
(22) The system or process according to any one of items 20 or 21, wherein the enzyme capable of catalyzing oxidation or dehydrogenation, optionally also DERA enzyme independently, are comprised within living whole cell, inactivated whole cell, homogenized whole cell, or cell free extract; or are purified, immobilized and/or are in the form of an extracellularly expressed protein, preferably are within living whole cell, inactivated whole cell or homogenized whole cell, more preferably are within living whole cell or inactivated whole cell, particularly are comprised within living whole cell.
(23) The system or process according to any of the items 20 to 22, wherein both said enzymes, i.e. DERA and the enzyme capable of catalyzing oxidation or dehydrogenation, are expressed by a same cell.
(24) The process according to item 17, or the system or process according to items 22 or 23, wherein the cell is a bacteria, yeast, insect cell or a mammalian cell, preferably is bacteria or yeast, more preferably is bacteria.
(25) The system or process according to item 24, wherein the bacteria is selected from the group of genera consisting of Escherichia, Corynebacterium, Pseudomonas, Streptomyces, Rhodococcus, Bacillus, Lactobacillus, Klebsiella, Enteorobacter, Acinetobacter, Rhizobioum. Methylobacterium, Kluyvera, Gluconobacter, Erwinia, Rahnella and Deinococcus. In a more particular sense the microorganisms may be selected from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans, Corynebacterium glutamicum, Escherichia coli, Bacillus licheniformis, and Lactobacillus lactis, most preferably from Escherichia coli, Gluconobacter oxydans, Acinetobacter calcoaceticus and Kluyvera intermedium, particularly is Escherichia coli.
(26) The system or process according to item 24, wherein the yeast is selected from the group of genera consisting of Saccharomyces, Pichia, Shizosaccharomyces and Candida, preferably Saccharomyces.
(27) The system or process according to item 24, wherein mammalian cell is Chinese hamster ovary cell or a hepatic cell, preferably is Chinese hamster ovary cell.
(28) The process according to any one of the previous items, further comprising subjecting said compound (I) to conditions sufficient to prepare a statin, or a pharmaceutically acceptable salt thereof, preferably lovastatin, pravastatin, simvastatin, atorvastatin, cerivastatin, rosuvastatin, fluvastatin, pitavastatin, bervastatin, or dalvastatin, or a pharmaceutically acceptable salt thereof, more preferably atorvastatin, rosuvastatin or pitavastatin, or a pharmaceutically acceptable salt thereof, particularly rosuvastatin, or a pharmaceutically acceptable salt thereof.
(29) A process for preparing a statin or a pharmaceutically acceptable salt, ester or stereoisomer thereof, comprising steps of:
   (i) bringing in contact the compound of formula (II),

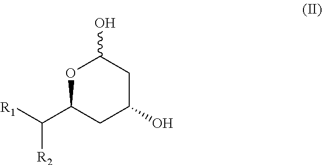

wherein R₁ and R₂ are defined as in any one of items 1 to 4, with an enzyme capable of catalyzing oxidation or dehydrogenation, to prepare a compound of formula (I) as defined in in any one of items 1 to 4; and
(ii) subjecting said compound (I) to conditions sufficient to prepare a statin;
(iii) optionally salifying, esterifying or stereoselectively resolving the product.
(30) The process according to previous item, wherein the compound of formula (II) is prepared by 2-deoxyribose-5-phosphate aldolase (DERA, EC 4.1.2.4) enzyme.
(31) The process according to previous item, wherein the compounds of formula (II) and (I) are prepared at least in part simultaneously.
(32) The process according to any one of items 29 to 31, wherein the statin is lovastatin, pravastatin, simvastatin, atorvastatin, cerivastatin, rosuvastatin, fluvastatin, pitavastatin, bervastatin, or dalvastatin, more preferably atorvastatin, rosuvastatin or pitavastatin, particularly rosuvastatin.
(33) The process according to any one of the items 29 to 32, wherein further special conditions as defined in any one of items 5 to 19, or 20 to 27, either alone or in combination, are further met.
(34) The process according to any one of items 29 to 33, further comprising formulating said statin, or a pharmaceutically acceptable salt thereof, preferably lovastatin, pravastatin, simvastatin, atorvastatin, cerivastatin, rosuvastatin, fluvastatin, pitavastatin, bervastatin, or dalvastatin, or a pharmaceutically acceptable salt thereof, more preferably atorvastatin, rosuvastatin or pitavastatin, or a pharmaceutically acceptable salt thereof, particularly rosuvastatin, or a pharmaceutically acceptable salt thereof, in a pharmaceutical formulation.
(35) An expression system comprising one or more cell types, the respective cell type(s) being genetically engineered to express, in the totality of cell type(s), both 2-deoxyribose-5-phosphate aldolase (DERA) enzyme and an enzyme capable of catalyzing oxidation or dehydrogenation.
(36) An expression system capable of translating 2-deoxyribose-5-phosphate aldolase (DERA) enzyme and an enzyme capable of catalyzing oxidation or dehydrogenation, and overexpressing both of the genes needed for said translation.
(37) The expression system according to item 35 or 36, wherein the enzyme capable of catalyzing oxidation or dehydrogenation is an oxidase or a dehydrogenase.
(38) The expression system according to any one of items 35 to 37, wherein the enzyme capable of catalyzing oxidation or dehydrogenation is capable of catalyzing aldose dehydrogenation.
(39) The expression system according to any one of items 35 to 38, wherein the enzyme capable of catalyzing oxidation or dehydrogenation is an aldose dehydrogenase.
(40) The expression system according to any one of items 35 to 38, wherein the enzyme capable of catalyzing oxidation or dehydrogenation is selected from broad spectrum sugar dehydrogenases.
(41) The expression system according to any one of items 35 to 40, wherein the enzyme capable of catalyzing oxidation or dehydrogenation is specific for oxidation at position C1
(42) The expression system according to any one of items 35 to 41, wherein the enzyme capable of catalyzing oxidation or dehydrogenation is aldose 1-dehydrogenase (EC 1.1.5.2).
(43) The expression system according to any one of items 35 to 42, further capable of expressing a cluster of genes for providing pyrroloquinoline quinine (PQQ).
(44) The expression system according to any one of items 35 to 43, wherein said expression system is a bacteria, yeast, insect cell or a mammalian cell, preferably is bacteria or yeast, more preferably is bacteria.
(45) The expression system according to item 44, wherein the bacteria is selected from the group of genus consisting of Escherichia, Corynebacterium, Pseudomonas, Streptomyces, Rhodococcus, Bacillus, Lactobacillus, Klebsiella, Enteorobacter, Acinetobacter, Rhizobioum. Methylobacterium, Kluyvera, Gluconobacter, Erwinia, Rahnella and Deinococcus. In a more particular sense the microorganisms may be selected from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans, Corynebacterium glutamicum, Escherichia coli, Bacillus licheniformis, and Lactobacillus lactis, most preferably from Escherichia coli, Gluconobacter oxydans, Acinetobacter calcoaceticus and Kluyvera intermedium, particularly is Escherichia coli.
(46) The expression system according to item 44, wherein the yeast is selected from the group of genus consisting of Saccharomyces, Pichia, Shizosaccharomyces and Candida, preferably Saccharomyces.
(47) The expression system according to item 44, wherein mammalian cell is Chinese hamster ovary cell or a hepatic cell, preferably is Chinese hamster ovary cell.
(48) The expression system according to any one of items 35 to 47, which is arranged to express the enzyme capable of catalyzing oxidation or dehydrogenation, DERA (deoxyribose 5-phosphate aldolase), PQQ dependant dehydrogenase and PQQ biosynthetic pathway genes simultaneously.
(49) The expression system according to any one of items 35 to 48, wherein the enzyme capable of catalyzing oxidation or dehydrogenation is selected from the group consisting of dehydrogenases encoded by dehydrogenase-encoding genes comprised within, or constituted by, any one of nucleotide sequences of SEQ ID NOS. 01, 03, 05, 07, 09, 23, 25, 27, 29, 31, 33, 35, 37, 39, 41, 43, 45, 47, 49, 51, 53, 55, 57, 59 and 61; or dehydrogenases defined by any one of amino acid sequences comprised within, or constituted by, SEQ ID NOS. 02, 04, 06, 08, 10, 24, 26, 28, 30, 32, 34, 36, 38, 40, 42, 44, 46, 48, 50, 52, 54, 56, 58, 60 and 62;
   or any dehydrogenase having nucleotide sequence identity or an amino acid sequence identity respectively of at least 50% to said sequences, preferably 70% to, more preferably 90% to said sequences, provided that the resulting sequence variants maintain dehydrogenase activity.
(50) The expression system according to any one of items 35 to 49, wherein the PQQ is provided by expression of a PQQ-synthesis encoding gene comprised within, or constituted by, any one of the nucleotide sequences of SEQ ID NOS. 11, 17, 63, 64, 65, 66, 67, 68, 69, 70; or by expression of a PQQ-synthesis gene encoding any one of the amino acid sequence SEQ ID NOS. 12, 13, 14, 15, 16, 18, 19, 20, 21 and 22; or by expression of a PQQ-synthesis encoding gene having a nucleotide sequence identity or an amino acid sequence identity respectively of at least 50% to said sequences, preferably 70% to, more preferably 90% to said sequences, provided that the resulting sequence variants maintain activity to produce PQQ.
(51) The expression system according to item 50, providing for PQQ gene cluster.
(52) Use of an enzyme capable of catalyzing oxidation or dehydrogenation for preparing a synthetic API or intermediate thereof.
(53) The use according to item 52, wherein the synthetic API or intermediate thereof is a substrate compound for the enzyme capable of catalyzing oxidation or dehydrogenation, said compound being a non-natural compound selected from the group consisting of substituted or unsubstituted dideoxyaldose sugars, synthetic non-natural alcohols, esters further hydroxylated and lactols further hydroxylated, preferably said non-natural compound comprises a lactol structural moiety.

According to this embodiment, the enzyme capable of catalyzing oxidation or dehydrogenation, more specifically an enzyme as defined in any one of items 5 to 13, can act upon a precursor compound comprising the corresponding lactol structural moiety.
(54) Use of an enzyme capable of catalyzing oxidation or dehydrogenation according to item 52 or 53 simultaneously with, or subsequently to, a DERA enzyme.
(55) Use according to any one of items 52 to 54, wherein conversion of ethanol, monosaccharide or acetaldehyde by the enzyme capable of catalyzing oxidation or dehydrogenation is excluded.
(56) Use of an aldose dehydrogenase enzyme for preparing a compound of formula (I), wherein the formula (I) is as defined in any one of item 1 to 4.
(57) Use of the aldose dehydrogenase enzyme according to item 56, wherein said enzyme is contacted with a compound of formula (II) as defined in item 1.
(58) Use according to any one of items 52 to 57, wherein any one of the conditions of items 2 to 51, either alone or in combination, is further met.
(59) Use of an aldose dehydrogenase enzyme for preparing statin or a pharmaceutically acceptable salt thereof, preferably lovastatin, pravastatin, simvastatin, atorvastatin, cerivastatin, rosuvastatin, fluvastatin, pitavastatin, bervastatin, or dalvastatin, or a pharmaceutically acceptable salt thereof, more preferably atorvastatin, rosuvastatin or pitavastatin, or a pharmaceutically acceptable salt thereof, particularly rosuvastatin, or a pharmaceutically acceptable salt thereof.
(60) Use of an expression system according to any one of items 35 to 51 for preparing a compound of formula (I), wherein the formula (I) is as defined in any one of items 1 to 4.
(61) Use of the expression system according to item 60, wherein any one of the conditions of items 2 to 34, either alone or in combination, is further met.
(62) Use of an expression system according to any one of items 35 to 51 for preparing lovastatin, pravastatin, simvastatin, atorvastatin, cerivastatin, rosuvastatin, fluvastatin, pitavastatin, bervastatin, or dalvastatin, more preferably atorvastatin, rosuvastatin or pitavastatin, particularly rosuvastatin.
(63) Use of the expression system according to item 62, wherein any one of the conditions of items 2 to 34, either alone or in combination, is further met.
(64) Use of a microorganism for preparing a compound of formula (I)

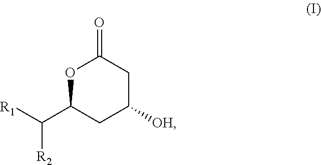

in which
R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, NR³CO(CH₂)ₙCH₃, CH₂—R⁵, optionally substituted mono- or bicyclic aryl, heterocyclic or alicyclic group; and
R₂ independently from R₁ denotes H, (CH₂)ₘ—CH₃, or aryl;
or both of R₁ and R₂ denote either X, OH or O((CH₂)ₙCH₃);
or R₁ and R₂ together denote ═O, ═CH—R⁵, or together form a ring —(CH₂)ₚ—, —(CH₂)ᵣ-(1,2-arylene)-(CH₂)ₛ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³; wherein
R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
R⁵ denotes optionally substituted mono- or bicyclic aryl, heterocyclic or alicyclic group,
X denotes F, Cl, Br or I;
n represents an integer from 0 to 10;
m represents an integer from 0 to 3;
p represents an integer from 2 to 6;
and at least one from r and s is 1;
or a pharmaceutically acceptable salt, or an ester, or a stereoisomer thereof, optionally with further processing of the compound of formula (I) to prepare a statin;
wherein the microorganism
(i) is selected from bacterial origin of the genera Klebsiella Enteorobacter, Acinetobacter, Rhizobioum. Methylobacterium, Kluyvera, Gluconobacter, Pseudomonas, Erwinia, Rahnella and Deinococcus; more particularly selected from the group of specific microorganisms Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans; preferably selected from the group consisting of: Gluconobacter oxydans, Acinetobacter calcoaceticus and Kluyvera intermedium; or
(ii) is Escherichia coli which is genetically engineered to be capable of expressing genes of the gene cluster for providing pyrroloquinoline quinine (PQQ) or is complemented with the addition of exogenous PQQ.

### DETAILED DESCRIPTION OF THE INVENTION

Surprisingly we found a process for preparing a compound of formula (I)

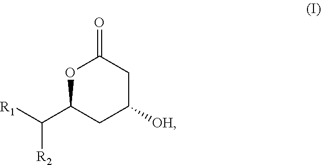

in which
R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, NR³CO(CH₂)ₙCH₃, CH₂—R⁵, or optionally substituted mono- or bicyclic aryl, heterocyclic or alicyclic group; and
R₂ independently from R₁ denotes H, (CH₂)ₘ—CH₃, or aryl;
or both of R₁ and R₂ denote either X, OH or O((CH₂)ₙCH₃);
or R₁ and R₂ together denote ═O, ═CH—R₅, or together form a ring —(CH₂)ₚ—, —(CH₂)ᵣ-(1,2-arylene)-(CH₂)ₛ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³; wherein
R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
R⁵ denotes optionally substituted mono- or bicyclic aryl, heterocyclic or alicyclic group,
X denotes F, Cl, Br or I;
n represents an integer from 0 to 10;
m represents an integer from 0 to 3;
p represents an integer from 2 to 6;
and at least one from r and s is 1;
or a pharmaceutically acceptable salt, ester, or stereoisomer thereof,
wherein a compound of formula (II),

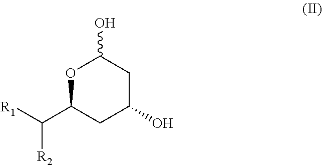

wherein R₁ and R₂ are defined as above, can be simply brought in contact with an enzyme capable of catalyzing oxidation or dehydrogenation, and optionally the product is salifyed, esterifyed or stereoselectively resolved.

The term “mono- or bicyclic aryl group” as used herein refers to any mono- or bicyclic, 5-, 6- or 7-membered aromatic or heteroaromatic ring, such as for example pyrolyl, furanyl, tiophenyl, phenyl, imidazolyl, pyridinyl, piridazinyl, indolyl, kinolinyl ftaliminyl and benzimidazolyl.

The term “aryl” as used herein, if not stated otherwise with respect to particular embodiments, includes reference to an aromatic ring system comprising 6, 7, 8, 9, 10, 11, 12, 13, 14, 15 or 16 ring carbon atoms. Aryl can be phenyl but may also be a polycyclic ring system, having two or more rings, at least one of which is aromatic. This term includes phenyl, naphthyl, fluorenyl, azulenyl, indenyl, anthryl and the like.

The term “mono- or bicyclic heterocyclic group” as used herein refers to any mono- or bicyclic, 5-, 6- or 7-membered saturated or unsaturated ring, wherein at least one carbon in the ring is replaced by an atom selected from the group of oxygen, nitrogen and sulphur. The non-limiting examples of mono- or bicyclic heterocyclic group are oksazolyl, thiazolyl, isothiazolyl, morfolinyl.

The term “heterocycle” as used herein includes, if not stated otherwise with respect to particular embodiments, a saturated (e.g. heterocycloalkyl) or unsaturated (e.g. heteroaryl) heterocyclic ring moiety having 3, 4, 5, 6, 7, 8, 9, 10, 11, 12, 13, 14, 15 or 16 ring atoms, at least one of which is selected from nitrogen and oxygen. In particular, heterocyclyl includes a 3- to 10-membered ring or ring system and more particularly a 5- or 6- or 7-membered ring, which may be saturated or unsaturated; examples thereof include oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, ehromenyl, isochromanyl, chromanyl and the like.

More specifically, a saturated heterocyclic moiety may have 3, 4, 5, 6 or 7 ring carbon atoms and 1, 2, 3, 4 or 5 ring heteroatoms selected from nitrogen and oxygen. The group may be a polycyclic ring system but more often is monocyclic, for example including azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolinidinyl and the like. Furthermore, the “heteroaryl” may include an aromatic heterocyclic ring system having 5, 6, 7, 8, 9, 10, 11, 12, 13, 14, 15 or 16 ring atoms, at least one of which is selected from nitrogen and oxygen. The group may be a polycyclic ring system, having two or more rings, at least one of which is aromatic, but is more often monocyclic. This term includes pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

The term “mono- or bicyclic alicyclic group” as used herein refers to any mono- or bicyclic, 5-, 6- or 7-membered alicyclic ring.

The term “salifyed” or “a pharmaceutically acceptable salts” in a context of the compound of formula (I) or (II), API or statin, which can be optionally substituted, as used herein refers to the compound or statin in a form of a salt, such as potassium, sodium, calcium, magnesium, hydrochloride, hydrobromide, or the like, that is also substantially physiologically tolerated. The compound of formula (I) or (II), API or statin, can be salifyed or brought in the form of a salt by mixing the compound of formula (I) or (II), API or statin or intermediate thereof, with an acid or a base, optionally in an aqueous or organic solvent, or a mixture thereof. Preferably the solvent is afterwards removed.

The term “esterifying” or “esters” in a context of the compound of formula (I) or (II), API or statin, as used herein refers to the compound of formula (I) or (II), or statin, with at least one ester bond in their structure. Such ester bond or esterifying the compound can be achieved by coupling the compound of formula (I) or (II), API or statin or intermediate thereof, in the event the compound or statin contains hydroxyl group, with an carboxylic acid or a phosphate group containing compound. In the event that the compound of formula (I) or (II), API or statin or intermediate thereof, contain carboxylic or phosphate group, it can be achieved by coupling it with a hydroxylic group of another compound.

The term “stereoselectively resolved” is used herein to refer to any method known to the skilled person in the field of separating a mixture of stereoisomers, preparatory chemistry of stereospecific compounds, or analytics. The stereoisomers can be obtained for example by HPLC, wherein stereoselective column is used. Stereoselective columns are known in the art.

### Enzymes, Organisms

The term “an enzyme capable of catalyzing oxidation or dehydrogenation” as used herein refers to any enzyme that catalyzes oxidation or dehydrogenation. The enzyme recognises and uses e.g. the compound of formula (II) as a substrate. Combinations of enzymes, multiunit enzymes, wherein different units catalyse optionally different reactions, fused or joined enzymes, or enzymes coupled to another structural or a non-catalytic compound, unit, subunit or moiety, are also contemplated within the present invention as long as the requirement of being capable of catalyzing oxidation or dehydrogenation is fulfilled. The enzyme can be for example an enzyme found in the electron transfer chain of the prokaryote or eukaryote cells or in the biochemical pathways of alcohols, aldehydes or sugars in eukaryote or prokaryote cells. Enzymes that would normally act upon natural substrates were unexpectedly found to recognise and oxidise rather complex synthetic compounds, in particular convert lactols into lactones or possibly into esters. It is an important aspect of the present invention that the reactions, which are meant to be used for the an enzyme capable of catalyzing oxidation or dehydrogenation, do normally not occur in the nature, because the substrate is different from the natural occurring ones, like for example exempting from using the ethanol, methanol, acetaldehyde, acetic acid, naturally occurring sugars or naturally occurring amino acids, or acids obtained from the sugars, like for example gluconic acid. It was however surprisingly found that synthetic substrates as disclosed herein, even if being rather structurally complex, can yet be easily processed by using the enzyme capable of catalyzing oxidation or dehydrogenation in order to finally obtain API, particularly statin, or intermediates thereof, including e.g. a lactone compound of formula (I). Generally, enzyme can be chosen from oxydoreductases.

According to the enzyme nomenclature, the enzyme applicable in the present invention belongs primarily to EC 1.1 (oxidoreductases acting primarily on the CH—OH group of donors), more specifically to, but not limited to subclasses: EC 1.1.1 (with NAD⁺ or NADP⁺ as acceptor), EC 1.1.2 (with a cytochrome as acceptor), EC 1.1.3 (with oxygen as acceptor), EC 1.1.5 (with a quinine or flavine or similar compound as acceptor). Any of the oxidoreductases known in the art may be used for the reaction regardless of their sequence identity.

In the following, enzymes will be described in further detail, which are in principle applicable in the present invention—illustrative experimental examples will be described later, and if necessary suitable screening and/or verification tests are also provided herein. Some of the particular enzymes had been described and optionally used prior to the invention, but in distinctively other contexts or fields than the present invention. References, the disclosures of which are incorporated herein, will be cited and listed below.

Enzymes having activity of oxidation/dehydrogenation of sugars have been widely used in the industry. Typical examples of oxidative fermentations are traditional production of D-gluconate (gluconic acid), L-sorbose and others. These processes were developed as a practical industry based on empirically found properties of some microorganisms before the clarification of the molecular mechanisms of the responsible enzymes [Adachi, 2007].

Sugar oxidising enzymes had been used in food processing as additives, in dairy and the lactoperoxidase system for food preservation, in breadmaking, for producing dry egg powder, as antioxidants/preservatives (oxygen scavengers), for reducing alcohol wine, as glucose assays and fuel cells [Wong, 2008]. Sugar oxidising enzymes had been used also as amperometric biosensors, e.g. for measuring glucose concentration in blood [Igarashi, 2004], for detection of heavy metals [Lapenaite, 2003], for detection of formaldehyde in air [Acmann, 2008], for detection of phenolic compounds in flow injection analysis [Rose, 2001], as a ultrasensitive bienzyme sensor for adrenaline [Szeponik, 1997], for determination of xylose concentration [Smolander, 1992] etc.

A well known enzyme capable of oxidation of six-membered sugars is Glucose oxidase, Gox (EC 1.1.3.4), which is commercially available from Sigma as an extract from Aspergillus niger. This enzyme has a very narrow substrate specificity [Keilin, 1952]. It is produced naturally in some fungi and insects where its catalytic product, hydrogen peroxide, acts as an anti-bacterial and anti-fungal agent. Gox is generally regarded as safe, and Gox from A. niger is the basis of many industrial applications [Wong, 2008]. Gox-catalysed reaction has also been used in baking, dry egg powder production, wine production, gluconic acid production, etc. Its electrochemical activity makes it an important component in glucose sensors, especially in diabetics, as is also the case with PQQ dependent sugar dehydrogenase, and potentially in fuel cell applications. Glucose oxidase is capable of oxidising monosaccharides, nitroalkanes and hydroxyl compounds [Wilson, 1992]. Using the reaction rate of glucose as reference (100%), only 2-deoxy-D-glucose (20-30%), 4-O-methyl-D-glucose (15%) and 6-deoxy-D-glucose (10%) are oxidized by glucose oxidase from A. niger at a significant rate [Pazur, 1964; Leskovac, 2005]. The activities of glucose oxidase against other substrates are typically poor, with reaction rates lower than 2% of glucose's [Keilin, 1948; Pazur, 1964; Leskovac, 2005].

In a preferred embodiment, the enzyme capable of catalyzing oxidation or dehydrogenation is a dehydrogenase. Particularly, the enzyme is capable of catalyzing sugar dehydrogenation, more particularly aldose dehydrogenation. Preferrably the enzyme is sugar dehydrogenase (EC 1.1). In a specific embodiment, the enzyme is an aldose dehydrogenase or a glucose dehydrogenase. The terms of the current art describing such enzymes may be different to the one provided by this invention, however it will be understood herein that substrate specificity and capability of the enzyme to catalyse the oxidation/dehydrogenation of compound (II) or other compounds contemplated herein are independent of terminology found in the current art. One example which can be found is the terminology for an enzyme found in E. coli (YliI, Adh, Asd) which is termed “soluble glucose dehydrogenase” by some authors, “aldose sugar dehydrogenase” or “soluble aldose dehydrogenase” by others. Another example is the terminology for the membrane bound glucose dehydrogenase found in E. Coli (mGDH, GCD, PQQMGDH) which is termed “PQQ dependant glucose dehydrogenase” by some authors or “membrane bound glucose dehydrogenase” or GCD by others.

The natural substrate for the sugar dehydrogenases (aldose dehydrogenases) are various sugars that get oxidized. The broad range of sugars that aldose dehydrogenase can act upon encompasses pentoses, hexoses, disaccharides and trisaccharides. Preferably, the enzyme capable of catalyzing oxidation or dehydrogenation is specific for oxidation at position C1. In the case of the aldose 1-dehydrogenase the enzyme oxidizes the aldehyde or cyclic hemiacetal to lactone.

In agreement to the above sugar oxidoreductases are divided into classes, according to electron acceptors (in some cases these are the cofactors these enzymes use in order to become functional, i.e. FAD, NAD(P)⁺ or PQQ). In terms of substrate specificity, which may vary strongly between the subclasses of sugar oxidoreductases, the use of PQQ dependent dehydrogenases (EC 1.1.5) are preferred according to the present invention.

FAD- (flavoprotein dehydrogenases) and PQQ-dependent sugar dehydrogenases (quinoprotein dehydrogenases), EC 1.1.5, use flavin adenine dinucleotide (FAD) or pyrroloquinoline quinine (PQQ) cofactors respectively, and are located on the outer surface of the cytoplasmic membrane of bacteria, facing the periplasmic space with their active sites. These are often termed membrane sugar dehydrogenases. Alternatively, especially in the PQQ-dependent sugar dehydrogenases group, many enzymes are found in soluble form, located in the periplasmic space. There is no limitation according to this invention in nature of the used sugar dehydrogenase in regard to solubility, however it may be preferred, relating to cloning, expression procedures and molecular tools available, that soluble periplasmic sugar dehydrogenases are selected, i.e. water-soluble ones. Construction of expression strain for efficient periplasmic expression of such enzyme is technically easier and thus preferable. On the other hand the membrane bound sugar dehydrogenases may be more challenging for efficient expression but may be preffered due to their more intimate connection to the respiratory chain, via the transfer of electrons from the PQQ to the ubiquinone pool. In the course of the present invention, examples are provided, e.g. using naturally occurring membrane bound sugar dehydrogenases as well as the overexpressed membrane bound sugar dehydrogenases for conversion of compound (II) to compound (I) in industrially useful yields.

### Respiratory Chain; Electron Acceptors

The electrons generated by the oxidation process are transferred from substrates via the enzyme cofactor (electron carrier) to the terminal ubiquinol oxidase with ubiquinone as a mediator in the respiratory chain of host organisms. The final acceptor of electrons is oxygen which is reduced to water by the respiratory chain oxidoreductases.

A respiratory chain is a series of oxidoreductive enzymes, having ability to transfer electrons from a reduced molecule in a cascade of finely tuned stepwise reactions, which are concluded by reduction of oxygen. Each step uses a difference in redox potential for useful work, e.g. transfer of protons across the cytoplasmic membrane, reduction of other molecules etc. Electron carriers have a major role in the respiratory chain as well as in overall cell's oxidoreductive processes.

Electrons can enter the respiratory chain at various levels. At the level of a NADH dehydrogenase which oxidizes NADH/NADPH (obtained by various oxidoreductive processes in the cell) with transfer of electrons to ubiquinone pool and release of protons to extracellular space. Alternatively oxidoreductases can transfer electrons directly to ubiquinon via enzyme bound cofactors (FAD,PQQ). The ubiquinoles are further oxidized by terminal oxidoreductases such as Cytochomes, Nirate reductases etc., whereby the electrons are coupled with intracellular protons to reduce oxygen (forming water) and ubiquinole bound protons are released into extracellular space. Any system which is capable of translocation of protons exploiting redox potential is often known as a proton pump. A cross membrane proton potential is thereby established and is the driving force for function of ATP synthases. These levels have successively more positive redox potentials, i.e. successively decreased potential differences relative to the terminal electron acceptor. Individual bacteria often simultaneously use multiple electron transport chains. Bacteria can use a number of different electron donors, a number of different dehydrogenases, different oxidases and reductases, and different electron acceptors. E.g., E. coli (when growing aerobically using glucose as an energy source) uses two different NADH dehydrogenases and two different quinol oxidases, for a total of four different electron transport chains operating simultaneously.

It is therefore clear that an oxidoreductase (for example sugar dehydrogenase) can only be functional when a electron acceptor is provided. In case of natural systems this is provided by the respiratory chain, alternatively artificial electron acceptors with appropriate redox potential compared to substrate/enzyme/cofactor cascade can be used. In the latter option the disadvantage is that the artificial electron acceptor has to be provided in rather large quantities, in other words normally in equimolar amount to the substrate being oxidized. In this aspect, the acceptor of electrons generated by the enzyme capable of catalyzing oxidation or dehydrogenation may be provided in the reaction mixture in order to promote electron flow and the oxidation or dehydrogenation of compound (II). The acceptor may be selected from but is not limited to: dichlorophenolindophenol (DCPIP), phenazine methosulfate (PMS), potassium ferricyanide (PF), potassium ferrioxalate, p-benzoquinone, phenyl-p-benzoquinone, duroquinone, silicomolybdate, vitamin K3, diaminodurene (DAD), N,N,N′,N′-tetramethyl-p-phenylenediamine (TMDP). Electron acceptor may also be oxygen. A person skilled in art will recognize and can e.g. use compounds listed as Hill reagents, dyes that act as artificial electron acceptors, changing colors when reduced, and find many additional candidate acceptors from literature.

### PQQ Dehydrogenases

It has been shown that PQQ has the ability to complex divalent cations in solution, which is a prerequisite for the catalytic activity of the bacterial quinoprotein dehydrogenases [Mutzel, 1991; Itoh, 1998; Itoh, 2000]. In order to make bacterial quinoprotein dehydrogenase (regardless of its origin) active, besides PQQ also divalent ions (e.g. Mg2+, Ca2+) must be present to achieve successful reconstitution of the holo-form of the enzyme [James, 2003]. Complexed divalent ions have besides their structural role also a role in maintaining PQQ's active configuration [Anthony, 2001].

After screening PQQ dependant aldose dehydrogenases, we surprisingly found that all tested enzymes (e.g. YliI aldose dehydrogenase from E. coli, Gcd membrane bound glucose dehydrogenase from E. coli, GDH from Acinetobacter calcoaceticus (soluble or membrane-bound form), GDH from Gluconobacter oxidans, GDH from Kluyvera intermedium) could oxidize compound (II) to compound (I)—(R1=H, R2=O—CO—CH₃) in the presence of an electron acceptor regardless of the acceptor being synthetic molecule or microorganism's respiratory chain. In fact all organisms tested, which contain PQQ dependent oxidoreductases, were found to successfully oxidize ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate in our experiments. Several were tested successfully also for other compound (II) molecules which are exemplified below.

In this sense, the preferred aspect of this invention deals with PQQ dependent dehydrogenases (quinoproteins, EC 1.1.5), more specifically PQQ dependant sugar 1-dehydrogenases (EC 1.1.5.2).

For example YliI is aldose sugar dehydrogenase from E. coli, which requires PQQ for its activity [Southall, 2006]. While E. coli lacks the ability to synthesize PQQ itself [Hommes, 1984; Matsushita, 1997], it shows positive chemotaxis effect towards PQQ, found in environment [de Jonge, 1998], and can use an externally supplied cofactor [Southall, 2006]. YliI aldose sugar dehydrogenase is a soluble, periplasmic protein, containing N-terminal signal sequence necessary for translocation into the periplasm through the cytoplasmic membrane. YliI aldose sugar dehydrogenase (Asd) fold contains six four stranded antiparallel β-sheets with PQQ-binding site lying on the surface of the protein in a shallow, solvent exposed cleft. YliI protein is a monomer, each binding two calcium ions, one of them lying in the PQQ binding pocket, and the other compressed between two of the six strands that make up the propeller fold [Southall, 2006].

It had been shown [Southall, 2006] that D-glucose, D-galactose, D-fructose, D-arabinose, D-fucose, D-mannose, D-lyxose, D-xylose, D-ribose, xylitol, myo-inositol, L-sorbose, mannitol, 2-deoxy-D-glucose, glucosamine, N-acetylglucosamine, glucose 1-phosphate, glucose 6-phosphate, maltose, α-lactose, D-sucrose, D-cellobiose, melibiose and maltotriose are accepted natural substrates of YliI aldose 1-dehydrogenase. None of the natural sugars with the stereochemistry at the C3 position, resembling compound (II), such as D-Allose, D-Altrose, D-Gulose or D-Idose, were tested by Suthall et al.

Besides YliI aldose sugar dehydrogenase, E. coli contains also a membrane-bound glucose dehydrogenase (mGDH), which is also a quinoprotein involved within the respiratory chain in the periplasmic oxidation of alcohols and sugars [Yamada, 2003]. This enzyme, also termed GCD or mGDH or PQQGDH, occurs like YliI in a form of apoenzyme, since E. coli lacks ability to synthesize pyrroloquinoline quinone (PQQ), the enyzme's prosthetic group. mGDH is a membrane-bound quinoprotein [Matsushita, 1993; Anthony, 1996; Goodwin, 1998] that catalyzes oxidation of D-glucose to D-gluconate on its C-terminal domain stretching into the periplasmic space. The electron transfer, mediated by PQQ is further driven by the N terminal, membrane integrated domain; the electron flow to the respiratory chain is channeled through ubiquinone pool to the ubiquinol oxidase [Van Schie, 1985; Matsushita, 1987; Yamada, 1993]. Active holo-form of the mGDH enzyme is obtained by the addition of PQQ and Mg2+ or Ca2+, or other bivalent metal ions. GCD is a monomeric protein that possesses N-terminal hydrophobic domain spanning the inner membrane [Yamada, 1993], and large C-terminal domain, located in the periplasmic space, containing binding sites for PQQ and Mg2+ or Ca2+ [Yamada, 1993; Cozier, 1999].

Surprisingly, mGDH enzyme is also able to catalyze oxidation of artificial substrate, a compound of formula (II). Cozier, et al. tested its activity towards D-allose, which was the only natural aldohexose with a similar stereochemistry on C-3 atom to that of compound (II) tested, and showed similar activity compared to D-glucose. It is noteworthy that D-allose differs from compound (II) in two additional OH-groups on C-2 and C-4, which makes the activity towards compound (II) equally surprising and unexpected.

Yet another example of PQQ dependant sugar dehydrogenase is Acinetobacter calcoaceticus GDH. At least two distinct quinoprotein glucose dehydrogenase from Acinetobacter calcoaceticus are known: the membrane-bound form (mGDH) and the soluble form (sGDH), which contains a 24-amino-acid N-terminal signal sequence needed for translocation through the cytoplasmic membrane into the periplasm. Both forms are different in all characteristics, e.g. substrate specificity, molecular size, kinetics, optimum pH, immunoreactivity.

The substrate specificity of sGDH is different from that of mGDH. sGDH oxidizes preferably D-glucose, maltose and lactose and less successfully D-fucose, D-xylose, D-galactose, while mGdh is less reactive with disaccharides; it oxidises preferably D-glucose, 6-deoxy-D-glucose, 2-deoxy-D-glucose, D-allose, D-fucose, 2-amino-D-glucose (glucosamine), 3-deoxy-D-glucose, D-melibiose, D-galactose, D-mannose, 3-O-methyl-D-glucose, D-xylose, L-arabinose, L-lyxose and D-ribose, yet less successfully maltose, and lactose [Cozier, 1999; Adachi, 2007].

The two possible reaction mechanisms for sGDH are: (A) The addition-elimination mechanism comprises general base-catalyzed proton abstraction followed by covalent addition of the substrate and subsequent elimination of the product; (B) Mechanism comprising general base catalyzed proton abstraction in concert with direct hydride transfer from substrate to PQQ, and tautomerization to PQQH₂ [Oubrie, 1999]. A similar mechanism is assumed to be the case for E. coli YliI aldose dehydrogenase enzyme.

Like the previously described PQQ dependant dehydrogenases, both sGDH and mGDH require calcium or magnesium for dimerization and function [Olsthoorn, 1997]. The present structures confirm the presence of three calcium binding sites per monomer [Oubrie, 1999].

As exemplified by this invention the diversity of these enzymes in sense of structural properties, localization, mechanisms of cofactor binding and electron transfer etc. does not influence efficacy of said diverse enzymes in catalysing oxidation of compound (II) to compound (I)

Current industrial application of PQQ dependent sugar dehydrogenases includes D-gluconate production (Gluconobacter oxydans) in classic fermentation processes as well as production of various natural sugars. Gluconobacter oxydans organism is well known for its important ability to incompletely oxidize natural carbon substrates such as D-sorbitol (producing L-sorbose for vitamin C synthesis), glycerol (producing dihydroxyacetone), D-fructose, and D-glucose (producing gluconic acid, 5-keto-, 2-keto- and 2,5-diketogluconic acid) for the use in biotechnological applications [Gupta, 2001].

As derived from the above description, PQQ dependent dehydrogenases are found in Acinetobacter calcoaceticus, an industrial microorganism used in vinegar production.

A more recent attention given to PQQ dependent sugar dehydrogenases is directed to development of different amperometric biosensors, e.g. for measuring glucose concentration in blood [D'Costa, 1986; Igarashi, 2004; Heller, 2008], for detection of heavy metals [Lapenaite, 2003], for detection of formaldehyde in air [Acmann, 2008], for detection of phenolic compounds in flow injection analysis [Rose, 2001], as a ultrasensitive bienzyme sensor for adrenaline [Szeponik, 1997], for determination of xylose concentration [Smolander, 1992], etc. PQQ dependent sugar dehydrogenases may have found its use also in nanotechnology as biofuel cells [Gao, 2010]. Soluble PQQ dependent glucose dehydrogenases have become the major group of enzymes used in biosensor systems for self monitoring of blood glucose, because these enzymes, unlike glucose oxidase, are independent of oxygen presence [Heller, 2008].

In the current art no process using PQQ dependant aldose dehydrogenases for production of unnatural compounds, especially in connection to active pharmaceutical compounds or their intermediates, exists or has been contemplated, but as disclosed and provided by the present invention such enzymes have turned out to be feasible and accomplishable to provide an effective and easy synthesis principle for useful unnatural compounds. Therefore the present invention opens a new field for use of these enzymes in further oxidoreductive reactions, used for purpose of synthesis of unnatural compounds especially belonging to classes of synthetic APIs and their intermediate compounds.

### Selection of Enzymes Particularly Useful for the Present Invention

With the information and experimental guidance provided herein, the skilled person will become aware and derive how to select the enzyme capable of catalyzing oxidation or dehydrogenation in order to convert e.g. the compound of formula (II) to the compound of formula (I) based on its substrate specificity or promiscuity, operational pH, temperature and ionic strength window, a need of additional ions or cofactors, or the like. Substrates and reaction conditions are normally chosen to give the optimal activity of the enzyme. However, the substrates and conditions to provide the least inhibitory effect on the cell that hosts the enzyme, or deteriorate stability of the product, can be leveraged against the substrates and conditions by which the optimal activity is reached. In principle, the substrates allowing an enhanced, preferably the best activity of the enzyme are preferred, or vica versa the enzyme having an enhanced, preferably the best specificity towards a desired compound substrate are preferred. It will be immediately apparent to the skilled person that reaction conditions include in one aspect that the temperature, pH, solvent composition, agitation and length of the reaction allow accumulation of the desired product. In addition to satisfy the enzyme activity, the skilled person will know with the disclosure provided herein to adapt the conditions in terms of applying proper pH, temperature and reaction time to prevent the product, e.g. lactone or ester, to deteriorate. If needed, specific cofactors, co-substrates and/or salts can be added to the enzyme in order to either allow or improve its activity. Cofactors are salts or chemical compounds. Often, said species are already included in the solvent mixture, especially if the enzyme is comprised within living whole cell, inactivated whole cell, homogenized whole cell, or cell free extract. Nevertheless, the cofactors, co-substrates and/or salts can be further added to the enzyme, solvent or reaction mixture. Depending on the enzyme, cupric, ferric, nickel, selenium, zinc, magnesium, calcium, molybdenum, or manganese ions, or nicotinamid adenine dinucleotide (NAD), nicotinamid adenine dinucleotide phosphate (NADP+), lipoamide, ascorbic acid, flavin mononucleotide, flavin adenine dinucleotide (FAD), coenzyme Q, coenzyme F420, pyrroloquinoline quinine, coenzyme B, glutathione, heme, tetrahydrobiopterin, or the like can be added to the enzyme, to the solvent or medium or to the reaction mixture comprising the enzyme. For example, with aldose-1-dehydrogenase, or preferably YliI or Gcd, calcium ions or magnesium ions and pyrroloquinoline quinine or similar electron acceptor is added to the reaction mixture, enzyme or solvent or medium. Specifically, suitable conditions are exemplified in the examples hereinafter.

A dehydrogenase for use in the present invention may be particularly chosen among any enzyme that has oxidative activity towards above substrate (II). In general any sugar 1-dehydrogenase known in the art can be used regardless of their sequence identity to the enzymes listed below, notably dehydrogenases. As noted, it is beneficial to choose an enzyme capable of catalyzing oxidation or dehydrogenation specifically at position C1. In the case of the aldose 1-dehydrogenase the enzyme oxidizes the aldehyde or cyclic hemiacetal to lactone.

Special variants of the enzymes, like for example enzymes found in the termoresistant microorganism strains, are also contemplated within the present invention. The same applies to a modified or improved versions of the naturally occurring enzymes, whose amino acid sequence or structure has been changed to attain better substrate specificity, higher activity, activity over broader temperature or pH range, resistance to the presence of organic solvent or high ionic strength of the solvent, or the like.

We have surprisingly found that two distinct sugar dehydrogenases originating from taxonomically diverse micoroorganisms and having only 21.8% amino acid sequence identity, performed equally successful in our experiments. Specifically, comparison was performed between SEQ ID NO. 02, representing amino acid sequence of GDH 01 aldose sugar dehydrogenase YliI from E. coli, and SEQ ID NO. 06, representing amino acid sequence of GDH 02 glucose dehydrogenase GdhB from A. calcoaceticus. Sequence comparison algorithm was made with default settings in AlignX module, component of Vector NTI Advance 11.0 software (Invitrogen), using clustal W algorithm at default settings.

In addition, even structurally highly distinct enzymes such as soluble aldose 1-dehydrogenase from E. coli with amino acid sequence SEQ ID NO. 02 and the membrane bound glucose dehydrogenase from E. coli with amino acid sequence SEQ ID NO. 04, were found to be equally successful in our experiments albeit at slightly different reaction conditions.

Owing to this surprising finding it is now reasonable to expect that proteins capable of converting compound (II) to compound (I) or similar reactions may be significantly diverse in the amino acid sequence. The yields of the reaction however may depend on each sugar dehydrogenase enzyme's substrate specificity.

Examples of suitable dehydrogenase enzyme include, but are not limited to enzymes in the sequence list, which are identified by their nucleotide sequences or respective codon optimized nucleotide sequences or amino acid sequences set forth in sequence listings.

GDH 01 is a dehydrogenase encoding gene comprised within nucleotide sequence of SEQ ID NO. 01 or an amino acid sequence of SEQ ID NO. 02.
GDH 02 is a dehydrogenase having a nucleotide sequence of SEQ ID NO. 03 or an amino acid sequence of SEQ ID NO. 04.
GDH 03 is a dehydrogenase having a nucleotide sequence of SEQ ID NO. 05 or an amino acid sequence of SEQ ID NO. 06.
GDH 04 is a dehydrogenase having a nucleotide sequence of SEQ ID NO. 07 or an amino acid sequence of SEQ ID NO. 08.
GDH 05 is a dehydrogenase having a nucleotide sequence of SEQ ID NO. 09 or an amino acid sequence of SEQ ID NO. 10.
GDH 06 is a dehydrogenase having a nucleotide sequence of SEQ ID NO. 23 or an amino acid sequence of SEQ ID NO. 24.
GDH 07 is a dehydrogenase having a nucleotide sequence of SEQ ID NO. 25 or an amino acid sequence of SEQ ID NO. 26.
GDH 08 is a dehydrogenase having a nucleotide sequence of SEQ ID NO. 27 or an amino acid sequence of SEQ ID NO. 28.
GDH 09 is a dehydrogenase having a nucleotide sequence of SEQ ID NO. 29 or an amino acid sequence of SEQ ID NO. 30.
GDH 10 is a dehydrogenase having a nucleotide sequence of SEQ ID NO. 31 or an amino acid sequence of SEQ ID NO. 32.
GDH 11 is a dehydrogenase having a nucleotide sequence of SEQ ID NO. 33 or an amino acid sequence of SEQ ID NO. 34.
GDH 12 is a dehydrogenase having a nucleotide sequence of SEQ ID NO. 35 or an amino acid sequence of SEQ ID NO. 36.
GDH 13 is a dehydrogenase having a nucleotide sequence of SEQ ID NO. 37 or an amino acid sequence of SEQ ID NO. 38.
GDH 14 is a dehydrogenase having a nucleotide sequence of SEQ ID NO. 39 or an amino acid sequence of SEQ ID NO. 40.
GDH 15 is a dehydrogenase having a nucleotide sequence of SEQ ID NO. 41 or an amino acid sequence of SEQ ID NO. 42.
GDH 16 is a dehydrogenase having a nucleotide sequence of SEQ ID NO. 43 or an amino acid sequence of SEQ ID NO. 44.
GDH 17 is a dehydrogenase having a nucleotide sequence of SEQ ID NO. 45 or an amino acid sequence of SEQ ID NO. 46.
GDH 18 is a dehydrogenase having a nucleotide sequence of SEQ ID NO. 47 or an amino acid sequence of SEQ ID NO. 48.
GDH 19 is a dehydrogenase having a nucleotide sequence of SEQ ID NO. 49 or an amino acid sequence of SEQ ID NO. 50.
GDH 20 is a dehydrogenase having a nucleotide sequence of SEQ ID NO. 51 or an amino acid sequence of SEQ ID NO. 52.
GDH 21 is a dehydrogenase having a nucleotide sequence of SEQ ID NO. 53 or an amino acid sequence of SEQ ID NO. 54.
GDH 22 is a dehydrogenase having a nucleotide sequence of SEQ ID NO. 55 or an amino acid sequence of SEQ ID NO. 56.
GDH 23 is a dehydrogenase having a nucleotide sequence of SEQ ID NO. 57 or an amino acid sequence of SEQ ID NO. 58.
GDH 24 is a dehydrogenase having a nucleotide sequence of SEQ ID NO. 59 or an amino acid sequence of SEQ ID NO. 60.
GDH 25 is a dehydrogenase having a nucleotide sequence of SEQ ID NO. 61 or an amino acid sequence of SEQ ID NO. 62.

Therefore a sugar dehydrogenase for use in the present invention may be any compound that has sugar 1-dehydrogenase activity toward compound (II). In one embodiment of the invention the sugar 1-dehydrogenase is a PQQ dependant sugar 1-dehydrogenase. Examples of suitable PQQ dependant sugar 1-dehydrogenase include but are not limited to GDH 01, GDH 02, GDH 03, GDH 04, GDH 05, GDH 06, GDH 07, GDH 08, GDH 09, GDH 10, GDH 11, GDH 12, GDH 13, GDH 14, GDH 15, GDH 16, GDH 17, GDH 18, GDH 19, GDH 20, GDH 21, GDH 22, GDH 23, GDH 24 and GDH 25, wherein each enzyme is identified by it's corresponding nucleotide sequence or respective codon optimized nucleotide sequence or amino acid sequence as set forth in sequence listing above.

The present invention provides sugar dehydrogenases having an amino acid sequence identity of at least 50% thereof; preferably at least 70% thereof, to any of dehydrogenases selected from GDH 01, GDH 02, GDH 03, GDH 04, GDH 05, GDH 06, GDH 07, GDH 08, GDH 09, GDH 10, GDH 11, GDH 12, GDH 13, GDH 14, GDH 15, GDH 16, GDH 17, GDH 18, GDH 19, GDH 20, GDH 21, GDH 22, GDH 23, GDH 24 and GDH 25. The amino acid sequence identities are determined by analysis with sequence comparison algorithm or by visual inspection. In one aspect, the sequence comparison IS made with default settings in AlignX module, component of Vector NTI Advance 11.0 software (Invitrogen), using clustal W algorithm at default settings.

A preferable sugar 1-dehydrogenase provided by this invention may be the sugar dehydrogenase originating from Escherichia coli identified as GDH01 in the above sequence listing and having corresponding nucleotide sequence SEQ ID NO. 01 and an amino acid sequence of SEQ ID NO. 02.

Equally preferable sugar 1-dehydrogenase provided by this invention may be the sugar dehydrogenase originating from Escherichia coli identified as GDH02 in the above sequence listing and having corresponding nucleotide sequence SEQ ID NO. 03 and an amino acid sequence of SEQ ID NO. 04.

Another preferable sugar 1-dehydrogenase provided by this invention may be selected from the sugar dehydrogenase originating from Acinetobacter calcoaceticus coli identified as GDH03 in the above sequence listing and having corresponding nucleotide sequence SEQ ID NO. 05 and an amino acid sequence of SEQ ID NO. 06.

Yet another preferable sugar 1-dehydrogenase provided by this invention may be selected from the modified sugar dehydrogenase originating from Acinetobacter calcoaceticus coli identified as GDH04 in the above sequence listing and having corresponding nucleotide sequence SEQ ID NO. 07 and an amino acid sequence of SEQ ID NO. 08.

The most preferable sugar 1-dehydrogenase provided by this invention may be the sugar dehydrogenase from originating from Escherichia coli identified as GDH02 in the above sequence listing and having corresponding nucleotide sequence SEQ ID NO. 03 and an amino acid sequence of SEQ ID NO. 04. The said sugar 1-dehydrogenase is also described in the art and within this invention as PQQ dependant sugar dehydrogenase, PQQ dependant glucose dehydrogenase, membrane bound glucose dehydrogenase, PQQ dependant aldose dehydrogenase, aldose dehydrogenase, aldose dehydrogenase quinoprotein or glucose dehydrogenase quinoprotein. This particular enzyme is encoded by gene gcd naturally occurring in E. coli and encodes a protein termed Gcd, mGDH or PQQGDH.

The present invention illustratively makes use of sugar dehydrogenases having an amino acid sequence identity of at least 21.8% thereof; 50% thereof; preferably at least 70% thereof, to the amino acid sequence SEQ ID NO. 02.

Within the present invention it is possible to screen for enzymes/organisms capable of oxidizing lactols of formula (II) or, depending on the desired synthetic API or its precursor compound as product, another non-natural substrate. In one aspect of this invention a person skilled in art will find additional candidate enzymes from literature, which could be applicable for the desired type of enzymatic conversion.

Oxidation/dehydrogenization activity towards compound (II) may be screened among different microorganisms and/or enzymes. The term “analysed material” as used herein refers to any microorganism and/or enzyme that can be used in screening method to screen for and identify microorganism and/or enzyme able to convert compound (II) to compound (I) as the living whole cell catalyst, resting whole cell catalyst, cell free lysate, partially purified or purified enzyme, immobilized enzyme or any other form of catalyst as provided by this invention of any microorganism regardless of it being native or genetically modified microorganism. For practical purposes it will be understood hereinafter that a term “analysed material” includes all preparations of candidate catalyst as described above. Several methods are provided in this disclosure that allow screening for and identification of oxidation/dehydrogenation activity towards compound (II) and thus method for screening and identifying organisms and/or enzymes useful to carry out the present invention.

To successfully perform said screening methods, “analysed material” may be obtained having regard to its cultivation properties. Cultivation may be performed to obtain biomass of “analysed material” in growth medium which satisfies the nutrient needs. Cultivation may be performed in liquid medium or on solid medium. Growth medium and conditions of cultivating may be chosen from but are not limited to Difco & BBL Manual, 2010 and to other protocols well known to person skilled in the art. Cultivated microorganisms may be prepared in different forms of catalyst as provided by this invention. In particular “analysed material” is brought in contact with compound (II) in such conditions that allow forming and accumulation of compound (I). These conditions include in one aspect that the “analysed material” is provided at sufficient load to be able to perform the oxidation/dehydrogenization, in another aspect that the substrate and electron acceptors are present in the reaction in an amount that displays minimal inhibition of the activity of the catalyst, in another aspect that the temperature, pH, solvent composition, agitation and length of reaction allow accumulation of desired product, in another aspect that said conditions do not have detrimental effect on product stability. Specifically such conditions may be defined as indicated by, or as modified or varied from, values or conditions disclosed in examples. “Analysed material” may be able to intrinsically provide all cofactors needed for activity towards compound (I) (naming PQQ), or “analysed material” possess the capability of converting compound (II) to compound (I) when PQQ is provided externally as described in this invention. In all screening methods provided herein to PQQ may preferably be added in concentrations described and exemplified. Bivalent metal ions such as calcium or magnesium ion, provided in the form of a salt, such as CaCl₂ or MgCl₂ facilitate reconstitution of PWW to the apo-enzyme resulting in the active from of aldose dehydrogenase. These may preferably be added to the enzyme in concentration described and exemplified.

It will become apparent to a person skilled in the art that quantification of PQQ presence can be determined by the above method and as described elsewhere in the present specification. In this case the material used for the method has to be depleted of PQQ or contain no PQQ already by its nature.

One such method for screening of and identifying candidate catalysts is to bring into contact the “analysed material” with a compound (II). Converting of compound (II) to compound (I) should be performed at optimal reaction conditions as described above. Detection of substrates converting to product in presence of “analysed material” can be achieved by any of the well known chromatographic methods known in the art. The non-limiting examples include liquid HPLC, GC, TLC analysis etc. An exemplified but not limiting method for monitoring compound (I) and corresponding compound (II) is gas chromatography analysis (chromatographic column: DB-1 100% dimethylpolysiloxane; temperature program: initial temperature: 50° C., initial time: 5 min, temperature rate: 10° C./min, final temperature: 215° C., final time: 10 min; injector: split/splitless injector; carrier gas: helium, initial flow: 10 mL/min; detector: flame ionization detector (FID), detector temperature: 230° C.). The prerequisite for carrying out such method is a presence of electron acceptor in the reaction mixture. For “analysed material” where natural electron acceptor is present (such as respiratory chain) no additional components (apart from oxygen in the air) are needed to be able to observe formation of compound (I) from compound (II) by using said chromatographic methods. In case the electron acceptor capable to relieve PQQ of it's electron pair is not available (such in cell free lysate or cell membrane fraction), artificial electron acceptor such as DCPIP is needed. However the described method being analytical procedure and only small quantities of artificial electron acceptor being needed, the preferred way to carry out screening method with any kind of “analysed material” is in presence of artificial electron acceptor. Illustrative and preferred conditions for carrying out the above method are defined by values and conditions disclosed in examples.

Another screening method provided by this invention is a method performed in presence of alternative artificial electron acceptors with appropriate redox potential compared to a substrate/enzyme/cofactor cascade that can be used. In this sense, the present invention provides a screening method using artificial electron acceptor which changes its optically measurable property or properties (such as color, absorbance spectra, etc.) when reduced. Such artificial electron may be provided in the reaction mixture (a dye-linked system) in order to promote electron flow, hence being indicative of the oxidation or dehydrogenation of compound (II). The acceptor/indicator may be selected from but is not limited to: 2,6-dichlorophenol indophenol (DCPIP), phenazine methosulfate (PMS), potassium ferricyanide (PF), potassium ferrioxalate, p-benzoquinone, phenyl-p-benzoquinone, duroquinone, silicomolybdate, vitamin K3, diaminodurene (DAD), N,N,N′,N′-tetramethyl-p-phenylenediamine (TMDP). A person skilled in the art will recognize compounds listed as Hill reagents, dyes that act as artificial electron acceptors, changing colors when reduced, and will find many additional candidate acceptors/indicators from literature. Preferably, 2,6-dichlorophenol indophenol (DCPIP) combined with phenazine methosulfate (PMS) may be used. An exemplified screening method contains following components in a reaction mixture: DCPIP combined with PMS as artificial electron acceptor, “analysed material” and compound (II). Oxidation/dehydrogenation activity of “analysed material” towards compound (II) is followed spectrophotometrically as reduction of absorbance of DCPIP which when oxidized is blue, turning color-less when reduced. When “analysed material” is capable of oxidation/dehydrogenization activity towards compound (II), electrons are transferred to artificial electron acceptor, which becomes reduced and thus reaction mixture turns color from blue to color-less.

One example of carrying out said method is to follow the following procedure: DCPIP and PMS are used in concentrations from about 0.01 mM to about 10 mM for both said artificial electron acceptors, in particular from about 0.05 mM to about 5 mM DCPIP combined with 0.01 mM to about 2 mM DCPIP. Preferably the amount of DCPIP in a screening method is provided in concentration from 0.1 mM to about 1 mM combined with PMS in concentration from 0.05 mM to about 0.5 mM. Most preferably the DCPIP combined with PMS is provided in the amount which allows observation of reduction of absorbance in timeline that can be spectrophotometrically followed. The compound (II) may be dissolved in appropriate aqueous solution and used in a screening method in concentrations from about 0.5 mM to about 1M preferably from about 10 mM to about 500 mM, most preferably 20 mM to 200 mM. Compound (II) may be dissolved in distilled water or in suitable buffered solution. Suitable buffers for adjusting pH value are made with acids, bases, salts or mixtures thereof in particular phosphoric acid and sodium hydroxide may be used. The aqueous suspension, in which the screening method is performed, may be buffered to pH 5.5 to 9.0, preferably to 6.0 to 8.5, more preferably 6.0 to 8.0. “Analysed material” is added to reaction mixture in the said aqueous suspension (particularly in a concentration range from about 0.05 g/L to about 50 g/L), optionally in buffered solution (in particularly in phosphate buffer pH 6.0 to 8.5). Screening and identifying of catalysts capable of converting compound (II) to compound (I) can be observed spectrophotometrically following absorbance reduction in time line, may be at wavelength between 380 nm and 750 nm, preferably at wavelength between 450 nm and 650 nm, more preferably between 550 nm and 650 nm.

This invention also provides an aldose dehydrogenase activity unit. The aldose dehydrogenase activity unit is defined as absolute value of reduction in absorbance unit per minute per wet weight of cultured microorganisms used for preparation of any “analysed material” (abs[mAU min⁻¹ mg⁻¹]). For comparative studies cell density of tested microorganisms may be quantified as wet weight in mg per mL of sample, protein concentrations and/or other indirect or direct methods for quantification well known to person skilled in the art.

Yet another screening method for identification of organisms capable of converting of compound (II) to compound (I), is the use of any known oxygen consumption measurement method known in the art. A nonlimiting example provided by this invention is the use measurement of the dissolved oxygen in the culture of the tested organism after addition of compound (II). More particularly the experimental setup may be composed of a stirred aerated vessel containing the liquid culture broth of the tested organism and a dissolved oxygen sensor. Upon addition of compound (II) one can observe increased oxygene consumption shown by a drop in dissolved oxygen values. The faster and the deeper the drop in dissolved oxygen values under standardized conditions, the higher oxidation rate of compound (II) is facilitated by the tested organism.

### PQQ

Pyrroloquinoline quinine (4,5-dihydro-4,5-dioxo-1H-pyyrolo-[2,3-f]quinoline-2,7,9-tricarboxylic acid: PQQ) is a molecule needed for functioning when using quinoproteins. PQQ, a redox cofactor, which is water soluble and heat-stable, is considered as the third type of coenzyme, after nicotinamide and flavin in biological oxidoreductions and was discovered by Hauge, 1964. To that time unknown redox cofactor was also found by Anthony and Zatman in alcohol dehydrogenase and was named by them as methoxantin [Anthony, 1967]. Later, PQQ has been reported to occur in dehydrogenase, oxidases, oxygenases, hydratases, and decarboxylases. The role of these quinoproteins is to catalyze the primary oxidation step of non-phosphorylated substrates, such as alcohols, aldehydes, or aldoses.

PQQ has been found in both prokaryotic (such as Klebsiella pneumoniae, Acinetobacter calcoaceticus, Methylobacterium extorquens, Kluyvera intermedia, Gluconobacter oxydans, Pseudomonas aeruginosa, Erwinia amylovora, Rahnella aquatilis, Deinococcus radiodurans) and eukaryotic organisms (such as Polyporus versicolor, Rhus vernicifera) [Goodwin, 1998; Hoelscher, 2006; Yang, 2010]

A generally accepted structure of PQQ is:

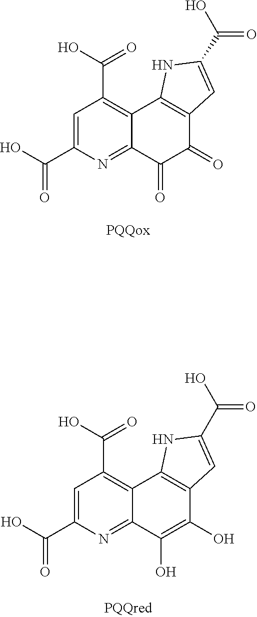

wherein the PQQox is the oxidized form of the cofactor and PQQred is the reduced from of the cofactor.

The number of genes involved in biosynthesis of PQQ varies between species, but in general it is known that for biosynthesis at least five or six genes are needed, usually clustered in the pqqABCDE or pqqABCDEF operon. The number and organization of the genes is variable as it can be seen in following examples. In Klebsiella pneumoniae, the PQQ biosynthetic genes are clustered in the pqqABCDEF operon, while in Pseudomonas aeruginosa the pqqF is separated from the pqqABCDE operon. In Acinetobacter calcoaceticus, there is a pqqABCDE but no pqqF gene is known. A facultative methylotroph Methylobacterium extorquens AM1 contains a pqqABC/DE operon in which the pqqC and pqqD genes are fused, while the pqqFG genes form an operon with three others genes.

Although much is known about the enzymes that use PQQ as a cofactor, relatively little is known about its biosynthesis. However, backbone of PQQ is constructed from glutamate and tyrosine. Most probably these amino acids are encoded in the precursor peptide PqqA. The length of the small peptide varies between different organisms (from 23 amino acids in K. pneumoniae to 39 in P. fluorescens, respectively) and in all variants in the middle of the PqqA peptide motif Glu-X-X-X-Tyr is conserved. The PqqB protein might be involved in its transportation into the periplasm and thus is not directly required for PQQ biosynthesis. Residues of PqqC protein are highly conserved within PqqC proteins, which are responsible for catalyzing the final step in PQQ formation, from different bacteria. Although the alignment of protein sequences of PqqD proteins from different organisms shows strictly conserved residues, the function of PqqD is not fully resolved. In Klebisella pneumoniae it was shown that PqqE recognizes the PqqA, which links the C9 and C9a, afterwards it is accepted by PqqF which cuts out the linked amino acids. In the said organism it was shown that the next reaction (Schiff base) is spontaneous, following dioxygenation. The last cyclization and oxidation steps are catalysed by PqqC [Puehrunger, 2008].

When a comparison of PqqF and PqqG proteins derived from Klebsiella pseudomonas within a protein database was performed, it was purported that said proteins share similarity with a family divalent cation-containing endopeptidases that cleave small peptides [Meulenberg, 1992]. While the PqqF and PqqG proteins of Methylobacterium extorquens show some similarity to the two subunits of mitochondrial processing peptidases [Springer, 1996], the PqqF of Klebsiella pneumoniae is most closely related to the Escherichia coli peptidase pitrilysin encoded by tldD gene [Meulenberg, 1992]. It has been proposed and experimentally shown that PQQ gene clusters comprising only pqqABCDE genes and lacking pqqF may be used to provide compete PQQ biosynthetic maschinery in E. coli. (Kim C. H. et al., 2003, Yang X.-P. et al. 2010). The pitrilysin protease (encoded by tldD gene) is apparently complementig for the activity of pqqF gene found in some microorganisms.

While E. coli lacks the ability to synthesize PQQ itself [Hommes, 1984; Matsushita, 1997], it shows positive chemotaxis effect towards PQQ, found in environment [de Jonge, 1998], and can use an externally supplied cofactor [Southall, 2006]. Thus PQQ biosynthesis genes could be recombinantly expressed in E. coli, what is one of the aspects described in this invention.

In relation to the above, in general, there are at least three ways of providing PQQ to PQQ-dependent dehydrogenases in situ:

First, the PQQ can be added to the living or resting cells containing aldose dehydrogenase enzyme or to the cell free lysates or purified aldose dehydrogenase enzyme. The reconstitution of holo-enzyme form to the active apo-enzyme is almost instantaneous, which was shown in one aspect of our invention. Calcium, magnesium or other bivalent metal ions are added to the mixture in order to facilitate the coupling of the enzyme with the PQQ. This may be achieved by addition of salts such as MgCl₂ or CaCl₂ to the enzyme mixture. Further, there are yet some other possibilities of addition of PQQ which does not necessarily need to be purified in form of dietary complements, media components such as yeast extract etc. The fact that quinoproteins have a very high affinity towards PQQ [de Jonge, 1998] allows that equimolar quantities to the quinoproteins are used. In praxis this means concentrations at the nano molar to micro molar level. It will become apparent to a person skilled in the art that optimization of the amount of PQQ needed for optimal activity of the aldose dehydrogenase enzyme is easily performed in order to reduce the cost of the process. As an non-limiting example, increasing amounts (starting from 0.1 nM) of PQQ are added to the aldose dehydrogenase catalyst and the added amount is optimal when the activity of said catalyst no more increases with additional PQQ provided.

In this sense the present invention provides a method of supplying the PQQ to the aldose dehydrogenase, more specifically to the living whole cell catalyst, resting or inactivated whole cell catalyst, cell free lysate or extract or any other form of catalyst as provided by this invention in concentration from about 0.1 nM to about 5 mM. In particular from about 1 nM to about 100 uM of PQQ can be provided. More preferably the PQQ is provided in concentration from 100 nM to about 5 uM. Most preferably the PQQ is provided in the minimal amount which allows maximal activity of the said catalyst The PQQ can be obtained from any source and provided to the catalyst as solid matter or stock solution of PQQ. In order to facilitate reconstitution of the aldose dehydrogenase by PQQ, calcium or magnesium ions are provided to the enzyme, preferably CaCl₂ or MgCl₂ in concentration from about 0.1 mM to about 50 mM, more preferably from about 1 mM to about 20 mM. MgCl₂ is the preferred option however different enzymes may vary in their preference to a specific bivalent ion.

Second, option is, that the host organism for the production of appropriate dehydrogenase has intrinsic PQQ biosynthetic capability, in other words, contains functional genes for PQQ biosynthesis already integrated in its genetic material. Non-limiting examples are: Klebsiella pneumoniae, Acinetobacter calcoaceticus, Methylobacterium extorquens, Kluyvera intermedia, Gluconobacter oxydans, Pseudomonas aeruginosa, Erwinia amylovora, Rahnella aquatilis, Deinococcus radiodurans and others.

When using such microorganism as host for expression of homogenous or heterogeneous aldose dehydrogenases, the expressed enzymes are coupled with PQQ to form their active form. Some of the said microorganisms have in addition to PQQ production ability, active PQQ dependant aldose dehydrogenase present, which are capable of converting compound (II) to compound (I). In several aspects of our invention this approach proved to be highly effective and successful as exemplified below.

In this aspect the present invention provides microorganisms with native ability to produce PQQ that can be used as hosts for homologous or heterologous expression of PQQ dependant aldose dehydrogenases. Said microorganisms are preferably selected among bacteria, more preferably industrially culturable bacteria and particularly from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans. In the most preferable embodiment Klebsiella pneumoniae, Acinetobacter calcoaceticus, Pseudomonas aeruginosa, Erwinia amylovora, Gluconobacter oxydans may be used.

In similar yet different embodiment the present invention provides microorganisms with natural capability to convert compound (II) to compound (I). No genetic modifications are needed with provided organisms in order to obtain a catalyst capable of performing the desired oxidation. Therefore this invention provides microorganisms for the presently disclosed purpose and use, selected among bacterial origin, more particularly from genera: Klebsiella Enteorobacter, Acinetobacter, Rhizobioum. Methylobacterium, Kluyvera, Gluconobacter, Pseudomonas, Erwinia, Rahnella and Deinococcus. In a more particular sense the microorganisms may be selected from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans, most preferably from: Gluconobacter oxydans, Acinetobacter calcoaceticus and Kluyvera intermedium.

Described above are non-limiting examples of microorganisms with desired properties to carry out this invention. Further, methods are disclosed and provided which allow screening for and identification of such microorganisms.

The third option is especially applicable to microorganisms which do not have intrinsic capability of biosynthesis of PQQ, such as Escherichia coli. It is well known in the art that some microorganims such as E. coli and most of higher organisms have PQQ-dependent enzymes encoded in their genomes and expresses in certain conditions but lack biosynthesis of PQQ [Matshushita, 1997]. It is contemplated in the art that such microorganisms obtain the PQQ as an essential nutrient, or with other words, a vitamin. Ways to establish biosynthesis of PQQ in such organisms to be used for the present invention will be apparent to a person skilled in the art. Approaches of providing biosynthesis of PQQ to such organisms are described (see e.g. Goosen, 1988; Yoshida, 2001; Kim, 2003; Khairnar, 2003; Hoelscher, 2006; Yang, 2010). As also provided by the present invention, it can be established by cloning of PQQ biosynthesis gene cluster from microorganisms which do posses PQQ biosynthesis machinery to the plasmid vector or to the bacterial chromosome and then allowing expression of such genes in the host organisms. Non-limiting examples of microorganims suitable for this purpose include Klebsiella pneumonia, Methylobacterium extorguens, Pseudomonas aeruginosa, Gluconobacter oxydans, Kluyvera intermedia, Erwinia amylovora and others. A term “heterologous expression of PQQ gene cluster” will be immediately understood by a person skilled in the art, as a well established term describing the above procedures.

For use in the present invention any PQQ gene cluster may be used, providing that said gene cluster encodes functional proteins as described above with capability of biosynthesis of PQQ either alone or in concert with the host organism's enzymes.

In one embodiment of the invention the pQQ gene cluster can be obtained from any living organism producing PQQ. In a more particular embodiment of the invention, the PQQ gene cluster can be obtained from any microorganisms selected among bacterial, more particularly from genera: Klebsiella Enteorobacter, Acinetobacter, Rhizobioum, Methylobacterium, Kluyvera, Gluconobacter, Pseudomonas, Erwinia, Rahnella and Deinococcus. In a more particular sense the microorganisms may be selected from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans, most preferably from: Gluconobacter oxydans, Acinetobacter calcoaceticus and Kluyvera intermedia.

Examples of suitable PQQ gene clusters are included, but are not limited to nucleotide sequences of clusters or included genes in the sequence list, which are identified by their nucleotide sequences or amino acid sequences set forth in sequence listings. In general any of the PQQ clusters providing functional genes known in the art may be used for the reaction regardless of their sequence identity to the listed PQQ clusters, genes comprised within and proteins encoded by said genes.

PQQ 01 is a PQQ encoding gene cluster from Gluconobacter oxydans 621H comprised within nucleotide sequence of SEQ ID NO. 68 and allows expression of genes pqqA, pqqB, pqqC, pqqD and pqqE encoding proteins PqqA, PqqB, PqqC, PqqD and PqqE with amino acid sequence SEQ ID NO. 12, 13, 14, 15, 16, respectively.

PQQ 02 is a PQQ encoding gene cluster from Kluyvera intermedia comprised within nucleotide sequence of SEQ ID NO. 69 and allows expression of genes pqqA, pqqB, pqqC, pqqD and pqqE encoding proteins PqqA, PqqB, PqqC, PqqD and PqqE with amino acid sequence SEQ ID NO. 18, 19, 20, 21, 22, respectively.

PQQ 03 is a gene cluster pqqABCDEF from Klebsiella pneumoniae 324 having a nucleotide sequence of SEQ ID. NO 63. and allows expression of genes pqqA, pqqB, pqqC, pqqD, pqqE and pqqF encoding proteins PqqA, PqqB, PqqC, PqqD, PqqE and PqqF. The above sequence is available as part of the genome sequence with accession number CP000964 at NCBI genome database having location between 2602846 and 2599706.

PQQ 04 is a gene clusters pqqABC/DE and pqqFG from Methylobacterium extorguens AM1 having nucleotide sequences of SEQ ID. NO 64 and SEQ ID. NO 65, respectively and allows expression of genes pqqA, pqqB, pqqC, pqqD, pqqE and pqqF encoding proteins PqqA, PqqB, PqqC, PqqD, PqqE and PqqF. The above sequence is available as part of the genome sequence with accession number CP001510 at NCBI genome database having location between 1825235 and 1821763 (pqqABC/DE), 2401055 and 2403792 (pqqEF).

PQQ 05 is a gene clusters pqqABCDE and pqqF from Pseudomonas aeruginosa PA7 having nucleotide sequences of SEQ ID. NO 66 and SEQ ID. NO 67, respectively and allows expression of genes pqqA, pqqB, pqqC, pqqD, pqqE and pqqF encoding proteins PqqA, PqqB, PqqC, PqqD, PqqE and PqqF. The above sequence is available as part of the genome sequence with accession number CP000744 at NCBI genome database having location between 3420385 and 3423578 (pqqABCDE), 3439512 and 3437221 (pqqF).

PQQ 06 is a gene cluster pqqABCDEF from Erwinia amylovora ATCC 49946 having a nucleotide sequence of SEQ ID. NO 70. and allows expression of genes pqqA, pqqB, pqqC, pqqD, pqqE and pqqF encoding proteins PqqA, PqqB, PqqC, PqqD, PqqE and PqqF. The above sequence is available as part of the genome sequence with accession number FN666575 at NCBI genome database having location between 597604 and 600850.

A person skilled in art would also recognize additional candidate gene clusters providing for PQQ synthesis, in publicly available databases (GenBank, Swiss-Prot/TrEMBL, RCSB PDB, BRENDA, KEGG, MetaCyc) using well established data mining tools.

The method for measuring activity of PQQ dependant aldose dehydrogenase provided by the present invention can be used, as exemplified by the invention herein, to screen for and identify organisms capable of producing PQQ regardless of their origin (native or genetically modified), and in addition allows, if desired, a semi quantitative method for estimating the quantity of produced PQQ. A PQQ dependant aldose dehydrogenase in any form, preferably expressed in E. coli (or any other microorganism unable to produce PQQ), can be used for reconstitution of active holo-enzyme. A calibration curve obtained by measuring activity of said PQQ dependant aldose dehydrogenase, supplemented with various quantities of PQQ, is compared to the activity of said PQQ dependant aldose dehydrogenase which was supplemented with analysed sample. Within the linear range of the method, the more PQQ is present in the analysed sample, the more activity is observed.

In a particular embodiment of this invention the PQQ gene clusters (or part thereof) are derived from Kluyvera intermedia or Gluconobacter oxydans. Particularly gene cluster from Gluconobacter oxydans 621H comprised within nucleotide sequence of SEQ ID NO. 68 may be used. Alternatively, a particular embodiment of this invention provides use of gene cluster from Kluyvera intermedia comprised within nucleotide sequence of SEQ ID NO. 69 The described gene cluster can be modified by methods known in the art, for example methods described in Sambrook and Russell, Molecular Cloning: A Laboratory Manual, 3′̂″ Ed., Cold Spring Harbor, N.Y. 2001, in order to allow expression of genes encoded in said cluster in E. coli. Any of numerous strains of E. coli can be used: for example E. coli K12 strains such as JM109, DH5, DH10, HB101, MG4100 etc. or E. coli B strains such as BL21, Rossetta, Origami etc. Genes can be introduced into the said host strain by any genetic method known in the art, for example by transfection, transformation, electroporation, conjugal transfer and others. Said gene clusters may be maintained in the said host microorganism in any form known in the art, for example encoded in a autonomously replicating plasmid or integrated into host's genome. Expression of the genes encoded in said gene clusters can be obtained either by utilizing the activity of native promoters controlling the expression of said genes or by replacing the promoters by promoters which may be more suitable for expression in said host microorganism. Methods for making such modifications are well known in the art.

In one aspect the invention provides gene cluster from Gluconobacter oxydans 621H comprised within nucleotide sequence of SEQ ID NO. 68, which is carried on a autonomously replicating plasmid comprised within the host E. coli strain. In this particular aspect the genes encoded on the cluster are expressed under control of their corresponding native promoters.

In one aspect the invention provides gene cluster from gene cluster from Kluyvera intermedia comprised within nucleotide sequence of SEQ ID NO. 69, which is carried on a autonomously replicating plasmid comprised within the host E. coli strain. In this particular aspect the genes encoded on the cluster are expressed under control of their corresponding native promoters. In the same particular aspect the genes used for provision of PQQ synthesis in E. coli are pqqABCDE and function of pqqF is provided by intrinsic activity of E. coli.

According to our findings availability of PQQ during the heterologous expression of PQQ dependent dehydrogenases has little effect on their correct folding, transport, cleavage of leader sequence and other posttranslational modifications. This means that there is little difference in dehydrogenase activity regardless of when, i.e. at which time or during which period the PQQ is provided to the dehydrogenase, e.g. during or after the cultivation and induction of expression. Other parameters are more relevant when establishing enhanced or even maximal PQQ dependent aldose dehydrogenase activity in periplasmic space or in the cellular membrane, where optimal coupling to cell's native electron acceptors (the respiratory chain) is allowed. One of these parameters are presence of appropriate leader sequence, directing the protein to the periplasm or to the membrane. Another such parameter is expression strength which can be controlled by temperature of cultivation, transcriptional promoter selected, codon usage in the PQQ dependent aldose dehydrogenase encoding gene, quantity of expression inducer etc. Yet another such parameter are intrinsic properties of selected PQQ dependent aldose dehydrogenase such as ability to fold correctly in heterologous host, toxicity to heterologous host, resistance to the host's degrading enzymes etc. All such parameters, which are useful for enhanced activity and optimization and methods to do so, will become apparent to persons skilled in the art.

### Further Exemplified and Modified or Alternative Embodiments of the Present Invention

Various further embodiments, modifications and alternatives to carry out the present invention will be become apparent from the above description.

Further exemplified, the present invention for example provides a particular process comprising the step of reacting a substrate (II) under dehydrogenase catalyzed oxidation conditions to form the corresponding lactone (I), wherein the dehydrogenase is selected in first embodiment from GDH 01 or GDH 02 or GDH 03 or GDH 04 or GDH 05, or any dehydrogenase having an amino acid sequence identity of at least 70% to those, more preferably 90% to those. In another embodiment the dehydrogenase is selected from GDH 06 or GDH 07 or GDH 08 or GDH 09 or GDH 10, or any dehydrogenase having an amino acid sequence identity of at least 70% to those, more preferably 90% to those.

In another specific aspect, this invention relates to a method of constructing and providing appropriate synthetic biological pathways, such as exemplified with E. coli as a host microorganism, wherein DERA (deoxyribose 5-phosphate aldolase), PQQ dependant dehydrogenase and, optionally, PQQ biosynthetic pathway genes are expressed simultaneously. The respiratory chain of the host organism are established and provided also.

Gcd aldose dehydrogenase meets all preferred features and is thus most preferred enzyme used. The Gcd encompasses any aldose dehydrogenase having an amino acid sequence identity to at least 50% of the Gcd described herein, preferably at least 70%. The amino acid sequence identities are determined by the analysis with a sequence comparison algorithm or by a visual inspection. In one aspect the sequence comparison algorithm is made with AlignX algorithm of Vector NTI 9.0 (InforMax) with settings set to default.

A further special aspect, the present invention relates to a process of oxidation or dehydrogenation of compound (II) using an enzyme as described above, comprising the provision of microorganism or microorganism-derived material used as a living whole cell catalyst, a resting whole cell catalyst, a cell free lysate, a partially purified or purified enzyme, an immobilized enzyme or any other form of catalyst, wherein the enzyme capable of catalyzing oxidation or dehydrogenation reaction as described above is expressed in said microorganism naturally, i.e. it being the microorganism's natural property. In said aspect such organism when cultivated and used as catalyst in said reaction can convert compound (II) to corresponding lactone without the need for additional genetic modification of said microorganisms. Said microorganism can be selected from vide diversity of bacteria as exemplified below. An organism with described properties can be selected from bacteria, more particularly proteobacteria, actinomycetales, mixobacteriaceae. More particularly said microorganism may be selected from Gamma proteobacteriaceae. Most preferably organism in this sense is selected from the group of Enterobacteriaceae, Rhizobium, Gluconobacter and Acinetobacter.

Given the disclosure provided herein it will be apparent to a skilled person how to search for an organism having a capability of oxidation or dehydrogenation of compound (II). One such method would be to provide to a culture of studied microorganism an amount of compound (II) and look for activity.

In a further particular embodiment, the definition of the compound of formula (II) and thus also of the compound of formula (I) can be limited in that R5 denotes the moiety selected from the formulae (III), (IV), (V), (VI), (VII), (VIII) and (IX).

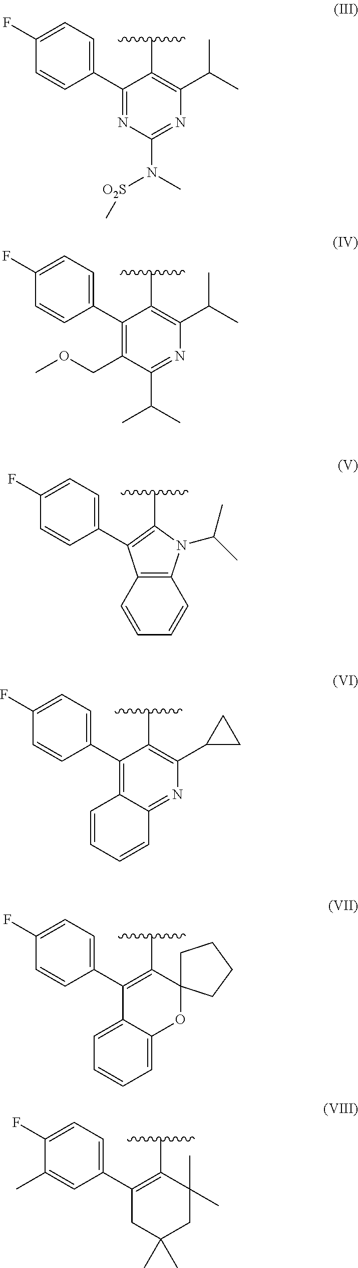 [SMILES: *c1c(-c2ccc(F)cc2)nc(N(C)S(C)(=O)=O)nc1C(C)C ; *c1c(C(C)C)nc(C(C)C)c(COC)c1-c1ccc(F)cc1 ; *c1c(-c2ccc(F)cc2)c2ccccc2n1C(C)C ; *c1c(C2CC2)nc2ccccc2c1-c1ccc(F)cc1 ; *C1=C(c2ccc(F)cc2)c2ccccc2OC12CCCC2 ; *C1=C(c2ccc(F)c(C)c2)CC(C)(C)CC1(C)C]

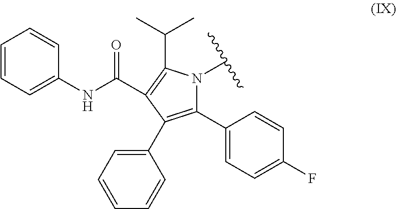 [SMILES: *n1c(-c2ccc(F)cc2)c(-c2ccccc2)c(C(=O)Nc2ccccc2)c1C(C)C]

In yet another particular embodiment, the definition of formula (II) and thus also of formula (I) can be specified to the definition, wherein

R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, or NR³CO(CH₂)ₙCH₃; and
R₂ independently from R₁ denotes H or (CH₂)ₘ—CH₃;
or both of R₁ and R₂ denote either X, OH or O(CH₂)ₙCH₃;
or R₁ and R₂ together denote ═O, —(CH₂)ₚ—, —(CH₂)ᵣ-(1,2-arylene)-(CH₂)ₛ—, wherein R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
X denotes F, Cl, Br or I;
n represents an integer from 0 to 10;
m represents an integer from 0 to 3;
p represents an integer from 2 to 6;
and at least one from r and s is 1.

The compound of formula (I) obtained by the process of the present invention can be used as an intermediate for preparing a statin. The skilled person will know how to put the process step of obtaining said compound according to the present invention in the context of a statin synthesis. In principle, there are two basic ways to arrive at the statin. According to a first route, a lactone is prepared from the lactol and then coupled to the statin backbone. Alternatively, first the statin backbone containing the aldehyde side moiety is prepared, which is subsequently converted to lactol, for example by using 2-deoxyribose-5-phosphate aldolase (DERA) enzyme, and then oxidized to lactone. For the specific case of atorvastatin, as an example, one can refer to schemes 2 to 4 of the WO 2006134482.

In a specific embodiment an enzyme 2-deoxyribose-5-phosphate aldolase (DERA, EC 4.1.2.4) is used for preparing the compound of formula (II), which is subsequently converted to lactone by the enzyme capable of catalyzing oxidation or dehydrogenation. Multiple wild type, variants or mutant version of DERA enzyme are know in the art, including, but not limited to, J. Am. Chem. Soc. 116 (1994), p. 8422-8423, WO 2005/118794 or WO 2006/134482. In preferred embodiment, 2-deoxyribose-5-phosphate aldolase enzyme is used for a synthetic step just preceding the step of bringing in contact the compound of formula (II) with the enzyme capable of catalyzing oxidation or dehydrogenation.

In another preferred embodiment, DERA is used to prepare the compound of formula (II) at least in part simultaneously to conversion of said compound to the compound of the formula (I) by the enzyme capable of catalyzing oxidation or dehydrogenation. It will be immediately understood that the enzymes necessary to catalyse the reaction of preparing the compound of formula (II) and the reaction of oxidizing said compound to formula (I) can be used within the reaction mixture, or can be added to the reaction mixture, simultaneously or subsequently, at once, intermittently or continuously. The embodiment having the compound of formula (II), and thus the starting material for the reaction with the enzyme capable of catalyzing oxidation or dehydrogenation, prepared by DERA is advantageous, because this arrangement is well compatible and it allows using aqueous solvents in the preceding step and thus makes it unnecessary to purify the compound of formula (II) prior to offering it to the enzyme capable of catalyzing oxidation or dehydrogenation for conversion to lactone. The preferred embodiment is thus to bring the compound of formula (II) in contact with the enzyme capable of catalyzing oxidation or dehydrogenation without prior isolation or purification of said compound. In this event, the complete reaction mixture of the preceding step can be used for the subsequent reaction, which reduces the number of process steps and simplifies the process. With obviating a purification step yield is also increased. In addition, because of better enzyme specificity over chemical oxidants, to most extent only the compound of formula (II) gets oxidised to lactone, while the rest of the present compounds do not change, hence reducing a tendency of impurity generation and thus improving subsequent working-up or purification procedures. All of the substrate in any enzyme reaction according to the present invention can be added to the reaction at once or can be added continuously over longer period, or in one batch or intermittent batches.

Significant improvement can be achieved when the compound of formula (I) is prepared at least in part simultaneously to preparing the compound of formula (II). At the same time it solves multiple drawbacks of using the reaction with the DERA enzyme individually. For example, aldehydes used as a starting material in preparing the compound of formula (II) by using DERA enzyme tend to inactivate the DERA enzyme during the course of reaction and thus reduce enzyme's activity. In addition, the lactol of formula (II) that builds up in the reaction mixture is toxic to the living microorganism. Therefore, it is highly desired to shorten the reaction step with DERA and to consume the starting aldehyde and/or the lactol as soon as possible, which is achieved when both enzymatic reaction steps are performed at least in part simultaneously. Namely, when both reaction steps are performed at least in part simultaneously, preferably completely simultaneously, the toxic lactol immediately enters into the consequent reaction and is transformed to the non-toxic lactone. Moreover, since the second reaction step typically is not a rate limiting step, as confirmed in examples hereinafter, and proceeds faster than the first step with DERA, the steady state equilibrium of the first reaction shifts in a direction of the product. This leads to reduced time for completion of the first step and thus protects DERA from being inactivated. It also protects living cells from being disrupted by high concentrations of lactol.

Important aspect of present invention deals with the intrinsic capability of a microorganisms to transfer electrons produced by oxidation/dehydrogenation of (II), to oxygen (a terminal electron acceptor) via its respiratory chain. This drives the reaction of enzyme catalysed oxidation/dehydrogenation of compound (II) in a whole cell system. It will be immediately apparent that the capability of acting as an electron sink is a significant and beneficial property of whole cell systems as described hereby in the invention.

Use of whole cells therefore avoids use of additional electron acceptors such as DCIP and others described above. An additional benefit of using whole cell processes is ability to provide all aspects of described synthetic biological pathway, i.e. the DERA enzyme, PQQ and a PQQ dependant sugar dehydrogenase, in one organism. As productivity and yields of such process are industrially suitable as exemplified below, use of whole cells is preferred as costs can be controlled at significantly lower level compared to other approaches, e.g. free enzyme process, immobilized enzyme, cell free lysate etc. Also the possibility to perform both DERA and oxidation/dehydrogenation step fully or partially simultaneously using one pot design leads to significant cost reductions when used in industrial scale.

In this sense of making use of whole cell system as electron sink, it is also advantageous to perform the process in the presence of oxygen, particularly where the oxygen is provided in quantity allowing at least 5%, preferably at least 15%, of dissolved oxygen at given process condition, wherein 100% dissolved oxygen is understood as saturated solution of oxygen at given process conditions and 0% is understood as oxygen free liquid and correlation between oxygen concentration and dissolved oxygen percent is linear between the 0% and 100% at said given process conditions. In this aspect process conditions are understood as liquid composition, temperature, pH, pressure, wherein the measurements in dynamic process are understood to be performed in a homogenous solid/liquid/gas multiphase system.

The presence of the oxygen in that amount makes the oxidation or dehydrogenation reaction irreversible, which secures the obtained lactone and further enhances shifting of the steady state equilibrium of the first reaction towards the product. This preferred embodiment thus increases yield and reduces time needed for the process.

The enzyme capable of catalyzing oxidation or dehydrogenation, and/or the DERA enzyme, i.e. respectively alone or in combination and optionally independently, can be comprised within single or multiple living whole cell(s), inactivated whole cell(s), homogenized whole cell(s), or cell free extract(s); or are respectively purified, immobilized and/or are in the form of an extracellularly expressed protein. Preferably one of the two enzymes, yet more preferably both enzymes are comprised within same living whole cell, same inactivated whole cell or same homogenized whole cell, more preferably are within same living whole cell or same inactivated whole cell, particularly are comprised within same living whole cell, because having the enzyme in a common whole cell or at least in the common inactivated whole cell, does not demand much handling with the enzyme prior it being used in the process, which reduces costs. Moreover, having the enzyme comprised in a living whole cell enables simple removal of the enzyme by filtration, which alleviates final purification steps at the industrial scale. In addition, it allows a reuse of the enzyme comprised within the living cell in subsequent batches.

Another advantage of using the enzyme in a whole cell or at least in the inactivated whole cell is possibility of providing PQQ cofactor intrinsically as described in detail above.

As an advantageous option, a whole cell system capable of translating 2-deoxyribose-5-phosphate aldolase (DERA) enzyme and an enzyme capable of catalyzing oxidation or dehydrogenation can be arranged to overexpress both of the genes needed for said translation. Means for overexpression are known to the person skilled in the art, and are sometimes referred to elsewhere herein.

According to a further aspect of the present invention, an expression system is provided comprising one or more cell types, the respective cell type(s) being genetically engineered to express, in the totality of cell type(s), both the 2-deoxyribose-5-phosphate aldolase (DERA) enzyme and an enzyme capable of catalyzing oxidation or dehydrogenation.

An expression system can be made up of appropriate organisms or cells and optionally further factors and additives, wherein reference is made to the disclosure provided herein.

In one aspect, this invention provides a method of constructing or providing synthetic biological pathway for use in the present invention, exemplified with E. coli as a host microorganism, wherein DERA (deoxyribose 5-phosphate aldolase), PQQ dependant dehydrogenase and, optionally, PQQ biosynthetic pathway genes are expressed simultaneously. Providing the respiratory chain of the host organism, said synthetic biological pathway has a capability of carrying out production of compound (I) from simple molecules such as compound (X), shown below, and acetaldehyde. This approach is advantageous since this approach joins previously separate steps of production of compound (II), purification of compound (II), and oxidation of compound (II) to compound (I). Additionally, the cultivation of organisms carrying having said synthetic biological pathway is performed in one industrial fermentation process which immediately provides material capable of converting molecules such as compound (IX) and acetaldehyde into compound of formula (I).

Another embodiment of the present invention is obtaining the compound of formula (I), or salts, esters or stereoisomers thereof, in a one-pot process by reacting the starting materials for the DERA enzyme reaction in the presence of 2-deoxyribose-5-phosphate aldolase (DERA) enzyme and an enzyme capable of catalyzing oxidation or dehydrogenation, and optionally salifying, esterifying or stereoselectively resolving the product. This embodiment contemplates to start from the compound of formula (X)

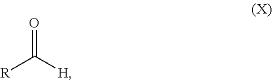

in which R denotes R₁—CH—R₂ moiety of formula (I), and R₁ and R₂ being as defined hereinabove; and subjecting said compound (X) to reaction with acetaldehyde in the presence of the two enzymes, namely aldolase (DERA) enzyme and the enzyme capable of catalyzing oxidation or dehydrogenation. This setup allows obtaining the compound of formula (I) in a single process step starting from relatively simple starting materials, e.g. acetaldehyde. The reaction is industrially suitable, as it proceeds to completion within few hours. It renders intermediate purification steps superfluous. In addition, it provides a possibility of having both enzymes added together—the product of the first enzymatic reaction forming a substrate of the second enzymatic reaction—preferably comprised within the same living whole cell, inactivated whole cell, homogenized whole cell, or cell free extract; or are purified, immobilized and/or are in the form of an extracellularly expressed protein, preferably are within the same living whole cell, inactivated whole cell or homogenized whole cell, more preferably are within the same living whole cell or inactivated whole cell, particularly are comprised within the same living whole cell.

This makes use of all the advantageous effects of combined enzymatic reactions, including, but not limited to the ones described herein. In one aspect, the total amount of substrates added to the mixture is such that the total amount of the substrate (X) added would be from about 20 mmol per liter of the reaction mixture to about 2 mol per liter of the reaction mixture, in particular from about 100 mmol per liter of the reaction mixture to about 1.5 mmol per liter of the reaction mixture, more particular from about 200 mmol per liter of the reaction mixture to about 700 mmol per liter of the reaction mixture. Acetaldehyde may be added by several means. In one aspect, acetaldehyde is added to the reaction mixture in one batch or more batches or alternatively continuously. Acetaldehyde may be premixed with the substrate of formula (X) and added to the reaction mixture. The total amount of acetaldehyde added to the reaction mixture is from about 0.1 to about 4 molar equivalents to the total amount of the acceptor substrate, in particular from about 2 to about 2.5 molar equivalents. In one aspect of the invention, the pH-value used for the reaction is from about 4 to about 11. In one embodiment, the pH used for reaction is from about 5 to about 10. In another embodiment, the pH-value used for reaction is from about 5 to about 8. Specifically the pH-value will be maintained by a suitable buffer in a range from 5.2 to 7.5. Alternatively the pH-value as stated above may be controlled by, but not limited to, controlled addition of acid or base according to need as will be obvious to the person skilled in the art.

In one aspect the pH used for the reaction described by the present invention may be optimized so that the compromise between optimal enzyme activity and optimal substrate and/or product stability is taken. It is understood herein that optimal enzymatic activity for different enzymes described in this invention may not be identical to optimal conditions for substrate/product stability. A person skilled in art may find it beneficial to sacrifice some enzyme activity by adjusting conditions to suite substrate and/or product stability (or vice versa) to obtain optimal product yields.

Specifically, aldolase enzyme, optionally at least in part together with the enzyme capable of catalyzing oxidation or dehydrogenation are prepared in an aqueous solution (particularly each in a concentration 0.1 g/L to 3 g/L), optionally in the presence of a salt (in particular NaCl in a concentration from 50 to 500 mM) optionally with addition of PQQ (particularly in concentration 250 nM to 5 uM) and CaCl₂, MgCl₂ or alternative Calcium or Magnesium salt) particularly in concentration from 0.1 to 20 mM. The aqueous solution may contain organic solvents miscible with water (in particular dimethyl sulfoxide in a concentration from 2 to 15% V/V), and may be buffered to pH 4 to 11. Some commonly used buffers can lower the yield of the aforementioned reaction that starts from the acetaldehyde by limiting the availability of aldolase-condensation intermediates, particularly first condensation reaction products as they may undergo a chemical reaction with a buffer. For example, bis-tris propane reacts with said intermediates ((S)-3-hydroxy-4,4-dimethoxybutanal) giving (S,Z)-2-(3-((1,3-dihydroxy-2-hydroxymethyl)propan-2-yl)(3-hydroxy-4,4-dimethoxybut-1-enyl)amino)propyl-amino)-2-(hydroxymethyl)propane-1,3-diol. Other buffers that may react similarly are bis-tris, tricin, tris, bicin or any other buffer having a primary, secondary or tertiary amino group. Thus suitable buffers for adjusting pH, if this adjustment is needed, are made with acids, bases, salts or mixtures thereof, in particular phosphoric acid and sodium hydroxide. In a particularly preferred embodiment, the buffer is a phosphate buffer. In particular, phosphate buffer, in a concentration 10 to 500 mM can be used. The aqueous solution can also be prepared by adding the aldolase enzyme, optionally at least in part together with the enzyme capable of catalyzing oxidation or dehydrogenation to water and maintaining the pH-value during the reaction by means of an automated addition of inorganic acids, bases, salts or mixtures thereof.

In one aspect according to the invention, the temperature used for the reaction starting from acetaldehyde is from about 10 to about 70° C. In one embodiment, the temperature used for the reaction is from about 20 to about 50° C. In another embodiment, the temperature used for the reaction is from about 25 to about 40° C.

In one aspect the temperature used for the reaction described by this invention may be optimized so that the compromise between optimal enzyme activity and optimal substrate and/or product stability is taken. It is understood herein that optimal enzymatic activity for different enzymes described in this invention may not be identical to optimal conditions for substrate/product stability. A person skilled in art may find it beneficial to sacrifice some enzyme activity by adjusting conditions to suite substrate and/or product stability (or vice versa) to obtain optimal product yields.

After the completion of the reaction, either enzyme can be removed from the reaction mixture, for example by the addition of at least about 1 vol. of acetonitrile to 1 vol. of reaction mixture. Alternatively the enzyme can be removed by any salting out method known in the art. In one embodiment the salting out is performed with the addition of ammonium sulfate of at least 5% m/V. In the embodiment, where the enzyme is comprised within living or inactivated cells, the enzyme may be removed by filtrating or centrifuging the reaction mixture.

In another embodiment the product is removed by liquid/liquid extraction to any of a number of water immiscible or poorly miscible solvents. The solvent may be selected from but is not limited to: methylene chloride, ethyl acetate, diethyl ether, propionyl acetate, methyl t-butyl ether (MTBE), nitromethane, pentane, hexane, heptane, 1,2-dichloroethane, chloroform, carbon tetrachloride, n-butanol, n-pentanol, benzene, toluene, o-, m-, p-xylene, cyclohexane, petroleum ether, triethylamine. Prior the liquid/liquid extraction with chosen organic solvent the pH of water solution of the product may be adjusted to values between 1 and 12, preferably between 2 and 8, more preferably between 3 and 5. Drying of water residues in organic phase after extraction completion may be performed with but is not limited to adding salts listed: sodium sulfate, magnesium sulfate (monohydrate), calcium sulfate, calcium chloride, copper sulfate.

In general, the aldolase enzyme and/or enzyme capable of catalyzing oxidation or dehydrogenation used can be prepared by any means known in the art, for example by methods of protein expression described in Sambrook et al. (1989) Molecular cloning: A laboratory Manual 2^{nd} Edition, New York: Cold Spring Harbor Laboratory Press, Cold Spring Harbor. Gene coding aldolase enzyme and/or enzyme capable of catalyzing oxidation or dehydrogenation can be cloned into an expression vector and the enzyme be expressed in a suitable expression host. Modified versions of known aldolase enzyme or enzyme capable of catalyzing oxidation or dehydrogenation may be necessary or may result depending on cloning conditions and are encompassed in the present invention.

### Cells and Organisms

One aspect of present invention provides a process of oxidation or dehydrogenation of compound (II) or other compounds recited herein using a microorganism in any form described herein having enzyme capable of catalyzing oxidation or dehydrogenation reaction natively expressed. In said aspect such organism when cultivated and used as catalyst can convert compound (II) to corresponding lactone without the need for additional genetic modification of said microorganisms. Methods of identifying such organisms is exemplified in hereby invention. Non-limiting examples of such organism can be selected from vide diversity of bacteria, more particularly Escherichia, Corynebacterium, Pseudomonas, Streptomyces, Rhodococcus, Bacillus, Lactobacillus, Klebsiella, Enteorobacter, Acinetobacter, Rhizobioum. Methylobacterium, Kluyvera, Gluconobacter, Erwinia, Rahnella and Deinococcus.

In referred embodiments, and in order to practice embodiments of the present invention in its best configuration, a specially adapted expression system capable of translating 2-deoxyribose-5-phosphate aldolase (DERA) enzyme and an enzyme capable of catalyzing oxidation or dehydrogenation, and overexpressing both of the genes needed for said translation, is provided. The term “overexpressing” as used herein refers to the expression under control of a strong promoter, or wherein the gene is expressed at high levels (compared to w.t. expression control) and is accumulated intracellularly or extracellularly. The process of obtaining such a modified expression is known to a person skilled in the art. For example, cloning methods described in Sambrook et al. (1989) Molecular cloning: A laboratory Manual 2^{nd} Edition, New York: Cold Spring Harbor Laboratory Press, Cold Spring Harbor, can be used. The genes for the enzymes can be for example cloned on the same or different vector and transformed into a cell. In an alternative, the expression system comprises separate cells, wherein first cell overexpresses the gene for aldolase enzyme and second cell overexpresses the gene for the enzyme capable of catalyzing oxidation or dehydrogenation. The present specification illustratively, without limitation facing common general knowledge, provides an example of making such expression system. The expression system is particularly suited for preparing statin or intermediate thereof.

The skilled person is aware of all the possible cell systems for either preparing or hosting of either DERA enzyme or the enzyme capable of catalyzing oxidation or dehydrogenation, either alone or in combination, and optionally independent from each other. In general, the cell system would be prokaryotic or eukaryotic. In a specific embodiment, the enzyme can be prepared synthetically. The cell for preparing or hosting either of the enzymes can be a bacteria, yeast, insect cell or a mammalian cell. Preferably the cell is bacteria or yeast and more preferably is bacteria, because bacteria or yeast cell are easier cultivated and grown. The bacteria can be selected from the group of genera consisting of Escherichia, Corynebacterium, Pseudomonas, Streptomyces, Rhodococcus, Bacillus and Lactobacillus, preferably from Escherichia and Lactobacillus, more preferably Escherichia, particularly is Escherichia Coli. In case of yeast, the cell can be selected from the group of genera consisting of Saccharomyces, Pichia, Shizosaccharomyces and Candida, preferably Saccharomyces. The examples of mammalian cells are Chinese hamster ovary cell or a hepatic cell, preferably is Chinese hamster ovary cell.

Another embodiment of the invention is a process for the preparation of compound (I), in particular an industrial fermentative process, wherein the process comprises the step of cultivation of a microorganism capable of oxidation of compound (II), wherein the said microorganism is brought in contact with compound (II).

Yet another embodiment of this invention is a process for the preparation of compound (I), in particular an industrial fermentative process, wherein the process comprises the step of cultivation of a microorganism, capable of oxidation of compound (II), wherein said microorganism is brought in contact with another microorganism having ability of enzymatic production of (II), particularly by catalysis of DERA and wherein substrates which allow production of compound (II) are provided to the reaction mixture.

A preferred embodiment of his invention is a process for the preparation of compound (I), in particular an industrial fermentative process, wherein the process comprises the step of cultivation of a microorganism, capable both of production as well as oxidation/dehydrogenation of compound (II) and wherein substrates which allow production of compound (II) are provided to the reaction mixture.

In particular embodiments, the process according to the present invention comprises the following steps:

Step a1) If not already known or provided, as disclosed elsewhere herein, this step includes identification of a microorganism capable of oxidation/dehydrogenation of compound (II) and/or generation of genetically modified strain of a microorganism to obtain capability of oxidation/dehydrogenation of compound (II) as described in this invention. Particularly organisms having sugar 1-dehydrogenase activity are preferred.
Step a2) If not already known or provided, as disclosed elsewhere herein, this step includes identification of a microorganism capable of production of compound (II) and/or generation of genetically modified strain of a microorganism to obtain capability of production of compound (II) as described in this invention or is known in the present art. Particularly organisms having aldolase catalytic ability are preferred.

It is particularly preferred that a microorganism is identified and/or genetically modified in order to obtain both properties described in step a1) and step a2). In this sense the invention specifically relates to a genetically modified strain of a microorganism wherein the genetic material of the strain comprises at least one over expressed gene coding for and enzyme capable of catalysing aldol condensation to form compound (II), more specifically a gene encoding DERA enzyme.

Procedures to identify and/or generate genetically modified microorganisms as described in step a1) and step a2) are exemplified in detail in this invention, however a skilled person will immediately find alternative procedures which may lead to the same desired properties of said microorganisms. For example, cloning methods described in Sambrook et al. (1989) Molecular cloning: A laboratory Manual 2^{nd} Edition, New York: Cold Spring Harbor Laboratory Press, Cold Spring Harbor, can be used. The genes for the enzymes can be for example cloned on the same or different vector and transformed into a cell. In an alternative, the expression system comprises separate cells, wherein first cell overexpresses the gene for aldolase enzyme and second cell overexpresses the gene for the enzyme capable of catalyzing oxidation or dehydrogenation. The present specification illustratively, without limitation and taking into account common general knowledge, provides an example of making such modified microorganism. The microorganism is particularly suited for preparing statin or intermediate thereof.

The skilled person is aware of all the possible cell systems for either preparing or hosting of either DERA enzyme or the enzyme capable of catalyzing oxidation or dehydrogenation or, optionally, the PQQ biosynthetic genes, either alone or in combination, and optionally independent from each other. In general, the cell system would be prokaryotic or eukaryotic. In a specific embodiment, the enzyme can be prepared synthetically. The cell for preparing or hosting either of the enzymes can be a bacteria, yeast, insect cell or a mammalian cell. Preferably the cell is bacteria or yeast and more preferably is bacteria, because bacteria or yeast cell are easier cultivated and grown.

The bacteria can be selected from the group of genera consisting of Escherichia, Corynebacterium, Pseudomonas, Streptomyces, Rhodococcus, Bacillus, Lactobacillus, Klebsiella, Enteorobacter, Acinetobacter, Rhizobioum. Methylobacterium, Kluyvera, Gluconobacter, Erwinia, Rahnella and Deinococcus. In a more particular sense the microorganisms may be selected from Klebsiella pneumoniae, Acinetobacter calcoaceticus, Methylobacterium extorquens, Kluyvera intermedia, Enterobacter, Gluconobacter oxydans, Pseudomonas aeruginosa, Erwinia amylovora, Rahnella aquatilis, Deinococcus radiodurans, Corynebacterium glutamicum, Escherichia coli, Bacillus licheniformis, Lactobacillus lactis, most preferably from: Escherichia coli, Gluconobacter oxydans, Acinetobacter calcoaceticus and Kluyvera intermedium. In case of yeast, the cell can be selected from the group of genera consisting of Saccharomyces, Pichia, Shizosaccharomyces and Candida, preferably Saccharomyces and Pichia.

It is particularly preferred that a microorganism is identified and/or genetically modified in order to obtain both properties described in step a1) and step a2). In this sense the invention specifically relates to a genetically modified strain of a microorganism wherein the genetic material of the strain comprises at least one over expressed gene coding for and enzyme capable of catalysing aldol condensation to form compound (II), more specifically a gene encoding DERA aldolase enzyme.

Accordingly, the present invention provides an exemplified method of constructing synthetic biological pathway, exemplified with E. coli as a host microorganism, wherein DERA (deoxyribose 5-phosphate aldolase), PQQ dependant dehydrogenase and optionally PQQ biosynthetic pathway genes are expressed simultaneously. Providing the respiratory chain of the host organism said synthetic biological pathway has a capability of carrying out production of compound (I) from simple molecules such as compound (X) and acetaldehyde.

One aspect described in this invention is to cultivate microorganisms described in step a1 and a2 simultaneously or independently.

### Step b) Preparation of Seed Medium

Cultivation of the microorganisms as described in the present invention can be carried out by methods known to a person skilled in art. Cultivation processes of various microorganisms are for example described in the handbook “Difco & BBL Manual, Manual of Microbiological Culture Media” (Zimbro M. J. et al., 2009, 2^{nd} Edition. ISBN 0-9727207-1-5). Preferably the production of seed microorganism which can be used in the main fermentation process for the production of (I) starts from a colony of said microorganism. In this respect the process according to the present application comprises the preparation of frozen stock of described microorganism. This preparation of frozen stock may be carried out using method known in the state of art, such as using a liquid propagation medium. Preferably this frozen stock of microorganism is used to produce a vegetative seed medium by inoculation to a vegetative medium.

The seed medium may be transferred aseptically to a bioreactor. In principle the cultivating of seed microorganism can be carried out under the conditions (e.g. pH and temperature) as in the main fermentation process (described under step c).

### Step c) Main Fermentation Process

Preferably the main fermentation process using a microorganism as described in the present application is carried out in a bioreactor in particular under agitation and/or aeration. Preferably, cultivation of a microorganism used for the process for the production of compound (I) as described in the present application is carried out under submerged aerobic conditions in aqueous nutrient medium (production medium), containing sources of assimilable carbon, nitrogen, phosphate and minerals. Additional compounds may be added to the production medium during or after the cultivation process in order to obtain appropriate enzymatic activities. These may include expression inducers, sources of cofactors and or compounds allowing maintenance of genetic elements (such as antibiotics).

Preferably the main fermentation process comprises the inoculation of production medium with seed microorganism obtained in step b) in particular by asepticall transfer into the reactor. It is preferred to employ the vegetative form of the microorganism for inoculation. The addition of nutrient medium (production medium) in the main fermentation process into the reactor can be carried out once or more batch-wise or in a continuous way. Addition of nutrient medium (production medium) can be carried out before and/or during the fermentation process.

The preferred sources of carbon in the nutrient media can selected from dextrin, glucose, soluble starch, glycerol, lactic acid, maltose, fructose, molasses and sucrose as exemplified below.

The preferred sources of nitrogen in the nutrient media are ammonia solution, yeast extract, soy peptone, soybean meal, bacterial peptone, casein hydrolysate, L-lysine, ammonium sulphate, corn steep liquor and other.

Inorganic/mineral salts such as calcium carbonate, sodium chloride, sodium or potassium phosphate, magnesium, manganese, zinc, iron and other salts may also be added to the medium.

Further known additives for fermentative process may be added in particular in the main fermentation process. To prevent excessively foaming of the culture medium anti-foaming agents could be added, such as silicone oil, fatty oil, plant oil and the like. Particularly a silicone-based anti-foaming agent may be added during the fermentation process to prevent excessively foaming of the culture medium. Expression inducers such as isopropyl β-D-1-tiogalaktopyranoside (IPTG), Arabinose, Tetracycline, indoleacrilate etc. may be added to the culture medium. Additionally, cofactors such as PQQ (pyrroloquinoline quinone), NAD(P), FAD may be added to improved the activity of involved enzymes. In a particular aspect IPTG and PQQ may be used.

The fermentative process could be performed in aerobic conditions with agitation and aeration of production medium. Agitation and aeration of the culture mixture may be accomplished in a variety of ways. The agitation of production medium may be provided by a propeller or similar mechanical device and varied to various extents according to fermentation conditions and scale. The aeration rate can be varied in the range of 0.5 to 2.5 VVM (gas volume flow per unit of liquid volume per minute (volume per volume per minute)) with respect to the working volume of the bioreactor.

The main fermentation process by the present process is carried out at a pH in the range of about 6.3 to 8.5 and temperature in the range of 18 to 37° C. Preferably the pH is in the range of about 6.5 to 8.3 and the temperature is in the range of about 21 to 31° C. Preferably, the cultures are incubated for 16 to about 300 hours, more preferably for about 30 to 70 hours.

It will be obvious to a person skilled in the art that different microorganisms may demand different growth conditions and that it is well described in the art how one can determine the optimal conditions for growth of specific organism. It will be also obvious to a person skilled in the art that different growth conditions may have a big effect on activity of the enzymes involved in the provided process. The present inventions provides detailed examples of methods which can be used to optimize the growth conditions to obtain maximal activities and reaction rates of said enzymes.

Another embodiment of in this invention encompasses cultivation of microorganisms described in step a) simultaneously or independently. Likewise it is also possible to conduct reactions for preparation of compound (II) and compound (I) separately or simultaneously, successively or in a one pot manner.

Specifically, dehydrogenase/oxidase enzyme is prepared after step c) in an aqueous solution (particularly in a concentration range from 0.1 g/L to 300 g/L), optionally in the presence of salt (in particular NaCl in concentration range from 50 to 500 mM), diluted or concentrated to said concentration range. When supplemented with fresh medium or components of the medium allowing viability of the organism living whole cell catalyst is obtained. When prepared in buffered aqueous solution or in used medium, resting whole cell catalyst is obtained. The aqueous solution may be buffered to pH 4.0 to 11, preferably to pH 5.0 to 10.0, more preferably to 5.0 to pH 8.0. Most preferably the solution is buffered to about pH5.2 to about pH 7.5. Suitable buffers can be prepared from: acids, bases, salts or mixtures thereof, and any other buffer system known in the art except those possessing primary, secondary or tertiary amino group. In particular, phosphate buffer, in concentration 10 to 500 mM may be used.

The aqueous solution can be prepared by adding the said dehydrogenase/oxidase enzyme to water and maintaining pH during the reaction by means of automated addition of inorganic or organic acids, bases, salts of mixtures thereof.

### Step d) Enzymatic Reaction

Several options to carry out this invention are provided. It is possible to conduct reactions for preparation of compound (II) and compound (I) separately or simultaneously, successively or in a one pot manner. In one embodiment, these variations are:

1. Compound (II) is Added into the Culture of Microorganism Having Compound (II) Oxidation/Dehydrogenation Capability.

After completion of preceding step of cultivating a microorganism capable of oxidation/dehydrogenation of compound (II) in main fermentation step, compound (II) is brought into contact with said microorganism. Said microorganism may intrinsically contain all needed cofactors or external PQQ addition may be used. PQQ may be added in concentration from about 0.1 nM to about 5 mM. In particular from about 1 nM to about 100 uM of PQQ can be provided. More preferably the PQQ is provided in concentration from 100 nM to about 10 μM. Most preferably the PQQ is provided in the minimal amount which allows maximal activity of the said catalyst. Practically this is in the range of 250 nM to 5 μM final concentration of PQQ. To facilitate reconstitution of the enzyme capable of oxidation/dehydrogenation with the externally provided PQQ, magnesium or calcium ions may be added. Preferably MgCl₂ or CaCl₂ are added in concentration from about 0.1 mM to about 50 mM, in particular from about 1 mM to about 20 mM, Most preferably in concentration from about 2 mM to about 20 mM. Optionally an artificial electron acceptor is added in equimolar concentration to compound II as described in this invention. Preferably the process is preformed by using microorganisms capability of accepting electrons formed during dehydrogenation/oxidation reaction into its intrinsic respiratory chain. Compound (II) may be provided as partially or fully isolated compound (II) obtained from previous reaction mixture containing DERA aldolase under aldolase-catalysed aldol condensation conditions or from other sources (organic synthesis). Compound (II) may be added in by any means and rates as described within this invention in a final concentration from about 20 mM to about 1M, preferably from about 50 mM to about 700 mM, most preferred concentrations are between 100 mM and 500 mM. During the process of dehydrogenation/oxidation of compound (II) additional nutrients in order to support microorganisms viability may be added in similar way as described in step c). Practically it may be beneficial to provide additional carbon source to said reaction mixture as described within hereby invention, optionally independently or in addition also a nitrogen source. General reaction conditions which allow dehydrogenation/oxidation of compound (II) in this specific aspect are defined as ‘aldose dehydrogenation/oxidation conditions’ as provided by this invention.

The compound (II) as substrate for oxidase/dehydrogenase to obtain compound (II) may be added to the reaction mixture in one batch or more batches. In one aspect, the total amount of substrate added to the mixture is such that the total amount of compound (II) added would be from about 20 mmol per liter of reaction mixture to about 1.5 mol per liter of reaction mixture, more particular from about 100 mmol per liter of reaction mixture to about 700 mmol per liter of reaction mixture. In preferred embodiment the substrates are added continuously to the reaction mixture by means of programmable pump at specific flow rate at any given time of the reaction. Optimally, the flow rate is determined as maximum flow rate where the substrate is not accumulating in the reaction mixture. In particular this allows minimal concentrations of undesired products. In another embodiment the inhibitory effect of substrate can be further minimized using correct addition strategy.

### 2. Sequential Reaction Using DERA Aldolase and Oxidation/Dehydroqenase Enzyme.

This aspect the invention provides a variant described as d1), however in this case the compound (II) is provided in situ directly in form of reaction mixture obtained by reacting DERA aldolase with compound (X) and acetaldehyde by methods known in the art. Preferably this approach uses the advantage of performing a one-pot reaction and thus significantly impacting the simplicity of the combined process. It is advantageous to use whole cell catalyst containing DERA aldolase in the preceeding step in accordance to ‘aldolase-catalysed aldol condensation conditions”

3. Simultaneous Reaction Using DERA Aldolase and Oxidation/Dehydrogenase Enzyme within Two Separated Catalysts.

Both catalysts are obtained by fermentation process simultaneously or independently (as described in step c) and catalysts are then transferred into a suitable vessel or reactor, preferably joined or left in same fermenter used for obtaining the catalyst. Another yet similar aspect of the process both enzymatic activities are present in a single microorganism which is preferably obtained as described in step c. In a specific embodiment a simultaneous process containing using DERA aldolase and enzyme capable of oxidation/dehydrogenation and thus providing compound (I). More preferably the acetyloxyacetaldehyde (CH₃CO₂CH₂CHO) as substrate for DERA aldolase to obtain compound (II) may be added to the reaction mixture continuously or alternatively the acetyloxyacetaldehyde (CH₃CO₂CH₂CHO) is added to the reaction mixture in one batch or more batches. In one aspect, the total amount of substrates added to the mixture is such that the total amount of acetyloxyacetaldehyde (CH₃CO₂CH₂CHO) added would be from about 20 mmol per liter of reaction mixture to about 1.5 mol per liter of reaction mixture, more particular from about 100 mmol per liter of reaction mixture to about 700 mmol per liter of reaction mixture. Acetaldehyde may be added by several means. In one aspect the acetaldehyde is added to the reaction mixture in one batch or more batches or alternatively continuously. Acetaldehyde may be premixed with acetyloxyacetaldehyde (CH₃CO₂CH₂CHO) and added to the reaction mixture. The total amount of acetaldehyde added to the reaction mixture is from about 0.1 to about 4 molar equivalents to total amount of acceptor substrate acetyloxyacetaldehyde (CH₃CO₂CH₂CHO), in particular from about 1 to about 3 molar equivalents, more preferably from about 2 to 2.5 molar equivalents. In particular, this allows minimal concentrations of undesired products. Optionally PQQ is added into the reaction mixture in concentrations from about 0.05 μM to about 10 mM, more preferably 0.1 uM to about 100 uM. To facilitate reconstitution of the enzyme capable of oxidation/dehydrogenation with the externally provided PQQ, magnesium or calcium ions may be added. Preferably MgCl₂ or CaCl₂ are added in concentration from about 0.1 mM to about 50 mM, in particular from about 1 mM to about 20 mM, Most preferably in concentration from about 2 mM to about 20 mM. In preferred embodiment the substrates are added continuously to the reaction mixture by means of programmable pump at specific flow rate at any given time of the reaction. The flow rate is determined as maximum flow rate where the substrates are not accumulating in the reaction mixture. In particular this allows minimal concentrations of undesired products. In another embodiment the inhibitory effect of substrates can be further minimized using correct addition strategy. In one aspect, the temperature used for dehydrogenase/oxidase-catalysed reaction is from about 10° C. to about 70° C. in one embodiment, the temperature used for dehydrogenase/oxidase reaction is from about 20 to about 50° C. In one embodiment the temperature used for dehydrogenase/oxidase reaction is from about 25° C. to about 40° C. The reaction is industrially suitable, as it proceeds to completion within few hours.

The term “aldose dehydrogenation/oxidation conditions” as used herein refers to any dehydrogenation/oxidation conditions known in the art that can be catalysed by any dehydrogenase/oxidase enzyme, as described herein. In particular the dehydrogenase/oxidase activity conditions are such that allow forming and accumulation of desired product. These conditions include in one aspect that the dehydrogenase/oxidase is an active enzyme provided at sufficient load to be able to perform the dehydrogenation/oxidation. In another aspect, that the compound (II) as substrate is present in the reaction mixture in an amount that displays minimal inhibition of the activity of the aldolase. Preferably, that the temperature, pH, solvent composition, agitation and length of reaction allow accumulation of desired product and thus forming from compound (II) corresponding compound (I), in another aspect that said conditions do not have detrimental effect stability. Specifically those conditions are defined by values disclosed in examples. An dehydrogenase/oxidase for use in the present invention may be any enzyme that has dehydrogenase/oxidase activity towards compound of formula (II). In a preferred embodiment, the dehydrogenase/oxidase enzymes include but are not limited to: GDH 01, GDH 02, GDH 03, GDH 04, GDH 05, GDH 06, GDH 07, GDH 08, GDH 09, GDH 10, GDH 11, GDH 12, GDH 13, GDH 14, GDH 15, GDH 16, GDH 17, GDH 18, GDH 19, GDH 20, GDH 21, GDH 22, GDH 23, GDH 24 and GDH 25, wherein each enzyme is identified by it's corresponding nucleotide sequence or respective codon optimized nucleotide sequence or amino acid sequence as set forth in sequence listing above. The dehydrogenase/oxidase catalyst described herein can be used in any biologically active form provided in this invention. Generally, catalyst able to obtain compound (I), will be provided in a suitable vessel or reactor, and the compound (II) will be added batch-wise or continuously, or provided by, at least in part simultaneous production of compound (II), preferably in same vessel under “aldolase-catalysed aldol condensation conditions”.

The term “aldolase-catalysed aldol condensation conditions” as used herein refers to any aldol condensation conditions known in the art that can be catalysed by any aldolase, as described for example in WO2008/119810 A2. In particular the aldolase-catalysed aldol condensation conditions are such that allow forming and accumulation of desired product, more preferably that the substituted acetaldehyde R₁CO₂CH₂CHO, more particularly acetyloxyacetaldehyde (CH₃CO₂CH₂CHO) as substrate and acetaldehyde are present in the reaction mixture in an amount that displays minimal inhibition of the activity of the aldolase, in another aspect that the temperature, pH, solvent composition, agitation and length of reaction allow accumulation of desired product and thus forming corresponding lactole (compound II). The DERA aldolase described therein can be used in any biologically active form provided in said invention. The substrates for DERA aldolase, the compounds of formula (X) are selected according to the corresponding compound (II). These products having a masked aldehyde group are key intermediates in WO2008/119810 A2 and WO2009/092702 A2 allowing further steps in preparation of statins, in particular, substrates yielding a product with aldehyde group are preferred. The compound (X) may be in particular acetyloxyacetaldehyde (CH₃CO₂CH₂CHO).

Optional Step e): Separation and/or Purification of Compound (I)

Isolation and/or purification of compound (I), which was produced in the main fermentation process from the said medium, may be carried out in a further separation step.

In the first step of the isolation, whole cell catalyst present in reaction mixture may be removed by any known type of filtration/flocculation/sedimentation/centrifugation procedure or further steps of the isolation are performed on whole cell containing reaction mixture. Preferably it is the object of this invention to omit the step of separating solid particles from the reaction mixture and perform direct extraction step immediately after completion of the reaction as described further on.

In one embodiment of the invention, the isolation of compound (I) may be carried out by adsorbtion to an adsorbent capable of binding compound (I) at significant levels and releasing compound (I) upon elution conditions such as replacement of the medium by a more nonpolar compound. Adsorbent may be selected from but not limited to: silica gel, zeolites, activated carbon, Amberlite™ XAD™ adsorbent resins, Amberlite™ and Amberlite™ FP ion exchange resins etc.

Alternatively liquid-liquid extraction may be carried out using water miscible solvents such as acetonitrile or methanol and supplementing the mixture with high concentration of salts, optionally Sodium chloride in a so coiled “salting out” extraction procedure. In this process separation of phases is observed and the compound (I) is preferentially distributed in solvent rich phase.

In a preferred embodiment the extraction solvent for the liquid/liquid extraction is chosen from any a number of water immiscible or poorly miscible solvents. The solvent may be selected from but is not limited to: methylene chloride, diethyl ether, propionyl acetate, methyl t-butyl ether (MTBE), nitromethane, pentane, hexane, heptane, 1,2-dichloroethane, chloroform, carbon tetrachloride, n-butanol, n-pentanol, benzene, toluene, o-, m-, p-xylene, cyclohexane, petroleum ether, triethylamine. Prior the liquid/liquid extraction with chosen organic solvent the pH of water solution of the product may be adjusted to values between 1 and 9, preferably between 2 and 8, more preferably between 2 and 5. In addition, in a preferred embodiment, the ionic strength of the aqueous phase is increased prior to liquid/liquid extraction by addition of any if inorganic/organic acids, salts or bases. Non-limiting examples of such acids, salts or bases are: phosphoric acid, and salts thereof, sulphuric acid and salts thereof, citric acid and salts thereof, hydrochloric acid and salts thereof etc. The increased ionic strength of the aqueous phase increases the distribution coefficient of compound (II) between the aqueous and organic phase therefore increasing efficacy of the extraction process.

Drying of water residues in organic phase after extraction completion may be performed with but is not limited to adding salts listed: sodium sulfate, magnesium sulfate (monohydrate), calcium sulfate, calcium chloride, copper sulfate.

In most preferred embodiment of the purification process, the pH of the reaction mixture is first corrected to value from about 1 to 9, preferably from about 2 to 8, more preferably from about 3 to 6. Most preferably pH is corrected to about 5 using an acid compound such as phosphoric, sulphuric or hydrochloric acid, etc. Sodium sulphate, disodium hydrogen phosphate, sodium chloride etc is added in concentration from 50 g/L to 300 g/L, most preferably from 100 to 200 g/L. Ethyl acetate is added at least 1 time by the addition of at least 1 volume of ethyl acetate to 1 volume of reaction mixture, preferably at least 3 times by the addition of 1 volume of ethyl acetate to 1 volume of reaction mixture. Most preferably the steps of adding ethyl acetate and separating the extract are carried out until no more than 5% of compound (I) is present in aqueous phase. Ethyl acetate fractions are collected, dried with any of the drying salts known in the art, preferably CaCl₂ or MgSO₄ or other methods of water stripping known in the art. In one aspect the obtained ethylacetate extract can be evaporated to yield isolated compound (I) in a form of yellow-amber oul at room temperature or can alternatively proceed into further steps in order to yield pharmaceutically useful compounds, preferably statins.

### Optional Step f): Further Processing

After obtaining the compound of formula (I), the compound of formula (I) can be further transformed to an API, preferably statin, or a pharmaceutically acceptable salt thereof, by subjecting said compound (I) to conditions sufficient to prepare the API, preferably statin. Thus, in an embodiment, a statin or salt, ester or stereoisomer thereof is prepared by (i) bringing in contact the compound of formula (II) as defined hereinabove with an enzyme capable of catalyzing oxidation or dehydrogenation, to prepare a compound of formula (I) as defined hereinabove, (ii) subjecting said compound (I) to conditions sufficient to prepare a statin; and (iii) optionally salifying, esterifying or stereoselectively resolving the product. Again, the compound of formula (II) can be prepared by using 2-deoxyribose-5-phosphate aldolase (DERA, EC 4.1.2.4) enzyme. The reaction setup can be arranged to introduce both enzymes substantially simultaneously or subsequently, at once or continuously, in one batch or in intermittent batches. Preferably the compounds of formula (II) and (I) are prepared at least in part simultaneously, more preferably substantially simultaneously. It is advantageous to use enzymes for preparing compounds of formula (I) and/or (II) in the case of enzymes, because the product immediately contains the correct spacious orientation of the substituents and no further purification or separation is needed. In the event that other stereoisomers are needed, the process can be combined with methods of stereospecific chemistry known to the skilled person. In preferred embodiment, the statin prepared is lovastatin, pravastatin, simvastatin, atorvastatin, cerivastatin, rosuvastatin, fluvastatin, pitavastatin, bervastatin, or dalvastatin, more preferably atorvastatin, rosuvastatin or pitavastatin, particularly is rosuvastatin.

The term “conditions sufficient to produce an API, preferably statin” as used herein refers to those means described in the art, including those means described herein for conversion of the compound of formula (I) further to the API, preferably statin. In a specific embodiment, where statin is prepared, the skilled person would choose the chemical route by selecting the proper R1 and R2, or R. In the event that R1, R2 and R are chosen to represent the statin skeleton, the compound of formula (I) is already a statin molecule ro an “advanced” intermediate thereof. Nevertheless, modifying the statin like for example by opening the lactone ring, forming the salt or the ester or resolving desired stereoisomers from the mixture of stereoisomers is also possible. In an alternative, if the statin is to be prepared by first providing a lactone and then coupling it to the statin skeleton, a reaction scheme 2 can be followed. After obtaining the compound of formula (I), its hydroxyl group in position 4 can be protected with the protecting group P (formula (XI)), which can be any conventionally used protecting group, in particular is silyl protecting group. Afterwards, the compound can be brought in the form of an aldehyde or its hydrate (formulas (XII) and (XIII), respectively).

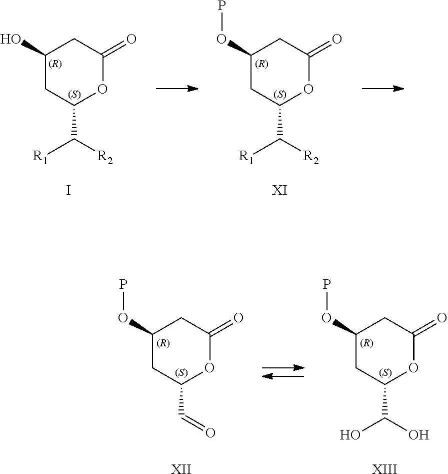

Compound of formula (XII), or hydrate thereof (XIII), obtained from I can be further used to prepare statin by reacting the compound of formula (XII), or hydrate thereof (XIII), under the condition of a Wittig coupling with a heterocylic or alicyclic derivative (statin skeleton) followed by hydrogenation when needed. In the specific embodiment related to preparing rosuvastatin, in the subsequent reaction step, (2S,4R)-4-(P-oxy)-6-oxo-tetrahydro-2H-pyran-2-carbaldehyde (XII) can be reacted under the conditions of a Wittig coupling (in the presence of a base) with a ((4-(4-fluorophenyl)-6-isopropyl-2-(N-methylmethylsulfonamido)pyrimidin-5-yl)methyl)triphenyl-phosphonium halide or any other ((4-(4-fluorophenyl)-6-isopropyl-2-(N-methylmethylsulfon-amido)pyrimidin-5-yl)methyl)phosphonium salt or alternatively di-i-propyl({4-(4-fluorophenyl)-6-isopropyl-2-[methyl(methylsulfonyl)amino]-5-pyrimidinyl}methylphosphonate or any other ({4-(4-fluorophenyl)-6-isopropyl-2-[methyl(methylsulfonyl)amino]-5-pyrimidinyl}methylphosphonate ester to give N-(5-((E)-2-(2S,4R)-4-(P-oxy)-6-oxo-tetrahydro-2H-pyran-2-yl)vinyl)-4-(4-fluoro-phenyl)-6-isopropylpyrimidin-2-yl)-N-methylmethanesulfonamide. As a base, lithium hexamethyldisilazane (LiHMDS), potassium hexamethyldisilazane (KHMDS), sodium hexamethyldisilazane (NaHMDS), lithium diisopropylamide (LDA), sodium hydride, butyllithium or Grignard reagents, preferably sodium hexamethyldisilazane may be used. When the source is the hydrate form XIII or a mixture of XII and the hydrate form XIII thereof, which is dissolved in ethers selected from THF, Et₂O, i-Pr₂O, ^{t}BuMeO; hydrocarbons selected from: pentane, hexane, cyclohexane, methylcyclohexane, heptane; aromatic hydrocarbons selected from toluene or the chlorinated derivatives thereof; chlorinated hydrocarbons selected from: chloroform and dichloromethane or in mixtures of those solvents, water released from the hydrate should be removed prior to the addition to the formed ylide solution. The preferred solvents for the reaction are anhydrous toluene and dichloromethane. The reaction can be performed at temperatures between −80° C. and 90° C. preferably at 0 to 90° C., more preferably at 80-90° C. The reaction is accomplished in 1-12 hours. Isolation of the crude product with extraction can be performed with AcOEt, ethers or alkanes, preferably with ^{t}BuMeO. The protecting group may be removed and the lactone opened to produce a rosuvastatin free acid or a salt thereof, optionally an amine, which may be converted to hemicalcium salt. The deprotection can be performed at temperatures between 0° C. to 80° C. Preferably at 20 or 40° C. in a suitable solvent, preferably a solvent selected from alcohols, acetic acid, THF, acetonitrile, methyltetrahydrofuran, dioxane, CH₂Cl₂, more preferably in alcohols and a mixture of THF/AcOH. The usual deprotecting reagents may be used, such as tetra-n-butylammonium fluoride, ammonium fluoride, AcCl, FeCl₃, TMSCl/HF.2H₂O, chloroethylchloroformate (CEC), Ph₃PCH₂COMeBr. The opening of the lactone preferably takes place in a 4:1 to 2:1 mixture of THF/H₂O as well as in pure THF at temperatures between 20° C. to 60° C. with a suitable alkali such as NaOH, KOH, ammonia or amines. The hydrolysis is accomplished in 30 minutes (at 60° C.) to 2 hours (at 20° C.). After the hydrolysis step, evaporation of THF can be conducted at temperatures between 10° C. to 50° C. under the reduced pressure, and conversion to the calcium salt, preferably by the addition of Ca(OAc)₂.xH₂O, which can be added in one portion or dropwise in 5 to 60 minutes, can be performed at temperatures between 0° C. to 40° C. After the addition of Ca(OAc)₂.xH₂O, the resulting suspension can be stirred at temperatures between 0° C. to 40° C. from 30 minutes to 2 hours. The details of such reaction are known in the art, including in, but not limited to, WO2008/119810.

The API, preferably statin, obtained by any of the aforementioned embodiments, can be formulated in a pharmaceutical formulation. The methods for preparing a pharmaceutical formulation with the API, preferably statin, are known to the person skilled in the art. Generally, one can chose among preparing formulations such as powder, granulate, tablet, capsule, suppository, solution, ointment, suspension, foam, patch, infusion, solution for injection, or the like. The formulation can be changed in order to modify specific aspects of the API like for example release, stability, efficacy or safety. Depending on the API, the skilled person knows how to select proper administration route for said API. Based on that, he can choose proper formulation. Then, the skilled person is in a position to select from the excipients needed for formulating the pharmaceutical formulation. Besides the API, which can be optionally combined with at least one another API, the skilled person can select from excipients and additives to formulate the pharmaceutical formulation. Suitable excipients may be, for example, binder, diluent, lubricant, disintegrant, filler, glidant, solvent, pH modifying agent, ionic strength modifying agent, surfactant, buffer agent, anti-oxidant, colorant, stabilizer, plasticizer, emulsifier, preservatives, viscosity-modifying agent, passifier, flavouring agent, without being limited thereto, which can be used alone or in combination. The method can involve, depending on the selected formulation, mixing, grinding, wet granulation, dry granulation, tabletting, dissolving, lyophilisation, filling into capsules, without being limited thereto.

### Application to APIs and Intermediates Thereof.

In further aspects of the present invention, an enzyme capable of catalyzing oxidation or dehydrogenation can be generally used for preparing an API or intermediate thereof being compatible with the enzymatic system(s) disclosed herein.

This aspect of the invention preferably relates to synthetic API or intermediate thereof respectively categorisable as substituted or unsubstituted dideoxyaldose sugars, lactols (optionally containing multiple hydroxyl groups) and synthetic non-natural alcohols as possible substrates, and (optionally further hydroxylated) lactons or esters as possible products. From the disclosure of the present invention and its technical character, it will be understood that compounds naturally occurring or arising in the biochemical pathways of sugars (e.g. glycolysis, pentose phosphate pathway, glycogenesis), fatty acids, or cellular respiratory chain, would be exempted from being regarded as either substrate for the enzyme capable of catalyzing oxidation or dehydrogenation or the API or intermediate thereof according to the present invention. In the same manner, naturally occurring amino acids, vitamins or cofactors are also exempted from the definition of the API or intermediate thereof according to the present invention. The substrates or products of such pathways in the nature include methanol, ethanol, formaldehyde, acetaldehyde, methanoic, acetic acid, monosaccharide, disaccharide, trisaccharide, glucuronic acid, especially methanol, ethanol, formaldehyde, acetaldehyde, methanoic, acetic acid, monosaccharide, glucuronic acid, and particularly ethanol, acetic acid and monosaccharide. In a preferred embodiment, the enzyme capable of catalyzing oxidation or dehydrogenation is used for preparing the compound of formula (I), wherein the formula (I) is as defined hereinabove. In this aspect, it is the most efficient to use the enzyme to act upon the compound of formula (II), wherein the formula (II) is as defined hereinabove. Specific alternatives of the use will be immediately apparent to the skilled person when other embodiments, aspects, or preferred features of the invention disclosed hereinabove are taken into account.

To give an illustrative further example, a possible useful further definition of the substrate and thus starting compound useful for the preparation of appropriate APIs or intermediates thereof is given by the following formula XIV, hence leading to a corresponding product compound defined by formula XV.

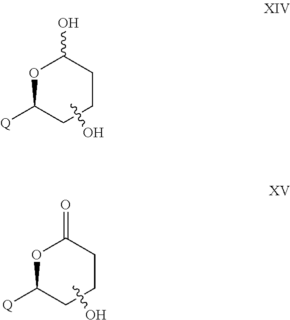

wherein Q is any desired structural moiety, for example selected from the groups of R₁, R₂ and R⁵ defined hereinabove, optionally with an intermediate linker molecule between R₁, R₂ or R⁵ and the lactol/lactone ring. As shown in the structural formulae, the non-lactol hydroxyl group not being oxidized can be positioned at any position of the lactol/lactone ring.

In a preferred embodiment, the enzyme capable of catalyzing oxidation or dehydrogenation is used for preparing an API or intermediate thereof simultaneously or subsequently with a DERA enzyme.

### EXAMPLES

The following examples are merely illustrative examples of the present invention and they should not be considered a limiting the scope of the invention in any way, as these examples and other equivalents thereof will become apparent o those versed in the art in the light of the present disclosure, and the accompanying claims.

### Example 1

### Preparation of Aldose Dehydrogenases Comprised within the Whole Cell

Several aldose dehydrogenases have been prepared as described following examples:

First, a membrane bound glucose dehydrogenase, a pyrroloquinone quinone (PQQ) dependent dehydrogenase, encoded by gene gcd (E. coli GeneBank #JW0120, locus tag b0124) was prepared.

The genomic DNA from E. coli DH5α was isolated using Wizard Genomic DNA Purification Kit (Promega, Madison, Wis., USA) according to manufacturer's instructions. Isolation of genomic DNA was made using overnight culture of E. coli grown on LB medium at 37° C. Amplification of gene gcd was performed by PCR using oligonucleotide primers GCGCCATATGGCAATTAACAATACAGGCTCGCG and GCGCGCTCAGCGCAAGTCTTACTTCACATCATCCGGCAG. Amplification was performed by Pfx50 DNA polymerase (Invitrogen, Calsbad, Calif., USA) as follows: an initial denaturation at 94° C. for 10 min, followed by 30 cycles of 45 s at 94° C., 45 s at 52° C., and 150 s at 68° C. Final elongation was performed 420 s at 68° C. A 2.4-kb DNA fragment containing gcd (SEQ ID NO. 03) was separated by agarose gel electrophoresis and purified. The product was ligated into plasmid pGEM T-Easy (Promega, Madison, Wis., USA) in a T4 ligase reaction. The plasmid construct was cleaved with restriction endonucleases NdeI and BlpI, the resulting fragments were separated on agarose gel electrophoresis and 2.4 kb fragment containing gcd was purified. An expression vector pET30a(+) (Novagen Inc., Madison, Wis., USA) was cleaved using the same aforementioned restriction endonucleases and purified. The 2.4 kb fragment containing gcd gene was assembled with the cleaved expression vector in a T4 ligase reaction. E. coli JM109 cells were transformed with the obtained ligation reaction and kanamycin resistant colonies were cultured. Afterwards plasmid DNA was isolated. The resulting construct was designated pET30/Gcd and sequenced for confirmation of the gene sequence. The cloning procedure was performed to allow expression of protein having sequence (SEQ ID NO. 04) containing necessary signals for incorporation into the cellular membrane The membrane bound glucose dehydrogenase expressing organism was prepared by transforming BL21 (DE3) competent cells with the said plasmid.

Second, a water-soluble aldose dehydrogenase, a pyrroloquinoline quinone (PQQ) dependent dehydrogenase encoded by gene yliI (E. coli GeneBank #ECK0827, locus tag b0837) was prepared.

The genomic DNA from E. coli DH5α was isolated using Wizard Genomic DNA Purification Kit (Promega, Madison, Wis., USA) according to manufacturer's instructions. Isolation of genomic DNA was made using overnight culture of E. coli grown on LB medium at 37° C. Amplification of gene yliI was performed by PCR using oligonucleotide primers GCGCCATATGCATCGACAATCCTTT and GCGCGCTCAGGCTAATTGCGTGGGCTAACTTTAAG Amplification was performed by Pfx50 DNA polymerase (Invitrogen, Calsbad, Calif., USA) as follows: an initial denaturation at 94° C. for 10 min, followed by 30 cycles of 45 s at 94° C., 45 s at 52° C., and 150 s at 68° C. Final elongation was performed 420 s at 68° C. A 1.2-kb DNA fragment containing yliI (SEQ ID NO. 01) was separated by agarose gel electrophoresis and purified. The product was ligated into plasmid pGEM T-Easy (Promega, Madison, Wis., USA) in a T4 ligase reaction. The plasmid construct was cleaved with restriction endonucleases NdeI and SalI, the resulting fragments were separated on agarose gel electrophoresis and 1.2 kb fragment containing yliI was purified. An expression vector pET30a(+) (Novagen Inc., Madison, Wis., USA) was cleaved using the same aforementioned restriction endonucleases and purified. The 1.2 kb fragment containing yliI gene was assembled with the cleaved expression vector in a T4 ligase reaction. E. coli JM109 cells were transformed with the obtained ligation reaction and kanamycin resistant colonies were cultured. Afterwards plasmid DNA was isolated. The resulting construct was designated pET30/YliI and sequenced for confirmation of the gene sequence. The cloning procedure was performed to allow expression of protein having sequence (SEQ ID NO. 02) including leader sequence for YliI translocation to periplasm. The aldose dehydrogenase expressing organism was prepared by transforming BL21 (DE3) competent cells with said plasmid.

Another water-soluble aldose dehydrogenase, found in Acinetobacter calcoaceticus, was used for alternative oxidation studies. A nucleotide sequence encoding a gene and leader sequence for transportation to periplasm (PQQ GdhB, A. calcoaceticus GeneBank #X15871) was optimized for expression in E. coli and DNA was chemically synthesized (Geneart, Regensburg, Germany) (SEQ ID. NO. 05). E. coli JM109 cells were transformed with artificial plasmid bearing nucleotide sequence gdhB and kanamycin resistant colonies were cultured. Afterwards plasmid DNA was isolated and the construct was cleaved with restriction endonucleases NdeI and HindIII, the resulting fragments were separated on agarose gel electrophoresis and 1.5 kb fragment was purified (SEQ ID NO. 05). An expression vector pET30a(+) (Novagen Inc., Madison, Wis., USA) was cleaved using the same aforementioned restriction endonucleases and purified. The 1.5 kb fragment containing gdhB gene was assembled in the cleaved expression vector in a T4 ligase reaction. E. coli JM109 cells were transformed with the obtained ligation reaction and kanamycin resistant colonies were cultured. Afterwards plasmid DNA was isolated. The resulting construct was designated pET30/GdhB and sequenced for confirmation of the gene sequence. The cloning procedure was performed with a target to allow expression of protein having sequence SEQ ID NO. 06. The aldose dehydrogenase from A. calcoaceticus expressing organism was prepared by transforming BL21 (DE3) competent cells with said plasmid.

Yet another water-soluble aldose dehydrogenase found in Acinetobacter calcoaceticus (PQQ GdhB, A. calcoaceticus GeneBank #X15871) with altered sequence and hence its increased thermal stability properties was used [Igarashi, 2003]. A nucleotide sequence encoding a gene and leader sequence for transportation to periplasm (PQQ GdhB, A. calcoaceticus GeneBank #X15871) was optimized for expression in E. coli and DNA was chemically synthesized (Geneart, Regensburg, Germany) (SEQ ID NO. 07). E. coli JM109 cells were transformed with artificial plasmid bearing nucleotide sequence gdhB_therm and ampicillin resistant colonies were cultured. Afterwards plasmid DNA was isolated and the construct was cleaved with restriction endonucleases NdeI and HindIII, the resulting fragments were separated on agarose gel electrophoresis and 1.5 kb fragment was purified (SEQ ID NO. 07). An expression vector pET30a(+) (Novagen Inc., Madison, Wis., USA) was cleaved using the same aforementioned restriction endonucleases and purified. The 1.5 kb fragment containing gdhB_therm gene was assembled in the cleaved expression vector in a T4 ligase reaction. E. coli JM109 cells were transformed with the obtained ligation reaction and kanamycin resistant colonies were cultured. Afterwards plasmid DNA was isolated. The resulting construct was designated pET30/GdhB_therm and sequenced for confirmation of the gene sequence. The alternative aldose dehydrogenase expressing organism was prepared by transforming BL21 (DE3) competent cells with said plasmid. The cloning procedure was performed with a target to allow expression of protein having sequence SEQ ID NO. 08. SEQ ID NO. 08 which compared to SEQ ID NO 06 has altered sequence coding Ser residue at position 231 to Lys residue.

Where stated, E. coli BL21(DE3) having an expression plasmid vector pET30a(+) (Novagen Inc., Madison, Wis., USA) was used as negative control. The control cells were prepared by transforming E. coli BL21 (DE3) competent cells with said plasmid and kanamycin resistant colonies were cultured.

The procedure of expressing the various enzymes in E. coli was undertaken as described in Procedure 1A. After expression, cells were harvested and whole cell catalyst was obtained as described in Procedure 1B. To obtain resting whole cell catalyst Procedure 10 was undertaken.

### Procedure 1A:

VD medium (30 mL; 10 g/L bacto yeast extract, 5 g/L glycerol, 5 g/L NaCl, 4 g/L NaH2PO4.2H2O, pH was adjusted with 1 M NaOH to pH=7.0) supplemented with kanamycin (25 μg/mL) was inoculated with a single colony E. coli BL21 DE(3) pET30/Gcd or E. coli BL21 DE(3) pET30/YliI or BL21 DE(3) pET30/GdhB or BL21 DE(3) pET30/GdhB_therm from a freshly streaked VD agar plate and pre-cultured to late log phase (37° C., 250 rpm, 8 h). The pre-culture cells (inoculum size 10%, v/v) were then transferred to fresh VD medium (100 mL; 10 g/L bacto yeast extract, 5 g/L glycerol, 5 g/L NaCl, 4 g/L NaH2PO4.2H2O, pH was adjusted with 1 M NaOH to pH=7.0) supplemented with kanamycin (25 μg/mL). For the induction of protein expression 0.1 mM isopropyl-β-D-thiogalactopyranoside (IPTG, purchased by Sigma Aldrich, Germany) was added. The cells were cultured in a rotary shaker at 25° C., 250 rpm for 16 h.

### Procedure 1B:

After expression (as described in Procedure 1A) cells were harvested by centrifugation (10 000 g, 5 min, 4° C.). Supernatant was discarded after centrifugation and pellet was resuspended in fresh VD medium (10 g/L bacto yeast extract, 5 g/L glycerol, 5 g/L NaCl, 4 g/L NaH2PO4.2H2O, pH was adjusted with 1 M NaOH to pH=6.0). Cells were resuspended in one tenth of initial culture volume.

### Procedure 1C

After discarding supernatant (with regard to Procedure 1A) the pellet was resuspended in phosphate buffer (50 mM KH2PO4, 150 mM NaCl, pH 6.0). Cells were resuspended in one tenth of initial culture volume.

### Example 2

### Preparation of Aldose Dehydrogenase Comprised within Cell Free Lysate

To obtain cell free lysate after expression of E. coli YliI, E. coli GdhB, E. coli Gcd or E. coli GdhB_therm (as described in Example 1, more particularly after undertaking Procedure 1A) cells were harvested by centrifugation (10 000 g, 5 min, 4° C.). Supernatant was discarded after centrifugation and pellet was resuspended in phosphate buffer (50 mM KH₂PO₄, 150 mM NaCl, pH 7.0) in one tenth of initial volume. Cells were supplemented with 1 mg/mL lysozyme solution. Lysis was performed at 37° C., 1 h. After lysis cell debris were removed by sedimentation (10 min, 20 000 g, 4° C.) to obtain a clear aqueous solution. Aldose dehydrogenase comprised within a cell free extract was thus obtained.

Alternatively the pellet was resuspended in a lytic buffer (50 mM NaH2PO4, pH 7.0, 300 mM NaCl, 2 mM DTT) using 200 g of pellet per 1 L of said buffer. Cells were sonified (3×15 s) using Branson digital sonifier and cell debris were removed by sedimentation (10 min, 20 000 g, 4° C.) to obtain a clear aqueous solution. Aldose dehydrogenase comprised within a cell free extract was thus obtained.

In the same manner as above, cell free lysates were prepared from whole cell Klyuvera intermedia and Gluconobacter oxydans (cultivations and preparations of whole cell catalysts are described in Example 9 and Example 10, respectively).

Culture of E. coli BL21(DE3) pET30, used as negative control was treated by the same procedures.

### Example 3

### Preparation of Periplasmic Cell Fraction Containing Aldose Dehydrogenase

To obtain specific release of periplasmic proteins after expression of E. coli YliI, E. coli Gcd, E. coli GdhB or E. coli GdhB_therm (as described in Example 1, more particularly after undertaking Procedure 1A) the following procedure was used. Cells from freshly expressed culture were pelleted by centrifugation and the growth medium was completely removed. The cell pellet was washed three times in 20 mM Tris-HCl (pH 7.5). The cell pellet was then centrifuged

(10 000 g, 5 min, 4° C.) before resuspended in one tenth of initial culture volume of hypertonic solution containing 20 mM Tris HCl (pH 7.5), 20% sucrose, and 0.5 mM EDTA. The cell suspension was incubated on ice for 10 min. After centrifugation (10 min, 12 000 g, 4° C.) cell pellet was resuspended gently by pipetting into hypotonic solution (50 mM Tris-HCl pH 7.0) in the same volume as hypertonic solution. Cells were incubated on ice for additional 10 min. Cells were pelleted by centrifugation (10 min, 20 000 g, 4° C.) and the supernatant was removed and regarded as periplasmic fraction.

Culture of E. coli BL21(DE3) pET30, used as negative control was treated by the same procedure.

### Example 4

### Preparation of Membrane Cell Fraction Containing Aldose Dehydrogenase

To obtain specific release of membrane bound proteins after expression of E. coli Gcd (as described in Example 1, more particularly after undertaking Procedure 1A) the following procedure was used. Cells from freshly expressed culture were pelleted by centrifugation and the growth medium was completely removed. The cell pellet was resuspended to initial volume in phosphate buffer (50 mM KH₂PO₄, 150 mM NaCl, pH 6.0) containing 0.05% (v/v) Triton X-100 and 1 mg/mL lysozyme and incubated for 30 minutes at 37° C. Cell debris was removed by centrifugation (30 min, 20 000 g, 4° C.) and the supernatant was separated and labeled as membrane fraction.

Culture of E. coli BL21(DE3) pET30, used as negative control was treated by the same procedure.

### Example 5

### Preparation of Deoxyribose-5-Phosphate Aldolase Enzyme (DERA) Comprised within the Whole Cell

The aldolase gene deoC (E. coli GeneBank #EG10221, locus tag b4381) comprised within whole cell catalyst can be obtained by a number of procedures, for example as described in WO2009/092702. Nevertheless, we provide a nonlimiting example of preparation of aldolase enzyme (DERA) comprised within the whole cell.

The genomic DNA from E. coli DH5α was isolated using Wizard Genomic DNA Purification Kit (Promega, Madison, Wis., USA) according to manufacturer's instructions. Isolation of genomic DNA was made using overnight culture of E. coli grown on LB medium at 37° C. Amplification of gene deoC was performed by PCR using oligonucleotide primers CCGGCATATGACTGATCTGAAAGCAAGCAG and CCGCTCAGCTCATTAGTAGCTGCTGGCGCTC Amplification was performed by Pfx50 DNA polymerase (Invitrogen, Calsbad, Calif., USA) as follows: an initial denaturation at 95° C. for 10 min, followed by 30 cycles of 45 s at 94° C., 45 s at 60° C., and 60 s at 68° C. Final elongation was performed 420 s at 68° C. The resulting fragments were separated by agarose gel electrophoresis and purified. A 0.9-kb DNA fragment containing deoC (SEQ ID NO. 09) was separated by agarose gel electrophoresis and purified. The product was ligated into plasmid pGEM T-Easy (Promega, Madison, Wis., USA) in a T4 ligase reaction. Thus obtained plasmid construct was cleaved with restriction endonucleases NdeI and BlpI, the resulting fragments were separated on agarose gel electrophoresis and 0.9 kb fragment containing deoC was purified. An expression vector pET30a(+) (Novagen Inc., Madison, Wis., USA) was cleaved using the same aforementioned restriction endonucleases and purified. The 0.9-kb fragment containing deoC gene was assembled with the cleaved expression vector in a T4 ligase reaction. E. coli JM109 cells were transformed with the obtained ligation reaction and kanamycin resistant colonies were cultured. Afterwards plasmid DNA was isolated. The resulting construct was designated pET30/DeoC and sequenced for confirmation of the gene sequence. The cloning procedure was performed with a target to allow expression of protein having sequence SEQ ID NO. 10. The DERA aldolase expressing organism was prepared by transforming BL21 (DE3) competent cells with said plasmid.

Expression of deoC: VD medium (50 mL; 10 g/L bacto yeast extract, 5 g/L glycerol, 5 g/L NaCl, 4 g/L NaH₂PO₄.2H₂O, pH=7.0) supplemented with kanamycin (25 μg/mL) was inoculated with a single colony E. coli BL21 DE(3) pET30/DeoC from a freshly streaked plate and cultured overnight (37° C., 250 rpm).

The procedure of expression of E. coli DeoC was undertaken as described in Procedure 1A, with a sole difference of IPTG inducer concentration being 0.5 mM. After expression cells were harvested and whole cell catalysts were obtained as described in Procedure 1B to obtain living whole cell catalysts. To obtain resting whole cell catalysts Procedure 10 was undertaken.

### Example 6

### Preparation of Aldolase Enzyme (DERA) and Quinoprotein Glucose Dehydrogenase Enzyme Comprised within a Whole Cell Culture of a Single Microorganism

Two examples of a genetically modified organisms having both aldolase activity (DERA) and glucose dehydrogenase activity were constructed.

In the first case, the gene yliI, having additional ribosomal binding site (RBS) was added into the construct pET30/DeoC. The resulting construct is bearing two different coding sequences (one encoding DERA and the other encoding Yli) organized in a single operon and under transcriptional control of IPTG inducible promoter.

Specifically, the plasmid DNA from E. coli JM109 pET30/YliI (construction is described in Example 1) was isolated using Wizard Plus SV Minipreps DNA Purification Kit (Promega, Madison, Wis., USA) according to manufacturer's instructions. Isolation of plasmid DNA was made using overnight culture of E. coli grown on LB medium at 37° C.

The isolated plasmid pET30/YliI was used as a template in a PCR reaction to amplify sequence containing gene yliI and RBS derived from expression vector pET30a(+) upstream of the coding region. PCR reaction was performed using oligonucleotide primers GCAGGCTGAGCTTAACTTTAAGAAGGAGATATACATATG and GCGCGCTCAGCCTAATTGCGTGGGCTAACTTTAAG Amplification of this fragment was performed by PCR using Pfu ULTRA II Fusion HS DNA 200 polymerase (Agilent, Santa Clara, Calif., USA) as follows: an initial denaturation at 98° C. for 3 min, followed by 30 cycles of 20 s at 98° C., 20 s at 55° C., and 90 s at 72° C. Final elongation was performed 180 s at 72° C. The resulting fragments were separated by agarose gel electrophoresis and 1.2 kb fragment containing yliI and RBS site was purified. The product was transferred to plasmid pGEM T-Easy (Promega, Madison, Wis., USA) and designated pGEM/RBS_yliI. The construct was cleaved with restriction endonuclease BlpI, then the resulting fragments were separated on agarose gel electrophoresis and 1.2 kb fragment containing yliI and RBS site was purified. The construct pET30a/DeoC (construction is described in Example 5) was cleaved using the aforementioned restriction endonuclease (BlpI) and purified. Shrimp Alkaline Phosphatase (SAP, purchased by Promega, Madison, Wis., USA) was used to dephosphorylate the 5′ phosphorylated ends of cleaved pET30a/DeoC according to manufacturer's instructions. Thus self-ligation of pET30a/DeoC was prevented. The fragments were assembled in a T4 ligase reaction. E. coli JM109 cells were transformed with the obtained ligation reactions and kanamycin resistant colonies were cultured and plasmid DNA was isolated. The resulting construct was designated pET30/DeoC_RBS_YliI and sequenced for confirmation of the gene sequences. Organisms expressing DERA aldolase and quinoprotein glucose dehydrogenases were prepared by transforming BL21 (DE3) competent cells with the described plasmid.

In the second case, the gene gcd, having not only additional ribosomal binding site (RBS) but also additional T7 promoter sequence, was added into the construct pET30/DeoC. The resulting vector is bearing two different coding sequences (one encoding DERA and the other encoding Gcd) under transcriptional control of IPTG inducible promoters.

Specifically, the plasmid DNA from E. coli JM109 pET30/Gcd (construction is described in Example 1) was isolated using Wizard Plus SV Minipreps DNA Purification Kit (Promega, Madison, Wis., USA) according to manufacturer's instructions. Isolation of plasmid DNA was made using overnight culture of E. coli grown on LB medium at 37° C.

The isolated plasmid DNA was used as a template in a PCR reaction to amplify sequence containing gene gcd and RBS sequence derived from expression vector pET30a(+) upstream of the coding region. PCR reaction was performed using oligonucleotide primers GCTGGCTCAGCCTCGATCCCGCGAAATTAATA and GCGCGCTCAGCGCAAGTCTTACTTCACATCATCCGGCAG Amplification of this fragment was performed by PCR using Pfu ULTRA II Fusion HS DNA 200 polymerase (Agilent, Santa Clara, Calif., USA) as follows: an initial denaturation at 98° C. for 3 min, followed by 30 cycles of 20 s at 98° C., 20 s at 55° C., and 90 s at 72° C. Final elongation was performed 180 s at 72° C. The resulting fragments were separated by agarose gel electrophoresis and 1.2 kb fragment containing gcd and RBS site was purified. The product was transferred to plasmid pGEM T-Easy (Promega, Madison, Wis., USA) and designated pGEM/T7p_RBS_gcd. The construct was cleaved with restriction endonuclease BlpI, then the resulting fragments were separated on agarose gel electrophoresis and 2.4 kb fragment containing gcd, RBS and T7 promoter sequence site was purified.

The construct pET30a/DeoC (construction is described in Example 5) was cleaved using the same aforementioned restriction endonuclease (BlpI) and purified. Shrimp Alkaline Phosphatase (SAP, purchased by Promega, Madison, Wis., USA) was used to dephosphorylate the 5′ phosphorylated ends of cleaved pET30a/DeoC according to manufacturer's instructions. Thus self-ligation of pET30a/DeoC was prevented. The fragments were assembled in a T4 ligase reaction. E. coli JM109 cells were transformed with the obtained ligation reactions and kanamycin resistant colonies were cultured and plasmid DNA was isolated. The resulting constructs was designated pET30/DeoC_T7p_RBS_Gcd and sequenced for confirmation of the gene sequences. The organism expressing DERA aldolase and quinoprotein glucose dehydrogenase was prepared by transforming BL21 (DE3) competent cells with the described plasmid.

Unless stated otherwise the expression of the enzymes encoded by the strains obtained by the above procedure was performed as described in Procedure 1A.

### Example 7

### Preparation of E. Coli Able to Synthesize Pyrroloquinone Quinone (PQQ)

Gluconobacter oxydans (ATCC 621H) gene cluster pqqABCDE (pqqA, pqqB, pqqC, pqqD, pqqE, GeneBank #CP000009, approximate location in the genome 1080978 . . . 1084164), which is involved in pyrroloquinoline quinone (PQQ) biosynthesis was expressed in E. coli.

The genomic DNA from Gluconobacter oxydans (ATCC 621H) was isolated using Wizard Genomic DNA Purification Kit (Promega, Madison, Wis., USA) according to manufacturer's instructions. Isolation of genomic DNA was made using culture of G. oxydans grown on mannitol medium (10 g/L bacto yeast extract, 3 g/L peptone, 5 g/L mannitol, pH 7.0) 48 h at 26° C. The said isolated genome was used as template for amplification of gene cluster pqqABCDE and its own promoter. Amplification was performed by PCR using oligonucleotide primers GCGCGGTACCGCACATGTCGCGGATGTTCAGGTGTTC (SEQ ID NO. 80) and GCGCGGATCCGGGCGGAGAGTTTGGAGAACCTCTTCA (SEQ ID NO. 81) and using Pfu ULTRA II Fusion HS DNA 200 polymerase (Agilent, Santa Clara, Calif., USA) as follows: an initial denaturation at 95° C. for 2 min, followed by 30 cycles of 20 s at 95° C., 20 s at 65° C., and 120 s at 72° C. Final elongation was performed 180 s at 72° C. A 3.4-kb DNA fragment of G. oxydans bearing the pqqABCDE operon and parts of the upstream and downstream sequences was separated by agarose gel electrophoresis and purified. The 3.4-kb product (SEQ ID NO. 11) was ligated to plasmid pGEM T-Easy (Promega, Madison, Wis., USA). E. coli JM109 cells were transformed with the obtained ligation reaction and ampicillin resistant colonies were cultured and plasmid DNA was isolated. The resulting plasmid construct was designated pGEMpqqA-E and sequenced for confirmation of the gene sequences. The cloning procedure was performed with a target to allow expression of genes under control of their native promoters and thus production of proteins PqqA, PqqB, PqqC, PqqD and PqqE derived from G. oxydans (SEQ ID NO. 12, 13, 14, 15, 16, respectively).

Synthesis of PQQ in E. coli: (Procedure 7A): LB medium (30 mL; 20 g/L Bacto LB broth) supplemented with ampicillin (100 μg/mL) was inoculated with a single colony E. coli JM109 pGEM/pqqA-E from a freshly streaked plate and pre-cultured to late log phase (37° C., 250 rpm, 8 h). The pre-culture cells were pelleted by centrifugation (10 000 g, 10 min) and washed three times with 50 mM phosphate buffer (pH 7.0). Pre-culture cells were resuspended in the same aforementioned buffer at the same volume as in the original pre-culture. The suspension (inoculum size 5%, v/v) was then transferred to glucose minimal medium (100 mL, 5 g/L D-glucose, 2 g/L sodium citrate 10 g/L K₂HPO₄, 3.5 g/L (NH₄)₂SO₄, pH 7.0). The culture was grown at 37° C., 250 rpm, 48 h. The cell culture was supplemented with 1 mg/mL lysozyme solution. Lysis was performed at 37° C., 1 h. After lysis cell debris were removed by sedimentation (10 min, 20 000 g, 4° C.) to obtain a clear aqueous solution. PQQ comprised within a cell free extract was thus obtained.

Method for confirming PQQ production in E. coli is described in Example 13.

### Example 8

### Preparation of Aldose Dehydrogenase Enzyme Comprised within a Whole Cell Culture of a Single Microorganism Able to Synthesize Pyrroloquinoline Quinone (PQQ)

The expression plasmid bearing gene yliI and gene cluster pqqA-E was constructed.

Construct pET3O/YliI (preparation is described in Example 1) was digested with restriction endonuclease SphI. Shrimp alkaline phosphatase (SAP, purchased by Promega, Madison, Wis., USA) was used to dephosphorylate the 5′ phosphorylated ends of cleaved pET30a/YliI according to manufacturer's instructions. Thus self-ligation of pET30a/YliI was prevented. A double stranded linker GCGCAAGCATGCGGATCCGGTACCAAGCTTGCATGCACACTA (SEQ ID NO. 82) (ordered at Invitrogen, Calsbad, Calif., USA) containing double restriction recognition sites SphI and recognition sites for restriction endonucleases BamHI, KpnI and HindIII was following cleaving with SphI introduced into the SphI site on the digested construct pET30/YliI.

The cleaved linker and cleaved construct were assembled in a T4 ligase reaction. The introduction of the linker inserts additional restriction endonucleases recognition sites into the plasmid. The construct pGEM/pqqA-E (according to Example 7) was cleaved with restriction endonucleases BamHI and KpnI, then the resulting fragments were separated on agarose gel electrophoresis and 3.4 kb fragment containing gene operon pqqABCDE was purified. Construct pET30/YliI with linker was cleaved using the aforementioned restriction endonucleases and purified. The fragments (cleaved pET30/YliI with linker and pqqA-E, respectively) were assembled in a T4 ligase reaction. E. coli JM109 cells were transformed with the obtained ligation reaction and kanamycin resistant colonies were cultured and plasmid DNA was isolated. The resulting plasmid construct was designated pET30/YliI+pqqA-E and sequenced for confirmation of the gene sequences.

The organism able to express aldose dehydrogenase and meanwhile synthesize pyrroloquinoline quinone was prepared by transforming BL21 (DE3) competent cells with said plasmid.

Expression of E. coli YliI and comprised within a whole cell culture of a single microorganism able to produce PQQ was undertaken as described in Procedure 1A. Corresponding to Procedures 1B and 1C living whole cell catalyst and resting whole cell catalyst, respectively, were prepared. Cell free lysate was prepared following procedure described in Example 2.

Method for confirming oxidation capability and PQQ production in E. coli is described in Example 13.

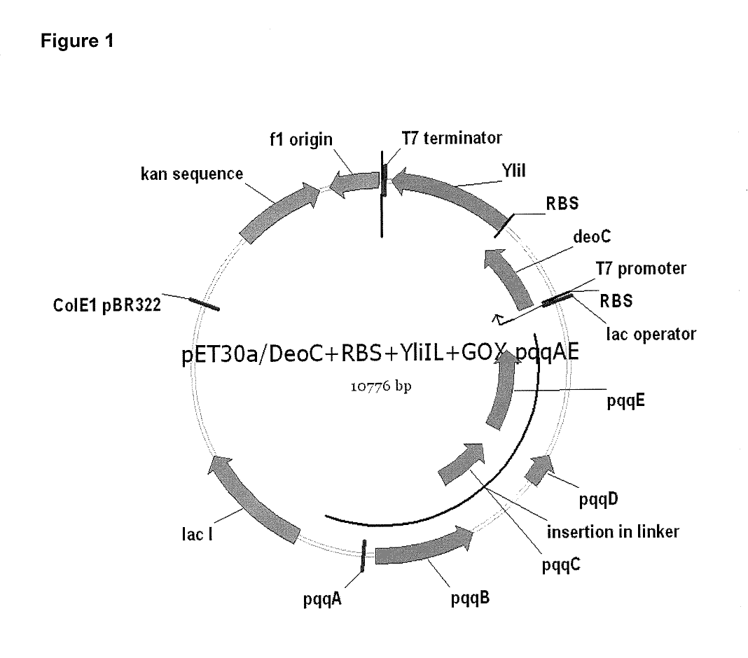

In addition a construct containing both DERA and aldose dehydrogenase YliI, as well as PQQ biosynthetic cluster from Gluconobacter oxydans (ATCC 621H) was assembled. The procedure was performed in analogy to the above procedure, however the vector pET30/DeoC_RBS_YliI (described in Example 6) was used as a starting point. The resulting plasmid was designated pET30/DeoC_RBS_YliI+pqqA-E (FIG. 1).

### Example 9

### Preparation of Kluyvera intermedia Whole Cell Catalyst

Cultivation of Kluyvera intermedia (ATCC 33421, formerly Enterobacter intermedium): VD medium (30 mL; 10 g/L bacto yeast extract, 5 g/L glycerol, 5 g/L NaCl, 4 g/L NaH₂PO₄.2H₂O, pH was adjusted with 1 M NaOH to pH=7.0) was inoculated with a single colony of Kluyvera intermedia from a freshly streaked VD agar plate and pre-cultured to late log phase (30° C., 250 rpm, 8 h).

The pre-culture cells (inoculum size 10%, v/v) were then transferred to fresh VD medium (100 mL; 10 g/L bacto yeast extract, 5 g/L glycerol, 5 g/L NaCl, 4 g/L NaH₂PO₄.2H₂O, pH was adjusted with 1 M NaOH to pH=7.0). The cells were maintained at 30° C., 250 rpm for 16 h.

Preparation of whole cell catalysts (Procedure 1B): After cultivation cells were harvested by centrifugation (10 000 g, 5 min, 4° C.). Supernatant was discarded after centrifugation and pellet was resuspended in fresh VD medium (10 g/L bacto yeast extract, 5 g/L glycerol, 5 g/L NaCl, 4 g/L NaH₂PO₄.2H₂O, pH was adjusted with 1 M NaOH to pH=6.0). Cells were resuspended in one tenth of initial culture volume. The aldose dehydrogenase comprised within the living whole cell was thus obtained in fresh medium.

Alternatively (Procedure 10), after discarding supernatant after centrifugation pellet was resuspended in phosphate buffer (50 mM KH₂PO₄, 150 mM NaCl, pH 6.0). Cells were resuspended in one tenth of initial culture volume. The aldose dehydrogenase comprised within the resting whole cell culture was thus obtained.

Cell free lysate was prepared by following the procedure described in Example 2.

### Example 10

### Preparation of Gluconobacter oxydans Whole Cell Catalyst

Cultivation of Gluconobacter oxydans (ATCC #621H): Mannitol medium (100 mL; 10 g/L bacto yeast extract, 3 g/L peptone, 5 g/L mannitol, pH was adjusted with 1 M NaOH to pH=7.0) was inoculated with a single colony Gluconobacter oxydans from a freshly streaked mannitol agar plate and cultured. The cells were maintained at 26° C., 250 rpm for 48 h.

Preparation of whole cell catalysts (Procedure 1B): After cultivation cells were harvested by centrifugation (10 000 g, 5 min, 4° C.). Supernatant was discarded after centrifugation and pellet was resuspended in fresh VD medium (10 g/L bacto yeast extract, 5 g/L glycerol, 5 g/L NaCl, 4 g/L NaH₂PO₄.2H₂O, pH was adjusted with 1 M NaOH to pH=6.0). Cells were resuspended in one tenth of initial culture volume. The aldose dehydrogenase comprised within the living whole cell was thus obtained in fresh medium.

Alternatively (Procedure 10), after discarding supernatant after centrifugation pellet was resuspended in phosphate buffer (50 mM KH₂PO₄, 150 mM NaCl, pH 6.0). Cells were resuspended in one tenth of initial culture volume. The aldose dehydrogenase comprised within the resting whole cell culture was thus obtained.

Cell free lysate was prepared following the procedure described in Example 2.

### Example 11

### A Method for Determination of Compound (II) Oxidation/Dehydrogenation Activity Using DCPIP as Electron Acceptor

This method is useful for determining aldose dehydrogenase activity (or activity of other dehydrogenases and/or oxidases). Accordingly the same method is used for screening and identifying enzymes and/or organisms capable of carrying out the reaction of oxidation/dehydrogenation of compound (II) resulting in compound (I).

Oxidation/dehydrogenation activity towards compound (II) can be measured using a living whole cell catalyst (regard to Procedure 1B), resting whole cell catalyst (regard to Procedure 1C), a lysate (preparation is described in Example 2), a periplasmic fraction (Example 3) or membrane fraction (Example 4) of any microorganism regardless of it being native or genetically modified microorganism. For practical purposes it will be understood hereinafter that a term “analyzed material” includes all preparations of catalysts as described in previous examples. The results obtained using different preparations are given separately in provided tables below.

For comparative studies cell density of tested microorganisms was quantified as wet weight in mg per mL of sample. The cells in a sample were separated from the broth by centrifugation (10 000 g, 10 min) and wet pellet was weighted. When lysate, periplasmic fraction or membrane fraction were used as a testing fraction, the data of wet cell weight of whole cell these fractions were derived from was taken into account.

1 mL of “analyzed material” was supplemented with 5 μM PQQ (Sigma Aldrich, Germany) and 10 mM MgCl₂ (Sigma Aldrich, Germany) and incubated at room temperature for 10 minutes. Prior to measurement pH value of “analyzed material” was adjusted to 8.0 with 1 M NaOH. Where stated otherwise (symbol “*” states for exception) the difference of measurement was pH value—more particularly, pH 6.0. Where stated, no additional, except intrinsic, PQQ was added into reaction mixture.

Screening method was performed in a 96-well microplate in order to screen for and identify “analyzed material” useful for converting ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate in presence of artificial electron acceptor 2,6-dichlorobenzenone-indophenole (DCPIP) combined with phenazine methosulfate (PMS).

The reaction mixture (total volume was 200 μL) contained phosphate buffer pH 8.0 (or phosphate buffer pH 6.0, where stated), 100 mM substrate ((2S,4R)-4,6-dihydroxytetrahydro-2H-pyran-2-yl)methyl acetate), 1 mM DCPIP (Sigma Aldrich, Germany), 0.4 mM PMS (Sigma Aldrich, Germany). “Analyzed material” (50 μL) was added to the reaction mixture. Where needed (due to rapid completion of the reaction), the “analyzed material” was diluted in phosphate buffer pH 8.0 or phosphate buffer pH 6.0, where stated. All tested “analyzed materials” were made in triplicates. Useful range in which the assay is linear was found to be between 10 and 400 mAU/sec.

Method was performed with spectrophotometer Spectra Max Pro M2 (Molecular Devices, USA). Absorbance at 600 nm was measured every 15 seconds for 15 minutes at 28° C. Results were collected and analyzed using software SoftMax Pro Data Acquisition & Analysis Software.

Activity of “analyzed material” and thus capability of carrying out the reaction of converting compound (II) to compound (I), specifically of converting ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate was defined as absolute value of reduction in absorbance unit per minute per wet weight of culture cells used for preparation of any “analyzed material” according to procedures 1C, 2, 3 or 4 present in the reaction mixture (abs[mAU min⁻¹ mg⁻¹]). Data are average values of three parallel measurements and are shown in a table below.

| | | Resting whole | | | | Periplasm | | Membrane | |
|---|---|---|---|---|---|---|---|---|---|
| | | cell catalyst | | Lysate | | fraction | | fraction | |
| | | (Procedure 1B) | | (Example 2) | | (Example 3) | | (Example 4) | |
| | | without | with | without | with | without | with | without | with |
| “analyzed material” | | PQQ | PQQ | PQQ | PQQ | PQQ | PQQ | PQQ | PQQ |
| 1* | E. coli BL21(DE3) pET30 | 90 | 270 | 72 | 90 | 75 | 130 | 99 | 355 |
| | (Example 1) | | | | | | | | |
| 2 | E. coli BL21(DE3) | 88 | 1897 | 71 | 1913 | 72 | 1944 | 98 | 345 |
| | pET30/Ylil | | | | | | | | |
| | (Example 1) | | | | | | | | |
| 3* | E. coli BL21(DE3) | 87 | 2403 | 75 | 346 | 75 | 302 | 101 | 2707 |
| | pET30/Gcd | | | | | | | | |
| | (Example 1) | | | | | | | | |
| 4 | E. coli BL21(DE3) | 91 | 1703 | 88 | 1655 | 74 | 1622 | 97 | 353 |
| | pET30/GdhB | | | | | | | | |
| | (Example 1) | | | | | | | | |
| 5 | E. coli BL21(DE3) | 88 | 1714 | 75 | 1687 | 72 | 1503 | 95 | 376 |
| | pET30/GdhB_therm | | | | | | | | |
| | (Example 1) | | | | | | | | |
| 6* | E. coli BL21(DE3) | 89 | 275 | 78 | 135 | 77 | 112 | 96 | 345 |
| | pET30/DeoC | | | | | | | | |
| | (Example 5) | | | | | | | | |
| 7 | E. coli BL21(DE3) | 92 | 1551 | 81 | 1581 | 75 | 1603 | 99 | 356 |
| | pET30/DeoC_RBS_Ylil | | | | | | | | |
| | (Example 6) | | | | | | | | |
| 8* | E. coli BL21(DE3) | 89 | 1987 | 82 | 365 | 79 | 299 | 95 | 2104 |
| | pET30/DeoC_T7p_RBS_Gcd | | | | | | | | |
| | (Example 6) | | | | | | | | |
| 9** | E. coli BL21(DE3) | 301 | 1895 | 405 | 1902 | 455 | 1911 | 123 | 355 |
| | pET30/Ylil + pqqA-E | | | | | | | | |
| | (Example 8) | | | | | | | | |
| 10 | Kluyvera intermedia | 1324 | 1330 | N.A. | N.A. | N.A. | N.A. | N.A. | N.A. |
| | (Example 9) | | | | | | | | |
| 11 | Gluconobacter oxydans | 1190 | 1205 | N.A. | N.A. | N.A. | N.A. | N.A. | N.A. |
| | (Example 10) | | | | | | | | |

Legend:
*pH value of reactions was 6.0
**no additional, except intrinsic, PQQ was added in column “without PQQ”.

For negative controls, at least one component (except electron acceptor DCPIP combined with PMS) of reaction mixture was replaced with phosphate buffer pH 8.0 or pH 6.0 where appropriate. No discoloration was observed in all negative control wells even after prolonged incubation. When aldose dehydrogenase clear aqueous solution was replaced with phosphate buffer, no discoloration was observed. When any of the substrates (((2S,4R)-4,6-dihydroxytetrahydro-2H-pyran-2-yl)methyl acetate, glucose, galactose or glycerol) were replaced with phosphate buffer, no discoloration was observed. When PQQ was not provided to the reaction mixture by any means described in this invention (except in mixtures 9, 10 and 11 where PQQ is provided intrinsically), no discoloration was observed.

### Example 12

### Determination of Properties of Aldose Dehydrogenase Contained in E. Coli in a Presence of DCPIP

a) For determination of activity of E. coli YliI aldose dehydrogenases on other substrates and different concentrations, additional experiments were performed.

50 μL of “analyzed material” (more particularly E. coli BL21 pET30/YliI reconstituted with 5 μL PQQ and 10 mM MgCl₂ and undertaking procedure 1B), 1 mM DCPIP, 0.4 mM PMS were placed in 96-well microtiter plate wells along with different concentrations (2.5-10 g/L) of substrates (((2S,4R)-4,6-dihydroxytetrahydro-2H-pyran-2-yl)methyl acetate, D-glucose and D-galactose). Final volume of reaction mixture was 200 μL, all components were dissolved in phosphate buffer pH 8.0.

Discoloration of DCPIP was observed in these wells. The discoloration rate was about two times faster when ((2S,4R)-4,6-dihydroxytetrahydro-2H-pyran-2-yl)methyl acetate was used as substrate than with control wells containing D-glucose or D-galactose as a substrate.

Discoloration was faster when 10 g/L of ((2S,4R)-4,6-dihydroxytetrahydro-2H-pyran-2-yl)methyl acetate used as substrate in contrast to 2.5 g/L said substrate. A slight substrate inhibition to the reaction rate was observed only when concentration of ((2S,4R)-4,6-dihydroxytetrahydro-2H-pyran-2-yl)methyl acetate in the reaction mixture was raised above 40 g/L.

To estimate possible influence of the product ((2S,4R)-4-hydroxy-6-oxotetrahydro-2H-pyran-2-yl)methyl acetate on the reaction rate, various amounts of said product were added to the reaction mixture before the initiation of the reaction. Slight reduction in reaction rates were observed only when more than 35 g/L of ((2S,4R)-4-hydroxy-6-oxotetrahydro-2H-pyran-2-yl)methyl acetate was added in addition of 10 g/L of ((2S,4R)-4,6-dihydroxytetrahydro-2H-pyran-2-yl)methyl acetate.

To investigate substrate specificity and pH optima of E. coli YliI aldose dehydrogenase the following experiments were performed:

One reaction mixture (total volume was 200 μL) contained phosphate buffer pH 8.0, 50 mM substrate (((2S,4R)-4,6-dihydroxytetrahydro-2H-pyran-2-yl)methyl acetate, D-glucose or glycerol), 1 mM DCPIP, 0.4 mM PMS. 50 μL of “analyzed material” (described in detail further on), supplemented with 5 μM PQQ (Sigma Aldrich, Germany) and 10 mM MgCl₂ was added to the mixture. The catalyst E. coli BL21 pET30/YliI culture obtained either as resting whole cell catalyst (obtained undertaking Procedure 1C) or as lysate (as described in Example 2). The initial pH value of reaction mixture was 8.0. All tested solutions were made in triplicates.

The second reaction mixture was prepared identically to the above with a sole difference: the pH value of assembled mixture being 6.0 (all compounds of reaction mixture were dissolved in phosphate buffer pH 6.0 and the initial pH value of reaction mixture was thus 6.0). All tested solutions were made in triplicates.

Differentiation of aldose dehydrogenases and thus converting substrates at different optimums in said reaction mixtures was determined with spectrophotometer Spectra Max Pro M2 (Molecular Devices, USA). Absorbance at 600 nm was measured every 15 seconds for 15 minutes at 28° C. Results were collected and analyzed using software SoftMax Pro Data Acquisition & Analysis Software. Experiment was performed as described in Example 11. In the table below are shown activities of “analyzed material” (abs[mAU min⁻¹ mg⁻¹])

| | E. coli BL21(DE3) pET30/Ylil | | | |
|---|---|---|---|---|
| | pH 6.0 | | pH 8.0 | |
| | resting | | resting | |
| Substrate | whole cell | lysate | whole cell | lysate |
| ((2S,4R)-4,6-dihydroxytetrahydro- | 270 | 273 | 1897 | 1913 |
| 2H-pyran-2-yl)methyl acetate | | | | |
| D-glucose | 350 | 210 | 356 | 321 |
| glycerol | 85 | 70 | 247 | 235 |

b) Further, the characteristics of structurally distinct, membrane bound glucose dehydrogenase from E. coli, the Gcd, were evaluated using the DCPIP method.

50 μL of “analyzed material” (more particularly E. coli BL21 pET30/Gcd reconstituted with 5 μL PQQ and 10 mM MgCl₂ and undertaking procedure 1B), 1 mM DCPIP, 0.4 mM PMS were placed in 96-well microtiter plate wells along with different concentrations (2.5-10 g/L) of substrates (((2S,4R)-4,6-dihydroxytetrahydro-2H-pyran-2-yl)methyl acetate, D-glucose and D-galactose). Final volume of reaction mixture was 200 μL, all components were dissolved in phosphate buffer pH 6.0.

Discoloration of DCPIP was observed in these wells. The discoloration rate was about two times slower when ((2S,4R)-4,6-dihydroxytetrahydro-2H-pyran-2-yl)methyl acetate was used as substrate than with control wells containing D-glucose or D-galactose as a substrate.

Discoloration was faster when 10 g/L of ((2S,4R)-4,6-dihydroxytetrahydro-2H-pyran-2-yl)methyl acetate used as substrate in contrast to 2.5 g/L said substrate. A slight substrate inhibition to the reaction rate was observed only when concentration of ((2S,4R)-4,6-dihydroxytetrahydro-2H-pyran-2-yl)methyl acetate in the reaction mixture was raised above 60 g/L.

To estimate possible influence of the product ((2S,4R)-4-hydroxy-6-oxotetrahydro-2H-pyran-2-yl)methyl acetate on the reaction rate, various amounts of said product were added to the reaction mixture before the initiation of the reaction. Slight reduction in reaction rates were observed only when more than 55 g/L of ((2S,4R)-4-hydroxy-6-oxotetrahydro-2H-pyran-2-yl)methyl acetate was added in addition of 10 g/L of ((2S,4R)-4,6-dihydroxytetrahydro-2H-pyran-2-yl)methyl acetate.

To investigate substrate specificity and pH optima of said E. coli membrane aldose dehydrogenase the above mentioned experiments were performed on the catalyst E. coli BL21 pET30/Gcd.

The results show that the overexpressed Gcd quinoprotein dehydrogenase in membrane is performing better at pH 6.0 when the substrate is ((2S,4R)-4,6-dihydroxytetrahydro-2H-pyran-2-yl)methyl acetate. Activity towards ((2S,4R)-4,6-dihydroxytetrahydro-2H-pyran-2-yl)methyl acetate is slightly slower than activity when glucose was used as substrate.

| | E. coli BL21(DE3) pET30/Gcd | | | |
|---|---|---|---|---|
| | pH 6.0 | | pH 8.0 | |
| | resting | | resting | |
| Substrate | whole cell | lysate | whole cell | lysate |
| ((2S,4R)-4,6-dihydroxytetrahydro- | 2403 | 2403 | 1105 | 1013 |
| 2H-pyran-2-yl)methyl acetate | | | | |
| D-glucose | 2840 | 2830 | 1260 | 1305 |
| glycerol | 215 | 202 | 102 | 100 |

The results clearly show that distinct quinoprotein dehydrogenases, even if derived from the same organism (E. coli), may have completely different activity optima and other properties, which points out the need for individual characterization and optimization of reaction conditions for each individual enzyme.

### Example 13

### Determination of Whole Cell Catalysis Capability in Bioreactor Using pO₂ Sensor

Laboratory bioreactors Infors ISF100 with maximal volume of 2 L were used for determination of whole cell catalysis capability of E. coli aldose dehydrogenases. The reactors were stirred, aerated, temperature and pH controlled as described below. When whole cell catalysts bearing holo aldose dehydrogenases were exposed to various substrates, unusual consumption of O₂ appeared. Using pO₂ sensor Hamilton OXYFERM FDA 225 PN 237452 the rate of pO₂ drop (oxygen consumption) in time after substrate was provided was found to correlate with the rate of whole cell catalysis capability.

Experiments were performed using whole cell catalyst E. coli BL21 pET30/Gcd (procedure of preparation in described in Example 1, Procedure 1A and moreover in Example 25).

Whole cell catalysts were dissolved in phosphate buffer (50 mM KH₂PO₄, 150 mM NaCl, pH 6.2) to concentrations of 5 g/L, 10 g/L and 20 g/L to the final volume (10 in the bioreactor.

The initial process parameters before substrate was added were as follows: 37° C., air flow rate 1.0 L/min (1.0 VVM), stirrer speed 1000 rpm, pH 6.2 and the dissolved oxygen concentration was kept at ≧80% of saturation. 5 μM PQQ and 10 mM MgCl₂ were provided into bioreactor broth. Feeding solution were 12.5% (v/v) ammonium hydroxide solution and the silicone antifoam compound synperonic antifoam (Sigma, A-5551) which were fed continuously.

Substrates (D-glucose and ((2S,4R)-4,6-dihydroxytetrahydro-2H-pyran-2-yl)methyl acetate) were added in one-shot in concentrations 0.25 g/L, 0.5 g/L and 1 g/L. When substrates were provided in the broth, oxygen consumption was followed in time.

The system described above is very complex in terms of mathematical description of dissolved oxygen levels over the time. On the one hand these are depending on the k_{La} (vessel properties, stirring, aeration, medium, temperature etc) and enzymatic kinetics on the aldose dehydrogenase on the other hand. In addition the oxygen probe delay kinetics play an important role. We have therefore found empirically, that oxygen consumption in the phase after the pulse feed of the substrate till the minimum of the dissolved oxygen in this dynamic system is best correlated to quadratic equation. A good indicator of the activity potential of the studied biocatalyst is the slope of this quadratic equation, measured at specific time value (for example t=15 seconds). The slope was calculated from the first derivative of the quadratic equation. This calculation procedure, showed a good reproducibility as well as linearity of the method

| | Concentration of whole cell | | |
|---|---|---|---|
| | catalyst BL21 (DE3) pET30/Gcd | | |
| | 5 g/L | 10 g/L | 20 g/L |
| D-glucose | 45813 | 114771 | 230631 |
| 2S,4R)-4,6-dihydroxytetrahydro- | 38652 | 87425 | 163562 |
| 2H-pyran-2-yl)methyl acetate | | | |

### Example 14

### Reconstitution of Holo-Enzyme Aldose Dehydrogenase with PQQ from Various Sources and Assay with DCPIP as Electron Acceptor

There are different possibilities of construction of holoenzyme with providing PQQ and appropriate divalent cations such as Mg²⁺ and Ca²⁺ to PQQ-dependent aldose dehydrogenases in situ and thus obtaining an active aldose dehydrogenase. For determination we used method with artificial electron acceptor (DCPIP combined with PMS) as described in Example 11.

To the whole cell catalysts E. coli BL21(DE3) pET30 or to whole cell catalysts E. coli BL21(DE3) pET30/YliI, E. coli BL21(DE3) pET30/Gcd, E. coli BL21(DE3) pET30/GdhB, E. coli BL21(DE3) pET30/GdhB_therm, E. coli BL21(DE3) pET30/DeoC, E. coli BL21(DE3) pET30/DeoC_RBS_YliI and pET30/DeoC_T7p_RBS_Gcd prepared according to Procedure 1B or 1C PQQ could be supplied as:
Procedure 14A: 5 μM PQQ (Sigma Aldrich, Germany), 10 mM MgCl₂ (Sigma Aldrich, Germany) and phosphate buffer pH 6.0 (5 mL) was added to living whole cell catalysts or to resting whole cell catalysts (5 mL) and incubated 10 min at room temperature.
Procedure 14B: 5 mL of supernatant of cultivated E. coli JM109 pGEM/pqqA-E (lysate was obtained as described in Procedure 7D) supplemented whole cell catalysts or resting cell catalysts (5 mL) in presence of 10 mM MgCl₂ and incubated 10 min at room temperature.
Procedure 14C: 5 mL of supernatant of cultivated Klyuvera intermedia or Gluconobacter oxydans (cultivation was described in Example 10 and Example 11, respectively) supplemented whole cell catalysts or resting cell catalysts (5 mL). Supernatant was obtained after centrifugation of cultures (10 000 g, 5 min, 4° C.) and incubated 10 min at room temperature in presence of 10 mM MgCl₂ in suspension.

Living whole cell catalysts or resting whole cell catalysts E. coli BL21(DE3) pET30/YliI+pqqA-E which have undertaken Procedures 1B or 10 (5 mL) were supplemented with phosphate buffer pH=6.0 and 10 mM MgCl₂ in same volume ratio and incubated 10 min at room temperature.

All reaction mixtures were tested in a 96-well microplate for their ability of converting ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate in presence of artificial electron acceptor DCPIP undertaking procedure as described in Example 11.

| | PQQ supply | | | | |
|---|---|---|---|---|---|
| | | | Procedure 14B: | Procedure 14C: | Procedure 14C: |
| | | | Complementation | Complementation | Complementation |
| | | | with supernatant | with supernatant | with supernatant |
| | No PQQ | Procedure 14A: | of E. coli JM109 | of cultivated | of cultivated |
| Construct | added | External PQQ | pGEM/pqqA-E | Kluyvera intermedia | Gluconobacter oxydans |
| *E. coli BL21(DE3) pET30 | 90 | 270 | 150 | 250 | 230 |
| E. coli BL21(DE3) pET30/Ylil | 88 | 1897 | 482 | 1570 | 1430 |
| *E. coli BL21(DE3) pET30/Gcd | 87 | 2403 | 340 | 2130 | 2020 |
| E. coli BL21(DE3) pET30/GdhB | 91 | 1703 | 541 | N.A. | N.A. |
| E. coli BL21(DE3) pET30/GdhB_therm | 88 | 1714 | 521 | N.A. | N.A. |
| E. coli BL21(DE3) pET30/DeoC | 89 | 275 | 110 | N.A. | N.A. |
| E. coli BL21(DE3) pET30/DeoC_RBS_Ylil | 92 | 1551 | 359 | 1226 | 980 |
| *E. coli BL21(DE3) | 89 | 1987 | 380 | 1530 | 1315 |
| pET30/DeoC_T7p_RBS_Gcd | | | | | |
| E. coli BL21(DE3) pET30/Ylil + pqqA-E | 501 | 1895 | N.A. | N.A. | N.A. |

Legend:
*pH value of reactions was 6.0

Addition of 5 μM PQQ and 10 mM MgCl₂ at the time of induction of expression of plasmids pET30, pET30/YliI, pET30/Gcd, pET30/GdhB, pET30a/GdhB_therm in E. coli BL21(DE3) cells (following Procedure 1A) revealed similar results as addition immediately before the reaction.

### Example 15

### Oxidation of lactol ((2S,4R)-4,6-dihydroxytetrahydro-2H-pyran-2-yl)methyl acetate with Various Aldose Dehydrogenases Comprised within Living Whole Cell Catalysts

Various living whole cell catalysts with aldose dehydrogenases (variety of them is described in Examples 1, 7, 8, 9 and 10, respectively) were tested for bioconversion of ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate.

Living whole cell catalysts E. coli BL21(DE3) pET30a, E. coli BL21(DE3) pET30a/YliI, E. coli BL21(DE3) pET30a/Gcd, E. coli BL21(DE3) pET30a/GdhB, E. coli BL21(DE3) pET30a/GdhB_therm (preparation is described in Example 1) were prepared as described in Procedure 1B. 9 mL samples of aforementioned living whole cell catalysts were transferred to 100 mL Erlenmeyer flasks, where the reaction was performed. Reaction mixture was supplemented with 1 μM PQQ (10 μL of 1 mM pre-prepared stock of PQQ, Sigma) and 10 mM MgCl₂ (2.5 μL of 4 M pre-prepared stock of MgCl₂, Sigma).

Living whole cell catalyst E. coli BL21(DE3) pET30a/YliI+pqqA-E (preparation is described in Example 7) were prepared as described in Procedure 1B. 9 mL samples of aforementioned cells were transferred to 100 mL Erlenmeyer flasks, where the reaction was performed.

Living whole cell catalysts Kluyvera intermedia (Example 10) and Gluconobacter oxydans (Example 11) were prepared as described in Procedure 1B. 9 mL samples of aforementioned cells were transferred to 100 mL Erlenmeyer flasks, where the reaction was performed.

((2S,4R)-4,6-dihydroxytetrahydro-2H-pyran-2-yl)methyl acetate was dissolved in phosphate buffer (50 mM KH₂PO₄, 150 mM NaCl, pH 6.0) to concentration of 2 mol/L. All samples of concentrated living whole cell catalyst were supplemented with 1 mL 2 mol/L ((2S,4R)-4,6-dihydroxytetrahydro-2H-pyran-2-yl)methyl acetate. Reaction was performed at 37° C., 250 rpm for 6 hours on a rotary shaker. At time 0′, 60′, 120′, 180′, 240′, 300′ and 360′, samples were taken. Each sample was diluted 50-times with acetonitrile for GC-MS analysis. Results at time 0′, 180′ and 360′ are shown in the table below.

The major product of the reaction had identical retention time and ion distribution as ((2S,4R)-4-hydroxy-6-oxotetrahydro-2H-pyran-2-yl)methyl acetate obtained by chemical oxidation as described in the art.

After 360 min reaction mixture was extracted five times with equal volume of ethyl acetate and all organic fractions were collected and dried over rotavapor. Finally a yellow oil was obtained.

The structure of resulting product was confirmed by NMR and is identical to what is reported in the art. ¹H NMR (300 MHz, acetone-d₆) δ 4.88 (m, 1H), 4.45 (d, J=3.0 Hz, 1H), 4.38 (hex, J=3.0 Hz, 1H), 4.23 (dd, J=3.5 Hz, J=12.0 Hz, 1H), 4.16 (dd, J=5.5 Hz, J=12.1 Hz, 1H), 2.68 (dd, J=4.3 Hz, J=17.5 HZ, 1H), 2.51 (dddd, J=0.8 Hz, J=2.0 Hz, J=3.3 Hz, J=17.5 Hz, 1H), 2.03 (s, 3H), 1.91 (m, 2H), ¹³C NMR (75 MHz, acetone-d₆) δ 170.8, 169.7, 74.2, 66.5, 62.7, 39.1, 32.3, 20.6.

| | Time [min] | | | | | |
|---|---|---|---|---|---|---|
| | 0′ | | 180′ | | 360′ | |
| | ((2S,4R)- | ((2S,4R)-4- | ((2S,4R)- | ((2S,4R)-4- | ((2S,4R)- | ((2S,4R)-4- |
| | 4,6-dihydroxy | hydroxy-6- | 4,6- | hydroxy-6- | 4,6- | hydroxy-6- |
| | tetrahydro- | oxotetrahydro- | dihydroxytetrahydro- | oxotetrahydro- | dihydroxytetrahydro- | oxotetrahydro- |
| | 2H- | 2H- | 2H- | 2H- | 2H- | 2H- |
| | pyran-2- | pyran-2- | pyran-2- | pyran-2- | pyran-2- | pyran-2- |
| | yl)methyl | yl)methyl | yl)methyl | yl)methyl | yl)methyl | yl)methyl |
| Living whole cell catalyst | acetate | acetate | acetate | acetate | acetate | acetate |
| E. coil BL21(DE3) pET30 | 38.20 g/L | 0.00 g/L | 19.17 g/L | 4.26 g/L | 10.12 g/L | 12.01 g/lL |
| E. coli BL21(DE3) pET30/Ylil | 37.880 g/L | 0.00 g/L | 13.01 g/L | 15.88 g/L | 1.65 g/L | 25.07 g/L |
| E. coli BL21(DE3) pET30/Gcd | 38.34 g/L | 0.00 g/L | 6.12 g/L | 21.74 g/L | 1.08 g/L | 28.65 g/L |
| E. coli BL21(DE3) pET30/GdhB | 38.10 g/L | 0.00 g/L | 12.88 g/L | 14.01 g/L | 1.15 g/L | 23.15 g/L |
| E. coil BL21(DE3) | 38.28 g/L | 0.00 g/L | 12.10 g/L | 14.52 g/L | 1.01 g/L | 24.95 g/L |
| pET30/GdhB_therm | | | | | | |
| E. coli BL21(DE3) | 37.87 g/L | 0.00 g/L | 16.55 g/L | 10.87 g/L | 6.15 g/L | 15.55 g/L |
| pET30/Ylil + pqqA-E | | | | | | |
| Kluyvera intermedia | 38.04 g/L | 0.00 g/L | 12.11 g/L | 13.85 g/L | 1.70 g/L | 23.45 g/L |
| Gluconobacter oxydans | 38.23 g/L | 0.00 g/L | 13.66 g/L | 13.12 g/L | 1.23 g/L | 25.56 g/L |

E. coli BL21(DE3) pET30 was used as negative control. Reaction was held at the same conditions as described above and at the end of the reaction small amount of ((2S,4R)-4-hydroxy-6-oxotetrahydro-2H-pyran-2-yl)methyl acetate was observed. However, all of the provided ((2S,4R)-4,6-dihydroxytetrahydro-2H-pyran-2-yl)methyl acetate did not remain untouched. The reason of conversion is activation of endogenous quinoprotein dehydrogenases (YliI and Gcd) present in E. coli forming holoenzyme when PQQ and MgCl₂ were provided.

### Example 16

### Sequential Reaction Using DERA Aldolase and Aldose Dehydroqenase (YliI or Gcd) for the Production ((2S,4R)-4-hydroxy-6-oxotetrahydro-2H-pyran-2-yl)methyl acetate from Acetyloxyacetaldehyde and Acetaldehyde

Living whole cell catalyst E. coli BL21(DE3) pET30/DeoC was prepared as described in Example 5 according to Procedure 1B. 5 mL of living whole cell catalyst was transferred to 100 mL Erlenmeyer flask.

Stock solution of acetyloxyacetaldehyde and acetaldehyde was prepared. 600.5 mg acetyloxyacetaldehyde (chemical synthesis of said compound was performed in our laboratory and revealed 85% purity) and 467.2 mg acetaldehyde (purchased by Fluka, USA) were dissolved in ice cold phosphate buffer pH 6.0 to final volume 10 mL.

At time 0′ 1 mL of said stock solution of acetyloxyacetaldehyde and acetaldehyde was added into reaction mixture. Reaction was performed at 37° C., 250 rpm for 3 hours on a rotary shaker.

5 mL of living whole cell catalyst E. coli BL21(DE3) pET30/YliI (preparation is described in Example 1 according to Procedure 1B) supplemented with 1 μM PQQ (Sigma Aldrich. Germany) and 10 mM MgCl₂ (Sigma Aldrich. Germany) was added to the same Erlenmeyer flask after 3 hours. The oxidation reaction was left for additional 3 hours at 37° C., 250 rpm on a rotary shaker.

At time 0′, 60′, 120′, 180′, 240′, 300′ and 360′ samples were taken. Each sample was diluted 50-times with acetonitrile for GC-MS analysis. The major product of the reaction had identical retention time and ion distribution as ((2S,4R)-4-hydroxy-6-oxotetrahydro-2H-pyran-2-yl)methyl acetate obtained by chemical oxidation as described in the art. After 360′ 12.3 g/L of ((2S,4R)-4-hydroxy-6-oxotetrahydro-2H-pyran-2-yl)methyl acetate was observed which represents 64% molar yield.

The same procedure as described above was performed using living whole cell catalyst E. coli BL21(DE3) pET30/Gcd (preparation is described in Example 1 according to Procedure 1B). After 360′ od addition of 5 ml of the living whole cell catalyst E. coli BL21(DE3) pET30/Gcd living whole cell catalyst to the reaction mixture with E. coli BL21(DE3) pET30/DeoC 14.45 g/L of ((2S,4R)-4-hydroxy-6-oxotetrahydro-2H-pyran-2-yl)methyl acetate was observed which represents 75% molar yield.

### Example 17

### Simultaneous Reaction Using DERA Aldolase and Aldose Dehydrogenase (Gcd) for the Production ((2S,4R)-4-hydroxy-6-oxotetrahydro-2H-pyran-2-yl)methyl acetate from Acetyloxyacetaldehyde and Acetaldehyde

Living whole cell catalysts E. coli BL21(DE3) pET30/Gcd and E. coli BL21(DE3) pET30/DeoC (preparation is described in Examples 1 and 5, respectively) were prepared as described in Procedure 1B. Both living whole cell catalysts were transferred to 100 mL Erlenmeyer flask in the same volume ratio (5 mL).

We set up two different reactions, where both living whole cell catalysts were present. First reaction mixture was supplemented with 5 μM PQQ (Sigma Aldrich, Germany) and 10 mM MgCl₂ (Sigma Aldrich, Germany), while the second one was used as a control where only apo aldose dehydrogenase enzyme was present.

Stock solution of acetyloxyacetaldehyde and acetaldehyde was prepared. 1.201 g acetyloxyacetaldehyde (chemical synthesis of said compound was performed in our laboratory and revealed 85% purity) and 934.4 mg acetaldehyde (purchased by Fluka. USA) were dissolved in ice cold phosphate buffer pH 6.0 to final volume 10 mL.

At time 0′ 1 mL of said stock solution of acetyloxyacetaldehyde and acetaldehyde was added into reaction mixture. Final concentrations of acetyloxyacetaldehyde and acetaldehyde were 100 mM and 210 mM, respectively. Reaction was performed at 37° C., 250 rpm for 6 hours.

At time 0′, 60′, 120′, 180′, 240′, 300′ and 360′ samples were taken. Each sample was diluted 50-times with acetonitrile for GC-MS analysis. The major product of the reaction had identical retention time and ion distribution as ((2S,4R)-4-hydroxy-6-oxotetrahydro-2H-pyran-2-yl)methyl acetate obtained by chemical oxidation as described in the art.

In first reaction mixture, where holo dehydrogenase was present, after 360′ 13.6 g/L of ((2S,4R)-4-hydroxy-6-oxotetrahydro-2H-pyran-2-yl)methyl acetate was observed which represents 71% molar yield. After 360′ only small amount of lactol ((2S,4R)-4,6-dihydroxytetrahydro-2H-pyran-2-yl)methyl acetate was observed, however substrates (acetaldehyde and acetyloxyacetaldehyde) and intermediate ((S)-(4-hydroxyoxtetan-2-yl)methyl acetate) were still present at the time.

At the same time, in the second reaction mixture, where only apo-dehydrogenase was present, and thus having only DERA aldolase active in the reaction produced only 9.55 g/L of lactol ((2S,4R)-4,6-dihydroxytetrahydro-2H-pyran-2-yl)methyl acetate.

These indications show that the reaction step by the aldose dehydrogenase (Gcd) is faster than the step catalyzed by the aldolase enzyme (DERA) and that presence of aldose dehydrogenase shifts the steady state equilibrium of the DERA reaction step towards the product and simultaneous reaction with aldose dehydrogenase (Gcd) in fact improves overall rates toward the lactone (compound I).

### Example 18

### Simultaneous Reaction Comprising DERA Aldolase and Aldose Dehydrogenase (YliI or Gcd) within a Living Whole Cell Catalyst of a Single Microorganism for the Production of ((2S,4R)-4-hydroxy-6-oxotetrahydro-2H-pyran-2-yl)methyl acetate from Acetyloxyacetaldehyde and Acetaldehyde

The below example provides a synthetic biological pathway provided within a living microorganism which is capable of producing highly enantiomerically pure ((2S,4R)-4-hydroxy-6-oxotetrahydro-2H-pyran-2-yl)methyl acetate from simple and inexpensive molecules: acetyloxyacetaldehyde and acetaldehyde.

Living whole cell catalysts E. coli BL21(DE3) pET30/DeoC_RBS_YliI and E. coli BL21(DE3) pET30/DeoC_T7p_RBS_Gcd (preparation is described in Example 6) was prepared as described in Procedure 1A. Said whole cell catalysts were separately concentrated by centrifugation (5 000 g, 10 min) and pellet was resuspended in one tenth of initial volume of the same supernatant. Exceeded supernatant was discarded. 10 mL of said concentrated living whole cell catalyst was transferred to 100 ml Erlenmeyer flask. To the cell broth were supplemented with 5 μM PQQ and 10 mM MgCl₂ and pH value of the broth was adjusted to 6.0 with ammonium solution.

Stock solution comprised of acetyloxyacetaldehyde and acetaldehyde was prepared. 1.201 g acetyloxyacetaldehyde (chemical synthesis of said compound was performed in our laboratory and revealed 85% purity) and 934.4 g acetaldehyde (purchased by Fluka. USA) were dissolved in ice cold phosphate buffer pH 6.0 to final volume 10 mL.

At time 0′ 1 mL of said stock solution of acetyloxyacetaldehyde and acetaldehyde was added into reaction mixtures. Final concentrations of acetyloxyacetaldehyde and acetaldehyde in reaction mixture were 100 mM and 210 mM, respectively. Reaction was performed at 37° C., 250 rpm for 6 hours.

At time 0′, 60′, 120′, 180′, 240′, 300′ and 360′ samples were taken. Each sample was diluted 50-times with acetonitrile for GC-MS analysis. The major product of the reaction had identical retention time and ion distribution as ((2S,4R)-4-hydroxy-6-oxotetrahydro-2H-pyran-2-yl)methyl acetate obtained by chemical oxidation as described in the art.

After 360′ 7.80 g/L of ((2S,4R)-4-hydroxy-6-oxotetrahydro-2H-pyran-2-yl)methyl acetate was observed which represents 41% molar yield when E. coli BL21(DE3) pET30/DeoC_RBS_YliI were present as catalysts. At the same time, when reaction was performed in presence of E. coli BL21(DE3) pET30/DeoC_T7p_RBS_Gcd after 360′ 13, 12 g/L of ((2S,4R)-4-hydroxy-6-oxotetrahydro-2H-pyran-2-yl)methyl acetate was observed which represents 69% molar yield.

### Example 19

### Simultaneous Reaction Comprising DERA Aldolase and Living Whole Cell Catalyst Kluyvera intermedia for the Production ((2S,4R)-4-hydroxy-6-oxotetrahydro-2H-pyran-2-yl)methyl acetate from Acetyloxyacetaldehyde and Acetaldehyde

Living whole cell catalysts E. coli BL21(DE3) pET30/DeoC and Kluyvera intermedia (preparation is described in Examples 1 and 11, respectively) were prepared as described in Procedure 1B. Both living whole cell catalysts were transferred to 100 mL Erlenmayer flask in the same volume ratio (5 mL).

Stock solution of acetyloxyacetaldehyde and acetaldehyde was prepared. 1.201 g acetyloxyacetaldehyde (chemical synthesis of said compound was performed in our laboratory and revealed 85% purity) and 934.4 mg acetaldehyde (purchased by Fluka. USA) were dissolved in ice cold phosphate buffer pH 6.0 to final volume 10 mL.

At time 0′ 1 mL of said stock solution of acetyloxyacetaldehyde and acetaldehyde was added into reaction mixture. Final concentrations of acetyloxyacetaldehyde and acetaldehyde in reaction mixture were 100 mM and 210 mM, respectively. Reaction was performed at 37° C., 250 rpm for 6 hours.

At time 0′, 60′, 120′, 180′, 240′, 300′ and 360′ samples were taken. Each sample was diluted 50-times with acetonitrile for GC-MS analysis. The major product of the reaction had identical retention time and ion distribution as ((2S,4R)-4-hydroxy-6-oxotetrahydro-2H-pyran-2-yl)methyl acetate obtained by chemical oxidation as described in the art.

After 360′ 10.40 g/L of ((2S,4R)-4-hydroxy-6-oxotetrahydro-2H-pyran-2-yl)methyl acetate was observed which represents 54% molar yield.

### Example 20

### Simultaneous Reaction Using DERA Aldolase and Aldose Dehydrogenase (Yli or Gcd) for the Production of ((4R,6S)-6-(chloromethyl)-4-hydroxytetrahydro-2H-pyran-2-one)

Living whole cell catalysts E. coli BL21(DE3) pET30/YliI, E. coli BL21(DE3) pET30/Gcd and E. coli BL21(DE3) pET30/DeoC (preparation is described in Examples 1 and 5, respectively) were prepared as described in Procedure 1B. 6 mL of living whole cell catalyst E. coli BL21(DE3) pET30/DeoC and 3 mL of E. coli BL21(DE3) pET30/YliI or E. coli BL21(DE3) pET30/Gcd were transferred to 50 mL polystyrene conical tubes (BD Falcon, USA). Reaction mixture was supplemented with 1 μM PQQ (Sigma Aldrich, Germany) and 10 mM MgCl₂.

Stock solution of chloroacetaldehyde and acetaldehyde was prepared. 817 μL of chloroacetaldehyde solution (50 wt. % in H₂O, purchased by Aldrich, USA) and 500.6 mg acetaldehyde (purchased by Fluka. USA) were dissolved in ice cold phosphate buffer pH 6.0 to final volume 5 mL.

At time 0′ 1 mL of said stock solution of chloroacetaldehyde and acetaldehyde was added into reaction mixture—total reaction volume was thus 10 mL and starting concentrations of chloroacetaldehyde and acetaldehyde in reaction mixture were 100 mM and 225 mM, respectively. Reaction was performed for 2 hours at 37° C., 200 rpm of shaking in water bath.

The progress of the reaction was monitored with gas chromatography. After 120 min reaction mixture was extracted three times with equal volume of ethyl acetate and all organic fractions were collected and dried over rotavapor. 66 mg (36.2% yield) of dried product (4R,6S)-6-(chloromethyl)-4-hydroxytetrahydro-2H-pyran-2-one remained after reaction combining E. coli BL21(DE3) pET30/YliI and E. coli BL21(DE3) pET30/DeoC, and 72 mg (39% yield) of dried product remained after reaction combining E. coli BL21(DE3) pET30/Gcd and E. coli BL21(DE3) pET30/DeoC. The products were analyzed with NMR. ¹H NMR (300 MHz, CDCl₃) δ 5.01 (m, 1H), 4.47 (m, 1H), 3.80 (m, 1H), 3.68 (m, 1H), 2.69 (d, J=3.6 Hz, 2H), 2.09 (m, 1H), 1.97 (m, 1H). ¹³C NMR (75 MHz, CDCl₃) δ 170.2, 74.8, 62.5, 46.6, 38.5, 32.8.

### Example 21

### Simultaneous Reaction Using DERA Aldolase and Aldose Dehydrogenase (YliI or Gcd) for the Production of ((4R,6S)-6-(dimethoxymethyl)-4-hydroxytetrahydro-2H-pyran-2-one)

Living whole cell catalysts E. coli BL21(DE3) pET30/YliI, E. coli BL21(DE3) pET30/Gcd and E. coli BL21(DE3) pET30/DeoC (preparation is described in Examples 1 and 5, respectively) were prepared as described in Procedure 1B. 6 mL of living whole cell catalyst E. coli BL21(DE3) pET30/DeoC and 3 mL of E. coli BL21(DE3) pET30/YliI or E. coli BL21(DE3) pET30/Gcd were transferred to 50 mL polystyrene conical tubes (BD Falcon, USA). Reaction mixture was supplemented with 1 μM PQQ (Sigma Aldrich, Germany) and 10 mM MgCl2.

Stock solution of dimethoxyacetaldehyde and acetaldehyde was prepared. 868 μL of 2,2-dimethoxyacetaldehyde solution (60 wt. % in H₂O, purchased by Fluka. USA) and 500.6 mg acetaldehyde (purchased by Fluka, USA) were dissolved in ice cold phosphate buffer pH 6.0 to final volume 5 mL.

At time 0′ 1 mL of said stock solution of dimethoxyacetaldehyde and acetaldehyde was added into reaction mixture—total reaction volume was thus 10 mL and starting concentrations of dimethoxyacetaldehyde and acetaldehyde in reaction mixture were 100 mM and 225 mM, respectively. Reaction was performed for 2 hours at 37° C., 200 rpm of shaking in water bath.

The progress of the reaction was monitored with gas chromatography. After 120 min reaction mixture was extracted three times with equal volume of ethyl acetate and all organic fractions were collected and dried over rotavapor. 53 mg (25.3% yield) of dried product (4R,6S)-6-(dimethoxymethyl)-4-hydroxytetrahydro-2H-pyran-2-one remained after reaction combining E. coli BL21(DE3) pET30/YliI and E. coli BL21(DE3) pET30/DeoC, and 67 mg (31.9% yield) of dried product remained after reaction combining E. coli BL21(DE3) pET30/Gcd and E. coli BL21(DE3) pET30/DeoC. The products were analyzed with NMR. ¹H NMR (300 MHz, CDCl₃) δ 4.74 (quint, J=3.5 Hz, 1H), 4.43 (m, 2H), 3.49 (s, 3H), 3.47 (s, 3H), 3.10 (br s, 1H), 2.72 (dd, J=3.5 Hz, J=17.8 Hz, 2H), 2.62 (m, 2H), 1.99 (m, 2H).

### Example 22

### Simultaneous Reaction Using DERA Aldolase and Aldose Dehydrogenase (YliI or Gcd) for the Production of ((4R,6S)-6-(benzyloxymethyl)-4-hydroxytetrahydro-2H-pyran-2-one)

Living whole cell catalysts E. coli BL21(DE3) pET30/YliI, E. coli BL21(DE3) pET30/Gcd and E. coli BL21(DE3) pET30/DeoC (preparation is described in Examples 1 and 5, respectively) were prepared as described in Procedure 1B. 6 mL of living whole cell catalyst E. coli BL21(DE3) pET30/DeoC and 3 mL of E. coli BL21(DE3) pET30/YliI or E. coli BL21(DE3) pET30/Gcd were transferred to 50 mL polystyrene conical tubes (BD Falcon, USA). Reaction mixture was supplemented with 1 μM PQQ (Sigma Aldrich, Germany) and 10 mM MgCl2.

Stock solution of benzyloxyacetaldehyde and acetaldehyde was prepared. 774.1 mg of benzyloxyacetaldehyde (purchased by Fluka, USA) and 500.6 mg acetaldehyde (purchased by Fluka, USA) were dissolved in ice cold phosphate buffer pH 6.0 to final volume 5 mL.

At time 0′ 1 mL of said stock solution of benzyloxyacetaldehyde and acetaldehyde was added into reaction mixture—total reaction volume was thus 10 mL and starting concentrations of benzyloxyacetaldehyde and acetaldehyde in reaction mixture were 100 mM and 225 mM, respectively. Reaction was performed for 2 hours at 37° C. 200 rpm of shaking in water bath.

The progress of the reaction was monitored with gas chromatography. After 120 min reaction mixture was extracted three times with equal volume of ethyl acetate and all organic fractions were collected and dried over rotavapor. 51 mg (19.6% yield) of dried product (4R,6S)-6-(benzyloxymethyl)-4-hydroxytetrahydro-2H-pyran-2-one remained after reaction combining E. coli BL21(DE3) pET30/YliI and E. coli BL21(DE3) pET30/DeoC, and 56 mg (21.5% yield) of dried product remained after reaction combining E. coli BL21(DE3) pET30/Gcd and E. coli BL21(DE3) pET30/DeoC. The crude products were dissolved in dichloromethane and reacted with TBDMSCl and imidazole for 24 h. Solution was concentrated and purified by chromatography to give TBDMSCl protected compound ((4R,6S)-6-(benzyloxymethyl)-4-hydroxytetrahydro-2H-pyran-2-one), which was analyzed with NMR. ¹H NMR (500 MHz, CDCl₃) δ 7.36 (m, 5H), 5.17 (s, 2H), 4.93 (quint, J=4.7 Hz, 1H), 4.38 (dd, J=3.4 Hz, J=11.8 Hz, 1H), 4.35 (quint, J=3.3 Hz, 1H), 4.27 (dd, J=4.7 Hz, J=11.8 Hz, 1H), 2.58 (d, J=3.3 Hz, 2H), 1.85 (m, 2H), 0.88 (s, 9H), 0.09 (s, 3H), 0.08 (s, 3H), ¹³C NMR (125 MHz, CDCl₃) δ 154.8, 134.8, 128.7, 128.6, 128.4, 73.2, 70.0, 68.8, 63.2, 39.0, 32.2, 25.6, 17.8, −5.0.

### Example 23

### Simultaneous Reaction Using DERA Aldolase and Aldose Dehydrogenase (YliI or Gcd) for the Production of ((4R,6R)-4-hydroxy-6-methyltetrahydro-2H-pyran-2-one)

Living whole cell catalysts E. coli BL21(DE3) pET30/YliI, E. coli BL21(DE3) pET30/Gcd and E. coli BL21(DE3) pET30/DeoC (preparation is described in Examples 1 and 5, respectively) were prepared as described in Procedure 1B. 6 mL of living whole cell catalyst E. coli BL21(DE3) pET30/DeoC and 3 mL of E. coli BL21(DE3) pET30/YliI or E. coli BL21(DE3) pET30/Gcd were transferred to 50 mL polystyrene conical tubes (BD Falcon, USA). Reaction mixture was supplemented with 1 μM PQQ (Sigma Aldrich, Germany) and 10 mM MgCl₂.

Stock solution of acetaldehyde was prepared. 445 mg of acetaldehyde (purchased by Fluka, USA) was dissolved in ice cold phosphate buffer pH 6.0 to final volume 5 mL.

At time 0′ 1 mL of said stock solution of acetaldehyde was added into reaction mixture—total reaction volume was thus 10 mL and its final concentration in reaction mixture was 200 mM. Reaction was performed for 2 hours at 37° C., 200 rpm of shaking in water bath.

The progress of the reaction was monitored with gas chromatography. After 120 min reaction mixture was extracted three times with equal volume of ethyl acetate and all organic fractions were collected and dried over rotavapor. 99 mg (34.1% yield) of dried product (4R,6R)-4-hydroxy-6-methyltetrahydro-2H-pyran-2-one remained after reaction combining E. coli BL21(DE3) pET30/YliI and E. coli BL21(DE3) pET30/DeoC, and 110 mg (37.9% yield) of dried product remained after reaction combining E. coli BL21(DE3) pET30/Gcd and E. coli BL21(DE3) pET30/DeoC. The products were analyzed with NMR. ¹H NMR (300 MHz, acetone-d₆) δ 4.76 (dq, J_{d}=11.2 Hz, J_{q}=3.2 Hz, 1H), 4.41-4.15 (m, 2H), 2.63 (dd, J=4.3 Hz, J=17.0 Hz, 1H), 2.46 (ddd, J=1.7 Hz, J=3.3 Hz, J=17.0 Hz, 1H), 1.92 (m, 1H), 1.71 (dd, J=3.0 Hz, J=14.3 Hz, 1H), 1.29 (d, J=6.4 Hz, 3H). ¹³C NMR (75 MHz, acetone-d₆) δ 170.6, 72.7, 63.1, 39.1, 38.2, 21.8.

### Example 24

### High Cell Density Production of Living Whole Cell Catalysts and One Pot Sequential Production of ((2S,4R)-4-hydroxy-6-oxotetrahydro-2H-pyran-2-ylmethyl acetate from Acetyloxyacetaldehyde and Acetaldehyde

The high cell density culture of living whole cell catalysts E. coli BL21(DE3) pET30/YliI+pqqA-E and E. coli BL21(DE3) pET30a/DeoC (preparation is described in Example 7 and Example 4, respectively) were prepared in “fed batch” bioprocess using laboratory bioreactors Infors ISF100 with maximal volume of 2 L. The reactors were stirred, aerated, temperature and pH controlled as described bellow. After consumption of initial substrates provided in the medium. ammonia and glucose are fed continuously to the process as nitrogen and carbon source, respectively.

The composition and preparation of the media was as follows:

13.3 g/L KH2PO4, 1.7 g/L citric acid, 60 mg/L Fe(III)citrate, 40 g/L D-glucose, 8 mg/L Zn(CH3COO)2 2H2O, 1 g/L (NH4)2HPO4, 2.7 g/L MgSO4 7H20 and 10 mL/L mineral solution.

Mineral solution was pre-prepared as follows:

### 1.5 g/L MnCl2 4H20, 0.3 g/L H3BO3, 0.25 g/L NaMoO4 2H2O, 0.25 g/L CoCl2 6H20, 0.15 g/L CuCl2 2H20, 0.84 g/L EDTA, 1 g/L Na2PO4 2H20

To prevent precipitation, the initial medium was prepared according to a special protocol: KH2PO4, Fe(III)citrate, mineral solution, Zn(CH3COO)2 2H2O and (NH4)2HPO4 were sequentially added as solutions to about half of the final volume. After autoclaving (20 min at 121° C.). sterile solutions of glucose, MgSO4 7H2O and kanamycin (25 mg/mL) were added after prior adjustment of the pH to 6.8 with 12.5% (v/v) ammonium hydroxide solution. Sterile distilled water was added to adjust the final volume (1 L) in the bioreactor. The above said solutions were sterilized separately by filtration (0.2 μm).

Feeding solutions were 12.5% (v/v) ammonium hydroxide solution, the silicone antifoam compound synperonic antifoam (Sigma, A-5551) and 50% (w/v) glucose.

Inoculums for both cultures were provided as 50 mL of shake flask culture in exponential growth phase. VD medium (50 mL; 10 g/L Bacto yeast extract, 5 g/L glycerol, 5 g/L NaCl, 4 g/L NaH2PO4*2H2O. pH was adjusted with 1 M NaOH to 7.0) was inoculated with a single colony of said whole cell catalyst from a freshly streaked VD agar plate and pre-cultured to late exp. phase (37° C., 250 rpm. 8 h).

The initial process parameters at inoculation were as follows: 25° C., air flow rate 1.5 L/min (1.5 VVM), stirrer speed 800 rpm, pH 6.8. During cultivation, the dissolved oxygen concentration was kept at ≧20% of saturation by a pO2/agitation rate control loop and a pO2/air flow ratio control loop. towards the end of bioprocess approaching to 0% and bioreactor capabilities reaching maximum (stirrer speed 2000 rpm, aeration 3 L/min).

The pH was kept at 6.8 during the whole process using a pH sensor controlled external pump which provides pulses of Ammonia solution to the bioreactor whenever the pH drops below 6.8.

After depletion of glucose present in the initial medium, a distinctive rise in pO₂ and pH level is observed (about 10-12 h into the process). At this time feeding with 50% (w/v) glucose started and was manually regulated as approximation as exponential curve (feeding started with 0.1 mL/min and ended with 0.6 mL/min in 24 h period. The process can be controlled by substrate supply; when glucose concentration is kept at dynamic zero, the glucose solution flow controls respiration rate of the culture. Technical limitations in regards to oxygen supply and heat transfer can be successfully overcome this way.

Induction for expression of protein YliI in the culture of E. coli BL21(DE3) pET30/YliI+pqqA-E. was performed by adding 0.05 mM IPTG (Sigma Aldrich, Germany) 6 hours after start of the feeding phase.

Induction for expression of protein DeoC in the culture of coil BL21(DE3) pET30a/DeoC, was performed by adding 0.1 mM IPTG (Sigma Aldrich, Germany) 6 hours after start of the feeding phase.

The overall length of the process is 34-42 h and wet weight of biomass at level between 200 and 240 g/L is obtained.

The high density culture of E. coli BL21(DE3) pET30/YliI+pqqA-E was cooled down to 15° C. and kept in the reactor with light steering and aeration (400 rpm, 0.5 L/min) until used for the reaction (5 h).

The high density culture of E. coli BL21(DE3) pET30a/DeoC was kept in the bioreactor and stirred with 800 rpm. Temperature was raised to 37° C. and 56.6 g of acetyloxyacetaldehyde and 120 mL of acetaldehyde (45.4 g) diluted in water were added with programmable pump to the reaction mixture. The whole quantity of acetyloxyacetaldehyde was added in with the constant flow rate in 30 minutes. Acetaldehyde was added continuously in 3 hours time span as described in the table below:

| | Time [min] | | | | | | | | |
|---|---|---|---|---|---|---|---|---|---|
| | 0 | 5 | 10 | 15 | 20 | 25 | 30 | 35 | 45 |
| Volume of added | 0 | 15 | 27 | 38 | 47 | 54 | 60 | 65 | 73 |
| acetaldehyde [mL] | | | | | | | | | |
| Flow [mL/min] | 3.000 | 2.400 | 2.200 | 1.800 | 1.400 | 1.200 | 1.000 | 0.800 | 0.600 |

| | Time [min] | | | | | | | | |
|---|---|---|---|---|---|---|---|---|---|
| | 60 | 75 | 90 | 105 | 120 | 135 | 150 | 165 | 180 |
| Volume of added | 82 | 90 | 96 | 102 | 106 | 110 | 114 | 117 | 120 |
| acetaldehyde [mL] | | | | | | | | | |
| Flow [mL/min] | 0.533 | 0.400 | 0.400 | 0.267 | 0.267 | 0.267 | 0.200 | 0.200 | 0 |

During the reaction the pH was kept at 5.8 using 12.5% (v/v) ammonium hydroxide solution and bioreactor's sensor dependent pH correction function. After 3 h or the reaction, some of the reaction mixture was removed from the reactor leaving 1 L of the reaction mixture steering with 800 rpm at 37° C. 0.5 L of high density culture of E. coli BL21(DE3) pET30/YliI+pqqA-E. preheated to 37° C. was added to the reaction mixture and aeration (1 L/min) was provided. The reaction mixture was left for 6 hours. and during that time. 0.1 mL/min of glycerol was added to the mixture and pH was maintained at 5.8 using 12.5% (v/v) ammonium hydroxide solution.

After the 6 hours the reaction was stopped. pH lowered to 5 using 5 M HCl solution and the whole volume of reaction mixture was transferred to simple glass vessel and mixed with 1.5 L of ethyl acetate to perform a “whole broth” extraction process. The organic phase was collected and another 1.5 L of ethyl acetate were added to the aqueous phase. The whole procedure was repeated 5 times. The collected organic phase fractions were joined, 200 g of anhydrous sodium sulphate was added (in order to bind the ethyl acetate dissolved water) and filtered off. The solvent was then removed by low pressure evaporation at 37° C. The remaining substance (82.6 g) was yellow to amber oil with consistency of honey at RT. Subsequent analysis using 1H NMR and GC-MS confirmed the structure of ((2S,4R)-4-hydroxy-6-oxotetrahydro-2H-pyran-2-yl)methyl acetate and the chromatographic purity was 52%. Overall molar yield of the reaction was estimated at 42.5%.

### Example 25

### High Cell Density Production of Living Whole Cell Catalysts and One Pot Sequential Production of ((2S,4R)-4-hydroxy-6-oxotetrahydro-2H-pyran-2-yl)methyl acetate from Acetyloxyacetaldehyde and Acetaldehyde

The high cell density culture of living whole cell catalysts E. coli BL21(DE3) pET30/Gcd and E. coli BL21(DE3) pET30a/DeoC (construction is described in Example 1 and Example 5, respectively) were prepared in a “fed batch” bioprocess using laboratory bioreactors Infors ISF-100 with maximal volume of 2 L. The reactors were stirred, aerated, temperature and pH controlled as described below. After consumption of initial substrates provided in the medium, ammonia and glucose are fed continuously to the process as nitrogen and carbon source, respectively.

The composition and preparation of the media was as follows:

13.3 g/L KH₂PO₄, 1.7 g/L citric acid, 60 mg/L Fe(III)citrate, 40 g/L D-glucose, 8 mg/L Zn(CH₃COO)₂.2H₂O, 1 g/L (NH₄)₂HPO₄, 2.7 g/L MgSO₄.7H₂O and 10 mL/L mineral solution.

Mineral solution was pre-prepared as follows:

1.5 g/L MnCl₂.4H₂0, 0.3 g/L H₃BO₃, 0.25 g/L NaMoO₄.2H₂O, 0.25 g/L CoCl₂.6H₂O, 0.15 g/L CuCl₂.2H₂O, 0.84 g/L EDTA, 1 g/L Na₂PO₄.2H₂O.

To prevent precipitation, the initial medium was prepared according to a special protocol: KH₂PO₄, Fe(III)citrate, mineral solution, Zn(CH₃COO)₂.2H₂O and (NH₄)₂HPO₄ were sequentially added as solutions to about half of the final volume. After autoclaving (20 min at 121° C.), sterile solutions of glucose, MgSO₄.7H₂O and kanamycin (25 mg/mL) were added after prior adjustment of the pH to 6.8 with 12.5% (v/v) ammonium hydroxide solution. Sterile distilled water was added to adjust the final volume (1 L) in the bioreactor. The above said solutions were sterilized separately by filtration (0.2 μm).

Feeding solutions were 12.5% (v/v) ammonium hydroxide solution, the silicone antifoam compound synperonic antifoam (Sigma, A-5551) and 50% (w/v) glucose.

Inoculums for both cultures were provided as 50 mL of shake flask culture in exponential growth phase. VD medium (50 mL; 10 g/L Bacto yeast extract, 5 g/L glycerol, 5 g/L NaCl, 4 g/L NaH₂PO₄.2H₂O. pH was adjusted with 1 M NaOH to 7.0) was inoculated with a single colony of said whole cell catalyst from a freshly streaked VD agar plate and pre-cultured to late exp. phase (37° C., 250 rpm, 8 h).

The initial process parameters at inoculation were as follows: 25° C., air flow rate 1.5 L/min (1.5 VVM), stirrer speed 800 rpm, pH 6.8. During cultivation, the dissolved oxygen concentration was kept at ≧20% of saturation by a pO2/agitation rate control loop and a pO2/air flow ratio control loop, towards the end of bioprocess approaching to 0% and bioreactor capabilities reaching maximum (stirrer speed 2000 rpm, aeration 3 L/min).

The pH was kept at 6.8 during the whole process using a pH sensor controlled external pump which provides pulses of ammonia solution to the bioreactor whenever the pH drops below 6.8.

After depletion of glucose present in the initial medium, a distinctive rise in pO₂ and pH level is observed (about 10-12 h into the process). At this time feeding with 50% (w/v) glucose started and was manually regulated as approximation as exponential curve (feeding started with 0.1 mL/min and ended with 0.6 mL/min in 24 h period. The process can be controlled by substrate supply; when glucose concentration is kept at dynamic zero, the glucose solution flow controls respiration rate of the culture. Technical limitations in regards to oxygen supply and heat transfer can be successfully overcome this way.

Induction for expression of protein Gcd in the culture of E. coli BL21(DE3) pET30/Gcd. was performed by adding 0.1 mM IPTG (Sigma Aldrich, Germany) 6 hours after start of the feeding phase.

Induction for expression of protein DeoC in the culture of coil BL21(DE3) pET30a/DeoC, was performed by adding 0.2 mM IPTG (Sigma Aldrich, Germany) 6 hours after start of the feeding phase.

The overall length of the process is 34-42 h and wet weight of biomass at level between 150 and 200 g/L is obtained.

The high density culture of E. coli BL21(DE3) pET30/Gcd was cooled down to 15° C. and kept in the reactor with light steering and aeration (400 rpm, 0.5 L/min) until used for the reaction (5 h).

592 mL of the high density culture of E. coli BL21(DE3) pET30a/DeoC was kept in the bioreactor and stirred with 1300 rpm. Temperature was raised to 37° C. and 39.30 g of acetyloxyacetaldehyde and 100 mL of acetaldehyde (48.46 g) diluted in water was prepared. The whole quantity of acetyloxyacetaldehyde was added in with the constant flow rate in 27 minutes. Acetaldehyde solution was added continuously with programmable pump to the reaction mixture in 90 min time span as described in the table below:

| Time [min] | 0 | 30 | 60 | 90 |
|---|---|---|---|---|
| Volume of added acetaldehyde | 0.00 | 52.50 | 71.75 | 77.00 |
| solution [mL] | | | | |
| Flow [mL/min] | 1.750 | 0.642 | 0.175 | 0.000 |

During the reaction the pH was kept at 6.2 using 12.5% (v/v) ammonium hydroxide solution and bioreactor's sensor dependent pH correction function. After 30 min of the reaction, additional 70 mL of high density culture of E, coli BL21(DE3) pET30a/DeoC was added. After 2 h or the reaction, 735 mL of the reaction mixture remaining was steered with 1400 rpm at 37° C. The GC-FID analysis showed the concentration of 75.6/L of ((2S,4R)-4,6-dihydroxytetrahydro-2H-pyran-2-yl)methyl acetate present at the conclusion of the reaction.

183 mL of high density culture of E. coli BL21(DE3) pET30/Gcd, preheated to 37° C., was added to the reaction mixture and aeration (1.4 L/min) was provided. MgCl₂ and PQQ were added to their final concentration 10 mM and 2 μM, respectively, and the reaction mixture was left stirring for 10 min in order to achieve full reconstitution of the Gcd enzyme with PQQ. The reaction mixture was left for 3 hours and during that time pH was maintained at 6.2 using 12.5% (v/v) ammonium hydroxide solution. After 65 min of the beginning of the second step reaction, steering speed was lowered to 1200 rpm and aeration flow rate to 1.2 L/min. Again, after 137 min of the beginning of the second step reaction, steering speed was lowered to 1000 rpm and aeration flow rate to 1.0 L/min. The GC-FID analysis showed the concentration of 58.6 g/L of ((2S,4R)-4-hydroxy-6-oxotetrahydro-2H-pyran-2-yl)methyl acetate predent at the conclusion of the reaction. The yield of the conversion of ((2S,4R)-4,6-dihydroxytetrahydro-2H-pyran-2-yl)methyl acetate to ((2S,4R)-4-hydroxy-6-oxotetrahydro-2H-pyran-2-yl)methyl acetate in this step, calculated from the GC-FID results was higher than 95%.

After 3 h the reaction was stopped, pH lowered to 4.0 using 5 M phosphoric acid solution and the whole volume of reaction mixture was transferred to simple glass vessel, in which 200 g/L of Na₂SO₄ was added, pH again corrected to 4.0 with 5 M phosphoric acid and mixed with 920 mL of ethyl acetate (1:1) to perform a “whole broth” extraction process. The organic phase was collected and another 920 mL of ethyl acetate was added to the aqueous phase. The whole procedure was repeated 5 times. The collected organic phase fractions were joined, ˜150 g of anhydrous magnesium sulphate was added (in order to remove the water from ethyl acetate phase) and filtered off. The solvent was then removed by low pressure evaporation at 40° C. The remaining substance (51.1 g), yellow to amber oil with consistency of honey at RT was analyzed using 1H NMR and GC-MS confirmed the structure of ((2S,4R)-4-hydroxy-6-oxotetrahydro-2H-pyran-2-yl)methyl acetate and the chromatographic purity (GC-FID) was 78.6%. Overall molar yield of the two sequential enzymatic reactions was calculated to be 81.6%.

### Example 26

### High Cell Density Production of Living Whole Cell Catalysts and One Pot Simultaneous Production of ((2S,4R)-4-hydroxy-6-oxotetrahydro-2H-pyran-2-yl)methyl acetate from acetyloxyacetaldehyde and acetaldehyde

The high cell density culture of living whole cell catalysts E. coli BL21(DE3) pET30/DeoC_T7p_RBS_Gcd (preparation is described in Example 6) was prepared in “fed batch” bioprocess using laboratory bioreactors Infors ISF-100 with maximal volume of 2 L. The reactors were stirred, aerated, temperature and pH controlled as described bellow. After consumption of initial substrates provided in the medium, ammonia and glucose are fed continuously to the process as nitrogen and carbon source, respectively.

The composition and preparation of the media was as follows:

13.3 g/L KH₂PO₄, 1.7 g/L citric acid, 60 mg/L Fe(III)citrate, 40 g/L D-glucose, 8 mg/L Zn(CH₃COO)₂.2H₂O, 1 g/L (NH₄)₂HPO₄, 2.7 g/L MgSO₄.7H₂O and 10 mL/L mineral solution.

Mineral solution was pre-prepared as follows:

1.5 g/L MnCl₂.4H₂O, 0.3 g/L H₃BO₃, 0.25 g/L NaMoO₄.2H₂O, 0.25 g/L CoCl₂.6H₂O, 0.15 g/L CuCl₂.2H₂O, 0.84 g/L EDTA, 1 g/L Na₂PO₄.2H₂O.

To prevent precipitation, the initial medium was prepared according to a special protocol: KH₂PO₄, Fe(III)citrate, mineral solution, Zn(CH₃COO)₂.2H₂O and (NH₄)₂HPO₄ were sequentially added as solutions to about half of the final volume. After autoclaving (20 min at 121° C.), sterile solutions of glucose, MgSO₄.7H₂O and kanamycin (25 mg/mL) were added after prior adjustment of the pH to 6.8 with 12.5% (v/v) ammonium hydroxide solution. Sterile distilled water was added to adjust the final volume (1 L) in the bioreactor. The above said solutions were sterilized separately by filtration (0.2 μm).

Feeding solutions were 12.5% (v/v) ammonium hydroxide solution, the silicone antifoam compound synperonic antifoam (Sigma, A-5551) and 50% (w/v) glucose.

Inoculums for both cultures were provided as 50 mL of shake flask culture in exponential growth phase. VD medium (50 mL; 10 g/L Bacto yeast extract, 5 g/L glycerol, 5 g/L NaCl, 4 g/L NaH₂PO₄.2H₂O. pH was adjusted with 1 M NaOH to 7.0) was inoculated with a single colony of said whole cell catalyst from a freshly streaked VD agar plate and pre-cultured to late exp. phase (37° C., 250 rpm, 8 h).

The initial process parameters at inoculation were as follows: 25° C., air flow rate 1.5 L/min (1.5 VVM), stirrer speed 800 rpm, pH 6.8. During cultivation, the dissolved oxygen concentration was kept at ≧20% of saturation by a pO2/agitation rate control loop and a pO2/air flow ratio control loop, towards the end of bioprocess approaching to 0% and bioreactor capabilities reaching maximum (stirrer speed 2000 rpm, aeration 3 L/min).

The pH was kept at 6.8 during the whole process using a pH sensor controlled external pump which provides pulses of ammonia solution to the bioreactor whenever the pH drops below 6.8.

After depletion of glucose present in the initial medium, a distinctive rise in pO₂ and pH level is observed (about 10-12 h into the process). At this time feeding with 50% (w/v) glucose started and was manually regulated as approximation as exponential curve (feeding started with 0.1 mL/min and ended with 0.6 mL/min in 24 h period. The process can be controlled by substrate supply; when glucose concentration is kept at dynamic zero, the glucose solution flow controls respiration rate of the culture. Technical limitations in regards to oxygen supply and heat transfer can be successfully overcome this way.

Induction for expression of proteins DERA and Gcd in the culture of E. coli BL21(DE3) pET30/DeoC+Gcd, was performed by adding 0.1 mM IPTG (Sigma Aldrich, Germany) 6 hours after start of the feeding phase.

The overall length of the process is 34-42 h and wet weight of biomass at level between 150 and 200 g/L is obtained.

690 mL of the high density culture of E. coli BL21(DE3) pET30a/DeoC+Gcd was kept in the bioreactor and stirred with 1300 rpm. Temperature was raised to 37° C., aeration (1.0 L/min) was provided, and 32.67 g of acetyloxyacetaldehyde and 100 mL of acetaldehyde (37.00 g) diluted in water was prepared. MgCl₂ and PQQ were added to their final concentration 10 mM and 5 μM, respectively, and the reaction mixture was left stirring for 10 min in order to achieve full reconstitution of the Gcd enzyme with PQQ. The whole quantity of acetyloxyacetaldehyde was added in with the constant flow rate in 35 min. Acetaldehyde solution was added continuously with programmable pump to the reaction mixture in 60 min time span as described in the table below:

| Time [min] | 0 | 30 | 60 |
|---|---|---|---|
| Volume of added acetaldehyde solution [mL] | 0.00 | 60.00 | 80.00 |
| Flow [mL/min] | 2.000 | 0.667 | 0.000 |

During the reaction the pH was kept at 6.2 using 12.5% (v/v) ammonium hydroxide solution and bioreactor's sensor dependent pH correction function. The reaction mixture was left for 3.5 h and during that time pH was maintained at 6.2 using 12.5% (v/v) ammonium hydroxide solution. The GC-FID analysis showed the concentration of 46, 1 g/L of ((2S,4R)-4-hydroxy-6-oxotetrahydro-2H-pyran-2-yl)methyl acetate predent at the conclusion of the reaction.

After 3.5 h the reaction was stopped, pH lowered to 4.0 using 5 M phosphoric acid solution and the whole volume of reaction mixture was transferred to simple glass vessel, in which 200 g/L of Na₂SO₄ were added, pH again corrected to 4.0 with 5 M phosphoric acid and mixed with 800 mL of ethyl acetate (1:1) to perform a “whole broth” extraction process. The organic phase was collected and another 800 mL of ethyl acetate was added to the aqueous phase. The whole procedure was repeated 5 times. The collected organic phase fractions were joined, ˜150 g of anhydrous magnesium sulphate was added (in order to remove the water from ethyl acetate phase) and filtered off. The solvent was then removed by low pressure evaporation at 40° C. The remaining substance (34.2 g), yellow to amber oil with consistency of honey at RT was analysed using 1H NMR and GC-MS confirmed the structure of ((2S,4R)-4-hydroxy-6-oxotetrahydro-2H-pyran-2-yl)methyl acetate and the chromatographic purity (GC-FID) was 76.3%. Overall molar yield of the two simultaneous enzymatic reactions was calculated to be 59.9%.

### REFERENCES

1. Achmann, S. et al. Direct detection of formaldehyde in air by a novel NAD+- and glutathione-independent formaldehyde dehydrogenase-based biosensor. Talanta 75, 786-91 (2008).
2. Adachi, O. et al. Biooxidation with PQQ- and FAD-Dependent Dehydrogenases. Modern Biooxidation: Enzymes, Reactions and Applications, Wiley-VCH, Weinheim 1-41 (2007).
3. Adachi, O. et al. New quinoproteins in oxidative fermentation. Biochimica et Biophysica Acta (BBA)-Proteins & Proteomics 1647, 10-17 (2003).
4. Ameyama, M. et al. Method of enzymatic determination of pyrroloquinoline quinone. Analytical biochemistry 151, 263-7 (1985).
5. Anthony C, Zatman L J (1967). “The microbial oxidation of methanol. The prosthetic group of the alcohol dehydrogenase of Pseudomonas sp. M27: a new oxidoreductase prosthetic group”. Biochem J 104 (3): 960-9. PMID 6049934. PMID 6049934
6. Anthony, C. Pyrroloquinoline quinone (PQQ) and quinoprotein enzymes. Antioxidants and Redox Signaling 3, 757-774 (2001).
7. Anthony, C. The quinoprotein dehydrogenases for methanol and glucose. Archives of biochemistry and biophysics 428, 2-9(2004).
8. Cline, A. & Hu, A. Enzymatic characterization and comparison of three sugar dehydrogenases from a pseudomonad. Journal of Biological Chemistry 240, 4493 (1965).
9. Cozier, G. E., Salleh, R. A. & Anthony, C. Characterization of the membrane quinoprotein glucose dehydrogenase from Escherichia coli and characterization of a site-directed mutant in which histidine-262 has been changed to tyrosine. Biochemical Journal 340, 639 (1999).
10. D'Costa, E. J., I. J. Higgins & A. P. F. Turner. 1986. Quinoprotein glucose dehydrogenase and its application in an amperometric glucose sensor. Biosensors. 2 71-87
11. Duine, J. A. Quinoproteins: enzymes containing the quinonoid cofactor pyrroloquinoline quinone, topaquinone or tryptophan-tryptophan quinone. European Journal of Biochemistry 200, 271-284 (1991).
12. Durand, F. et al. Designing a highly active soluble PQQ-glucose dehydrogenase for efficient glucose biosensors and biofuel cells. Biochemical and biophysical research communications 402, 750-4 (2010).
13. Gao, F., Viry, L., Maugey, M., Poulin, P., Mano, N., Engineering hybrid nanotube wires for high-power biofuel cells, Nat. Commun. 1 (2010), doi:10.1038/ncomms1000
14. Goodwin P. M, Anthony C., “The biochemistry, physisiology and genetics of PQQ and PQQ-containing enzymes,” Adv. Microb. Physiol. (1998) 40:1-80
15. Goosen N. et al., “Acinetobacter calcoaceticus Genes Involved in Biosynthesis of the Coenzyme Pyrrolo-Quinoline-Quinone: Nucleotide Sequence and Expression in Escherichia coli K-12,” J. Bacteriol. (1989) 171:447-455
16. Gupta, A. et al. Gluconobacter oxydans: its biotechnological applications. Journal of molecular microbiology and biotechnology 3, 445-56 (2001).
17. Hauge J G (1964). “Glucose dehydrogenase of bacterium anitratum: an enzyme with a novel prosthetic group”. J Biol Chem 239: 3630-9. PMID 14257587. PMID 14257587
18. Heller, A. and Feldman, B., Electrochemical glucose sensors and their applications in diabetes management, Chem. Rev. 108 (2008), pp. 2482-2505. Full Text via CrossRef|View Record in Scopus|Cited By in Scopus (77)
19. Hoelscher T., Goerisch H., “Knockout and Overexpression of Pyrroloquinoline Quinone Biosynthetic Genes in Gluconobacter oxydans 621H,” J Bacteriology (2006) 188; 21:7668-7676
20. Hommes, R. W. J., Postma, P. W., Neijssel, 0. M., Tempest, D. W., Dokter, P. & Duine, J. A. (1984). Evidence for a glucose dehydrogenase apo-enzyme in several strains of Escherichia coli. FEMS Microbiol Lett 24, 329-333.
21. Igarashi, S. et al. Molecular engineering of PQQGDH and its applications. Archives of biochemistry and biophysics 428, 52-63(2004).
22. Igarashi, S., Hirokawa, T. & Sode, K. Engineering PQQ glucose dehydrogenase with improved substrate specificity. Site-directed mutagenesis studies on the active center of PQQ glucose dehydrogenase. Biomolecular engineering 21, 81-9(2004).
23. Jonge, R. D., Mattos, M. D. & Stock, J. Pyrroloquinoline quinone, a chemotactic attractant for Escherichia coli. Journal of 178, 1224-1226 (1996).
24. Keilin, D. & Hartree, E. F. Properties of glucose oxidase (notatin): Addendum. Sedimentation and diffusion of glucose oxidase (notatin). The Biochemical journal 42, 221-9 (1948).
25. Keilin, D. & Hartree, E. F. Specificity of glucose oxidase (notatin). The Biochemical journal 50, 331-41 (1952).
26. Keilin, D. & Hartree, E. F. The use of glucose oxidase (notatin) for the determination of glucose in biological material and for the study of glucose-producing systems by nanometric methods. The Biochemical journal 42, 230-8 (1948).
27. Khairnar N. P. et al., “Pyrroloquinoline-quinone synthesized in Escherichia coli by pyrroloquinoline-quinone synthase of Deinococcus radiodurans plays a role beyond mineral phosphate solubilization,” Biochemical and Biophysical Research Communications (2003) 312:303-308
28. Kim C. H. et al., “Cloning and Expression of Pyrroloquinoline Quinone (PQQ) Genes from a Phosphate-Solubilizing Bacterium Enterobacter intermedium,” Current Microbiology (2003) 47:457-461
29. Kujawa, M. et al. Properties of pyranose dehydrogenase purified from the litter-degrading fungus Agaricus xanthoderma. The FEBS journal 274, 879-94 (2007).
30. Lapėnaitė, I., Kurtinaitienė, B. & Pliu\check{s}kys, L. Application of PQQ-GDH Based Polymeric Layers in Design of Biosensors for Detection of Heavy Metals. (2003)
31. Lapenaite, I., Ramanaviciene, A. & Ramanavicius, A. Current trends in enzymatic determination of glycerol. Critical Reviews in Analytical Chemistry 36, 13-25 (2006).
32. Leskovac V, Trivic S, Wohlfahrt G, Kandrac J, Pericin D (2005) Glucose oxidase from Aspergillus niger: the mechanism of action with molecular oxygen, quinones, and one-electron acceptors. Int J Biochem 37:731-750
33. Lidstrom, M. E. Genetics of bacterial quinoproteins. Methods in enzymology. San Diego Calif. 258, 217-227 (1995).
34. Linton, J., Woodard, S. & Gouldney, D. The consequence of stimulating glucose dehydrogenase activity by the addition of PQQ on metabolite production by Agrobacterium radiobacter NCIB 11883. Applied Microbiology and Biotechnology 25, 357-361 (1987).
35. Magnusson, O. T. et al. The structure of a biosynthetic intermediate of pyrroloquinoline quinone (PQQ) and elucidation of the final step of PQQ biosynthesis. Journal of the American Chemical Society 126, 5342-3(2004).
36. Martin, E. J. S. Assay for D-allose using a NAD cofactor coupled D-allose dehydrogenase. U.S. Pat. No. 5,567,605 (1996).
37. Matsushita, K. et al. Escherichia coli is unable to produce pyrroloquinoline quinone (PQQ). Microbiology (Reading, England) 143 (Pt 1, 3149-56 (1997).
38. Meulenberg J. J., “nucleotide sequence and structure of the Klebsiella pneumoniae pqq operon,” Mol. Gen. Genet. (1992) 232:284-294
39. Mitchell, R. & Duke, F. Kinetics and equilibrium constants of the gluconic acid-gluconolactone equilibrium. Ann. NY Acad. Sci. 172, 129-138 (1970).
40. Olsthoorn, a J. & Duine, J. a On the mechanism and specificity of soluble, quinoprotein glucose dehydrogenase in the oxidation of aldose sugars. Biochemistry 37, 13854-61 (1998).
41. Olsthoorn, a J., Otsuki, T. & Duine, J. a Ca2+ and its substitutes have two different binding sites and roles in soluble, quinoprotein (pyrroloquinoline-quinone-containing) glucose dehydrogenase. European journal of biochemistry/FEBS 247, 659-65 (1997).
42. Oubrie, a & Dijkstra, B. W. Structural requirements of pyrroloquinoline quinone dependent enzymatic reactions. Protein science: a publication of the Protein Society 9, 1265-73 (2000).
43. Oubrie, a et al. Structure and mechanism of soluble quinoprotein glucose dehydrogenase. The EMBO journal 18, 5187-94 (1999).
44. Oubrie, a Structure and mechanism of soluble glucose dehydrogenase and other PQQ-dependent enzymes. Biochimica et Biophysica Acta (BBA)-Proteins & Proteomics 1647, 143-151 (2003).
45. Pazur J H, Kleppe K (1964) The oxidation of glucose and related compounds by glucose oxidase from Aspergillus niger. Biochemistry 3:578-583
46. Puehringer S., Metlitzky M. et al., “The pyrroloquinoline quinine biosynthesis pathway revisited: A structural approach,”, BMC Biochemistry (2008) 9:8
47. Rose, a, Scheller, F. & Wollenberger, U. Quinoprotein glucose dehydrogenase modified thick-film electrodes for the amperometric detection of phenolic compounds in flow injection analysis. Fresenius& #39; journal of 369, 145-52 (2001).
48. Schie, B. J. van et al. Energy transduction by electron transfer via a pyrrolo-quinoline quinone-dependent glucose dehydrogenase in Escherichia coli, Pseudomonas aeruginosa, and Acinetobacter calcoaceticus (var. Iwoffi). Journal of bacteriology 163, 493-9 (1985).
49. Schmid, R. & Urlacher, V. B. Modern biooxidation: enzymes, reactions and applications. Engineering (Vch Verlagsgesellschaft Mbh: 2007).
50. Sierks, M. R. et al. Active site similarities of glucose dehydrogenase, glucose oxidase, and glucoamylase probed by deoxygenated substrates. Biochemistry 31, 8972-7 (1992).
51. Smolander, M., Livio, H.-L., Rasanen, L., Mediated amperometric determination of xylose and glucose with an immobilized aldose dehydrogenase electrode, Biosensors and Bioelectronics, Volume 7, Issue 9, 1992, Pages 637-643.
52. Sode, K. et al. Effect of PQQ glucose dehydrogenase overexpression in Escherichia coli on sugar-dependent respiration. Journal of biotechnology 43, 41-4 (1995).
53. Sode, K. et al. Thermostable chimeric PQQ glucose dehydrogenase. FEBS letters 364, 325-7 (1995).
54. Sode, K., Ootera, T., Shirahane, M., Witarto, A. B., Igarashi, S., Yoshida, H., Increasing the thermal stability of the water-soluble pyrroloquinoline quinone glucose dehydrogenase by single amino acid replacement, Enzyme Microb. Technol. 26 (2000) 491-496.
55. Southall, S. M. et al. Soluble aldose sugar dehydrogenase from Escherichia coli: a highly exposed active site conferring broad substrate specificity. The Journal of biological chemistry 281, 30650-9 (2006).
56. Springer A. L. et al., “Characterisation and nucleotide sequence of pqqE and pqqF in Methylobacterium extorquens AM 1,” J Bacteriol. (1996) 178:2154-2157
57. Szeponik, J. et al. Ultrasensitive bienzyme sensor for adrenaline. Biosensors and 12, 947-52 (1997).
58. Tanaka, S. et al. Increasing stability of water-soluble PQQ glucose dehydrogenase by increasing hydrophobic interaction at dimeric interface. BMC biochemistry 6, 1 (2005).
59. Volc, J. et al. Pyranose 2-dehydrogenase, a novel sugar oxidoreductase from the basidiomycete fungus Agaricus bisporus. Archives of microbiology 167, 119-25 (1997).
60. Wilson, G. S. et al. Progress toward the Development of an Implantable Sensor for Glucose. 1617, 1613-1617 (1992).
61. Wong, C. M., Wong, K. H. & Chen, X. D. Glucose oxidase: natural occurrence, function, properties and industrial applications. Applied microbiology and biotechnology 78, 927-38 (2008).
62. Yamada, M. et al. Escherichia coli PQQ-containing quinoprotein glucose dehydrogenase: its structure comparison with other quinoproteins. Biochimica et Biophysica Acta (BBA)-Proteins & Proteomics 1647, 185-192 (2003).
63. Yang X.-P. et al., “Pyrroloquinoline quinine biosynthesis in Escherichia coli through expression of the Gluconobacter oxydans pqqABCDE gene cluster,” J Ind Microbiol Biotechnol (2010) 37:575-580
64. Yoshida H. et al., “Secretion of water soluble pyrroloquinoline quinine glucose dehydrogenase by recombinant Pichia pastoris,” Enzy Microbial Tech (2002) 30:312-318
65. Zheng, Y. J. & Bruice, T. C. Conformation of coenzyme pyrroloquinoline quinone and role of Ca2+ in the catalytic mechanism of quinoprotein methanol dehydrogenase. Proceedings of the National Academy of Sciences of the United States of America 94, 11881-6 (1997).
66. Gijsen, H. Unprecedented asymmetric aldol reactions with three aldehyde substrates catalyzed by 2-deoxyribose-5-phosphate aldolase. Journal of the American Chemical 8422-8423 (1994)

### PATENT REFERENCES CITED

WO2009156083, JP2009232872, EP2251420, US2009148874, MX2007000560, JP2006314322, JP2006217811, WO2006/134482 WO2008/119810, WO2005/118794, WO2006/134482, WO2009/092702

1. A process for preparing a compound of formula (I) 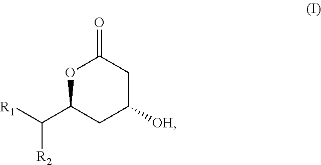
in which
R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, NR³CO(CH₂)ₙCH₃, CH₂—R⁵, optionally substituted mono- or bicyclic aryl, heterocyclic or alicyclic group; and
R₂ independently from R₁ denotes H, (CH₂)ₘ—CH₃, or aryl;
or both of R₁ and R₂ denote either X, OH or O((CH₂)ₙCH₃);
or R₁ and R₂ together denote ═O, ═CH—R⁵, or together form a ring —(CH₂)ₚ—, —(CH₂)ᵣ-(1,2-arylene)-(CH₂)ₙ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³; wherein
R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
R⁵ denotes optionally substituted mono- or bicyclic aryl, heterocyclic or alicyclic group,
X denotes F, Cl, Br or I;
n represents an integer from 0 to 10;
m represents an integer from 0 to 3;
p represents an integer from 2 to 6;
and at least one from r and s is 1;
or a pharmaceutically acceptable salt, or an ester, or a stereoisomer thereof,
the process comprising bringing in contact a compound of formula (II), 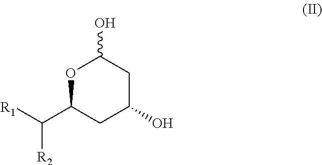
wherein R₁ and R₂ are defined as above, with an enzyme capable of catalyzing oxidation or dehydrogenation, and optionally salifying, esterifying or stereoselectively resolving the product.

2. The process according to claim 1, wherein
R⁵ denotes a moiety selected from the formula (III), (IV), (V), (VI), (VII), (VIII) and (IX);

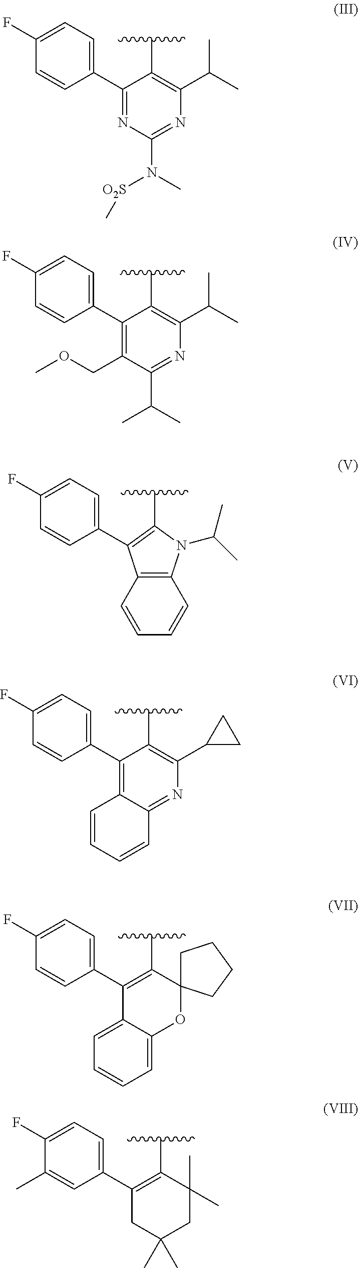 [SMILES: *c1c(-c2ccc(F)cc2)nc(N(C)S(C)(=O)=O)nc1C(C)C ; *c1c(C(C)C)nc(C(C)C)c(COC)c1-c1ccc(F)cc1 ; *c1c(-c2ccc(F)cc2)c2ccccc2n1C(C)C ; *c1c(C2CC2)nc2ccccc2c1-c1ccc(F)cc1 ; *C1=C(c2ccc(F)cc2)c2ccccc2OC12CCCC2 ; *C1=C(c2ccc(F)c(C)c2)CC(C)(C)CC1(C)C]

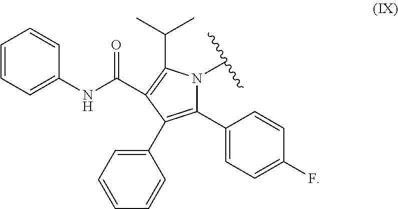 [SMILES: *n1c(-c2ccc(F)cc2)c(-c2ccccc2)c(C(=O)Nc2ccccc2)c1C(C)C]

3. The process for preparing a compound of formula (I) according to claim 1, in which
R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, or NR³CO(CH₂)ₙCH₃, optionally substituted mono- or bicyclic aryl, heterocyclic or alicyclic group; and
R₂ independently from R₁ denotes H, (CH₂)ₘ—CH₃, or aryl;
or both of R₁ and R₂ denote X, OH or O(CH₂)ₙCH₃;
or R₁ and R₂ together denote ═O, or together form a ring —(CH₂)ₚ—, —(CH₂)ᵣ-(1,2-arylene)-(CH₂)ₛ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³; wherein
R³ and R⁴ independently from each other, or together, denote H, (CH₂)ₘ—CH₃, or together form a ring —(CH₂)ₚ—, —(CH₂)ᵣ-(1,2-arylene)-(CH₂)ₛ—, —(CO)ᵣ-(1,2-arylene)-(CO)ₛ—;
X denotes F, Cl, Br or I;
n represents an integer from 0 to 10;
m represents an integer from 0 to 3;
p represents an integer from 2 to 6;
and at least one from r and s is 1.

4. The process according to claim 1, wherein the enzyme capable of catalyzing oxidation or dehydrogenation is an aldose dehydrogenase.

5. The process according to claim 1, wherein the enzyme capable of catalyzing oxidation or dehydrogenation is a pyrroloquinoline quinine (PQQ) dependent dehydrogenase.

6. The process according to claim 1, wherein the aldose dehydrogenase enzyme is YliI aldose dehydrogenase or mGDH glucose dehydrogenase.

7. The process according to claim 1, wherein the enzyme capable of catalyzing oxidation or dehydrogenation is selected from the group consisting of dehydrogenases encoded by dehydrogenase-encoding genes comprised within, or constituted by, any one of nucleotide sequences of SEQ ID NOS. 01, 03, 05, 07, 09, 23, 25, 27, 29, 31, 33, 35, 37, 39, 41, 43, 45, 47, 49, 51, 53, 55, 57, 59 and 61; or dehydrogenases defined by any one of amino acid sequences comprised within, or constituted by, SEQ ID NOS. 02, 04, 06, 08, 10, 24, 26, 28, 30, 32, 34, 36, 38, 40, 42, 44, 46, 48, 50, 52, 54, 56, 58, 60 and 62;
or any dehydrogenase having a nucleotide sequence identity or an amino acid sequence identity respectively of at least 50% to said sequences, provided that the resulting sequence variants maintain dehydrogenase activity.

8. The process according to claim 1, wherein 2-deoxyribose-5-phosphate aldolase (DERA, EC 4.1.2.4) enzyme is used for preparing the compound of formula (II).

9. The process according to claim 8, wherein the 2-deoxyribose-5-phosphate aldolase enzyme is used for a synthetic step preceding, or alternatively simultaneously at least in an overlapping time period with, bringing in contact the compound of formula (II) with the enzyme capable of catalyzing oxidation or dehydrogenation.

10. The process according to claim 1, wherein the enzyme capable of catalyzing oxidation or dehydrogenation, optionally also the DERA enzyme independently, are comprised within living whole cell, inactivated whole cell, homogenized whole cell, or cell free extract; or are purified, immobilized and/or are in the form of an extracellularly expressed protein.

11. A reaction system comprising a 2-deoxyribose-5-phosphate aldolase (DERA) enzyme and an enzyme capable of catalyzing oxidation or dehydrogenation, and the reaction system being capable of, or being arranged for, converting a compound of formula (IX), 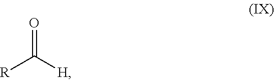
in which R denotes R₁—CH—R₂ moiety of formula (I), with acetaldehyde into a compound of formula (I) 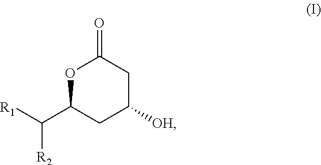
wherein in formula (IX) and formula (I), R₁ and R₂ are as defined as follows: R₁ independently from R₂ denotes H, X, N₃, CN, NO₂, OH, (CH₂)ₙ—CH₃, O—(CH₂)ₙ—CH₃, S—(CH₂)ₙ—CH₃, NR³R⁴, OCO(CH₂)ₙCH₃, NR³CO(CH₂)ₙCH₃, CH₂—R⁵, optionally substituted mono- or bicyclic aryl, heterocyclic or alicyclic group; and
R₂ independently from R₁ denotes H, (CH₂)ₘ—CH₃, or aryl;
or both of R₁ and R₂ denote either X, OH or O((CH₂)ₙCH₃);
or R₁ and R₂ together denote ═O, ═CH—R⁵, or together form a ring —(CH₂)ₚ—, —(CH₂)ᵣ-(1,2-arylene)-(CH₂)ₛ—, wherein
any one of CH₂ or CH₃ groups denoted above may optionally be further substituted by X, N₃, CN, NO₂, OH, (CH₂)ₙ—CH₃, aryl, O—(CH₂)ₙ—CH₃, OCO(CH₂)ₙCH₃, NR³R⁴, NR³CO(CH₂)ₙCH₃; or
each CH₂ linking carbon atoms can be replaced by O, S or NR³.

12. The process according to claim 8, wherein both DERA and the enzyme capable of catalyzing oxidation or dehydrogenation, are expressed by one or more cells, wherein the type of cell is selected from the group consisting of bacteria, yeast, insect cell and mammalian cells.

13. The process according to claim 8, further providing for the presence of Pyrroloquinoline quinine (PQQ).

14. The process according to claim 13, wherein providing for the presence of Pyrroloquinoline quinine (PQQ) is accomplished by a measure selected from the group consisting of:
(i) PQQ is added from externally;
(ii) a host organism is used which, beyond providing for the presence of dehydrogenase activity, further has intrinsic PQQ biosynthetic capability; and
(iii) a microorganism is used, which does not have intrinsic capability of biosynthesis of PQQ, but which is genetically engineered to express PQQ-synthesis related gene cluster.

15. The process according to claim 14, wherein the microorganism used according to measure (iii) is genetically engineered to provide for an expression of a PQQ-synthesis encoding gene comprised within, or constituted by, any one of the nucleotide sequences of SEQ ID NOS. 11, 17, 63, 64, 65, 66, 67, 68, 69, 70; or expression of a PQQ-synthesis gene encoding any one of the amino acid sequence of SEQ ID NOS. 12, 13, 14, 15, 16, 18, 19, 20, 21 and 22; or is genetically engineered to provide for expression of a PQQ-synthesis encoding gene having a nucleotide sequence identity or an amino acid sequence identity respectively of at least 50% to said sequences, provided that the resulting sequence variants maintain activity to produce PQQ.

16. The process according to claim 1, further comprising subjecting said compound (I) to conditions sufficient to prepare a statin or a pharmaceutically acceptable salt thereof, optionally salifying, esterifying or stereoselectively resolving the statin product.

17. The process according to claim 16, wherein the statin is selected from the group consisting of lovastatin, pravastatin, simvastatin, atorvastatin, cerivastatin, rosuvastatin, fluvastatin, pitavastatin, bervastatin, and dalvastatin, and pharmaceutically acceptable salts thereof.

18. A process for preparing a pharmaceutical composition, the process comprising carrying out a process according to the process of claim 16, and formulating said statin, or a pharmaceutically acceptable salt thereof, with at least one pharmaceutically acceptable excipient to obtain said pharmaceutical composition.

19. An expression system capable of translating 2-deoxyribose-5-phosphate aldolase (DERA) enzyme and an enzyme capable of catalyzing oxidation or dehydrogenation, and overexpressing both of the genes needed for said translation.

20. An expression system capable of translating 2-deoxyribose-5-phosphate aldolase (DERA) enzyme and an enzyme capable of catalyzing oxidation or dehydrogenation, wherein said translation is arranged in one or more cell types, the respective cell type(s) being genetically engineered to express, in the totality of cell type(s), both said 2-deoxyribose-5-phosphate aldolase (DERA) enzyme and said enzyme capable of catalyzing oxidation or dehydrogenation.

21. The expression system according to claim 19, wherein the enzyme capable of catalyzing oxidation or dehydrogenation is selected from the group consisting of dehydrogenases encoded by dehydrogenase-encoding genes comprised within, or constituted by, any one of nucleotide sequences of SEQ ID NOS. 01, 03, 05, 07, 09, 23, 25, 27, 29, 31, 33, 35, 37, 39, 41, 43, 45, 47, 49, 51, 53, 55, 57, 59 and 61; or dehydrogenases defined by any one of amino acid sequences comprised within, or constituted by, SEQ ID NOS. 02, 04, 06, 08, 10, 24, 26, 28, 30, 32, 34, 36, 38, 40, 42, 44, 46, 48, 50, 52, 54, 56, 58, 60 and 62; or any dehydrogenase having nucleotide sequence identity or an amino acid sequence identity respectively of at least 50% to said sequences provided that the resulting sequence variants maintain dehydrogenase activity.

22. The expression system according to claim 19, further providing for an expression of a PQQ-synthesis encoding gene comprised within, or constituted by, any one of the nucleotide sequences of SEQ ID NOS. 11, 17, 63, 64, 65, 66, 67, 68, 69, 70; or expression of a PQQ-synthesis gene encoding any one of the amino acid sequence of SEQ ID NOS. 12, 13, 14, 15, 16, 18, 19, 20, 21 and 22; or by expression of a PQQ-synthesis encoding gene having a nucleotide sequence identity or an amino acid sequence identity respectively of at least 50% to said sequences, provided that the resulting sequence variants maintain activity to produce PQQ.

23. (canceled)

24. A method of preparing a synthetic API or intermediate thereof, the method comprising subjecting a substrate compound to oxidation by an enzyme capable of catalyzing oxidation or dehydrogenation, the substrate compound being a non-natural compound selected from the group consisting of substituted or unsubstituted dideoxyaldose sugars, synthetic non-natural alcohols, esters further hydroxylated and lactols further hydroxylated.

25.-26. (canceled)

27. The reaction system according to claim 11, wherein both DERA and the enzyme capable of catalyzing oxidation or dehydrogenation, are expressed by one or more cells, wherein the type of cell is selected from the group consisting of bacteria, yeast, insect cell and mammalian cells.

28. The reaction system according to claim 11, further providing for the presence of Pyrroloquinoline quinine (PQQ).

29. The reaction system according to claim 28, wherein providing for the presence of Pyrroloquinoline quinine (PQQ) is accomplished by a measure selected from the group consisting of:
(i) PQQ is added from externally;
(ii) a host organism is used which, beyond providing for the presence of dehydrogenase activity, further has intrinsic PQQ biosynthetic capability; and
(iii) a microorganism is used, which does not have intrinsic capability of biosynthetis of PQQ, but which is genetically engineered to express PQQ-synthesis related gene cluster.

30. The reaction system according to claim 29, wherein the microorganism used according to measure (iii) is genetically engineered to provide for an expression of a PQQ-synthesis encoding gene comprised within, or constituted by, any one of the nucleotide sequences of SEQ ID NOS. 11, 17, 63, 64, 65, 66, 67, 68, 69, 70; or expression of a PQQ-synthesis gene encoding any one of the amino acid sequence of SEQ ID NOS. 12, 13, 14, 15, 16, 18, 19, 20, 21 and 22; or is genetically engineered to provide for expression of a PQQ-synthesis encoding gene having a nucleotide sequence identity or an amino acid sequence identity respectively of at least 50% to said sequences, provided that the resulting sequence variants maintain activity to produce PQQ.


SEQUENCE LISTING (82 sequences)

SEQ ID NO: 1 - DNA, 1134 bp, Artificial
  GDH 01; Sequence based on nucleotide sequence encoding aldose sugar dehydrogenase YliI (originates from Escherichia coli)
gcgccatatgcatcgacaatcctttttccttgtgccccttatttgtctttcttccgctctctgggcggctcctgcaacggtaaatgtcgaagtactgcaagacaaactcgaccatccctgggcactggcctttttacccgataatcacggtatgttaatcactctgcgcggcggcgagttgcgtcactggcaagcaggaaaaggattatctgcgccgctttccggagttccggacgtttgggcgcacgggcagggcggcctgctggacgtggttttagcgcctgattttgctcagtctcgccgcatctggttaagttattccgaagttggcgatgatggcaaagccggaactgctgtgggttatggccgcttaagtgatgatctctcaaaagtgaccgacttccgcaccgtcttccgccagatgccaaaactgtctaccggcaaccattttggcgggcggctggtattcgacggtaaaggttatctttttattgctctgggcgaaaacaatcagcgcccgacggcgcaggatctggataaattacagggcaaactggtgcgtctgaccgaccagggcgaaatcccggatgataatccttttataaaggaatccggtgtgcgcgccgagatctggtcttatggcattcgtaatccgcaaggaatggcgatgaatccgtggagtaatgcactgtggctgaatgaacatggcccgcgcggtggtgatgaaattaatatcccgcaaaaaggcaaaaactacggctggccgctggcaacctggggaatcaactattcaggctttaagataccggaagcgaaaggggagatcgtcgccgggaccgagcaacctgttttttactggaaagattcgcccgctgtgagcggcatggccttctataacagcgataaattcccccagtggcagcaaaaattatttattggcgcgctgaaagataaagatgtcattgtgatgagcgtcaacggcgacaaagtgacagaagatggccgtattttaacggacagagggcagcgaattcgtgatgttcgcactggacccgacggttatttatacgttctcaccgacgagtccagtggggaattacttaaagttagcccacgcaattagcctgagcgcgc

SEQ ID NO: 2 - protein, 371 aa, Escherichia coli
MHRQSFFLVPLICLSSALWAAPATVNVEVLQDKLDHPWALAFLPDNHGMLITLRGGELRHWQAGKGLSAPLSGVPDVWAHGQGGLLDVVLAPDFAQSRRIWLSYSEVGDDGKAGTAVGYGRLSDDLSKVTDFRTVFRQMPKLSTGNHFGGRLVFDGKGYLFIALGENNQRPTAQDLDKLQGKLVRLTDQGEIPDDNPFIKESGVRAEIWSYGIRNPQGMAMNPWSNALWLNEHGPRGGDEINIPQKGKNYGWPLATWGINYSGFKIPEAKGEIVAGTEQPVFYWKDSPAVSGMAFYNSDKFPQWQQKLFIGALKDKDVIVMSVNGDKVTEDGRILTDRGQRIRDVRTGPDGYLYVLTDESSGELLKVSPRN

SEQ ID NO: 3 - DNA, 2416 bp, Artificial
  GDH 02; Sequence based on nucleotide sequence encoding membrane bound glucose dehydrogenase Gcd (originates from Escherichia coli)
gcgccatatggcaattaacaatacaggctcgcgacgattactcgtcacgctaacagccctttttgcagcgctttgcgggctgtatctactcattggcggaggctggctggtcgcgattggcggctcctggtactaccctatcgctggccttgtgatgctcggcgtcgcctggatgctgtggcgcagtaaacgcgccgcgctttggctatacgcagccctgctgctcggcaccatgatttggggcgtctgggaagttggtttcgacttctgggcgctgactccgcgcagcgacattctggtcttcttcggcatctggctgatcctgccgtttgtctggcgtcgcctggtcattcctgccagcggcgcagttgccgcactggtggtcgcactgctgattagcggtggtatcctgacctgggccggatttaacgatccgcaggagatcaacggcaccttaagcgccgatgccacacctgctgaagctatctcccccgtagccgatcaggactggcctgcctatggtcgtaatcaggaaggtcaacgcttttcgccgctgaaacaaattaacgccgataacgtccataatctgaaagaagcctgggtgttccgtactggcgatgtgaagcagccgaacgatccgggtgaaatcaccaatgaagtgacgccgattaaagtgggcgacaccctttacctgtgtaccgctcaccagcgcctgtttgcgcttgatgccgccagcggcaaagagaaatggcattacgatcctgagctgaaaaccaacgagtctttccagcacgtaacctgccgtggtgtctcttatcatgaagccaaagcagaaaccgcttcgccggaagtgatggcggattgcccgcgtcgtatcattcttccggtcaatgatggtcgactgattgcgattaacgctgaaaacggcaaactgtgcgaaaccttcgccaataaaggcgtgctcaatctgcaaagcaatatgccagacaccaaaccgggtctgtatgaaccgacttcgccaccgattatcaccgataaaaccatcgtgatggccggttcagttaccgataacttctcaacccgcgaaacgtctggcgtgatccgtggttttgatgtcaacaccggggagctgctgtgggcttttgatcccggcgcgaaagatccgaacgcaatcccgtctgacgaacacacctttacctttaactcgccaaactcctgggcaccagcggcctatgacgcgaagctggatctggtctatctgccgatgggcgtgaccacgccggatatctggggcggtaaccgcacaccggaacaggaacgttatgccagctcgattctggcgctgaatgccactaccgggaaactggcgtggagctaccagaccgttcaccacgacctgtgggacatggatcttccggcacagccgacgctggcggacatcaccgttaatggtcagaaagtgccagttatttacgctccggcgaaaaccggcaacatttttgtgctcgatcgtcgtaatggcgaactggtggttccggcaccggaaaaaccggttccccaaggtgcagcgaaaggcgattacgtaaccccaactcaaccgttttctgaactgagcttccgtccgacgaaagatttgagcggtgcggatatgtggggagccaccatgtttgaccaactggtgtgccgcgtgatgttccaccagatgcgctatgaaggcattttcaccccgccatctgaacagggtacgctggtcttcccgggtaacctggggatgttcgaatggggcgggatttccgttgatccaaatcgtgaagtggcgattgccaacccaatggcactgccgtttgtttcgaaactgatcccgcgtggtcctggcaacccgatggagcagccgaaagatgccaaaggcacgggtacggaatccggcattcagccacagtacggtgtaccgtatggtgtcacgctcaacccgttcctctcaccatttggtctgccatgtaaacagccagcatggggttatatctcggcgctggatctgaaaactaatgaagtggtgtggaagaaacgtattggtacgccgcaggacagtatgccgttcccgatgccggttccggtgccgttcaatatgggtatgccgatgctgggcgggccaatctccacggcgggtaacgtgctgtttatcgccgctacggcagataactacctgcgcgcttacaacatgagcaacggtgaaaaactgtggcagggtcgtttaccagcgggtggtcaggctacgccaatgacctatgaagtgaatggtaagcagtatgtggtgatctccgcaggcggtcacggttcatttggtacgaagatgggcgactatattgtggcttatgcgctgccggatgatgtgaagtaagacttgcgctgagcgcgc

SEQ ID NO: 4 - protein, 796 aa, Escherichia coli
MAINNTGSRRLLVTLTALFAALCGLYLLIGGGWLVAIGGSWYYPIAGLVMLGVAWMLWRSKRAALWLYAALLLGTMIWGVWEVGFDFWALTPRSDILVFFGIWLILPFVWRRLVIPASGAVAALVVALLISGGILTWAGFNDPQEINGTLSADATPAEAISPVADQDWPAYGRNQEGQRFSPLKQINADNVHNLKEAWVFRTGDVKQPNDPGEITNEVTPIKVGDTLYLCTAHQRLFALDAASGKEKWHYDPELKTNESFQHVTCRGVSYHEAKAETASPEVMADCPRRIILPVNDGRLIAINAENGKLCETFANKGVLNLQSNMPDTKPGLYEPTSPPIITDKTIVMAGSVTDNFSTRETSGVIRGFDVNTGELLWAFDPGAKDPNAIPSDEHTFTFNSPNSWAPAAYDAKLDLVYLPMGVTTPDIWGGNRTPEQERYASSILALNATTGKLAWSYQTVHHDLWDMDLPAQPTLADITVNGQKVPVIYAPAKTGNIFVLDRRNGELVVPAPEKPVPQGAAKGDYVTPTQPFSELSFRPTKDLSGADMWGATMFDQLVCRVMFHQMRYEGIFTPPSEQGTLVFPGNLGMFEWGGISVDPNREVAIANPMALPFVSKLIPRGPGNPMEQPKDAKGTGTESGIQPQYGVPYGVTLNPFLSPFGLPCKQPAWGYISALDLKTNEVVWKKRIGTPQDSMPFPMPVPVPFNMGMPMLGGPISTAGNVLFIAATADNYLRAYNMSNGEKLWQGRLPAGGQATPMTYEVNGKQYVVISAGGHGSFGTKMGDYIVAYALPDDVK

SEQ ID NO: 5 - DNA, 1450 bp, Artificial
  GDH 03; Nucleotide sequence based on sequence encoding gene gdhB (originates from Acinetobacter calcoaceticus)
atggacaaacacctgctggcgaaaatcgcgctgctgtctgcggttcagctggttaccctgtctgcgttcgcggacgttccgctgaccccgtctcagttcgcgaaagcgaaatctgaaaacttcgacaaaaaagttatcctgtctaacctgaacaaaccgcacgcgctgctgtggggtccggacaaccagatctggctgaccgaacgtgcgaccggtaaaatcctgcgtgttaacccggaatctggttctgttaaaaccgttttccaggttccggaaatcgttaacgacgcggacggtcagaacggtctgctgggtttcgcgttccacccggacttcaaaaacaacccgtacatctacatctctggtaccttcaaaaacccgaaatctaccgacaaagaactgccgaaccagaccatcatccgtcgttacacctacaacaaatctaccgacaccctggaaaaaccggttgacctgctggcgggtctgccgtcttctaaagaccaccagtctggtcgtctggttatcggtccggaccagaaaatctactacaccatcggtgaccagggtcgtaaccagctggcgtacctgttcctgccgaaccaggcgcagcacaccccgacccagcaggaactgaacggtaaagactaccacacctacatgggtaaagttctgcgtctgaacctggacggttctatcccgaaagacaacccgtctttcaacggtgttgtttctcacatctacaccctgggtcaccgtaacccgcagggtctggcgttcaccccgaacggtaaactgctgcagtctgaacagggtccgaactctgacgacgaaatcaacctgatcgttaaaggtggtaactacggttggccgaacgttgcgggttacaaagacgactctggttacgcgtacgcgaactactctgcggcggcgaacaaatctatcaaagacctggcgcagaacggtgttaaagttgcggcgggtgttccggttaccaaagaatctgaatggaccggtaaaaacttcgttccgccgctgaaaaccctgtacaccgttcaggacacctacaactacaacgacccgacctgcggtgaaatgacctacatctgctggccgaccgttgcgccgtcttctgcgtacgtttacaaaggtggtaaaaaagcgatcaccggttgggaaaacaccctgctggttccgtctctgaaacgtggtgttatcttccgtatcaaactggacccgacctactctaccacctacgacgacgcggttccgatgttcaaatctaacaaccgttaccgtgacgttatcgcgtctccggacggtaacgttctgtacgttctgaccgacaccgcgggtaacgttcagaaagacgacggttctgttaccaacaccctggaaaacccgggttctctgatcaaattcacctacaaagcgaaataagctcagcaagctt

SEQ ID NO: 6 - protein, 478 aa, Artificial
  GDH 03;  Amino acid sequence of protein based on GdhB protein (originates from Acinetobacter calcoaceticus)
MDKHLLAKIALLSAVQLVTLSAFADVPLTPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKSIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 7 - DNA, 1450 bp, Artificial
  GDH 04; Nucleotide sequence based on sequence encoding gene gdhB originating from Acinetobacter calcoaceticus
atggacaaacacctgctggcgaaaatcgcgctgctgtctgcggttcagctggttaccctgtctgcgttcgcggacgttccgctgaccccgtctcagttcgcgaaagcgaaatctgaaaacttcgacaaaaaagttatcctgtctaacctgaacaaaccgcacgcgctgctgtggggtccggacaaccagatctggctgaccgaacgtgcgaccggtaaaatcctgcgtgttaacccggaatctggttctgttaaaaccgttttccaggttccggaaatcgttaacgacgcggacggtcagaacggtctgctgggtttcgcgttccacccggacttcaaaaacaacccgtacatctacatctctggtaccttcaaaaacccgaaatctaccgacaaagaactgccgaaccagaccatcatccgtcgttacacctacaacaaatctaccgacaccctggaaaaaccggttgacctgctggcgggtctgccgtcttctaaagaccaccagtctggtcgtctggttatcggtccggaccagaaaatctactacaccatcggtgaccagggtcgtaaccagctggcgtacctgttcctgccgaaccaggcgcagcacaccccgacccagcaggaactgaacggtaaagactaccacacctacatgggtaaagttctgcgtctgaacctggacggtaaaatcccgaaagacaacccgtctttcaacggtgttgtttctcacatctacaccctgggtcaccgtaacccgcagggtctggcgttcaccccgaacggtaaactgctgcagtctgaacagggtccgaactctgacgacgaaatcaacctgatcgttaaaggtggtaactacggttggccgaacgttgcgggttacaaagacgactctggttacgcgtacgcgaactactctgcggcggcgaacaaatctatcaaagacctggcgcagaacggtgttaaagttgcggcgggtgttccggttaccaaagaatctgaatggaccggtaaaaacttcgttccgccgctgaaaaccctgtacaccgttcaggacacctacaactacaacgacccgacctgcggtgaaatgacctacatctgctggccgaccgttgcgccgtcttctgcgtacgtttacaaaggtggtaaaaaagcgatcaccggttgggaaaacaccctgctggttccgtctctgaaacgtggtgttatcttccgtatcaaactggacccgacctactctaccacctacgacgacgcggttccgatgttcaaatctaacaaccgttaccgtgacgttatcgcgtctccggacggtaacgttctgtacgttctgaccgacaccgcgggtaacgttcagaaagacgacggttctgttaccaacaccctggaaaacccgggttctctgatcaaattcacctacaaagcgaaataagctcagcaagctt

SEQ ID NO: 8 - protein, 478 aa, Artificial
  GDH 04; Amino acid sequence of protein based on GdhB protein (originates from Acinetobacter calcoaceticus)
MDKHLLAKIALLSAVQLVTLSAFADVPLTPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGKIPKDNPSFNGVVSHIYTLGHRNPQGLAFTPNGKLLQSEQGPNSDDEINLIVKGGNYGWPNVAGYKDDSGYAYANYSAAANKSIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 9 - DNA, 799 bp, Artificial
  GDH 05; Nucleotide sequence based on gene deoC (originates from Escherichia coli)
ccggcatatgactgatctgaaagcaagcagcctgcgtgcactgaaattgatggacctgaccaccctgaatgacgacgacaccgacgagaaagtgatcgccctgtgtcatcaggccaaaactccggtcggcaataccgccgctatctgtatctatcctcgctttatcccgattgctcgcaaaactctgaaagagcagggcaccccggaaatccgtatcgctacggtaaccaacttcccacacggtaacgacgacatcgacatcgcgctggcagaaacccgtgcggcaatcgcctacggtgctgatgaagttgacgttgtgttcccgtaccgcgcgctgatggcgggtaacgagcaggttggttttgacctggtgaaagcctgtaaagaggcttgcgcggcagcgaatgtactgctgaaagtgatcatcgaaaccggcgaactgaaagacgaagcgctgatccgtaaagcgtctgaaatctccatcaaagcgggtgcggacttcatcaaaacctctaccggtaaagtggctgtgaacgcgacgccggaaagcgcgcgcatcatgatggaagtgatccgtgatatgggcgtagaaaaaaccgttggtttcaaaccggcgggcggcgtgcgtactgcggaagatgcgcagaaatatctcgccattgcagatgaactgttcggtgctgactgggcagatgcgcgtcactaccgctttggcgcttccagcctgctggcaagcctgctgaaagcgctgggtcacggcgacggtaagagcgccagcagctactaatgagctgagcgg

SEQ ID NO: 10 - protein, 259 aa, Escherichia coli
MTDLKASSLRALKLMDLTTLNDDDTDEKVIALCHQAKTPVGNTAAICIYPRFIPIARKTLKEQGTPEIRIATVTNFPHGNDDIDIALAETRAAIAYGADEVDVVFPYRALMAGNEQVGFDLVKACKEACAAANVLLKVIIETGELKDEALIRKASEISIKAGADFIKTSTGKVAVNATPESARIMMEVIRDMGVEKTVGFKPAGGVRTAEDAQKYLAIADELFGADWADARHYRFGASSLLASLLKALGHGDGKSASSY

SEQ ID NO: 11 - DNA, 3362 bp, Artificial
  Nucleotide sequence based on the pqqABCDE operon (originates from Gluconobacter oxydans)
cgcgggtacctcgcatctgccggttatcggagaccaaggagaaaaatcatggcctggaacacaccgaaagttaccgaaatcccgctgggcgcagaaatcaactcgtatgtctgcggcgagaagaaataagccgctttcccggggacccgtccttgaggaataatggcacggccgctcccccatggagcggccgttttcgttcatgggtgctctgtggtgccccagtcagacggtttgtgaaaaaatgattgatgtcatcgtgcttggcgcggcggcagggggcggttttccgcagtggaactccgcagcacccggctgtgtggccgcccgcacgcgacagggcgcgaaagcccggacccaggcctcccttgccgtcagtgccgacggaaagcgctggttcattctcaacgcctcgcccgatctgcggcagcagatcatcgatacgccggccctgcatcatcagggcagcctgcgtggaacgcccattcagggcgtcgtcctgacctgcggcgagatcgacgccataaccgggcttctgaccctgcgtgagcgtgagccttttaccctgatgggcagcgactcgacccttcagcagcttgcggacaatccgatcttcggtgcgctcgatccggaaatcgtcccacgtgttccgctcattctcgatgaagccacgtccctgatgaacaaggacgggattccgtccggtcttttgctcacggccttcgccgttccgggcaaggcgccgctttacgcggaagccgcagggtcacgcccggacgagacgctgggcctttccattacggatggatgcaagacgatgctcttcattcccggctgtgcgcagatcacgtcggaaatcgtggaacgggtagcggcagccgatctcgtgttctttgacgggacactgtggcgggatgacgaaatgatccgcgccgggttgagcccgaagagcggacagcggatgggacatgtgtccgtgaatgatgccgggggaccggtcgaatgtttcacgacatgcgaaaaaccccgtaaagtgttgattcatatcaacaactccaatccaattctgttcgaagacagccccgaacgcaaagacgtcgaacgcgccggatggacggttgcggaagacggcatgactttcagactggacacaccatgacgctcctcacacctgaccagcttgaagcacagcttcgccagatcggggccgagcggtatcacaaccggcacccgttccatcgcaagctgcatgacggcaagctggacaaggcacaggttcaggcttgggcgctgaaccgctattattatcaggcccgcatcccggcgaaggatgcgacgcttctcgcacgtctgccgacggccgaactgcgccgcgaatggcgtcgccggatcgaggaccatgacggcacggagcccggaacgggcggtgttgcgcgctggctgatgctgacggatggtctggggctggaccgggattatgtggaaagcctcgatggtctgcttccagccacgcgcttctcggtcgatgcctatgtgaacttcgtgcgggaccagtcgattctggcggccattgcgtcgtcgctgacggaactgttttcgcccacgatcatcagcgagcgcgtctcggggatgctgcggcactacgactttgtgtcggaaaagacgctggcctatttcacgccgcgcctgacgcaggccccgcgggattccgatttcgcgctggcctatgtccgcgaaaaggcccgcacgccggagcagcagaaagaagtcctgggagcgctggagttcaagtgctccgtgctgtggacgatgctggatgcgctcgactacgcctatgtggaaggccacattccgccgggggctttcgttccatgacggaggccccgcatgtcgtggcggaggggacggttctctcctttgcccgggggcatcgtctccagcacgatcgtgtgcgggacgtgtggatcgtgcaggcgcctgaaaaagcatttgtagttgagggcgccgcgccgcatattctgcggctgctggatgggaagcgcagcgtcggcgagatcatccagcagcttgcaatcgagttttccgccccgcgtgaggtcattgcgaaagatgtcctcgcgcttctttctgaactgacagaaaagaacgtcctgcacacatgacactcccttcgccgccgatgagccttctggctgaactgacgcatcgatgcccgctttcctgcccctactgctccaatccgcttgaactcgaacgcaaggcggcagaactcgacacggccacctggactgccgtactggagcaggcggccgagcttggggtgctccaggttcatttctctggcggcgagcctatggcgcggcctgatctggtcgaactggtctccgtcgcacggagactcaacctgtattccaacttgatcacgtccggcgtgttgctggacgaaccgaaactggaagctctcgacagggcggggctggatcacatccagctctctttccaagacgtgacggaggcgggagccgagcgtatcggcggtctcaagggagcgcaggcccgcaaggttgcggcggcgcggctcatccgcgcgtccggcattccgatgacgctcaattttgtggtgcacagggaaaatgtcgcccgtatccccgagatgttcgccctggcgcgggaactcggagcggggcgggtggagatcgcgcatacccagtattatggctgggggctgaaaaaccgtgaggcgcttcttcccagccgggatcagctggaggaatccacacgcgccgtggaagcggagcgcgctaagggtggtttgtccgttgattatgtgacgccggactatcatgcagaccggcccaagccctgcatggggggatggggccagcgtttcgtgaatgtcacaccttcgggccgggtcctgccgtgtcatgcagccgaaatcattccggatgtcgcattcccgaatgtgcaggatgtgaccctgtccgaaatctggaacatctcaccgctgttcaacatgttccgcgggacggactggatgccggagccctgccgctcctgcgagcgcaaggagcgtgactggggcgggtgtcgctgtcaggcgatggcgctgacggggaatgccgcgaataccgatcccgtatgcagtctctccccctatcacgatcgggtggagcaggccgtcgagaacaacatgcagccagaaagcacgttgttctacaggcgttatacgtaatcgccgtaaatattgactttgctaagatggagaaaggatgtgccgttcaggacaatcttgatatatcgagcaataatatctgtttctatctgaagaggttctccaaactctccgcccggatccgcgc

SEQ ID NO: 12 - protein, 26 aa, Gluconobacter oxydans
MAWNTPKVTEIPLGAEINSYVCGEKK

SEQ ID NO: 13 - protein, 302 aa, Gluconobacter oxydans
MIDVIVLGAAAGGGFPQWNSAAPGCVAARTRQGAKARTQASLAVSADGKRWFILNASPDLRQQIIDTPHHQGSLRGTPIQGVVLTCGEIDAITGLLTLREREPFTLMGSDSTLQQLADNPIFGALDPEIVPRVPLILDEATSLMNKDGIPSGLLLTAFAVPGKAPLYAEAAGSRPDETLGLSITDGCKTMLFIPGCAQITSEIVERVAAADLVFFDGTLWRDDEMIRAGLSPKSGQRMGHVSVNDAGGPVECFTTCEKPRKVLIHINNSNPILFEDSPERKDVERAGWTVAEDGMTFRLDTP

SEQ ID NO: 14 - protein, 239 aa, Gluconobacter oxydans
MTLLTPDQLEAQLRQIGAERYHNRHPFHRKLHDGKLDKAQVQAWALNRYYYQARIPAKDATLLARLPTAELRREWRRRIEDHDGTEPGTGGVARWLMLTDGLGLDRDYVESLDGLLPATRFSVDAYVNFVRDQSILAAIASSLTELFSPTIISERVSGMLRHYDFVSEKTLAYFTPRLTQAPRDSDFALAYVREKARTPEQQKEVLGALEFKCSVLWTMLDALDYAYVEGHIPPGAFVP

SEQ ID NO: 15 - protein, 96 aa, Gluconobacter oxydans
MTEAPHVVAEGTVLSFARGHRLQHDRVRDVWIVQAPEKAFVVEGAAPHILRLLDGKRSVGEIIQQLAIEFSAPREVIAKDVLALLSELTEKNVLHT

SEQ ID NO: 16 - protein, 358 aa, Gluconobacter oxydans
MTLPSPPMSLLAELTHRCPLSCPYCSNPLELERKAAELDTATWTAVLEQAAELGVLQVHFSGGEPMARPDLVELVSVARRLNLYSNLITSGVLLDEPKLEALDRAGLDHIQLSFQDVTEAGAERIGGLKGAQARKVAAARLIRASGIPMTLNFVVHRENVARIPEMFALARELGAGRVEIAHTQYYGWGLKNREALLPSRDQLEESTRAVEAERAKGGLSVDYVTPDYHADRPKPCMGGWGQRFVNVTPSGRVLPCHAAEIIPDVAFPNVQDVTLSEIWNISPLFNMFRGTDWMPEPCRSCERKERDWGGCRCQAMALTGNAANTDPVCSLSPYHDRVEQAVENNMQPESTLFYRRYT

SEQ ID NO: 17 - DNA, 4279 bp, Artificial
  Nucleotide sequence based on the pqqABCDE operon (originates from Kluyvera intermedia)
gcgcggtaccaagcttcgctgccgcaaaacagcagatgttccagcagatgtgccaaacccggccagcgatccgcttcatccagactgccggcctgcacgcgcatcagcgccgccgcctcacgcgcgtcgggctgatgataaagatggcagcgcaggccgttggcgagcgtgagctggcgcgcgtgcatcaggtgacgcgctgcttaaaagatcagttcggagttgctgcgattgcggaactgcagctgctggattttgatgtcgctgcagttggcttcacgtcgtgcttcgaggatcttgccgtgatgcggcgatttgctgcacaccggatcggtgttatcggcatcgccggtcagcataaaggcctgacagcgacagccgccaaagtctttcgctttttcatcgcaggagcgacagggttccggcatccagtcaaagccgcgatagcggttaaaaccaaacgagttgtaccagatatcgtccagcgtgcgctccagcaccgacgggaacgcaaccggcagctgacgcgcgctgtggcacggcaacgcggtgcagtcggcgtgacgctgaggaagatcgatccccatccgcccatgcagggtttcggccgctcttcgtagtaatccggcgtcacgaacagcaggattggtgagattgccgctggcgcccatgcgctgacgataatcggccaccaccgcttcggcgttggcgatctgctcgcgcgtcggcagcaatccttcacgattgagttgcgcccagccatagaattggcaagtcgccagctcgacatcgtcggcatccagttcgatacagagatcgatgattttgtcgatctggtcgatattgtggcgatgcagcacgaaattcagcaccatcggatagccgtgcgctttcaccgctttcgccatctccagcttctgctggaaggccttttttgacccggccagcgccgcgttcagcgtttcatcgctggcctggaagctgatctggatatgatcgagtccggcgtcggcaaaggcgtcgagttttttcgccgtcaggccgatgccggaggtgatcaggttggtgtagaaaccgagatcgcgcgcggcgcgaatcaactccggcaggtctttacgggttagcggttcgccgccggaaaaaccgagctgcacgctgcccatggcgcgcgcctgacgaaacacctcaatccactgttcggtggtcagctctttcttctgctgggaaaaatccagcggattggagcaatatggacactgcagcgggcagcgataggtcagttctgccagcagccacagcggtggcgtcacgcttttactcgggttcacgacactgtatccatttttgttcaatggcggattgcaggaactctttgacgtcgtcgccgacaccgcccgcgtccgggaaacgcgcatccagcgtggcaacgatcgccgccacatcctgtttgccatccaccagttcgaggatcgccacggcggtctcgttcagtttggccatgccttctggatagagcaccacatggctatcctgcgccgcttcccactgcatgcggtagccgcgacggaaagccggaatcacattatctttcatgttgttataccagtcttgtgctgtgccaggacacctgatcggtgaccgttatctttcatgttgttataccagtcttgtgctgtgccaggacacctgatcggtgaccgtatgataaggcggacgttgcagcgcatacgccatggtcatggcatcgagcatggtccagagaatatcgagtttaaactgcaggatctccagcatgcggttctgctgttcggcgcgggtaaacacctcaagcgccagcgccagcccatgctccacatcgcggttcgcctggctcaaacggctgcggaagtagaaatagccttcctctttgatccacggatagtgctgcggccagctgtcgagacgcgactgatgaatctgcggcgcaaacagctcggtgagcgagctacaggccgcttcctgccagttagcacggcgcgcgaagttcacgtaggcgtccaccgcaaaacgcacgcccggcagcaccagcttctctgacagcaacacgtcacgttccagccccaccgcctcacccaactgcagccaggcttcaatcccgccttcgtgaccgttgctgccgtcgtgatcgaggatgcgctgcacccatttacgccgcgtcgccgcatccgggcaattcgccatgatcgccgcatctttcagcggaatgttggtctgataatagaagcggttcgccacccagccctgaatctgctcgcgcgtcgcctcgccattgtgcatggcaatgtgatacgggtgatgtatgtggtagaacgcgcctttatcgcgcagcgcctgctcaaacgcggcgggcgataatgcttcagtgatgatcatgatgcaaaccgcgcagcgcctgctcaaacgcggcgggcgataatgcttcagtgatgatcatgatgcaaactcctgcagatgaatcgccatgccatcccaactcacttcaatgccctgctgcgtcaaataggcgcgctgcggggattgctcattaaggatcggattggtgttgttaatgtgaatcaaaatcttgcgtttggccggcagcgcagccagcaacgccattaaaccttgttcttcggccagcgctaagtggcccatcgctttgccagtatttttgccaacgccggtggcgagcagctcgtcgtcctgccacaccgtgccatcaataacaggcaatcggctctttgcagccacggcatggatcaccgcatccggctcgccgagtcccggcgcgtagagcagcgtttggccattgccggtggtttcatgaacagcgccacgttgtggtccggcagggcgatcgcgatacggcgaatacggcggcgcgttgctgagaatcggaatcgcggtgaactgcaaactgtggcagacatcgacgcggaacggctccagcggcgttaacgcatgatgctgcaagccgccattccagtgctgcaacatggtgaaaatcgggaaaccgctggtgagatcgccatgcacttccggcgtgcaccacacctgatgcggacagccttcgcgcaggctcaacaagccagtggtgtgatcgatttggctgtcggtgagaatgatgctgccaatcgccgtgccgcgcagcacaccctttttgatcagttctggcgtgtgggcaatttgctggctgatgtccggcgaggcgttgcacagcacccaatcttcaccgttgtcgctgacaatgattgaagattgggtgcgcgcctgcgcctgaatactgccgttacgcaggccgctgcaattggcgcagttacagttccactgcgggaagccgccgcccgccgctgaaccgaggactttaataaacatgaacgttgaccaaagagagaaaacaaaatgcccgcacgaacggcgggcagaagtcttagcggttggagatgtacagagtcacttccaagcccagacgcaggtcaataaacgcaggttttttccacataatggtcttcctcataaaacaaaaacgcagagaaatctctgctgaagaggattgcggctgtgacttagatcacatcgcaatccgtacgcgccgcaggatgttgcgccaatgtagcgagcagatcaaccctgattcggtaaggaatggtgctaatcctgccaaatttgttgcaacacgcgctgccagttgccccatgcgagctgctcaagtgctgtttgatcgtagtcgaacgaacgcaacaacgcataaagatgcgggtaatccgctcacgtctttcaagcgcctcaggcacgctgatgccatcaaaatcggagcccagcgccacatgatcgcggcccattatcgcgatcaggtgctcaacatgtttaacaatttcgatcaatgcggtgtcgctgtcgcgttttccgtcgctgcgcagaaaagcgttgccaaaattcacgcccaccacgccgccgctgtcgcggatggcgcgcagctgatcgtcggtgagattgcgcggctgcgcgcagagcgcgtgcgcattggagtgcgtcgccaccagcggcgcggttgacaggcgcgcggtgtcccagaaggctttctcgttcatgtgcgacatatcgatcagcatgcgatggcgattggcagcggcgatcagctgctcaccttgttcggtcaagcctggcccgctgtccggtgagttggggaatgatccgctgacgccttcgccaaagcgattgggcagattccagaacggcccgatgctgcgtggatccgcgc

SEQ ID NO: 18 - protein, 23 aa, Kluyvera intermedia
MWKKPAFIDLRLGLEVTLYISNR

SEQ ID NO: 19 - protein, 307 aa, Kluyvera intermedia
MFIKVLGSAAGGGFPQWNCNCANCSGLRNGSIQAQARTQSSIIVSDNGEDWVLCNASPDISQQIAHTPELIKKGVLRGTAIGSIILTDSQIDHTTGLLSLREGCPHQVWCTPEVHGDLTSGFPIFTMLQHWNGGLQHHALTPLEPFRVDVCHSLQFTAIPILSNAPPYSPYRDRPAGPQRGAVHETTGNGQTLLYAPGLGEPDAVIHAVAAKSRLPVIDGTVWQDDELLATGVGKNTGKAMGHLALAEEQGLMALLAALPAKRKILIHINNTNPILNEQSPQRAYLTQQGIEVSWDGMAIHLQEFAS

SEQ ID NO: 20 - protein, 251 aa, Kluyvera intermedia
MIITEALSPAAFEQALRDKGAFYHIHHPYHIAMHNGEATREQIQGWVANRFYYQTNIPLKDAAIMANCPDAATRRKWVQRILDHDGSNGHEGGIEAWLQLGEAVGLERDVLLSEKLVLPGVRFAVDAYVNFARRANWQEAACSSLTELFAPQIHQSRLDSWPQHYPWIKEEGYFYFRSRLSQANRDVEHGLALALEVFTRAEQQNRMLEILQFKLDILWTMLDAMTMAYALQRPPYHTVTDQVSWHSTRLV

SEQ ID NO: 21 - protein, 92 aa, Kluyvera intermedia
MKDNVIPAFRRGYRMQWEAAQDSHVVLYPEGMAKLNETAVAILELVDGKQDVAAIVATLDARFPDAGGVGDDVKEFLQSAIEQKWIQCREPE

SEQ ID NO: 22 - protein, 374 aa, Kluyvera intermedia
VNPSKSVTPPLWLLAELTYRCPLQCPYCSNPLDFSQQKKELTTEQWIEVFRQARAMGSVQLGFSGGEPLTRKDLPELIRAARDLGFYTNLITSGIGLTAKKLDAFADAGLDHIQISFQASDETLNAALAGSKKAFQQKLEMAKAVKAHGYPMVLNFVLHRHNIDQIDKIIDLCIELDADDVELATCQFYGWAQLNREGLLPTREQIANAEAVVADYRQRMGASGNLTNPAVRDAGLLRRAAETLHGRMGIDLPQRHADCTALPCHSARQLPVAFPSVLERTLDDIWYNSFGFNRYRGFDWMPEPCRSCDEKAKDFGGCRCQAFMLTGDADNTDPVCSKSPHHGKILEARREANCSDIKIQQLQFRNRSNSELIF

SEQ ID NO: 23 - DNA, 1116 bp, Escherichia coli
atgcatcgacaatcctttttccttgtgccccttatttgtctttcttccgctctctgggcggctcctgcaacggtaaatgtcgaagtactgcaagacaaactcgaccatccctgggcactggcctttttacccgataatcacggtatgttaatcactctgcgcggcggcgagttgcgtcactggcaagcaggaaaaggattatctgcgccgctttccggagttccggacgtttgggcgcacgggcagggcggcctgctggacgtggttttagcgcctgattttgctcagtctcgccgcatctggttaagttattccgaagttggcgatgatggcaaagccggaactgctgtgggttatggccgcttaagtgatgatctctcaaaagtgaccgacttccgcaccgtctttcgccagatgccaaaactgtctaccggcaaccattttggcgggcggctggtattcgacggtaaaggttatctttttattgctctgggcgaaaacaatcagcgcccgacggcgcaggatctggataaattacagggcaaactggtgcgtctgaccgaccagggcgaaatcccggatgataatccttttataaaggaatccggtgcgcgcgccgagatctggtcttatggcattcgtaatccgcaaggaatggcgatgaatccgtggagtaatgcactgtggctgaatgaacatggcccgcgcggtggtgatgaaattaatatcccgcaaaaaggcaaaaactacggctggccgctggcaacctggggaatcaactattcaggctttaagataccggaagcgaaaggggagatcgtcgccgggaccgagcaacctgttttttactggaaagattcgcccgctgtgagcggcatggccttctataacagcgataaattcccccagtggcagcaaaaattatttattggcgcgctgaaagataaagatgtcattgtgatgagcgtcaacggcgacaaagtgacagaagatggccgtattttaacggacagagggcagcgaattcgtgatgttcgcactggacccgacggttatttatacgttctcaccgacgagtccagtggggaattacttaaagttagcccacgcaattag

SEQ ID NO: 24 - protein, 371 aa, Escherichia coli
MHRQSFFLVPLICLSSALWAAPATVNVEVLQDKLDHPWALAFLPDNHGMLITLRGGELRHWQAGKGLSAPLSGVPDVWAHGQGGLLDVVLAPDFAQSRRIWLSYSEVGDDGKAGTAVGYGRLSDDLSKVTDFRTVFRQMPKLSTGNHFGGRLVFDGKGYLFIALGENNQRPTAQDLDKLQGKLVRLTDQGEIPDDNPFIKESGARAEIWSYGIRNPQGMAMNPWSNALWLNEHGPRGGDEINIPQKGKNYGWPLATWGINYSGFKIPEAKGEIVAGTEQPVFYWKDSPAVSGMAFYNSDKFPQWQQKLFIGALKDKDVIVMSVNGDKVTEDGRILTDRGQRIRDVRTGPDGYLYVLTDESSGELLKVSPRN

SEQ ID NO: 25 - DNA, 2391 bp, Escherichia coli
ttacttcacatcatccggcagcgcataagccacaatatagtcgcccatcttcgtaccaaatgaaccgtgaccgcctgcggagatcaccacatactgcttaccattcacttcataggtcattggcgtagcctgaccacccgctggtaaacgaccctgccacagtttttcaccgttgctcatgttgtaagcgcgcaggtagttatctgccgtagcggcgataaacagcacgttacccgccgtggagattggcccgcccagcatcggcatacccatattgaacggcaccggaaccggcatcgggaacggcatactgtcctgcggcgtaccaatacgtttcttccacaccacttcattagttttcagatccagcgccgagatataaccccatgctggctgtttacatggcagaccaaatggtgagaggaacgggttgagcgtgacaccatacggtacaccgtactgtggctgaatgccggattccgtacccgtgcctttggcatctttcggctgctccatcgggttgccaggaccacgcgggatcagtttcgaaacaaacggcagtgccattgggttggcaatcgccacttcacgatttggatcaacggaaatcccgccccattcgaacatccccaggttacccgggaagaccagcgtaccctgttcagatggcggggtgaaaatgccttcatagcgcatctggtggaacatcacgcggcacaccagttggtcaaacatggtggctccccacatatccgcaccgctcaaatctttcgtcggacggaagctcagttcagaaaacggttgagttggggttacgtaatcgcctttcgctgcaccttggggaaccggtttttccggtgccggaaccaccagttcgccattacgacgatcgagcacaaaaatgttgccggttttcgccggagcgtaaataactggcactttctgaccattaacggtgatgtccgccagcgtcggctgtgccggaagatccatgtcccacaggtcgtggtgaacggtctggtagctccacgccagtttcccggtagtggcattcagcgccagaatcgagctggcataacgttcctgttccggtgtgcggttaccgccccagatatccggcgtggtcacgcccatcggcagatagaccagatccagcttcgcgtcataggccgctggtgcccaggagtttggcgagttaaaggtaaaggtgtgttcgtcagacgggattgcgttcggatctttcgcgccgggatcaaaagcccacagcagctccccggtgttgacatcaaaaccacggatcacgccagacgtttcgcgggttgagaagttatcggtaactgaaccggccatcacgatggttttatcggtgataatcggtggcgaagtcggttcatacagacccggtttggtgtctggcatattgctttgcagattgagcacgcctttattggcgaaggtttcgcacagtttgccgttttcagcgttaatcgcaatcagtcgaccatcattgaccggaagaatgatacgacgcgggcaatccgccatcacttccggcgaagcggtttctgctttggcttcatgataagagacaccacggcaggttacgtgctggaaagactcgttggttttcagctcaggatcgtaatgccatttctctttgccgctggcggcatcaagcgcaaacaggcgctggtgagcggtacacaggtaaagggtgtcgcccactttaatcggcgtcacttcattggtgatttcacccggatcgttcggctgcttcacatcgccagtacggaacacccaggcttctttcagattatggacgttatcggcgttaatttgtttcagcggcgaaaagcgttgaccttcctgattacgaccataggcaggccagtcctgatcggctacgggggagatagcttcagcaggtgtggcatcggcgcttaaggtgccgttgatctcctgcggatcgttaaatccggcccaggtcaggataccaccgctaatcagcagtgcgaccaccagtgcggcaactgcgccgctggcaggaatgaccaggcgacgccagacaaacggcaggatcagccagatgccgaagaagaccagaatgtcgctgcgcggagtcagcgcccagaagtcgaaaccaacttcccagacgccccaaatcatggtgccgagcagcagggctgcgtatagccaaagcgcggcgcgtttactgcgccacagcatccaggcgacgccgagcatcacaaggccagcgatagggtagtaccaggagccgccaatcgcgaccagccagcctccgccaatgagtagatacagcccgcaaagcgctgcaaaaagggctgttagcgtgacgagtaatcgtcgcgagcctgtattgttaattgccat

SEQ ID NO: 26 - protein, 796 aa, Escherichia coli; identical to SEQ ID NO: 4

SEQ ID NO: 27 - DNA, 2427 bp, Gluconobacter oxydans
atgagcacaatttcccggcccaggctctgggccctgataacggccgcggtattcgcgctttgcggcgcgatccttaccgttggcggcgcatgggtcgctgccatcggcggccctctttattatgtcatccttggcctggcacttctcgccacggctttcctctcattccggcgcaatccggctgccctctatctgttcgcagtcgtcgtcttcggaacggtcatctgggaactcaccgttgtcggtctcgacatctgggccctgatcccgcgctcggacatcgtcatcatcctcggcatctggctgctgctgccgttcgtctcgcgcgccagatcggtggcacgcggacgaccgtcctgccgctcgccggccgttggcgttgcggttctggccctgttcgccagcctcttcaccgacccgcatgacatcagcggcgaactgccgacgcagatcgcaaacgcctcccccgccgacccggacaacgttccggccagcgagtggcacgcttatggtcgtacgcaggccggtgaccgctggtccccgctgaaccagatcaatgcgacgaacgtcagcaacctcaaggtcgcatggcatatccacaccaaggatatgatgaactccaacgacccgggcgaagcgacgaacgaagcgacgccgatcgagttcaacaacacgctttatatgtgctcactgcatcagaagctgtttgcggttgatggtgccaccggcaacgtcaagtgggtctacgatccgaagctccagatcaaccctggcttccagcatctgacctgccgtggcgtcagcttccacgaaacgccggccaatgccatggattccgatggcaatcctgctccgacggactgcgccaaggactccatcctgccggtcaatgatggccgtctggttgaagtcgatgccgacacgggcaagacctgctccggcttcggcaacaatggcgagatcgacctgcgcgttccgaaccagccttacacgacgcctggccagtacgagccgacgtccccgccggtcatcacagacaagctgatcatcgccaacagcgccatcaccgataacggttcggtcaagcaggcttcgggcgccacgcaggcattcgacgtctacaccggcaagcgcgtctgggtgttcgatgcgtccaacccggatccgaaccagcttccggatgagagccaccctgtcttccacccgaactcgccaaactcctggatcgtgtcgtcctacgacgccaacctgaacctcgtgtacatcccgatgggcgtggggactcccgaccagtggggcggtgaccgcacgaaggattccgagcgtttcgctccgggtatcgttgcgctgaacgccgatacgggcaagctcgcctggttctaccagaccgttcatcacgatctgtgggacatggagcttccgtcccagccgagcctcgtggatgtgacacagaaggacggcacgcttgttccggccatctacgctccgaccaagaccggcgacattttcgtcctcgaccgtcgtaccggcaaggaaatcgtcccggctccggaaaccccggttccccagggtgctgctccgggtgaccacaccagcccgacccagccgatgtcgcagctgaccctgcgtccgaagaacccgctgaacgactccgatatctggggcggcacgatcttcgaccagatgttctgcagcatctatttccacaccctccgctacgaaggccccttcacgccgccgtcgctcaagggctcgctcatcttcccgggtgatctgggaatgttcgaatggggtggtctggccgtcgatccgcagcgtcaggtggctttcgccaacccgatttccctgccgttcgtctctcagcttgttccccgcggaccgggcaacccgctctggcctgaagaaaatgccaagggcacgggtggtgaaaccggcctgcagcacaactatggcatcccgtatgccgtcaacctgcatccgttcctggatccggtgctgctgccgttcggcatcaagatgccgtgccgcacgccgccctggggctatgtcgccggtattgacctgaagaccaacaaggtcgtctggcagcaccgcaacggcaccctgcgtgactcgatgtatggcagctccctgccgatcccgctgccgccgatcaagatcggtgtcccgagcctcggtggcccgctctccacggctggcaatctcggcttcctgacggcgtccatggattactacatccgtgcgtacaacctgacgacgggcaaggtgctgtggcaggaccgtctgccggctggtgctcaggcaacgccgatcacctatgccatcaacggcaagcagtacatcgtgacctatgcaggcggacacaactcgttcccgacccgcatgggcgacgacatcatcgcctacgccctgcccgatcagaaatga

SEQ ID NO: 28 - protein, 808 aa, Gluconobacter oxydans
MSTTSRPGLWALITAAVFALCGAILTVGGAWVAAIGGPLYYVILGLALLATAFLSFRRNPAALYLFAVVVFGTVIWELTVVGLDIWALIPRSDIVIILGIWLLLPFVSRQIGGTRTTVLPLAGAVGVAVLALFASLFTDPHDISGDLPTQIANASPADPDNVPASEWHAYGRTQAGDRWSPLNQINASNVSNLKVAWHIHTKDMMNSNDPGEATNEATPIEFNNTLYMCSLHQKLFAVDGATGNVKWVYDPKLQINPGFQHLTCRGVSFHETPANATDSDGNPAPTDCAKRIILPVNDGRLVEVDADTGKTCSGFGNNGEIDLRVPNQPYTTPGQYEPTSPPVITDKLIIANSAITDNGSVKQASGATQAFDVYTGKRVWVFDASNPDPNQLPDDSHPVFHPNSPNSWIVSSYDRNLNLVYIPMGVGTPDQWGGDRTKDSERFAPGIVALNADTGKLAWFYQTVHHDLWDMDVPSQPSLVDVTQKDGTLVPAIYAPTKTGDIFVLDRRTGKEIVPAPETPVPQGAAPGDHTSPTQPMSQLTLRPKNPLNDSDIWGGTIFDQMFCSIYFHTLRYEGPFTPPSLKGSLIFPGDLGMFEWGGLAVDPQRQVAFANPISLPFVSQLVPRGPGNPLWPEKDAKGTGGETGLQHNYGIPYAVNLHPFLDPVLLPFGIKMPCRTPPWGYVAGIDLKTNKVVWQHRNGTLRDSMYGSSLPIPLPPIKIGVPSLGGPLSTAGNLGFLTASMDYYIRAYNLTTGKVLWQDRLPAGAQATPITYAINGKQYIVTYAGGHNSFPTRMGDDIIAYALPDQK

SEQ ID NO: 29 - DNA, 2406 bp, Acinetobacter calcoaceticus
atgaatcaacctacttcaagatcaggtttaacgacttttaccgtaattattattgggttactggcgttattcctgttaattggaggtatttggctcgctacactaggcggttcaatttactacattatagctggagtattactcctcattgttgcatggcaactctacaagcgtgcttctactgctttgtggttttatgctgcattaatgctaggtaccattatctggagtgtctgggaagttggaacagacttttgggcgcttgcaccacgtttagatattttaggtattcttggtttatggttattggttccggctgtaactcgtggaatcaacaaccttggatcaagtaaagttgccttatcttcaactttagcaattgcaatcgtgttgatggtttattctatcttcaatgatccgcaagaaattaatggtgaaatcaaaacacctcaaccagaaacagctcaagccgtgcctggtgttgctgaaagtgattggccagcttatggtcgtactcaagcaggcgtgcgttattctccattgaaacagatcaatgatcaaaacgtaaaagacttgaaagttgcttggactttacgtactggcgatcttaagacagataacgactctggcgaaacgactaatcaggttacaccgattaaaatcggtaataacatgtttatctgtacagctcaccagcagttaattgctattgatcctgctacaggtaaagaaaaatggcgttttgatccgaaacttaaaacggataaatcgttccagcatctaacttgtcgtggtgtgatgtactacgatgcaaacaatacaactgagtttgcaacgagtcttcaaagcaagaaatctagctctacacaatgtccacgtaaggtatttgtaccagtcaacgatggccgtttagtggctgtaaatgctgacactggtaaagcatgtactgactttggtcaaaatggtcaagtgaacttacaagagttcatgccatatgcttatccaggcggttataacccgacatctcctggtatcgtgactggttcaactgttgttattgctggttctgtaacagataactactcaaataaagagccatctggtgtgattcgtggatacgacgttaacactggtaaacttctttgggtatttgacactggcgcagcggatccaaatgcaatgccgggcgaaggtactacttttgttcacaactcaccaaatgcgtgggcacctttagcatacgatgccaaacttgacatcgtttatgtaccaacaggtgtaggtacaccagacatctggggtggtgaccgtaccgagctgaaagagcgttatgcaaactcaatgttagcgattaatgcttctactggtaaattagtgtggaacttccagacaacccatcacgatttatgggatatggatgtaccatcacaaccatctttagctgatatcaaaaacaaagctggccaaactgttcctgcaatctatgtattgacgaaaacaggtaatgcctttgtgcttgatcgccgtaatggtcaaccgattgttcctgtaactgagaaaccagttccacaaacagttaaacgtggaccacaaactaaaggtgagttctattcaaaaactcagccattctctgacttgaacttggcgccacaagataaattgactgataaagacatgtggggtgccactatgcttgatcagctcatgtgtcgtgtatctttcaaacgtctaaattacgatggtatttatacgccaccatctgaaaacggtactttagttttccctggtaacttaggtgtatttgaatggggcggtatgtcagttaaccctgatcgtcaggttgctgtaatgaacccgattggtctgccattcgtcagtcgtttaattcctgctgatccaaaccgtgcacaaactgcaaaaggtgcgggaactgagcagggcgtacaaccgatgtacggcgtaccatatggtgttgaaattagcgcattcttatctccgcttggtttaccttgtaaacaaccggcttggggctatgtagctggcgttgatttgaaaactcatgaagtggtatggaaaaaacgtattggtacaatccgtgacagtttaccgaacttgttccagttgccagctgtgaaaattggtgtgcctggtttaggtggttcaatttctactgccggtaatgtcatgtttgttggtgcaactcaagataactacttacgtgcgtttaacgttactaacggtaagaaactttgggaagcgcgtttaccagcaggtggacaagcaacaccaatgacttatgaaatcaatggtaagcaatatgttgtaatcatggctggtggtcatggttcatttggtacgaaaatgggcgactatttagtggcttatgccttaccagataacaaataa

SEQ ID NO: 30 - protein, 801 aa, Acinetobacter calcoaceticus
MNQPTSRSGLTTFTVIIIGLLALFLLIGGIWLATLGGSIYYIIAGVLLLIVAWQLYKRASTALWFYAALMLGTIIWSVWEVGTDFWALAPRLDILGILGLWLLVPAVTRGINNLGSSKVALSSTLAIAIVLMVYSIFNDPQEINGEIKTPQPETAQAVPGVAESDWPAYGRTQAGVRYSPLKQINDQNVKDLKVAWTLRTGDLKTDNDSGETTNQVTPIKIGNNMFICTAHQQLIAIDPATGKEKWRFDPKLKTDKSFQHLTCRGVMYYDANNTTEFATSLQSKKSSSTQCPRKVFVPVNDGRLVAVNADTGKACTDFGQNGQVNLQEFMPYAYPGGYNPTSPGIVTGSTVVIAGSVTDNYSNKEPSGVIRGYDVNTGKLLWVFDTGAADPNAMPGEGTTFVHNSPNAWAPLAYDAKLDIVYVPTGVGTPDIWGGDRTELKERYANSMLAINASTGKLVWNFQTTHHDLWDMDVPSQPSLADIKNKAGQTVPAIYVLTKTGNAFVLDRRNGQPIVPVTEKPVPQTVKRGPQTKGEFYSKTQPFSDLNLAPQDKLTDKDMWGATMLDQLMCRVSFKRLNYDGIYTPPSENGTLVFPGNLGVFEWGGMSVNPDRQVAVMNPIGLPFVSRLIPADPNRAQTAKGAGTEQGVQPMYGVPYGVEISAFLSPLGLPCKQPAWGYVAGVDLKTHEVVWKKRIGTIRDSLPNLFQLPAVKIGVPGLGGSISTAGNVMFVGATQDNYLRAFNVTNGKKLWEARLPAGGQATPMTYEINGKQYVVIMAGGHGSFGTKMGDYLVAYALPDNK

SEQ ID NO: 31 - DNA, 1437 bp, Acinetobacter calcoaceticus
atgaataaacatttattggctaaaattgctttattaagcgctgttcagctagttacactctcagcatttgctgatgttcctctaactccatctcaatttgctaaagcgaaatcagagaactttgacaagaaagttattctatctaatctaaataagccgcatgctttgttatggggaccagataatcaaatttggttaactgagcgagcaacaggtaagattctaagagttaatccagagtcgggtagtgtaaaaacagtttttcaggtaccagagattgtcaatgatgctgatgggcagaatggtttattaggttttgccttccatcctgattttaaaaataatccttatatctatatttcaggtacatttaaaaatccgaaatctacagataaagaattaccgaaccaaacgattattcgtcgttatacctataataaatcaacagatacgctcgagaagccagtcgatttattagcaggattaccttcatcaaaagaccatcagtcaggtcgtcttgtcattgggccagatcaaaagatttattatacgattggtgaccaagggcgtaaccagcttgcttatttgttcttgccaaatcaagcacaacatacgccaactcaacaagaactgaatggtaaagactatcacacctatatgggtaaagtactacgcttaaatcttgatggaagtattccaaaggataatccaagttttaacggggtggttagccatatttatacacttggacatcgtaatccgcagggcttagcattcactccaaatggtaaattattgcagtctgaacaaggcccaaactctgacgatgaaattaacctcattgtcaaaggtggcaattatggttggccgaatgtagcaggttataaagatgatagtggctatgcttatgcaaattattcagcagcagccaataagtcaattaaggatttagctcaaaatggagtaaaagtagccgcaggggtccctgtgacgaaagaatctgaatggactggtaaaaactttgtcccaccattaaaaactttatataccgttcaagatacctacaactataacgatccaacttgtggagagatgacctacatttgctggccaacagttgcaccgtcatctgcctatgtctataagggcggtaaaaaagcaattactggttgggaaaatacattattggttccatctttaaaacgtggtgtcattttccgtattaagttagatccaacttatagcactacttatgatgacgctgtaccgatgtttaagagcaacaaccgttatcgtgatgtgattgcaagtccagatgggaatgtcttatatgtattaactgatactgccggaaatgtccaaaaagatgatggctcagtaacaaatacattagaaaacccaggatctctcattaagttcacctataaggctaagtaa

SEQ ID NO: 32 - protein, 478 aa, Acinetobacter calcoaceticus
MNKHLLAKIALLSAVQLVTLSAFADVPLTPSQFAKAKSENFDKKVILSNLNKPHALLWGPDNQIWLTERATGKILRVNPESGSVKTVFQVPEIVNDADGQNGLLGFAFHPDFKNNPYIYISGTFKNPKSTDKELPNQTIIRRYTYNKSTDTLEKPVDLLAGLPSSKDHQSGRLVIGPDQKIYYTIGDQGRNQLAYLFLPNQAQHTPTQQELNGKDYHTYMGKVLRLNLDGSIPKDNPSFNGVVSHIYTLGHRNPQGLAFTPNGKLLQSEQGPNSDDEINLIVKGGNYGWPNVAGYKDDSGYAYANYSAAANKSIKDLAQNGVKVAAGVPVTKESEWTGKNFVPPLKTLYTVQDTYNYNDPTCGEMTYICWPTVAPSSAYVYKGGKKAITGWENTLLVPSLKRGVIFRIKLDPTYSTTYDDAVPMFKSNNRYRDVIASPDGNVLYVLTDTAGNVQKDDGSVTNTLENPGSLIKFTYKAK

SEQ ID NO: 33 - DNA, 1059 bp, Vibrio parahaemolyticus
ttggctcttggcgcgattggagcagtgctctcaacttccgtatacgcttggcaagccgagcttgttgcgtctggcctcaaggtaccatgggggatgagctctattgacgacaaccaattgcttgtcacacaacgccacggcgaaataggtgtggttaaccttgatgatggcttctatcgtactgtcgctacaccggcacaggtgagtgctgttggtcagggaggcttgcttgatgttgccaagtctcctttcgaagacaatacgttctattttacctattcaaagcaagtggaagatgcatacgaaacggcattagcttccgctcaatatcaacaaggtaagctgattggatggaaagagcttctcgtgactcagtctggatctgatacgggcaggcattttggtagccgaatcacctttgacgaacgctacgtgtatatgtctgtcggcgatagaggcgttcgcgacaacgggcaaaatcagagaacacacgctggctcgatcctgcgcctattaccaaacgggctagccccagaagacaacccgtttaccaaagtaaactcagcgttaaacgagatatggagctatggtcaccgcaatcctcaaggactcttttacgaccgcagcactcaacagctatggtctatcgagcatggtccgcgcggcggagatgaaatcaacttgattgtcaaaggagaaaactatggctggcctgttacctcccacggcaaagagtattgggggccaatctcagttggtgaagccaaaaccctgccgggtattcaaacaccgaagaaagtgtatgtgccttctatcgcacctagctctctcattctttacagaggggataagtacccatccctaaatggcaaactgatatctggcgcattgaagctcacccacctcaatgtcgtcacactcgatgagcaagcaaatattgttgctgaagagcggctactagaatccttaggagagcgaatacgagatctggaagtaacaccagtaggagagattctgtttagcactgacagtgggaatatctaccgtctcgtggagtaa

SEQ ID NO: 34 - protein, 352 aa, Vibrio parahaemolyticus
MALGAIGAVLSTSVYAWQAELVASGLKVPWGMSSIDDNQLLVTQRHGEIGVVNLDDGFYRTVATPAQVSAVGQGGLLDVAKSPFEDNTFYFTYSKQVEDAYETALASAQYQQGKLIGWKELLVTQSGSDTGRHFGSRITFDERYVYMSVGDRGVRDNGQNQRTHAGSILRLLPNGLAPEDNPFTKVNSALNEIWSYGHRNPQGLFYDRSTQQLWSIEHGPRGGDEINLIVKGENYGWPVTSHGKEYWGPISVGEAKTLPGIQTPKKVYVPSIAPSSLILYRGDKYPSLNGKLISGALKLTHLNVVTLDEQANIVAEERLLESLGERIRDLEVTPVGEILFSTDSGNIYRLVE

SEQ ID NO: 35 - DNA, 2339 bp, Rhizobium leguminosarum
tcacggcaacgtataggcgatcacataatcgccgggcgtggtgccgaccgagccgtggccgccggcgaccatgacgacatattgcttgttgtcatcggtcatataggtcatcggcgtcgcctggccgccggcgggaagccgtccctgccagagctcccggccactcgtcacgtcataggcgcgcaggtagttgtcgaccgcggcccccaggaaggcgacgccgcccttggtcagcatcggcccgccgatgccgggcacgcccaccttgaagggcaggggcaatggcgtcatgtcgtggacggtgccgttcttgtgcatataggcgatcttgccggtgcgcaggtcgacgccggcgacataaccccatggcggcgcctggcaggggatttggagcagcccgaggaagggacccatgaagacgccgtagggcgcgccgtcattgcggttgagcccctgttcgctgcccttctcgtcctggcctctcggaggaatgtcggcggccggcaccaggcgcgaggtaaaggcgagataggtcggcatgccgaacatgatctgccgctccggatccaccgccaccgagccccagttgaaggtgccgaaattgccgggatagacgatcgtccctttcagcgaaggcggcgtaaagcggccctcatagtggtagcggtggaagtcgatgcggcaggccatctgatcgaacagcgatacgccccacatgtctctttcctggagcggctttggcgagaaggtgaggtccgagatcggctgcgtcggcgaggtatgatcgcccgagaccgcgccgcctggcgctggtatttccttgatcgggatgatcggctcgccgctgcgccggtcgagcacatagatatcgccctgtttggtcgggccgaccaaggccggaaccacggtgccgtcttgcttcgtcaggtcgatcagcgccggctgggccggcacgtccatgtcccagagatcgtgatgcactgtctggcgcacccagcgcaactggccggtggcgatatcgagcgcgacgatagaggaggagaatttctcgacattgtcgctgcggccgatgccgatctggtcgggtacttggttgccgagcgggatgtagaccatgccgagcgcctcgtcgacgctgaagaccgaccaactgttcggcgagttggtcgtataggtctggccctcggtgatcggcgtcgtcacatcgggattgccggaatcccaattccagaccagggcgccggtattgatgtcgaaggcgcggatgacgccggattgctcctcagtcgaataattgtcgttcaccgccccgccgacgatgattttgcccgccaccgcgaccggcggcgaagtggaataatagtatccggccgggttgaaccgcatgccggtttccaaatgcagcacgccctggtcggcaaagctggtgcagaccttcccgtccgccgcatcgagcgcgatcagccgggcgtctgaggtcggcaggtagacgcgctcggcgcagggctggccggcggcaacggttggatcggcataataggtgacgccgcggcaggtctgatgctgccggtcggggttcatgcccgagttggcgtcgtatttccatttctccttgccggtcttggcgtcgagcgcgatcgcccagttatgcggcgtgcagagatagagcgtgtccttcaccttcagcggcgtcacctgataggtcgtctcgccgacatcgtccggccgcttgacgtcgccggtctggtatcgccacgcttccttgagggtggagacgttttcggcggtgatctggtcgagtggcgaatagcgctggccgaagggcgtgcggccgtattgatgccattcgccgtccgggacgctgccgccgaaggcgggattggcggcgaccgtatccttgggcagctcgccggcgagatcatgggggtcagtcgtcatcgaatagagggcgacaaggatggcgagaatgacaggcgcggcgagcggccaggggtttgcggcataggtgatgcccgttgggctgcgcaggccaagcggccggcggatccatggcgtcagcagccagagcccgagcaggatgatcatgccgccgcggggcccaagctgccaccagtcgaagccgacctcccaaatcgcccaggcgagtgctgcgacgacgagcaccgcatgacccagagcgccaccgccttgcgcatcagcagcagcccggcggtaatcaggaacatcagcccggcgaacagatagaagacgctgccgccgagcgtgacgagccagagcccgccgccgctgagagtgagcccgacgatgatgaagaagatggaggtgacgatgatcgccat

SEQ ID NO: 36 - protein, 779 aa, Rhizobium leguminosarum
MAIIVTSIFFIIVGLTLSGGGLWLVTLGGSVFYLFAGLMFLITAGLLLMRKAVALWVYAVLVVAALAWAIWEVGFDWWQLGPRGGMIILLGLWLLTPWIRRPLGLRSPTGITYAANPWPLAAPVILAILVALYSMTTDPHDLAGELPKDTVAANPAFGGSVPDGEWHQYGRTPFGQRYSPLDQITAENVSTLKEAWRYQTGDVKRPDDVGETTYQVTPLKVKDTLYLCTPHNWAIALDAKTGKEKWKYDANSGMNPDRQHQTCRGVTYYADPTVAAGQPCAERVYLPTSDARLIALDAADGKVCTSFADQGVLHLETGMRFNPAGYYYSTSPPVAVAGKIIVGGAVNDNYSTEEQSGVIRAFDINTGALVWNWDSGNPDVTTPITEGQTYTTNSPNSWSVFSVDEALGMVYIPLGNQVPDQIGIGRSDNVEKFSSSIVALDIATGQLRWVRQTVHHDLWDMDVPAQPALIDLTKQDGTVVPALVGPTKQGDIYVLDRRSGEPIIPIKEIPAPGGAVSGDHTSPTQPISDLTFSPKPLQERDMWGVSLFDQMACRIDFHRYHYEGRFTPPSLKGTIVYPGNFGTFNWGSVAVDPERQIMFGMPTYLAFTSRLVPAADIPPRGQDEKGSEQGLNRNDGAPYGVFMGPFLGLLQIPCQAPPWGYVAGVDLRTGKIAYMHKNGTVHDMTPLPLPFKVGVPGIGGPMLTKGGVAFLGAAVDNYLRAYDVTSGRELWQGRLPAGGQATPMTYMTDDNKQYVVMVAGGHGSVGTTPGDYVIAYTLP

SEQ ID NO: 37 - DNA, 2244 bp, Salmonella enterica
ttatttcgcgtcgtcaggcaaagcataggccacgatatagtcgcccatcttcgtaccaaacgaaccgtggcctccggcagaaacgaccacgtactgcttaccgtttacctcataggtcatcggcgttgcctggcctccggcgggtaaacgtccctgccacagtttttccccgttgctcatgttataagcgcgcaggtagttgtcggcggtcgcggcaatgaacagcacattaccggcggttgagattggcccgcccagcattggcatccccatgttgaacggcaccggaaccggcatcgggaacggcatactgtcgcgcggcgtccctatacgtttcttccacacgatttcattggtcttcagatccaacgcggaaatatagccccaggccggttgtttacacggcaggccaaacggcgaaaggaacggattcagcgtgacgccgaatggtacgccgtactgtggctgaatacccgcttctgtaccagtacctttcgcgtctttcggcggctccatcggattacccgggccgcgtgggatcagcttcgagacaaacggcagcgccatcgggttagcgatagcgacctgacggtctggatcgacggaaataccgccccattcaaacatccccaggttgccagggaataccaatgtcccctgctcggacggcggggtgaagataccttcatagcgcaactggtggaacatgacgcggcacaccagttggtcgaacatggtcgcgccccacatatccgcgccggagagatcttttttcgggcggaaggttaaatcagagaacggctgagttttggcgacgtaatcgccttttgccgcgccctgcgggaccggtttttccggggcgggcacgaccagctcgccattacgtctgtccagcacaaagatattaccggttttcgctggcgcatagataaccggaacggtagtaccgtcaacggtaatatccgccagcgtcggctgtgccggcaggtccatatcccacagatcgtgatggacggtctgataactccaggccagtttcccggtggtggcattcagcgccaggatggagctggcataacgttcctgctccggcgtgcggttaccgccccagatatccggcgtggtcacgcccatcggcagatagaccagatccagtttcgcatcataggcggcaggcgcccaggagttcggcgagttaaaggtaaaagcgtgctcatcggctggaatcgcgttcggatctttcgcccccggatcgaaggcccacatgagtttaccgctattaacatcaaaaccacggataacgccggacgtttcgcgggtagagaagttatccgtgaccgaaccggcaatgacaattgttttatcggtgataatcggcggcgacgtcggttcatacagtcccggcgtggtatccggcatattggtttgcagattcaggacgcctttgttggcgaaggtttcacacagcttgccggtttcggcattcaccgcgaaaaggcgaccgtcgttgaccgggaggataatacggcgcggacagtcggcgatgacctccggcgaggcggtatccgctttggcctcatgataagagacgccgcggcaggtcacatgctggaaagacgaatcggtcttcaactgaggatcaaaatgccacttctctttaccgctggcggcgtccagcgcgaacagacgctggtgagctgtacacagataaagcgtatcgccgactttaatcggggtcacttcgttggtgatttcgcccggatcgttgggttgtttcagatcgccggtgcggaagacccacgcttctttcagttggtgaacgttatccgcagtgatctgtttcagcggcgaatagcgttggccttcctgattacgtccataggccggccagtcttcgtcggcgatcgacgagcttgttgcggcgggcgtggcgtcggcgcgaagcgtaccgttgatctcctgcgggtcgttgaagccagcccaggtcagaatgccgccgctaatcagcagcgccacgaccagcgccgccactgcgccgctggaaggaaccaccagccgatgccagacaaacggcaatattagccagatgccgaaaaacaccagaatatcgctacgcggcgtgagcgcccagaagtcgaacccgacttcccacacgccccaaatcattgtcgccagcagcagggcggcatacagccacagtgcggcacgtttactgcgccatagcaatccggccacaaccagcat

SEQ ID NO: 38 - protein, 747 aa, Salmonella enterica
MLVVAGLLWRSKRAALWLYAALLLATMIWGVWEVGFDFWALTPRSDILVFFGIWLILPFVWHRLVVPSSGAVAALVVALLISGGILTWAGFNDPQEINGTLRADATPAATSSSIADEDWPAYGRNQEGQRYSPLKQITADNVHQLKEAWVFRTGDLKQPNDPGEITNEVTPIKVGDTLYLCTAHQRLFALDAASGKEKWHFDPQLKTDSSFQHVTCRGVSYHEAKADTASPEVIADCPRRIILPVNDGRLFAVNAETGKLCETFANKGVLNLQTNMPDTTPGLYEPTSPPIITDKTIVIAGSVTDNFSTRETSGVIRGFDVNSGKLMWAFDPGAKDPNAIPADEHAFTFNSPNSWAPAAYDAKLDLVYLPMGVTTPDIWGGNRTPEQERYASSILALNATTGKLAWSYQTVHHDLWDMDLPAQPTLADITVDGTTVPVIYAPAKTGNIFVLDRRNGELVVPAPEKPVPQGAAKGDYVAKTQPFSDLTFRPKKDLSGADMWGATMFDQLVCRVMFHQLRYEGIFTPPSEQGTLVFPGNLGMFEWGGISVDPDRQVAIANPMALPFVSKLIPRGPGNPMEPPKDAKGTGTEAGIQPQYGVPFGVTLNPFLSPFGLPCKQPAWGYISALDLKTNEIVWKKRIGTPRDSMPFPMPVPVPFNMGMPMLGGPISTAGNVLFIAATADNYLRAYNMSNGEKLWQGRLPAGGQATPMTYEVNGKQYVVVSAGGHGSFGTKMGDYIVAYALPDDAK

SEQ ID NO: 39 - DNA, 2046 bp, Solibacter usitatus
atgatccgatcgctagcgttcgccctcgcctccgttcgactgcttgccgcctcggattggccggggtacggtggcggccccgcggggattcgtcactcgcccttgaagcagatcaatcgctccaacgtgtcgcacctcaaggtcgcgtggacgtacgacaccaaggatgggccgggcgatccacagacgcagccgatcgtggtcgatggcgttctctatggcgtaactccgacccacaaagccatcgcgctaaacggcgccaccggaaagctgctgtggaccttcgactccggcattcgcggacgtggtcccaatcgctcggtagtctggtggtcgccgcgaacggagcggcggctcttcgtcgcggtgcagagctttatctacgcgctcgacccagccaccgggaagcccatggcggatttcggcgagcagggccgcatcgatctccgccagggcctggggcgcgatgccgccaagcagtcctacgtcctgaccagcccgggaattatttatcgcgatctgttgatcgtgggcggccgcctgccggaagcgctgccggcgccgcccggcgacatccgcgcctatgatgtccgcaccggcaaacagcgctggaccttccacacgattccgcatccgggcgagcccggctacgagacctggccctccgatgcctggacctactccggcggcgccaacaactgggccggcatggcgctcgacgagaagcgtggcatcgtctacgtccccaccggctcagccgccagcgatttttatggcgccgaccgcctgggcgacaacctctacgccaactgcctgctggcgctgaacgccgccaccggcgagcgcgtctggcacttccaggcggtgcatcatgacatctgggatcgtgactttccctccccgcccgcgctggtcaccgtccggcacgaaggccgcacggtcgatgccgttgtgcagacctccaagcagggctgggtctacctgttcaatcgcgccaccggcaagccgttgttccccatcgagagccggccctatcccgtcagcgagctgccgggcgaacgtgctgccgccgaacagtcgctcccgaccaggccggccgcctttgcgcgccaggctctgactgaagacgtgctcacccgccgcaccgatgcaacccacgcgtgggcgcttgccgagttccacaagctgcgcagcgccggacagttcctgcccttcggggcagggaatgagaccgtcatttttccaggcttcgatggcggcgccgagtggggcggtccggccttcgatccctccacgggattgctgtacgtcaacgccaatgaaatggcctggagggcgggcctcaaggaatcccagccggaaaactcagcccggcaactgtatgtggcgcagtgcggcacctgccatggcgacgacctcaagggctcgcctccgcagttcccggcgttgaacgaccttgccggaaagcgctcggcccaggaaatcggcgcaatcattcgcgagggcggcgggcgcatgcctgctttcccccggcttcgtcccgacgacatcaccgcgctcagcgattatctgctccgcggccgcagcaaggagctggagagcaccgcctcactggatcgcgaaccgcgatttcgttttaccggctatcacaagttcgtcgatcccgatggctatccggcaatcgcgccgccgtggggcacgctcaacgccatcaacctcaataccggcgaatacgcgtggcgcatcccgctcggcgaatatcccgacctcgcggccaaaaacacgggatcggaaaactacggcggccccatcgtgaccgccggcggcctcgtgtttatcgcggccaccaactatgaccgcaagttccgagccttcgacaaggccaccggcgaactgctctggcagactacgctcccgatggccggcaacgccacacccatcacctatgaaatcgccggccgccagtacgtcgtgatctacgcgaccggcggcaagtccggcaagtggggtccttcgggcggaatctacatggcgttctcactgtaa

SEQ ID NO: 40 - protein, 681 aa, Solibacter usitatus
MIRSLAFALASVRLLAASDWPGYGGGPAGIRHSPLKQINRSNVSHLKVAWTYDTKDGPGDPQTQPIVVDGVLYGVTPTHKAIALNGATGKLLWTFDSGIRGRGPNRSVVWWSPRTERRLFVAVQSFIYALDPATGKPMADFGEQGRIDLRQGLGRDAAKQSYVLTSPGIIYRDLLIVGGRLPEALPAPPGDIRAYDVRTGKQRWTFHTIPHPGEPGYETWPSDAWTYSGGANNWAGMALDEKRGIVYVPTGSAASDFYGADRLGDNLYANCLLALNAATGERVWHFQAVHHDIWDRDFPSPPALVTVRHEGRTVDAVVQTSKQGWVYLFNRATGKPLFPIESRPYPVSELPGERAAAEQSLPTRPAAFARQALTEDVLTRRTDATHAWALAEFHKLRSAGQFLPFGAGNETVIFPGFDGGAEWGGPAFDPSTGLLYVNANEMAWRAGLKESQPENSARQLYVAQCGTCHGDDLKGSPPQFPALNDLAGKRSAQEIGAIIREGGGRMPAFPRLRPDDITALSDYLLRGRSKELESTASLDREPRFRFTGYHKFVDPDGYPAIAPPWGTLNAINLNTGEYAWRIPLGEYPDLAAKNTGSENYGGPIVTAGGLVFIAATNYDRKFRAFDKATGELLWQTTLPMAGNATPITYEIAGRQYVVIYATGGKSGKWGPSGGIYMAFSL

SEQ ID NO: 41 - DNA, 2169 bp, Klebsiella pneumoniae
atggcaactggcaacgcgccgcgcggattcccccggatcctgcagtggcttctcgccggactgatgctcatcatcggtctggctgtgggtattcttggggcaaaactggccctcgtcggcggtacactgtacttcgcgctgatgggcgtggtcatggtcatcgcggcggtgttgattttccgcaaccgccgcggcggtattctgttatacgccgtggcgtttatcgcctcggtgatctgggcgattagtgacgctggctggaactactggccgctcttctcgcgcctgtttgcgctcggcgtactggccttcctggccgccctggtctggcctttcctcgccagcccaccggcgaaaaaaggcccggcctatggcgtcgccgccgttctcgccgtggcgctggcggtgagctttggctggatgttcaaatccgcaccgctggtcagcgcgactgaagcggtaccagtcaagcccgtcgcgccaggcgaacagcagaaaaactgggcgcactggggtaacaccacccacggcgaccgtttcgccgccctcgaccaaatcaacaaacaaaacgtcaaccagctgcaggttgcctgggtagcgcatactggcgatatcccacagagcaacggctcgggcgcagaagatcagaacaccccgctgcagattggcgatactctctatgtctgtaccccttacagtaaagtgctggcgctggatgtggacagcggaaaagagaaatggcgctatgactccaaatcctcttcacccaactggcagcgctgccgcggcttagggtattacgaagatagccaggcgcaaaccgcgctagcgtcaggcacgcagccggccgcctgttcccgtcgtctgttcctgccgaccatcgacgcccgcctgatcgccattgatgccgataccggcaaactgtgcgaaaacttcggcgatggcggtattgtcgacctcagcgtcggcatgggtgaagtgaaagcgggttactatcagcagacctccaccccgctggtggcgggcaatgtggtggtggtgggcggccgcgtcgcggataactactccactggcgaaccgccgggcgtggtccgcgcgtttgatgtccacaccggcaaactggcatgggcgtgggatccgggcaatccagcgctgaccggtgttccgccggaaggccagacctacacgcgcggtacgccaaacgtctggtcggcgatgtcctacgacgcgaagctgaatctgatctatctgcccaccggcaacgccacgccagatttctttggcggcgaacgtaccgcgctggacgataaatacagctcctctatcgtggctgtggatgccaccaccggccaggtgcgctggcacttccagaccacacaccacgatcggtgggactttgacctgccgtctcagccgctgctgtacgatctgcctgacggcaagggcggcaccaccccggtgctggtgcagaccagcaagcagggcatgatctttatgctcaaccgcgagaccggcgagccggtggccaaagtggaagagcgtccggtgccggcgggcaatgtcaaaggtgaacgctactcgccgacgcaaccttactcggtagggatgccgatgatcggcaaccagacgttgaccgagtccgatatgtggggagcgacaccgatcgacctgctgctgtgccgtattcagttcaaagagatgcgccatcagggcgtctttaccccgccaggtgaagaccgttccctgcagttcccgggctcgctcggcggcatgaactggggcagtgtttcgctggatccaaacaacagcctgatgtttgtcaacgatatgcgtctgggcctggccaactatatggtgccgcgcgcgaaggtggcgaaagacgccagcgggatcgagatgggcatcgtgccgatggagggcacgccgtttggcgcgatacgcgaacgtttcctgtcgccgctgggcattccgtgccagaagccgccgttcggcaccatgtccgcggtagatctgaaaaccgggaaactggtgtggcaggtgccggttggcaccgtagaagacaccgggccgctgggcatacgtatgcacatgccaattccaatcggcatgccgacccttggcgcatcgctagcaacgcagtccggactgctgttcttcgccggcacctag

SEQ ID NO: 42 - protein, 722 aa, Klebsiella pneumoniae
MATGNAPRGFPRILQWLLAGLMLIIGLAVGILGAKLALVGGTLYFALMGVVMVIAAVLIFRNRRGGILLYAVAFIASVIWAISDAGWNYWPLFSRLFALGVLAFLAALVWPFLASPPAKKGPAYGVAAVLAVALAVSFGWMFKSAPLVSATEAVPVKPVAPGEQQKNWAHWGNTTHGDRFAALDQINKQNVNQLQVAWVAHTGDIPQSNGSGAEDQNTPLQIGDTLYVCTPYSKVLALDVDSGKEKWRYDSKSSSPNWQRCRGLGYYEDSQAQTALASGTQPAACSRRLFLPTIDARLIAIDADTGKLCENFGDGGIVDLSVGMGEVKAGYYQQTSTPLVAGNVVVVGGRVADNYSTGEPPGVVRAFDVHTGKLAWAWDPGNPALTGVPPEGQTYTRGTPNVWSAMSYDAKLNLIYLPTGNATPDFFGGERTALDDKYSSSIVAVDATTGQVRWHFQTTHHDRWDFDLPSQPLLYDLPDGKGGTTPVLVQTSKQGMIFMLNRETGEPVAKVEERPVPAGNVKGERYSPTQPYSVGMPMIGNQTLTESDMWGATPIDLLLCRIQFKEMRHQGVFTPPGEDRSLQFPGSLGGMNWGSVSLDPNNSLMFVNDMRLGLANYMVPRAKVAKDASGIEMGIVPMEGTPFGAIRERFLSPLGIPCQKPPFGTMSAVDLKTGKLVWQVPVGTVEDTGPLGIRMHMPIPIGMPTLGASLATQSGLLFFAGT

SEQ ID NO: 43 - DNA, 2346 bp, Rhodobacter sphaeroides
ttgaccgctaccctgaccgccatcgtccttgccctcgccgggctgggcctcctcatcccgggcgcctggctggccgttctgggcgggagctggttctacctcgtggccggagccctcatgctgctgacggcatggcaggtgttccgccgctcctcctccgccgaatggatctatggcggcctcatcctcggcgcgctcgtctggtccgtctgggaagtgggcttcgactggtgggaactcattccccgcggcggcatcatcgcgcttctgggcatctggctctacctgcccttcatgcggcggcagctgctgaccgaggacggccgccccgcgcccggcctgcccttggccctgccccttctggcggccatcggggccgccatctggtcatggaccggagacgaagcgggacatgcaggctcgctgccgacggaagtggtcagcgccgagcccgatctcggcccctccgcgcctcccggcgagtggcaccagtacggccgcacgcagtacgggcagcgctacagcccgctcgaacagatcaacatccagaatgtcgccgagctcgagcaggtctggcagtatcagaccggcgatgtgaagctgccgcaggacgtgaccgagaccacctatcaggtcacgccgctgaaggtcgccgaccgtctctacatctgcaccccgcacgatctggccatcgcgctcgatgcggcgaccggcaaggaagcctggcgcttcgacgcgcgctccgggctcgagtccgaccggcagcaccagacctgccgcggcgtgacctactggcgggatccggcacgcgccgagggagagctctgcgccgagcgggtctatctgccgacggccgatgcgcggctgatcgcgctcgacgcaaagtccggcgcggtctgcaccttcttcgccgacgagggtacgctccatctcgagaacgggatgccctacaccccggcaggcttctactattccacctccccgcccgtggcggtcggaggccggatcatcatcggcggcgcggtcaacgacaatttctcggtctattcgcagtcgggcgtgatccgggccttcgatgccaacaccggggcgctcctgtggaactgggacagcgccaaccccgacaagaccgcccccatcgactggatgaacggcgagacctataccgcaaattcgcccaactcctggtcggtcttctcggtcgatgaggaacgcggcctcgtctacatcccgctcggcaaccaggtccccgaccagctgggcttcaaccgctcgcccgccgtcgaggaacattcctcctcggtcgtggcgctcgacgtggcgacgggtcagaaggcctgggtgttccagaccgtgcaccatgacctgtgggacatggacgtgcccgcccagcccgttctgatcgatctcgacatcgacggtcagacggtgcccgcgctcgtgcagccgacgaagcagggcgacatctatgtgctcaaccgcgagacgggcgagccgatcctgcccgtcaccgaagagcccgccccgcaggagggcgctctgcgcgaagagacccctgccccgacgcagcccacctcggcgctgagcttcaaacccgaggcgctgcgcgagaaggacatgtggggcgtcacgctctacgaccagctcgcctgccggatccagtatcaccggttgaactacgagggccgctacacgcccccgtcgctgaacggcaccatcgtctatccgggcaatttcggcaccttcaactggggctccgtcgccgtcgatcccgagcggcaggtgatgttcggcatgcccacctacctgcccttcacgagccagctggttccggccgaggacatcccgccgcccggcgcagatcagaaggccagcgaacagggcctgaaccgcaacgagggcgcgccctacggcgtcatcatggggcccttcctcgggccgctcggcgtgccctgctcggcgccgccgtggggcttcgtcgcgggggcggacctccgcaccggagagatcgcctacatgcaccgcaacggcaccgtgcgcgacatgacgcccctgccgctgcccttcaaggtgggcgtgccggggatcggcgggccgatcgtcacgcgcggcggcgtggccttcctcggagcggcggtcgacgactacctgcgcgcctacgatgtgaccacgggcgatcagctctggcaggcgcggctgcccgcgggcggccagtcgacgccgatgacctacgagcaggacggccggcagttcgtggtgatcgtggcgggcgggcacggctcggtcggcacgaaaccgggcgactatgtgatcgcctacgccctgcccgacggctcggagggttga

SEQ ID NO: 44 - protein, 781 aa, Rhodobacter sphaeroides
MTATLTAIVLALAGLGLLIPGAWLAVLGGSWFYLVAGALMLLTAWQVFRRSSSAEWIYGGLILGALVWSVWEVGFDWWELIPRGGIIALLGIWLYLPFMRRQLLTEDGRPAPGLPLALPLLAAIGAAIWSWTGDEAGHAGSLPTEVVSAEPDLGPSAPPGEWHQYGRTQYGQRYSPLEQINIQNVAELEQVWQYQTGDVKLPQDVTETTYQVTPLKVADRLYICTPHDLAIALDAATGKEAWRFDARSGLESDRQHQTCRGVTYWRDPARAEGELCAERVYLPTADARLIALDAKSGAVCTFFADEGTLHLENGMPYTPAGFYYSTSPPVAVGGRIIIGGAVNDNFSVYSQSGVIRAFDANTGALLWNWDSANPDKTAPIDWMNGETYTANSPNSWSVFSVDEERGLVYIPLGNQVPDQLGFNRSPAVEEHSSSVVALDVATGQKAWVFQTVHHDLWDMDVPAQPVLIDLDIDGQTVPALVQPTKQGDIYVLNRETGEPILPVTEEPAPQEGALREETPAPTQPTSALSFKPEALREKDMWGVTLYDQLACRIQYHRLNYEGRYTPPSLNGTIVYPGNFGTFNWGSVAVDPERQVMFGMPTYLPFTSQLVPAEDIPPPGADQKASEQGLNRNEGAPYGVIMGPFLGPLGVPCSAPPWGFVAGADLRTGEIAYMHRNGTVRDMTPLPLPFKVGVPGIGGPIVTRGGVAFLGAAVDDYLRAYDVTTGDQLWQARLPAGGQSTPMTYEQDGRQFVVIVAGGHGSVGTKPGDYVIAYALPDGSEG

SEQ ID NO: 45 - DNA, 2391 bp, Enterobacter sp.
atggctgaaacaaaaactcaacagtcgcgtctactggtaactttaacagcagcgttcgcagcgttttgcgcactctatctgttgatcggtggcgtctggctggtcgcattaggcggctcctggtattacccgattgcgggtctggttatggttggcgtaaccgttctgctcttaaaaggtaaacgatccgcactgtggctctacgctgcactgcttcttgtcacaatgttttggggagtctgggaagttggcttcgacttctgggcgctgacaccgcgcagcgacatcctggtcttctttgggatctggctgatcctgccgttcgtctggcgtagcctgagggttccttccagcggtgcagttgcggcgctggtggtttccctgctgattaccggcggcatgctaacctgggctgggtttaacgatccgcaggaagtgaaaggcacgctgagcgcagattccacacccgctgcggcgatctctgacgttgcagacggtgactggccggcttatggtcgcaaccaggaaggccaacgttattctccgctgaagcaaatcaacgcggataacgttaaaaacctgaaggaagcctgggtgttccgcaccggcgaccttaagcagccaaacgacccgggcgaattgaccaacgaagtgacgccaatcaaagtcggcaacatgctttatctgtgtactgcacaccagcgtctgtttgcgcttgatgcggcgaccggtaaagagaaatggcacttcgacccgcagttgaattctaatccgtcgttccagcacattacctgccgtggcgtgtcctatcacgaagcccgcgcggataatgccagcccggaagtggttgctgattgcccgcgtcgcatcatgctgccggtcaacgatggtcgactgttcgccatcaacgccgaaaccggcaaactgtgcgaaacctttggcaacaaaggcattctgaatctgcagaccaatatgccggataccacgccgggtctgtacgaaccgacttccccgccgattatcaccgataaaacaattgtgattgccggttcggtcacggataacttctcgactcgcgaaacgtccggcgttatccgtggtttcgacgtaaacacgggcaaactgctgtgggccttcgatccgggcgcgaaagatcctaacgcgattccgtcggatgaacacaccttcaccttcaactcgccaaactcatgggcgcctgcggcgtatgacgcgaagctagacctggtgtacctgccaatgggtgtgaccacgccagatatctggggcggaaaccgcacgccagagcaagagcgttacgccagcgcgattgtggcgctgaatgcgacgaccgggaaactggcctggagctatcagaccgtacaccacgacctgtgggatatggatatgccatcccagccgacgctggcggatatcaccgttaacggtaaaaccgttccagtgatttatgccccggcgaaaaccggcaacatctttgtactcgaccgtaccaacggcaaactggtggttccggcaccggaaaaaccggttccgcaaggtgcagccaaaggcgactatgtctctaaaacccagcctttctcggacctgagcttccgtccaaagaaagacctgaccggtgccgatatgtggggcgccaccatgttcgaccagctggtgtgccgcgtgatgttccatcagctgcgctatgaaggcatcttcacgccgccgtctgagcagggcacgctggtcttcccgggtaatctggggatgttcgaatggggcggtatctcggttgatcctaaccgtcaggtggcgattgcaaacccgatggcgttgccgttcgtctctaagcttatccctcgcggcccgggtaacccgatggagcagccgaaagatgcaaaaggtagcggtacagaagcgggcattcagccgcagtacggcgtaccgtttggcgtgacgctgaaccccttcctgtcgccgtttggcctgccgtgtaaacagcctgcatggggttatatttctgcgctggatctgaaaaccaatcaggttgtgtggaaaaaacgtattggtacgccacaggacagcatgccgttcccgatgccgattcccgtgccgttcaatatggggatgccaatgctgggtggtccgatttccaccgccggtaacgtcctgttcatcgcggcgaccgcagataactacctgcgcgcgtacaacatgagcaacggtgaaaagctgtggcaaggccgtctgcctgcgggtgggcaagcgacgccgatgacctatgaagtggatggcaagcagtatgttgttatctctgcgggcggtcacggttcgtttggtacgaagatgggcgactatattgtcgcgtatgctctgcctgacgatgtgaaataa

SEQ ID NO: 46 - protein, 796 aa, Enterobacter sp.
MAETKTQQSRLLVTLTAAFAAFCALYLLIGGVWLVALGGSWYYPIAGLVMVGVTVLLLKGKRSALWLYAALLLVTMFWGVWEVGFDFWALTPRSDILVFFGIWLILPFVWRSLRVPSSGAVAALVVSLLITGGMLTWAGFNDPQEVKGTLSADSTPAAAISDVADGDWPAYGRNQEGQRYSPLKQINADNVKNLKEAWVFRTGDLKQPNDPGELTNEVTPIKVGNMLYLCTAHQRLFALDAATGKEKWHFDPQLNSNPSFQHITCRGVSYHEARADNASPEVVADCPRRIMLPVNDGRLFAINAETGKLCETFGNKGILNLQTNMPDTTPGLYEPTSPPIITDKTIVIAGSVTDNFSTRETSGVIRGFDVNTGKLLWAFDPGAKDPNAIPSDEHTFTFNSPNSWAPAAYDAKLDLVYLPMGVTTPDIWGGNRTPEQERYASAIVALNATTGKLAWSYQTVHHDLWDMDMPSQPTLADITVNGKTVPVIYAPAKTGNIFVLDRTNGKLVVPAPEKPVPQGAAKGDYVSKTQPFSDLSFRPKKDLTGADMWGATMFDQLVCRVMFHQLRYEGIFTPPSEQGTLVFPGNLGMFEWGGISVDPNRQVAIANPMALPFVSKLIPRGPGNPMEQPKDAKGSGTEAGIQPQYGVPFGVTLNPFLSPFGLPCKQPAWGYISALDLKTNQVVWKKRIGTPQDSMPFPMPIPVPFNMGMPMLGGPISTAGNVLFIAATADNYLRAYNMSNGEKLWQGRLPAGGQATPMTYEVDGKQYVVISAGGHGSFGTKMGDYIVAYALPDDVK

SEQ ID NO: 47 - DNA, 1431 bp, Bacillus cereus
ttgacaaaagttaaagttagtttacgacccattgttcataatataaatttaccaaccgtattgaaaacaacaatacttccaggcgaatcgaccgaaagactatttatcgcaacccaattaggagagattttttacatagggaatggagttataaagatatttttagatattcgtcacctaattattaaattaggtacatttgaagaaggtgtttctagtagcggttatgatgaacgcggattgcttggacttgcgttccatccacaattttatcaaaatgggttattttatcttcattattcagtagctggaactcaagggccaggtgcattttctgagcaatttaaaccgaatccttgtgaccctaaaacgctaaatttaaagtgggttaatagaaatacgcaatatgatcacattgatacagtagaagaatggactttacaatctaatggtcaagctcaaaagcgacgaacattactgaatgtaagaagaccattttttaatcataacggagtcaatagtttaaacttttcacctgagactggaaaacttgtttttacaaatggagatggcggatcaggttatgatccatttaatttaagccaagatgatttagaaatagcaggtaaaataattgaaatcgatgtaagtaaaaatacatttataaataaccctcctgtagttacacgcttcgatgaacttcctttatctatacaagaaacacttacagtaattgcaaaaggagtacgtaatataaccggcatttcatttcaaaggttttataatcaatatatcaaatacgccggaaatgtcggacaagatattgtagagtctattttttcatttgttcaatataaacctataccggttacagaacttgttcaaatgcattttatgaggttaactcccaatcaagatggggttatcaattttgggtggcgaggatgggaaggggatttacctacttcttttatacgacattgttctgagaatcagactttggatgagagaacaatggtttattatgatgaaacaatacaaacttcagtcaagcgtattctgcctctacttagttattttcataaagattctagaacagataagtttggaggaacttcacttacaggagttcagccatatatgggaaatgcaattccaaatttaacaggtagcgtagtgtttactgaccttgctaagaaagaagattctcgaccgccagttaaaggtgttttagcctatactagagcaggtacagacggtaaacatgctgactttcatgctattgaaaccaattatgattttggtacgcaagcagcttattatatgagtttaggaacaaatttgaatcaaactaaattatatttaggagtttacagttctatgaaagtaactgattttaataaaggtaccatttttgaaattattccctga

SEQ ID NO: 48 - protein, 476 aa, Bacillus cereus
MTKVKVSLRPIVHNINLPTVLKTTILPGESTERLFIATQLGEIFYIGNGVIKIFLDIRHLIIKLGTFEEGVSSSGYDERGLLGLAFHPQFYQNGLFYLHYSVAGTQGPGAFSEQFKPNPCDPKTLNLKWVNRNTQYDHIDTVEEWTLQSNGQAQKRRTLLNVRRPFFNHNGVNSLNFSPETGKLVFTNGDGGSGYDPFNLSQDDLEIAGKIIEIDVSKNTFINNPPVVTRFDELPLSIQETLTVIAKGVRNITGISFQRFYNQYIKYAGNVGQDIVESIFSFVQYKPIPVTELVQMHFMRLTPNQDGVINFGWRGWEGDLPTSFIRHCSENQTLDERTMVYYDETIQTSVKRILPLLSYFHKDSRTDKFGGTSLTGVQPYMGNAIPNLTGSVVFTDLAKKEDSRPPVKGVLAYTRAGTDGKHADFHAIETNYDFGTQAAYYMSLGTNLNQTKLYLGVYSSMKVTDFNKGTIFEIIP

SEQ ID NO: 49 - DNA, 1176 bp, Micromonospora sp.
gtgagcccgcgtcttgcgtacccccgtgtcagccgcctccggacggcgctggcggcgtcctgcgcggcgttgctcctgggcgccgccggctgctccctcggcgagcccgagccggacccggccggcgcaccgcccaacctgcccacaccgtccggcaccgcgagtcccggcggggccggtcagcaggttgtcgccaccgtgctggccaaggggctggaggtgccgtggggcatcgcgttcctgcccgacggcggggcgctggtcaccgagcgggacaagggacggatcctccaggtcggcccggagtccggcccggacgggctggccgtccggccggtccagaccgtgccggacgtggccgccggcggcgagggcggcctgctgggcatcgcggtctcccccgcgtacgccaaggaccgcacggtcttcgtctactacacggccgaggacgacaaccggatcgcgaagatgcagctcgggcaggcgccgaagccgatcctgaccggcatcccgaagtccggcacgcacaacggcggcgggctcggcttcggcccggacggccacctctacgcgagcaccggcgacgccgggcgcaccgagaacgcccaggacgccaagagcctcggcggcaagatcctccggatcacgccggacggcaagcctgcgccgggcaaccccaccgccggatcgccggtctggtcgacgggccaccgcaacgtccagggcttcgcctggacgccggacaagaagatgtacgcggtggagttcggccagaacacctgggacgagatcaaccagatcaacaagggcgggaactacggctggccccgggtcgaggggcgcggcgacgacaagcgctacgtgaacccgatcacacagtggcccaccggtgacgcctcctgttccggcctggcggcggcggaccgtctgctcgtcgccgcctgcctgcgcggccagcgactctggctggtggagctggccggcaacggcacagtcctcggtcagccgcgcgacctgctcaacggccggtacggtcggctacgggccgtcgcggcggcgccggacggctcgctctgggtgagcacgtcgaaccgggacgggcggggtacgcccaccgccgaggacgaccgcctcctgcggctcgtcttcgccgacgggggcgccggccggagctga

SEQ ID NO: 50 - protein, 391 aa, Micromonospora sp.
MSPRLAYPRVSRLRTALAASCAALLLGAAGCSLGEPEPDPAGAPPNLPTPSGTASPGGAGQQVVATVLAKGLEVPWGIAFLPDGGALVTERDKGRILQVGPESGPDGLAVRPVQTVPDVAAGGEGGLLGIAVSPAYAKDRTVFVYYTAEDDNRIAKMQLGQAPKPILTGIPKSGTHNGGGLGFGPDGHLYASTGDAGRTENAQDAKSLGGKILRITPDGKPAPGNPTAGSPVWSTGHRNVQGFAWTPDKKMYAVEFGQNTWDEINQINKGGNYGWPRVEGRGDDKRYVNPITQWPTGDASCSGLAAADRLLVAACLRGQRLWLVELAGNGTVLGQPRDLLNGRYGRLRAVAAAPDGSLWVSTSNRDGRGTPTAEDDRLLRLVFADGGAGRS

SEQ ID NO: 51 - DNA, 2475 bp, Xanthomonas campestris
atgactgatcaatcctctaaacgcgggctcggcacttggctgttggtggcctatgccgtggtgctcgccgtgcttggtgcggcgctcgcctatgaaggcgggcgcctggtggccgttggtggctcctggtactacgtgctggccggtatcgccgtgctggtggccggtgtgctgctggcactgggcaaacgcgctggcctgtggctgtttggcgctacgttggcggccaccatcgtgtgggcgctgtgggaagtgggcctggatggttggggcctgatcccacgtctggcctggatctcggtgctcggcctggtgctgttgccgttctggggcgtggcgcggcggcgcatgcagccgttgtccgggctcggctatgccgtggtcaccggcgtgctgccggtgctgggcgcagcgctgatcctgtggccgctgctggtgccgcgcaatgtggaactggccgatgcatccaagcagccagccgatgcggcaacgccgttcagccgtggcagcgtgcccagcccggacgggaacgtggcggccaaccacgatgccagtaactggaccgcctacgccggttccaacctgtccaaccattacacccccggcgcgcagatcacgccggagaacgtcaagggcctcaaggtggcctgggaattccataccggcgatctcaagccgaaggattccaagctgggctatgcgttccagaacaccccgctcaaggtcggcgacctgctctacatctgcaccccgacccagaaggtgatcgcggtggaggcggccaacggcaaggagcgctggcgcttcgacccgcagaccaatccgaaggcgatggccggcgtggccgccaccacctgccgtggtgtgtcgtactaccaggcgccggaaggcactgccgagtgcccgacgcggatcttctggccgatggtcgatggccgcctgggtgcgctggatgcgcagaccggcaagctctgcgccagcttcggtaacaacggctatgtggacctcaatgccggcaccggcaacaccaagccgggcttcgtcggcccgacctcgccgccggtggtcatgcgcggtgtggtgatccagcccaccgggcaggtgcgcgacggtcaggaacgcgatgcgccgtccggcgtggtgcgcggcttcgacgcgctcaccggccagctgcgctgggcctgggatctgggcaacccggccatcaccgccgaaccgccggccggccagacctatacccgctccaccccgaacgtgtggtcgctgatggccgccgatgacgagctggggctggtgtatctgcccaccggcaacgctgccggcgacttcttcggcaagggccgcaccccgcaggaagaggaatacaccgcctcgctggtggccgtggatgcggcaaccggcaaggagcgctggcacttccgtaccgtcaatcacgatctgtgggactacgacatcggcccgcagccgaacctggttgattggccggttgccggcggtggcacccgccccgccgtgatccaggccaccaagtccggccaggtgttcgtgctcgaccgggccaccggccagccgatcatgccggtcaagcagattgcggtaccgcagggcaccgatcatggcgactggaccgcatccacccagccggtgtcgccgggcatgcccaacaccgtgggtgcgcccagccgtgactacgagaccatcgttgaatctgacgcgtggggcatgaccccgttcgaccagctcgcttgccgcatcgagttcaagaagctgcggtacgaaggcatgttcaccccgccaagcctgcagggctcgctgtcgttcaccggcaaccatggcggcatcaactggggcggcgtgtcggtcgatctgcagcgcggcatcatggtgatgaacagcaatcgcctgccctataccgagcacgtctacccgcgcacggtgatgaacgagctgggcgtggtgtcggtgttcaacggcagcagcaagaccaagggctacatggcgcaggaaggcctggcctatggcgcgcgcaaggagccgtggatgtcgccgctcaacacgccgtgcgtggcaccgccgtggggctatatctccggcgtggatctgcgcacgcagcaggtgatctggcgccgtccgctgggcaccggttacgaccagggcccaatgggcatcccgtccaagaccaagttcgagatcggcaccccgaacaacagcggctcgctggccactgccggcggcgtcaccttcatcggtgccagcctggacaacttcatccgcggctttgacacccgcaccggcaagcaggtctgggagacccgcgtgcctgcgggcccgcaggccgcgccgctgagctacaccatcgatggcaagcagtacatcgttgccgcggtgggtggccatgaccgcatggaaaccaagtccggcgacagcgtcattgcctgggcactgcccgacgacgcggccgccagcgccaaataa

SEQ ID NO: 52 - protein, 824 aa, Xanthomonas campestris
MTDQSSKRGLGTWLLVAYAVVLAVLGAALAYEGGRLVAVGGSWYYVLAGIAVLVAGVLLALGKRAGLWLFGATLAATIVWALWEVGLDGWGLIPRLAWISVLGLVLLPFWGVARRRMQPLSGLGYAVVTGVLPVLGAALILWPLLVPRNVELADASKQPADAATPFSRGSVPSPDGNVAANHDASNWTAYAGSNLSNHYTPGAQITPENVKGLKVAWEFHTGDLKPKDSKLGYAFQNTPLKVGDLLYICTPTQKVIAVEAANGKERWRFDPQTNPKAMAGVAATTCRGVSYYQAPEGTAECPTRIFWPMVDGRLGALDAQTGKLCASFGNNGYVDLNAGTGNTKPGFVGPTSPPVVMRGVVIQPTGQVRDGQERDAPSGVVRGFDALTGQLRWAWDLGNPAITAEPPAGQTYTRSTPNVWSLMAADDELGLVYLPTGNAAGDFFGKGRTPQEEEYTASLVAVDAATGKERWHFRTVNHDLWDYDIGPQPNLVDWPVAGGGTRPAVIQATKSGQVFVLDRATGQPIMPVKQIAVPQGTDHGDWTASTQPVSPGMPNTVGAPSRDYETIVESDAWGMTPFDQLACRIEFKKLRYEGMFTPPSLQGSLSFTGNHGGINWGGVSVDLQRGIMVMNSNRLPYTEHVYPRTVMNELGVVSVFNGSSKTKGYMAQEGLAYGARKEPWMSPLNTPCVAPPWGYISGVDLRTQQVIWRRPLGTGYDQGPMGIPSKTKFEIGTPNNSGSLATAGGVTFIGASLDNFIRGFDTRTGKQVWETRVPAGPQAAPLSYTIDGKQYIVAAVGGHDRMETKSGDSVIAWALPDDAAASAK

SEQ ID NO: 53 - DNA, 2412 bp, Pseudomonas fluorescens
atgagcactgaaggtgcttcgagtcgaagccgtcttctgccgaacctgctcggcattctgcttctgctgatgggcctggccatgctggccgggggaatcaagctgagtacgctcggcggctcgctgtattacctgctggccggtatcggcatcacgctgaccggcattctgatgctgatgcgccgtcgcgcagcattgggcctgtacgccatcgtgctgttcgccagcactgtctgggcgctgtgggaagttggtctggactggtggcaactggtgccgcgtctggcgctgtggtttgtcctcggtttcgtgatgctgctgccgtggttccgtcgtccgctgctgctcgcaggccctgcgccgatgggcaccggcggtttgaccgtggctgtgattctggccggcgtcactgccctggccagcctgttcacccacccgggcgaaaccttcggcgaactgggtcgcgacaccgccgacaccaccagcaccgccccgcaaatgccggacggcgactggcaggcctacggccgcaccgaattcggtgaccgctactcgccactgaaacagatcaccccggccaacgtcggcaagctgcaagaagcctggcgcatccagaccggcgacctgccgactgccgacgacccggtcgagctgaccaacgaaaacaccccgctgaaagccaacggcatgctttacgcctgcactgcgcacagcaaagtgctggcactggacccggacaccggcaaggaactatggcgcttcgatccgcagatcaaaagcccggaaggcttcaagggcttcgcccacatgacctgccgtggcgtgtcgtactacgacgaagccgcgtacgccaagtctgaaaacgcggcgtccaccgtgatctccgaggccggcaaagccgtcgcccaggcctgcccgcgtcgcctgtacctgcctaccgccgatgcccgcctgatcgcactgaacgccgacaccggcaagatctgcgaaggcttcggcaccaacggtgtggttgacctgacccagggcatcggcccgttcaccgctggcggttactactccacctctcctgccgcgatcacccgtgatctggtgatcatgggcggtcacgtcaccgacaacgaatcgaccaacgagccgtccggcgtgatccgcgcctacgacgtgcgcgacggccatctggtctggaactgggacagcaacaacccggacgccaccgaacccttggccccgggccagacctacagccgcaactcggccaacatgtggtcgctggccagcgtcgatgaaaaactcggcatggtctatctgccattgggcaaccagacgcctgaccagtggggcggcgatcgcacccccggtgccgagaaattcagcgccggcctggtcgcgctggacctggcgaccggcaaggtgcgctggaactaccagttcacccaccacgacctgtgggacatggacgtcggcagccagccaaccctgctggacatgaaaaccgccgacggcatcaagcctgcgctgatcgccccgaccaagcagggcagcctgtacgtcctcgaccgtcgtgacggcacgccgatcatcccgatccgcgagatcccggtgccgcaaggcgccgtgaaaggcgaccacaccgccccgacccaggcccgttcggacctgaacctgctggccccggaactgaccgaaaaagccatgtggggcgccagcccgttcgaccagatgttgtgccgcatccagttcaaggaactgcgctacgaaggccaatacacgcctccgtcggaacagggcagcctgatctacccgggtaacgtcggcgtgttcaactggggcggtgtttctgtcgacccggttcgccagatgctgttcaccagcccgaactacatggctttcgtctccaagatgatcccacgtgccgacgtacccgccgacagcaagcgcgaaagcgaaacttccggcatccagaaaaacaccggtgcgccgtatgccgtgaccatgcacccgttcatgtcgccactgggcgtaccttgccaggccccggcctggggctacgtcgccggtatcgacctgaccaccggcaaagtcgtctggaaacgcaaaaacggtaccagccgcgacagctcgccaatcccgatcggcttcaccctgggcgtgccaagcatgggcggctcgatcgtcactgccggcggcgtcggcttcctcagcggcacgctggaccagtacctgcgcgcctacgacgtgaacaccggtaaagaactgtggaaatcgcgtctgccggccggcggccaggcgaccccaatgacctacaccggcaaggacggcaagcaatacgtcctgctcgtggtgggtggtcacggctcgctgggcaccaagatgggtgactatgtgattgcctacaaactgccggaataa

SEQ ID NO: 54 - protein, 803 aa, Pseudomonas fluorescens
MSTEGASSRSRLLPNLLGILLLLMGLAMLAGGIKLSTLGGSLYYLLAGIGITLTGILMLMRRRAALGLYAIVLFASTVWALWEVGLDWWQLVPRLALWFVLGFVMLLPWFRRPLLLAGPAPMGTGGLTVAVILAGVTALASLFTHPGETFGELGRDTADTTSTAPQMPDGDWQAYGRTEFGDRYSPLKQITPANVGKLQEAWRIQTGDLPTADDPVELTNENTPLKANGMLYACTAHSKVLALDPDTGKELWRFDPQIKSPEGFKGFAHMTCRGVSYYDEAAYAKSENAASTVISEAGKAVAQACPRRLYLPTADARLIALNADTGKICEGFGTNGVVDLTQGIGPFTAGGYYSTSPAAITRDLVIMGGHVTDNESTNEPSGVIRAYDVRDGHLVWNWDSNNPDATEPLAPGQTYSRNSANMWSLASVDEKLGMVYLPLGNQTPDQWGGDRTPGAEKFSAGLVALDLATGKVRWNYQFTHHDLWDMDVGSQPTLLDMKTADGIKPALIAPTKQGSLYVLDRRDGTPIIPIREIPVPQGAVKGDHTAPTQARSDLNLLAPELTEKAMWGASPFDQMLCRIQFKELRYEGQYTPPSEQGSLIYPGNVGVFNWGGVSVDPVRQMLFTSPNYMAFVSKMIPRADVPADSKRESETSGIQKNTGAPYAVTMHPFMSPLGVPCQAPAWGYVAGIDLTTGKVVWKRKNGTSRDSSPIPIGFTLGVPSMGGSIVTAGGVGFLSGTLDQYLRAYDVNTGKELWKSRLPAGGQATPMTYTGKDGKQYVLLVVGGHGSLGTKMGDYVIAYKLPE

SEQ ID NO: 55 - DNA, 3525 bp, Rhodopirellula baltica
atgaggtctcttatcagcactatgtcgtcgccaattgctttcccctctcgctcgttttcccgttctttgcgtcggtcgcggcgtccgtcttccttcattcgctccgcgatcgggctcggcctcatcgtcgctgcgatgccggtctgtgccgttctcagtggtaccgattcgatcggctcggtgagtgccgacgagccagctgccgcggcggtgacgaataccaaacccgaagcgctggaaccggaaatcgccgaggcgtccgaagaagcggcccaggcgatggcgggattcaagatccctgaaggctgggaaatcaaacttttcgcggccgagccccaagtcgccaacatcgtcgcgtttggcgtggattcgaagggccgagtctacgtttgcgaaagctaccggcagaatcgcggcgtgacggacaaccgaggtcacgacgacgaatggttgctggctgatttgtcggccgagacagttcaagaccgaattgattaccacaaacggttgctcggtgaagcggcgatcacttacgcacaacatgacgatcggatccgccggttaacggacaccgacggcgatggtgtcgccgatgagtccaccgtcgtggccgatggcttcaacggtttggaagaggggaccggcgcgggagttttgatcaacggttcggatatctattacacctgcattcccaaactgtggaaattgaccgacgcggatgacgacggcgtcgcggaagattcgcaagtgttgtcggatgggtatggcgtccgagtcgctttccgtggtcacgacatgcacgggttgatccgaggctatgacgggcgtttgtacttctcgatcggagaccgtggttaccacgtgacgactcccgaaggaaagctgttgtccaacccagccgtcggcgctgtgttccgttgcgaaatggacggcagccagttggaagtttattgcaacggtttgcggaacccgcaggagttggccttcaacgacatcggcgactggttcaccgtcgacaacaactccgacagtggcgacaaagctcgtttggttcatctgttggaaggtggcgacacgggttggcggatgcactatcagtacttgcctgatcgcggacctttcaatcgtttgaagatttgggagccgcaccacaacgaacaaccggctcaccttgttccgccgatcatcaacttcaccgacggaccatcgggactcgccttttatccgggcaccggattcggggaactactcgacaatcaatttctgatttgtgatttccgtggcggcccatccaatagcggcatccggtcgttctcggtggatcccgatggggccttttacaaaatgggcgccgatgaccagccgatttggaacattctcgcgaccgacgttgcgttcactccttcgggtgagcttttggtcagtgactgggtcgatggttgggacggacttggcaaagggcgcttgtacaagctgagtgacccggcacagcaagacaccgatgttgtgacggaagtcaaggaactgctcagtggtgatttcacctccgcatcggtggaggatctgaccgagcaacttcgccacatcgatcgtcgagtgcgtttgaatgctcagtgggaactcgccagccgcggtgaaacgaaacccctgatcgacatcgcgtcagcgactgacttggatgctcgtttgcgtttacacggcatttggggatgcgagcacgcggtgcgtttggatggttcgaagcaagcggatgtgttggccgccaatcgcggttggttgaccgattccgacccggtcattcgtgccgccgcctgtgctttggccggcgatcggaacgatgccgaatcagtcgcgaagatcagcgagttgttggctgacgaatcgcctcgcgtgaagtacttcgccgcgatgtcattgtccgaattgttgacttcgcaaactggcaacgcttctgtcaatcagcaagcgatcggcagtgtcttgcagattttggcgaagaacgacaacaacgatcctgccctccgacacgcgtgcacgtttttcctgcgacgtgttgcatcggaagacttgttggcgggattggcgacgcacagcagtgttcccgttcgccgagccgccatcgccgctcttcgtggtcaaggcagcgagaaggtgacggcatttttgtccgacgccagttcactggtcttggcggaagccgcgatggcaattcacgaccgaccaattccggctggcgtggacgagttggcgaagctgatttcggtcgcggatttgccgctgaattcggaggcgttgttgcgtcgcgtgttgacggccaattaccgaattggcacggctgactccgctgcggctctagcgtccttcgctgcgtcgggtgaaaagccgacgtgggctcgaatcgaagcgatcgacatgttggccaattgggccactccggaaccgcgtgaccgagtcacgaacgaatatcgacctttggaaagtcgtcctgaaatcgtcgctcgcgaagcgttggccaagcacatcgaagtcttgatgatcaccgaccaaacggttcgcgaaaaagcgatcgatgttggatccaaacttgggatcaagaaaatcgctccgcagttgattgcacgcaccaatgacgctcaatcgcgacccgcatcacgagcgtcggcattgacggcgttggctcgtttgcaacccgctcaagcggtccagatcgctcgggcgatcgatatcgagcaaccgactcagttggtcaccgccgcactctccgttctgggcaagcacgatggccccgcgtccgttgatcgcttcattgccgcgacggcgaccgagagccaactcgttcgaggattggcttgggacttgttggccaacgtggagtcgaaagaggccctcgagcacatcatcaaaggtgtgaacgcttacctcgaaaatgatcttccggccgatgttcagttgaacctggtcgaagccgccgcgaaacgtttgcctgaagactggaacgcgaagatcgccgctcatcgagagcaattggcgacgacggaaccattggcgaagtggatggattcgttgcacggtggtgatcccgacaagggagcgaagctgttctttggcaagaccgaactttcgtgtgtgcgttgccacaaagtggatcgcgcgggtggtgaagttggtccggtcctgacaacgattggcaaaacgcgagaccgtcgcacgttgctggaagcgatcgctctgccggatgcaaagatcgcagaaggcttcgagacggctgtaatcgcggacgaagatggacaggtcttcaccgggattgtgggagcggagaccgacgatgtgattgaactgatcgccgccgacgggtcacggtctcggatcgagaaggactacatcatcgctcggaagaaaggcaagtcatcgatgccggctggattgactgagcagatgaccgcccgcgaattgcgtgatttggttgcctatttggcgagccttcaagtcgacccacgggccggcgaggaagaaggccacgaatga

SEQ ID NO: 56 - protein, 1174 aa, Rhodopirellula baltica
MRSLISTMSSPIAFPSRSFSRSLRRSRRPSSFIRSAIGLGLIVAAMPVCAVLSGTDSIGSVSADEPAAAAVTNTKPEALEPEIAEASEEAAQAMAGFKIPEGWEIKLFAAEPQVANIVAFGVDSKGRVYVCESYRQNRGVTDNRGHDDEWLLADLSAETVQDRIDYHKRLLGEAAITYAQHDDRIRRLTDTDGDGVADESTVVADGFNGLEEGTGAGVLINGSDIYYTCIPKLWKLTDADDDGVAEDSQVLSDGYGVRVAFRGHDMHGLIRGYDGRLYFSIGDRGYHVTTPEGKLLSNPAVGAVFRCEMDGSQLEVYCNGLRNPQELAFNDIGDWFTVDNNSDSGDKARLVHLLEGGDTGWRMHYQYLPDRGPFNRLKIWEPHHNEQPAHLVPPIINFTDGPSGLAFYPGTGFGELLDNQFLICDFRGGPSNSGIRSFSVDPDGAFYKMGADDQPIWNILATDVAFTPSGELLVSDWVDGWDGLGKGRLYKLSDPAQQDTDVVTEVKELLSGDFTSASVEDLTEQLRHIDRRVRLNAQWELASRGETKPLIDIASATDLDARLRLHGIWGCEHAVRLDGSKQADVLAANRGWLTDSDPVIRAAACALAGDRNDAESVAKISELLADESPRVKYFAAMSLSELLTSQTGNASVNQQAIGSVLQILAKNDNNDPALRHACTFFLRRVASEDLLAGLATHSSVPVRRAAIAALRGQGSEKVTAFLSDASSLVLAEAAMAIHDRPIPAGVDELAKLISVADLPLNSEALLRRVLTANYRIGTADSAAALASFAASGEKPTWARIEAIDMLANWATPEPRDRVTNEYRPLESRPEIVAREALAKHIEVLMITDQTVREKAIDVGSKLGIKKIAPQLIARTNDAQSRPASRASALTALARLQPAQAVQIARAIDIEQPTQLVTAALSVLGKHDGPASVDRFIAATATESQLVRGLAWDLLANVESKEALEHIIKGVNAYLENDLPADVQLNLVEAAAKRLPEDWNAKIAAHREQLATTEPLAKWMDSLHGGDPDKGAKLFFGKTELSCVRCHKVDRAGGEVGPVLTTIGKTRDRRTLLEAIALPDAKIAEGFETAVIADEDGQVFTGIVGAETDDVIELIAADGSRSRIEKDYIIARKKGKSSMPAGLTEQMTARELRDLVAYLASLQVDPRAGEEEGHE

SEQ ID NO: 57 - DNA, 2382 bp, Erwinia tasmaniensis
atgcatagtaaagcatcaagaatactgatcctgctaacggttatattcgccgcgctaagcggcttatatctgttaatcggcggtatctggttggctaaacttggcggttctctctactacatcatcgctggtgtggttctgttggtcaccgcattcctgctgcaccgccgtcgcggttcagcgctgctgctgtacgcgctgttcctgctgggtacgaccgtctggtcgctgtgggaagtgggctctgacttctgggcgctgactccgcgtctggatatcaccttcttcctcggactgtggttggtattgccatttatctggcgtgagctgaacggcacagggtcattctcccgcgtggccctttctgccgtgctggtctttgtcgtcgcggtcctcgcctattcaatatttaacgatccgcaggaaatcaacgggtcactgaatgctgaacaggcggaagcggtgccggctgaagatggcgtcgcaccgggcgactggccggcctatggccgtacccagggcgggacgcgctactctcctctgaagcagataaatgataaaaacgtgggtgaactgcaggaagcctggactttccagaccggtgacctgaaatccccaagcgatccgggtgagatcaccaatgaggccacgccgatcaaaatcggcaatgcgctctacctgtgcaccgcgcatcagcagctgttcgcgctggatgcggccaccggtaagaagaagtggatgttcgatccgaagctgaagccgaatccaaccttccagcacgtcacctgtcgcggcgtttcttattatcagaccccgcaggcggctgcgacgccggccggaaccgaaccggctctctgctcacgtcgtattctgctgccggtcaatgacggcagcatgtatgcgctggacgcagaaaccggcgcgctgtgcgaacagtttggtgacaaaggcagactgaacctgcagagcaacatgccgtatgcaaaagtgggttcttacgagccgacttcaccgccgatcgtcacggccaccagcattgtaatggcgggcgcggttaccgataactactccactaaacagccgtctggcgtggtgcgtggttttgacgtgaacaccggcaaactgctgtgggcattcgacagcggagcgaaagatccgaatctgctgccgcatgacgatcagaagtatacgccgaactcacctaactcgtgggcaccggcagcctatgatgacaagctggacctggtctatctgccgatgggtgtgtcaacgccggatatctggggcggtcatcgtaccccggaaatggagcgtttcgccaacggcgtgctggcgctgaatgccaccaccggtaaactggcatggttctatcagaccgtgcatcacgacctgtgggatatggacgtacctgcacagccgactctggccgatatcgacgacaaagacggacacaaggtgccggtgatttatatcccgaccaaaaccggcgatatctttgtgctgaaccgcaccaacggccagcccgtggtgccagcgccggaaatggtggtgccgggtggccctgctaagggcgatcgtctttcgccgacccagccatactctgaactgagcttccgtccaaaagcccatctggcgggtaaagacatgtggggtgcaaccatctacgatcagctggtctgccgcgttatgttccaccagctgcgctatgaaggtccgttcactccgccatccgagcagggtacattggtattcccaggcaacctcggtatgtttgagtggggcggtatcgcggtggatggcgatcgtcaggttgccatcactaacccaatggcgctgccgttcgtttcccgcctgatcccacgcgggccgggcaatcccattgagccggatgaaaatgataagggcggcaccggtagcgagaaaggtattcagccgcagtacggtttgccgtacggcgtgacgctgaacccattcctctctccgatcggcttaccttgtaagcagccttcatggggctatatttctgcggttgatctgaagaccaacgaaatcgtgtggaaaaagcgtattggcaccgtgcgcgacagctcaccgcttccgcttccgtttaaaatgggcatgccaatgctgggtgggccagttgccaccgcgggcaacctgttctttattgcggcaacggcagataactacctgcgtgcgtttaacgtgactaacggcaaacagctgtgggaagcgcgcctgcccgcgggtggtcaggcaacgccaatgacgtatgaagttaacggcaagcagtacgtgctgatttttgcgggtggacatggctcgtttggcaccaagctgggcgactatgtgaaggcctatgcactgccggacagcaagtaa

SEQ ID NO: 58 - protein, 793 aa, Erwinia tasmaniensis
MHSKASRILILLTVIFAALSGLYLLIGGIWLAKLGGSLYYIIAGVVLLVTAFLLHRRRGSALLLYALFLLGTTVWSLWEVGSDFWALTPRLDITFFLGLWLVLPFIWRELNGTGSFSRVALSAVLVFVVAVLAYSIFNDPQEINGSLNAEQAEAVPAEDGVAPGDWPAYGRTQGGTRYSPLKQINDKNVGELQEAWTFQTGDLKSPSDPGEITNEATPIKIGNALYLCTAHQQLFALDAATGKKKWMFDPKLKPNPTFQHVTCRGVSYYQTPQAAATPAGTEPALCSRRILLPVNDGSMYALDAETGALCEQFGDKGRLNLQSNMPYAKVGSYEPTSPPIVTATSIVMAGAVTDNYSTKQPSGVVRGFDVNTGKLLWAFDSGAKDPNLLPHDDQKYTPNSPNSWAPAAYDDKLDLVYLPMGVSTPDIWGGHRTPEMERFANGVLALNATTGKLAWFYQTVHHDLWDMDVPAQPTLADIDDKDGHKVPVIYIPTKTGDIFVLNRTNGQPVVPAPEMVVPGGPAKGDRLSPTQPYSELSFRPKAHLAGKDMWGATIYDQLVCRVMFHQLRYEGPFTPPSEQGTLVFPGNLGMFEWGGIAVDGDRQVAITNPMALPFVSRLIPRGPGNPIEPDENDKGGTGSEKGIQPQYGLPYGVTLNPFLSPIGLPCKQPSWGYISAVDLKTNEIVWKKRIGTVRDSSPLPLPFKMGMPMLGGPVATAGNLFFIAATADNYLRAFNVTNGKQLWEARLPAGGQATPMTYEVNGKQYVLIFAGGHGSFGTKLGDYVKAYALPDSK

SEQ ID NO: 59 - DNA, 2400 bp, Burkholderia xenovorans
atgcacagttccgcctcaattcgcgtagcgcatttgcttagcgttctgtttgcctcattgtcgggggtgtacctgctggtcggcgggatctggctagcggcgaccggcggatcgctgtactacgttttcgccggtatcgtcatgctcgttacggcggttctgatttatcggcgttccccactagcactcggcctctacgcattactgctgttgtgcacgatcgtgtggtcgctgtgggaggtcggcacggatttctgggctcttgccccgcgtctggacgtactggtcgtcttcggcatctggctgctgttgccgtatgtctatcgcgcgttcgagccgtccgcaaaaacgcatggtctcgcgctcggggccacgttgatcgtgagcgtcgcagtgctggtttttgcagtcttcaacgacccacaggaaatcaacggcacgctggcgagcgcgcctgcccaggcggcggcgacgcccgccggcgagcgcgtcgccgagggcgactggcccgcctacggccgtacccagcacggtacgcgctactctccgctcaagcagatcaacgcggataacgtcaaggatctgaaggtcgcgtggatattccggaccggcgacatgaagcgctccaccgacccaggtgaaatcacgaacgaagttaccccgatcaaggtgggcgacatgctgtatctgtgctcgccgcaccagatactgttcgcgcttgacgctgccaccggccaggaaaaatggcgcttcgatcccaagctgaagaccgatcccagttttcagcatgtcacgtgccgtggggtgtcgtaccatgattcgaccgcttcgggcagtaacaacgacagcccggaccaggccccggctacggcgcctgcaatctgcgcgcgccgcgtctacctgcctgtcaacaacgggcatctgtacgcgctcgacgcgcagaccggccagctatgcccggacttcgccaatcacggcgacctcgacctgcaagtgggccagcccgtgacgaccgcgggtcaatatgaacccacttcgccgccggtcatcaccgggaaggtgatcgtaatcgcaggctcagtcgaggataactattcgacgcgggagccctcgggagtgatccggggtttcgatgtcaacaccggcaaactgttgtgggcgttcgatccgggcgcgaagaacccggaagcggtgctcggcgcaggccagcattattcggtcaactcgccgaactcctgggcaccggctgtttatgacgcaaagctcgatcttgtctatctgccgatgggcgtaagcacgcccgacatctggggcggctaccgcacgccggagcaggaacgctttgcgagcgggctgctcgcgctcaacgcgacgaccggcaagctcgcctggttttatcagacggtccaccacgacctgtgggacatggacctgccggcgcagccgacgctcgccgatatcacggaccggtcgggcaacctcgtgccggcggtctacgccccggcaaagacgggcaacattttcgttcttgaccgccgcacaggcacgccgatcgttcctgctccggagaagccggttccgcaaggcgcggcgcaaggcgatcacgtgtcgcccacccagccgttttcggcgctgaccttccggccggacaggaaactgaccggcgcggacatgtggggcgcaacgatgtttgaccagctcgtgtgccgagtgatgttccagcgcctgaactatgacggcacgtttacccctccgtcggtgaagggcaccctggtttttccgggtaacctcgggatgttcgagtggggcggcatagcagtggataccgatcgccagatcgcgattgcgaacccgatcgcactgccgttcgtgtccaggttgatgccgcgtgggccgggcaacccgatcgaaccggccgccggcggcacgggcggcagcggtaccgaatccggtatccagccgcagtacggcgtgcccttcggtgtgacgttgaatgccttcatgtcgccgctcggcctgccgtgcaagcagccggcgtggggctacatatccgcggtcgatctgaagaccaaccagattgtctggaaaaagcgcattggcaccgtgcgtgacagttcaccgctgccgctgccgttcaagatggggatgccgatgctcggcggcccgatgacaacggccggcgacgtgttcttcatcggggccaccgccgacaactacatccgcgcgttcgacaccaacaccggcaagcagttgtggcaagcacgtttgcccgcgggtggccaggcgacgccaatgacctacgaggcgaaagggaagcagtacgtggtcatcgcggccggcggccacggctcgttcggcaccaggttgggcgactacgtgatggcttatgcgctgccttga

SEQ ID NO: 60 - protein, 799 aa, Burkholderia xenovorans
MHSSASIRVAHLLSVLFASLSGVYLLVGGIWLAATGGSLYYVFAGIVMLVTAVLIYRRSPLALGLYALLLLCTIVWSLWEVGTDFWALAPRLDVLVVFGIWLLLPYVYRAFEPSAKTHGLALGATLIVSVAVLVFAVFNDPQEINGTLASAPAQAAATPAGERVAEGDWPAYGRTQHGTRYSPLKQINADNVKDLKVAWIFRTGDMKRSTDPGEITNEVTPIKVGDMLYLCSPHQILFALDAATGQEKWRFDPKLKTDPSFQHVTCRGVSYHDSTASGSNNDSPDQAPATAPAICARRVYLPVNNGHLYALDAQTGQLCPDFANHGDLDLQVGQPVTTAGQYEPTSPPVITGKVIVIAGSVEDNYSTREPSGVIRGFDVNTGKLLWAFDPGAKNPEAVLGAGQHYSVNSPNSWAPAVYDAKLDLVYLPMGVSTPDIWGGYRTPEQERFASGLLALNATTGKLAWFYQTVHHDLWDMDLPAQPTLADITDRSGNLVPAVYAPAKTGNIFVLDRRTGTPIVPAPEKPVPQGAAQGDHVSPTQPFSALTFRPDRKLTGADMWGATMFDQLVCRVMFQRLNYDGTFTPPSVKGTLVFPGNLGMFEWGGIAVDTDRQIAIANPIALPFVSRLMPRGPGNPIEPAAGGTGGSGTESGIQPQYGVPFGVTLNAFMSPLGLPCKQPAWGYISAVDLKTNQIVWKKRIGTVRDSSPLPLPFKMGMPMLGGPMTTAGDVFFIGATADNYIRAFDTNTGKQLWQARLPAGGQATPMTYEAKGKQYVVIAAGGHGSFGTRLGDYVMAYALP

SEQ ID NO: 61 - DNA, 2361 bp, Pantoea citrea
atgtctcgggtctctactatacttcatgggttaacacggatattttcgctattctgtgccgcatggctgctgattggcggcgtgtggctattatctgtcggagggagtgcttactaccttatctgcggtatagccatggcgatattttctatattgttatggaagcgcagaagcagtgcattttacctctattcgcttatcctgattctgtcagccctctgggcatggcgggaggcggggactgacttctggaacctggttccacgtctggatatctggatactgtttggtatctggttgatattgccgttcagttaccggcgttttaacactgccggtaaaaaaccattgctggcaatggtgattggcctcgggattaatgccctgctgctgctgggcgcgtcactgcacgacccacaggaaatcaacggtgtgcttaatgtcagtgataagccgccagccgaatctgcggcgtctgcagcagactggccggcctatggacgtactcaggaaggggtccgttattctccgctgacacaaatcaacgataaaaacgttcaacaattacaggttgcctggcagttccataccggtgaccataaaacagcgaatgacccgggtgagatcactaatgaagtgacaccgctgaaagtcggtaatatgctgtacctctgtacaccgcatcagatactgattgccctggatgctgccagtggcagagagaaatggcgttttgatccgcagctcaaatctgatccgacattccagcatattacctgccggggtgtttcctatcatgaaatcaaatctgttcagggcgactcatcagcgccggccgcctgttcacggcgtattttcctgccggttgatgacggtcgtctgttcgcggttgatgccttaaccggacaacgttgcagtaattttgctaataatggtgaactgaacctgcaacacctgcagccgaacgcttatccgggagggtatgagccaacctcaccgcccatcattactgataaagtggtgatcattgccggttctgtcactgataacttgtctacccgtgaaccatcaggggtcatccgcggtttcgatatcgacagcgggaaactcttatgggtatttgatccgggagcaaaagaccctaatgccgtgcctgctgacgggcagacatttgtggcgaactcaccaaactcctgggcgccggcggcttatgacgcgcagagggatattatttatctgcctatgggggtttcgaccccggatatctggggcggtgccgtaacgcgttgcaggaacgttttgccagcggtttactggctctgcatgcctcaactggcaagctggcatggttttaccagacggtacaccatgcaatcattctgggacatggatttaccctcacagccaaccctggcagatattaccgatgagcagggtaagaccgtgccggtggtctatgtgccggccaaaacgggtaatatttttgttctgaaccgtgacaccggaaaacccgtcgttcctgcccctgaaacaccggtaccgcagggaccggcgaaaggcgaccatctgtctccaacccagcctttttctgagctgactttccgtcctaagaataagctgcagggtagggatatgtggggcgcaaccatgtttgaccagctgatgtgccgggtgatgttccataaactgcgctatgaagggccgtttaccccgccgtctgagcagggtaccctggttttcccaggtgatttcgggatgtttgaatggggcggtatctctgttaacaccgatcagcagtttgcgattgctaatccgatggcgatgccttttatttctaaactgatcccacgcggaccaggcaatcccatagaacccggggctgatggtgccgccggttccggttcagagtccggggtacagcatatgtatggtgtgccttacggagttgaactgaatccgttcctgtcaccgttgggcttaccttgtctgcaaccttcatggggctttgtctcagcgatcaatttacgtaatcaccagatcatctggaaaaaacggattggtactgttcgtgacagtgcgccggtacctctaccctttaaaatgggtgttccaatgttgggcggtccggtgacaacggcaggtaatatcttctttgttgctggcacactggacaactacctgcgggcgtacagtgtccgtgacggcaaactgctgtggcaggctcgtctgccggcaggtggacaagcgacaccaatgacgtatgaggttgatggtaagcaatatgtcgtgattatggccggcggacatggttcttttgggacccggctgggcgattcactgatcgcttataaactaccgtaa

SEQ ID NO: 62 - protein, 786 aa, Pantoea citrea
MSRVSTILHGLTRIFSLFCAAWLLIGGVWLLSVGGSAYYLICGIAMAIFSILLWKRRSSAFYLYSLILILSALWAWREAGTDFWNLVPRLDIWILFGIWLILPFSYRRFNTAGKKPLLAMVIGLGINALLLLGASLHDPQEINGVLNVSDKPPAESAASAADWPAYGRTQEGVRYSPLTQINDKNVQQLQVAWQFHTGDHKTANDPGEITNEVTPLKVGNMLYLCTPHQILIALDAASGREKWRFDPQLKSDPTFQHITCRGVSYHEIKSVQGDSSAPAACSRRIFLPVDDGRLFAVDALTGQRCSNFANNGELNLQHLQPNAYPGGYEPTSPPIITDKVVIIAGSVTDNLSTREPSGVIRGFDIDSGKLLWVFDPGAKDPNAVPADGQTFVANSPNSWAPAAYDAQRDIIYLPMGVSTPDIWGGAVTRCRNVLPAVYWLCMPQLASWHGFTRRYTMQSFWDMDLPSQPTLADITDEQGKTVPVVYVPAKTGNIFVLNRDTGKPVVPAPETPVPQGPAKGDHLSPTQPFSELTFRPKNKLQGRDMWGATMFDQLMCRVMFHKLRYEGPFTPPSEQGTLVFPGDFGMFEWGGISVNTDQQFAIANPMAMPFISKLIPRGPGNPIEPGADGAAGSGSESGVQHMYGVPYGVELNPFLSPLGLPCLQPSWGFVSAINLRNHQIIWKKRIGTVRDSAPVPLPFKMGVPMLGGPVTTAGNIFFVAGTLDNYLRAYSVRDGKLLWQARLPAGGQATPMTYEVDGKQYVVIMAGGHGSFGTRLGDSLIAYKLP

SEQ ID NO: 63 - DNA, 5498 bp, Klebsiella pneumoniae
catgtggaagaaacctgcttttatcgatttacgtctcggtctggaagtgacgctgtacatttctaaccgttaatcgccccgcccgccgttcgcgcgggcaccttcattcattacccggtccgtcttcatgttcattaaagtcctcggctccgccgccggcggcggtttcccgcaatggaactgcaactgcgccaactgtcagggtctgcgcaacggcaccattcaggccagtgcccgcacccagtcgtcgatcatcgtcagcgataacggcaaagagtgggtgctgtgcaatgcctcgccggatatcagccagcagattgcccatacccccgagttaaataaacccggcgtactgcgcgggacgtctatcggcggcattattctcaccgacagccagatcgaccacaccaccgggttgctgagcctgcgcgaaggctgcccgcaccaggtgtggtgcacgccggaggttcatgaggatctctccaccggcttcccggtgtttaccatgctgcgacactggaacggcggcctggtgcatcatcccatcgcgccgcagcagccttttaccgttgacgcctgccctgatttgcagtttaccgccgtgcctatcgccagcaacgcgccgccctattcgccgtatcgcgaccggccgctgccaggccataacgtggcgctgtttatcgaataccgccgcaacgggcagacgctgttctatgccccggggctgggtgagccggatgaagcccttctgccgtggctgcaaaaagcggactgtctgctgatcgatggcaccgtctggcaggatgacgagctgcaggccgccggcgtcgggcgcaataccggccgtgatatgggacacctggcgctcagcgatgagcacgggatgatggccctgctggcctctctgccggcaaaacgcaaaattctcattcatattaataacaccaacccgatccttaacgagctgtctccccagcgccaggcgctaaaacaacaggggattgaagtgagctgggacgggatggcaatcacccttcaggataccgcatgctgatcaccgacacgctgtcgccgcaggcctttgaagaggctctgcgggctaaaggcgccttctaccatattcaccacccttaccacatcgccatgcataacggcgaagcgacccgcgagcaaattcagggttgggtggcgaaccggttttattaccagaccaccattccgctgaaagacgcggcgattatggctaactgcccggatgcgcagacccggcgcaaatgggtgcagcggatcctcgaccacgacggtagccacggcgaagatggcgggattgaagcctggctgcggctgggggaagcagtcggtttgagccgcgacgacctgctcagcgagcgtcacgtgctgcccggcgtgcgcttcgcggtggatgcctatcttaatttcgctcgtcgcgcctgctggcaggaggcggcctgcagctccctgaccgagctgttcgccccacagatccatcagtcgcgcctcgacagctggccgcagcactatccgtggatcaaagaggaaggctatttttacttccgcagtcgtctgagccaggctaaccgcgacgttgagcatggtctggcgctggcgaagacctactgtgacagcgctgaaaaacagaaccggatgctggagatcctgcagtttaagctcgacatcctgtggtcgatgctcgatgccatgaccatggcctacgctctgcaacgcccgccctatcacacggtcaccgacaaggcggcctggcacacgacccgactggtgtaatcatgcaaaaaacgtccatcgttgcctttcgtcgcggctaccgactgcagtgggaagccgcccaggagagccatgtgatcctctatccggagggaatggctaaactcaatgagaccgccgcggcgatcctcgagctggtcgatggccggcgcgacgtcgcggcgattatcgccatgcttaacgaacgtttcccggaagccggcggcgtcgatgacgacgtcgtcgagttcctgcagatcgcctgtcaacagaagtggatcacctgccgtgagccagaataaacccgccgtcaatccgccgctgtggctgctggcggagctgacctaccgctgcccgctgcagtgtccctactgttccaatccgctggacttcgcccggcaggaaaaggagctgaccaccgaacaatggatcgaggtctttcgccaggcgcgagcgatgggcagcgtacagctgggcttttccggcggcgagccgctgacccgtaaagatctgccggagctgatccgcgccgcgcgcgacctcgggttctataccaacctgatcacctcgggaattgggctaaccgagagcaaactcgacgccttcagcgaggccggactggaccatatccagattagcttccaggccagcgatgaggtgctcaacgccgctcttgccggcaataaaaaagccttccagcagaagctggcgatggccagagcggtgaaagcgcgcgactacccgatggtgctgaacttcgtcctccaccggcataacatcgaccagctcgataaaattatcgagctgtgcattgagctggaagccgatgacgtcgagctcgccacctgccagttttacggctgggcgtttcttaatcgcgaggggttactgccgacccgggaacagatcgcccgcgccgagcaggtggtcgccgattaccggcagaaaatggccgccagcggtaacctcaccaacctgctattcgtcaccccggactattacgaggaacgcccgaaaggctgtatgggcggctggggatcgattttcctcagcgtcactccggaaggcactgcgttgccgtgccacagcgcgcgccagctgccggtggcgttcccgtcggtgctggagcagagtctggaatcgatctggtatgactcgttcggcttcaaccgttatcgcgggtatgactggatgccggagccgtgccgctcctgtgatgaaaaagagaaagacttcggcggctgccgctgtcaggcctttatgctgaccggcagcgccgataacgccgacccggtgtgcagcaaatccccacatcatcacaaaatccttgaggcccggcgcgaagcggcctgcagcgacatcaaagtcagccagctgcagttccgcaaccgtacccgctcgcagcttatctacaaaacccgggaactgtaatgacgctggcgacccgcactgtcactctgccgggcggcctgcaggctaccctggttcatcagccgcaggccgatcgcgcggcggccctggtgcgggttgccgccggcagccaccatgaaccgtcgtgcttccccggtctggcgcacctgctggaacacctgctgttttacggcggtgagcgctaccgcaatgatgaacggctgatgagctgggtgcagcgccaggcagggaatgtgaatgcctccaccctgtcccgccacagcgcgttctttttcgaggtcgccgccgaggatctggctgacggcgtcgcgcgcctgcaggagatgctgcaggcgccgctgctgctcagggacgatattcaacgcgaagtcgcggttatcgacgccgaaaacggcctgatccaacagcatgagttgtcgcgacgggaagccgccgtgcgtcacgccgccatcgcgcccgcggcgtttcgccgctttcaggtcggcgacgccgggtcgctgggggaggatttcctcgcgctacaggcggccttacgtgactttcaccgcagccactacgtcgcccgccggatgcaactctggctgcaggggccgcagtcgctggaggtgctcggcgaactggcgacccgtttcgccactgggcttgccccgggcgaggcaccgccgccagcgccgccgctcagtctgggcgagccccctcaactgcagttggccgtctccagccagcccgcgctgtggcgctgcccgctgatcgccttaagtgacaatgtcacgttactgcgcgagtttttgctggatgaagcccccggtagcctgatggccggcctgcgccagcgcgggatggccgaggacgtggcgctgaactggctgtatcaggatcagcacttcggctggctggcgctgattttcgccagcgaccggccggaacaggtcgaccggcagataacccactggttgcaggcgctacagcagacgacgcctgagcagcagcaacactactatcagctgtcccggcgccgttttcaggcgctgtcgcccctcgatcagctgcgccagcgggcattcggctttgcccccggggcgccgcccaccgggttcgccgatttttgcgccgccctgctggccgcccccacggtcagcctggcctgccagacgcagccccccggagcaacggtagccacccagggctttagcctgccgctcagccgctggtcgccacgtccggtctctgacccggcgctggcattcgctttttatccgcaggccgctggcgagctcgtggccgaaagcccggcggaagccgcgccactgcgtcacctcccgtcaccgggagagccgccgacgctcctgctgcgaccgcccttctactgctcgcccacgccggccggggggctggcgcgcggggaacagctgcgtccattacttgccgccctgcgccatgccgggggacacggcgagtggcatctgttcgacggcagctggcagctgatcctgcagttgcctgcgtccggccaatggccggaggcgattctgcaggccatcgtgcggcagctcgcgctcccggtcgccccgctgcccccaccgccggagagtattgcgatccgtcatctcatggcccagctccccgaacggctgggtacgtcagcgcaccaggaaggttggctggcggccctgattggcggcagcgcggaagatgcgcagtgggtagcgcgtcagctgagccggcttaccgtcccggttaatccgccgatgcccgctccggccacctgccgcggcggcgtcgagcggctggcttatccccggggcgacacggcgctactggtctttcttccgctgccggaaggcgcttcattggcggccctgcgggtgctggcgcagttctgcgagccgccgtttttccagcgcctgcgggtggagcagcagataggctatgtggtgagctgccgctatcagcgcgttgccgatcgcgacggactgctgatggcgctccagtccccggatcgccgccccggggagctactccgctgctgtaaaacctttctgcgccagctggcccccgtggatgaggcgaccttcaggttgttacagcagcggctggccgctcaggcccgcgcccgggtagagccgcaggtgcgcgccctggacgcgctgcgccaggagtataacttgccgggggtgacgccgcaggcggctgacgcgctgcgcgttgaagaggtggtcgccctgtggcatgagatgacccgccggcgtcgtcgctggcgggtgctgttcacgacagggagttaa

SEQ ID NO: 64 - DNA, 3473 bp, Methylobacterium extorguens
atgaagtgggctgcccccatcgtttccgagatctgcgtcggcatggaagtcacgagctacgagtcggccgagatcgacaccttcaactaaggtgatttgagccgggttggggttgcaggcatcagcgggttttcaccatgcatgtcgtaatcctgggctcggctgcgggcggcggcgttcctcaatggaactgccgctgctccatctgctccctggcctgggcgggcgattcccgcgtcaggccgcgcacgcagtcgagcatcgcagtctctcctgacggggaacgctggctcctgctgaacgcctctcccgatatccgtcagcagatccaggccaatccgcagatgcatccgcgcgagggcctgcgccactcgccgatccacgcggtgctgctgacgaacggcgacgtcgatcacgttgcgggcctgctgaccctgcgcgagggccagcccttcacgctctacgcgacacccggcatcctggcctccgtctccgacaaccgcgtcttcgacgtgatggccgccgacgtggtgaagcggcagacgatcgccctcaacgagaccttcgagccggtgcccggcctctcggtgacgctgttctccgtccccggcaaggtgccgctctggctggaagacgcctcgatggagatcggggcggagaccgaaaccacggtcggcacgatgatcgaggccgggggcaagcgcctcgcctacatccccggctgcgcccgggtgacggaggatctcaaagcccgcatcgccggcgcagacgcgctcctgttcgacggcacggtgctggaggacgacgacatgatccgcgccggtgtcggcaccaagaccggctggcgcatgggccatatccagatgaacggcgagaccggctcgatcgcgtctctcgccgatatcgagatcggccgacgggtcttcgttcacatcaacaacaccaatccggtcctgatcgaggattcgtacgagcgcgcgagcgtcgaggcgcgcggctggaccgtcgcccatgacggcctgaccctcgatctctgatcaggctgatgtcttgggaagagcccggtctggaaatttagtgccggactgaatatgttttgcacgatccaatcgtgcggcagcggccctgccccgatcggtaccgggccccatttaaaaataaatccaggaaacgcgactcgaagctcgggggaaaccgaacgccatgaccgcccaattcccgccgcccgtcccggacaccgagcaacgcctgctgagccacgaggagcttgaggcggcgctccgcgatatcggtgcacggcgctaccacaacctccacccgttccaccggctgctgcacgacggcaagctgtcgaaggatcaggtccgggcctgggcgctcaaccgctactattatcaggcgatgattccggtgaaggatgcagcgctgctggctcgcctgccggatgcgcagcttcgccgaatctggcgccagcgcatcgtcgatcacgacggcgaccatgagggcgacggcggcatcgagcgttggctcaagcttgccgaaggcgtcggcttcacccgcgactacgtgctctcgaccaagggcatcctgtcggcgacccgcttctcggtcgatgcctatgtccacttcgtctccgagcgcagcctgctcgaagccatcgcctcctcgctgaccgagatgttctcgccgacgatcatctccgagcgcgtcgccgggatgctgaagaactacgacttcatcaccaaggacacgctggcctatttcgacaagcgcctgacccaggccccgcgcgacgccgatttcgccctcgactacgtcaagcggcacgccaccacgcctgagatgcagcgggcggcgatagatgcgttgacgttcaagtgcaacgtgctctggacgcaactcgatgcgctctacttcgcctatgtcgcccccggcatggtgccgccggatgcttggcagccgggcgagggccttgttgccgagacgaactccgccgaggacagccccgccgctgcggccagccccgccgcgacgacagctgaacccacggccttctcgggcagtgacgtgccgcgcctgccccgcggcgtgcgcctgcgcttcgacgaggtccgcaacaagcacgtgctgctcgcccccgagcgcaccttcgacctcgacgacaacgccgtcgcggtcctcaagctcgtcgatggccggaacacggtttcgcagatcgcccagattctgggtcagacctacgacgccgacccggccatcatcgaagccgacatcctcccgatgctggccggcctcgcgcaaaaaagggttctggagcgatgaatgcaccgacacccgccccctcccccgtggacgtcattccggcgccggtgggtctgctcgccgagctgacgcaccgctgcccgctgcgctgcccatactgctcgaacccgctggagctcgaccggcgctcggccgagctggacacgcagacgtggctgcgggtgctgacggaggcggcggggctcggtgtgctgcacgtccacctgtcgggcggtgaaccgaccgcccgccccgacatcgtcgagatcacggccaaatgcgccgaactcggcctgtactcgaacctgatcacctccggcgtcggcggtgccttagcgaagctcgacgcgctctacgacgtcggcctcgaccacgtgcagctctccgtccaaggggtggacgcggccaacgcggaaaagatcggcggccttaagaacgcgcagccgcagaagatgcaattcgctgcccgggtcaccgaactcggcctgccgctgacgctgaactcggtgatccaccgcggcaacatccacgaggtgccgggcttcatcgacctcgcggtcaagctcggcgccaagcggctggaggtggcccatacccagtattacggctgggcctatgtgaaccgcgccgcgctgatgccggataagagccaggtcgacgagtcgatccgcatcgtcgaggccgcgcgcgagcgcctcaagggtcagctcgtcatcgacctcgtggttccggactactacgccaagtacccgaaggcctgcgccggcggctggggccgcaagctgatgaacgtgacgccgcagggcaaggtgctgccctgccacgccgcagaaaccatccccggcctcgaattctggtacgtcaccgaccacgcgctcggcgagatctggacgaagtccccggcctttgccgcctatcgcggcacgtcctggatgaaggagccctgccgctcctgcgaccggcgcgagaaggattggggcgggtgccgctgccaggcgctggcgctcacgggcgacgcggccaacaccgatccggcctgctccctttcgccgctgcacgcgaaaatgcgggatcttgccaaggaagaggctgccgagaccccgcccgattatatataccgcagcatcgggacgaatgtgcaaaacccgttgagcgaaaaggcacccctttga

SEQ ID NO: 65 - DNA, 2738 bp, Methylobacterium extorguens
atgcacctctaccggaaggccatcgcgcccgtgggaccgcagcgaggcctgaatcacgccggccgtgtggacgccgccccgttcgggcgctccgaggcgggcggcccggaggtctccgccttcgtgctcgacaacgggctcgacgtggtggtggtgcccgatcaccgggcgccggttgccacgcacatggtctggtaccgcaacggctcggccgatgatccgatcggccagtccggtatcgcccacttcctcgaacacctgatgttcaagggcaccgagcggcacccggccggcgccttctcgaaagcggtctcgtcgctcggcggccaggagaacgccttcaccagctacgattacaccgcctatttccagcgcgtcgcccgcgaccacctctcgacgatgatggccttcgaggccgaccggatgagcggcctcgtgctcgacgacgccgtggtggcgcccgagcgcgacgtggtgctggaggagcgccggatgcgggtcgagaccgatccgtcggcgcagctctccgaggcgatgtccgcctcgctgttcgtgcaccatccctacggcatcccgatcatcggctggatgcacgagatcgaggagctgaaccgcacccacgccatcgactattacaagcgcttctacacccctgagaacgcgatcctcgtggtggccggcgacgtgacgccggacgaggtgcggcgtctggccgaggatacctacggccgggtgacgccgcagggcgcgcggccgctgcgcactcgcccgcgcgagccggagccgcgggcgatgcgtcggatcgcggtggccgacccgaaggtcgagcagccgaccctgcagcgcctctacctcaccccctcctgcatgaccgcccgcgacggcgagggctacgccctcgaactgctcgccgaggtcgtcggcggcggctcgacctcgttcctctaccgcaagctggtgctggagatgggcgtcgcggtgaatgccggcgcttggtacatgggctcggcgatggatgacacgcgctttgccgtctacgccgtgccggccgagggcgtgaccctggaggccctcgaagagcatatcgatcgcgtgctgcgccgcgtccccgaggcgctcggtgcggaggcgatcgagcgggccaagatccggctcatggccgagacggtctattcctccgattcgcagagctcgctcgcgcgcatctacggctcggcgctcgccatcggcgagaccgtcgaggaggtgcgccgctggccggtcgagatcgaagcggtcacccatgaccggctcgtcgcggtcgccgcccgctatctcgtgcccgcccgctcggtgaccggctacctgaccaaggcgcgcgacccggacgtggcgatcgcctgagacggccctgattcttcgcgccgccctcgatggcggcgccgtttgcgaaaagacgcattaaggaacttccgatgaatctcgccgagaccggcacccgcaccgcgacgtcgccgacccaggcgttgccgctcgcggccgcccccggcatcgaggcttggcacgtcgcctcgccggtggtgccgatgatcgcgctctccttcaccttcgagggcggcgcggcgcaggatgcggagggcaaggcgggcaccgcgcagatgatggcgcggctgctcgacgagggcgcgggcgatctcgactcggatgccttccaggaggcgctcgcggcccgcgcgatcgagctgagcttccacaccggccccgattccatcggcggctcgctcaagacgctgctcacgcatgccgacgaggcgatccgtctgctggccctgtcgctggccgagccgcgcttcgatcaaccctcgatcgagcgcgtgcgggcgcagatgatcgccagcctgcgctaccagcagaacgatcccggcgtgctggcctcccgccgctacttccgcgaggccttcccggggcatgcctacggccgctcctcgtcgggcaccatcgagaccctgtcggcgatcacccgagacgacctcgtcgccctgcaccgggccgtgatcggtcgcggcagcctcaaggtcgcggctgtgggcgccttcgacgaggcgacgatcaccggcatgatcgcccgcgccttcggcgctctgcccgaggccggcccgctcaaagccattccgccgaccgcgatcaacgaactcggccgccgcatcgtcgtcgatctcgacgtgccgcaatcggtgatccgcttcggcatgccgggcgtggcgtggcgcgaccccgacttcatcccggcctatgtgctgaaccacatcctgggcggcggcgccttcacctcgcgcctgttccaggaagtgcgcgagaagcgcggcctggcctactcggtcggcacctctctgacctcgcaccgcgccgtcgccatgacctggggctacaccgccaccaagaacgagcgcgtcgtcgaggccctcgacgtgatcggtgacgagatccagcgcctcatcaccgacggcccctccgacgaggagttgcagaaggccaaggactatctcaccggctcctacgcgctcggcttcgacacctcgaccaagatcgccaaccaattggtgcagatcgccttcgaggggctcggcatggactacatcgcccgccgcaacgacctcgtggcgagcgtgacccaggccgacatccgccgggccggcgcccgcacgctcggcgacggcaagatgctggtcgtcgccgcggggcggcccacggggctgtag

SEQ ID NO: 66 - DNA, 3194 bp, Pseudomonas aeruginosa
atgtggaccaagcccagcttcaccgacctgcgtctcggtttcgaagtgaccctctacttcgccaaccgctgacccatccggccccggtcatccggggcctccctccagcccgcggggcagacccatgcacatccgtattctcggttcggccgccggcggcggctttccccagtggaactgcaactgccgcaactgtcgcggggtccgcgacggcagcgtagcggcccagccacgcacgcaatcctccatcgccctgtcggacgacggcgagcgctggatcctctgcaacgcctcgcccgacatccgtgtccagatcgccgccttcccggccctgcagccggcgcgtcggccgcgcgatacggcgatcggcgcgatcgtcctgctcgacagccagatcgaccacaccaccggcctgctcagcctgcgcgaaggctgcccgcacgaagtctggtgcacgcagatggtccaccaggacctcagcgaaggctttccgctgttccgcatgctttcccactggaacggcggcctgcgccaccggccgatcgccctcgatggcgagcctttcgccatcccggcctgtccgcgcctgcgcttcaccgcgatccccctgcgcagcagcgcgccaccgtattccccgcatcgcggcgacccgcatccgggcgacaacatcggcctgttcgtcgaggacctcgacagcgccggcacactgttctacgcgccgggcctcggcgaggtggacgaggcgctgctcgaatggatgcgccgcgccgactgcctgctggtggacggcacgctctggcgcgacgacgagatgctggcctgcgaggtcggcgacaagctcggccggcagatgggccacctggcgcagagcgggccgggcggcatgctcgaggtactggcgaaggtgccggccgcgcgcaaggtgctcatccatatcaacaacaccaatcccatcctcgacaccgcttcggccgagcgcgccgaactggatgccagtggcatcgaagtggcctgggatggcatgcacatccagctgtagggggaccgacatgagccgtgccgccatggaccgcgccgagttcgaacgggcgctgcgcgacaaggggcgctactaccatatccaccatccgttccatgtcgcgatgtacgagggccgtgccagtcgcgaacagatccagggctgggtggcgaaccgcttctactaccagctcaacatcccgctgaaggacgcggcgatcctggccaactgtcccgaccgcgaggtccgccgcgagtgggtccagcgcatcctcgaccacgacggcgcccccggcgaggcgggaggcatcgaggcctggctgcgcctggcggaggcggtgggcctggagcgcgagcaggtgctgtccgaagaacgggtgctgcccggcgtgcgcttcgccgtcgacgcctatgtcaacttcgcccgtcgcgccagctggcaggaggcggcgagcagttcgctgaccgaactcttcgccccgcagatccaccagtcgcggctggacagctggccgcgccactatccgtggatcgaggcggccggctacgagtatttccgcagccgcctggcccaggcccggcgtgacgtcgagcacggcctgcggatcaccctggagcactatcggacccgcgaggcacaggaacgcatgctggagatcctgcaattcaagctggacgtgctgtggagcatgctcgacgcgatgagcatggcctacgagctggaacgtccgccgtaccatacggtgacccgtgagcgggtctggcaccgggggctggcgccatgagcctgccatcgctcgacagcgtgccggtcctgcgccggggcttccgcttccagttcgaaccggcccaggactgccatgtgctgctctatcccgaagggatggtcaagctcaacgacagcgccggggagatcctcaagctggtcgatggccgccgcgacgtggcggccatcgtcgcggcgctgcgcgaacgttttcccgaagttcccggcatagacgaagacatcctgacgttcctcgaggtggcccatgcgcaattctggatcgagctgcagtgaaagcgtggggccgccgctctggctgctggccgagctgacctaccgctgcccgttgcagtgcccgtactgctcgaacccgctggaattcgcccgcgagggcgccgagttgggcaccgcggaatggatcgaggtgttccgccaggctcgcgagctgggcgccgcccagctcggtttctccggcggcgagccgctgctgcgccaggacctcgccgaactgatcgaggcggggcgcggcctgggcttctacaccaacctgatcacctccggcatcggcctcgacgaggcacgcctggcgcgcttcgccgaggccgggctggaccacgtgcagatcagcttccaggccgccgacgaagaggtgaacaacctgctcgccggctcgcgcaaggccttcgcccagaaactggcgatggcccgcgcggtgaaagcccatggctacccgatggtgctcaacttcgtcacccaccggcacaacatcgacaacatcgagcggatcatccagctgtgcatcgagctggaggccgactacgtggaactggccacctgccagttctacggttgggccgcgctgaaccgcgccgggttgctgccgacccgcgcccaactggagcgcgccgagcggatcaccgccgaataccgccagcggctggccgccgaaggcaatccgtgcaagctgatcttcgtcacccccgactactacgaggaacggccgaaggcctgcatgggcggctgggccagcgtgttcctcgacattaccccggacggcaccgcgctgccgtgccacagcgcgcggcaactgccgctgaagttccccaacgtgcgcgagcacagcctgcgccacatctggtacgagtcgttcggcttcaaccgctatcgcggcgacgcctggatgcccgagccgtgccgctcctgcgaggaaaaggagcgtgaccacggcggctgccgctgccaggcgttcctccttaccggcgacgccgacgccaccgacccggtctgcgccaagtcggcccgccacgacctgatcctcgacgcccggcgccaggccgaggaggcgccgctgggcctggacgcgctgacctggcgcaatcagcgcgcctcgcgcctgatctgcaaggcctga

SEQ ID NO: 67 - DNA, 2292 bp, Pseudomonas aeruginosa
gtgctgcccaatggcctgcgcctgcacctggcgcatgacccggcggcatcgcgcgccgctgcctggctgcgggtggcggcgggcagccacgacgagccgaccgcgcatcccggcctggcacacttcctcgagcacctgctgttcctcggcggcgcggcgtttcctggcgacgagcgtctcatgccgtggttgcaggtgcgcggcggccaggtcaacgccagcacccggggcaggagcaccgactatttcttcgaggtggcggcggagcaccttggcgccggtctggcacgcctgttcgacatgctcgtgcgaccgctgctggatatcgacgcgcaacggcgcgagcgcgaagtgctggaagccgagtacctggcgcgcgcggccgacgagcagaccctgatcgatgcggcgctggccctcggcctgcccgccgggcatcccctgcggcgtttcgttgccgggcgccgcgacagcctggcgctggagaccgatgcgttccagcgggcgctgcgcgaattccatgccgcccactatcacgccggcaattgccagctatggctgcaagggccgcaggcactggacgagttggagaggctggcgcggcgtgcctgcgccgacctgccgggtcgctcgccaggcgcgagtccgtcgccgccgccgttgtcacccttcgccggcgcggcgctagcgctgcgcctgccgggtccgccgcgtctggtgctgggctttgccatcgacgctttgcggggggctgacgaacagaccctgctggcattcgccgaattgcttggcgatcgctcgccgggcgggttgctggcggcactcggcgaacagggcctgggcgaatcggtggcgctgcgggtggtccatcgggacgcgcggcaggcgctcctggcgctgaccttcgaactgttcgacggcagcgcggcggcggcgctggaggagacctttttcgactggctgcgcgccctgcgcgacgatgccgcgagcctgctggcggcgcgccggccgttgctggcggagcccactgcgccgctggagcgactgcgccagcgcgtgcttggcctgccagcggagattcgcccggcctgcctggatgcgctgcgcgccgaacgctgcctgcggttgcacctggacagcgaactcgacggcgccgaagcgcgctggtcggcgggcttccgcctgagcgtggcgcccgtcgccgcagcgccgccgccgctggcagcgcagcggcatgcctggcgcttcgaactgccgccgccgccgagcaccgccgccgagggcgcgctgttcctgcgctggcgctttcccggcatatccgcacggtcacgcttcctggcgctgcgccaggcgttgcgtccgctgtgcggccaggcgcgcctgcggggggtggagatgggcctggaagcgctcggcgaagactggacgctgagcctgctcggagcacgcgaccgcctcgaggccgccgtgcgaccggccctggcccggctcctcgcggcgccggccgactggcgcgcaagcggcgagcgcctgtcgttcgccgagcggcgccgcagcgccactggcctgccgatccggcaactgctggacgccttgcccggcgtactcggcgagccgctggcggaggtcgacgacgactggcggcggacgcgctgggacggcctggtcatgcaggccgcgatgcctgatccacgatgggtgcccgggcaggctaccggcaagcgccttgagcctctgccagcgatgcccggacggcatcgccgcgagctggccgtggacggtgagtcggcgttgctgctgttctgtccgctgcccgcgccggaggtgccgatggaggcggcctggcgcctgctggcgcgcctgcacgagccggccttccagcggcgcctgcgcgacgagttgcaactgggctacgcgctgttctgcggcttccgtgaggtcggcgcacggcgcggcctgctgttcgccgcgcaatctccgcgtgcctgcccggaacgcctgctggagcacacggagatcttcctccagcgttccgtcgaggcgctcgcgcaactgccggcgcaacgcctggccggcctgcgcgatgccctcgccgacgacctgcgccgggcgccggggacttttgccgagcgggcgcgacacgcctgggcggagcatctcggcggtggccagggccgctcccggctgctcgccgaggctgctcgcggcctgggcgtcgacgatctgcttgccgcccaggccgcgctgctggaggcgcacggcggctggtgggtgctttccagccgacgctga

SEQ ID NO: 68 - DNA, 3187 bp, Gluconobacter oxydans
atggcctggaacacaccgaaagttaccgaaatcccgctgggcgcagaaatcaactcgtatgtctgcggcgagaagaaataagccgctttcccggggacccgtccttgaggaataatggcacggccgctcccccatggagcggccgttttcgttcatgggtgctctgtggtgccccagtcagacggtttgtgaaaaaatgattgatgtcatcgtgcttggcgcggcggcagggggcggttttccgcagtggaactccgcagcacccggctgtgtggccgcccgcacgcgacagggcgcgaaagcccggacccaggcctcccttgccgtcagtgccgacggaaagcgctggttcattctcaacgcctcgcccgatctgcggcagcagatcatcgatacgccggccctgcatcatcagggcagcctgcgtggaacgcccattcagggcgtcgtcctgacctgcggcgagatcgacgccataaccgggcttctgaccctgcgtgagcgtgagccttttaccctgatgggcagcgactcgacccttcagcagcttgcggacaatccgatcttcggtgcgctcgatccggaaatcgtcccacgtgttccgctcattctcgatgaagccacgtccctgatgaacaaggacgggattccgtccggtcttttgctcacggccttcgccgttccgggcaaggcgccgctttacgcggaagccgcagggtcacgcccggacgagacgctgggcctttccattacggatggatgcaagacgatgctcttcattcccggctgtgcgcagatcacgtcggaaatcgtggaacgggtagcggcagccgatctcgtgttctttgacgggacactgtggcgggatgacgaaatgatccgcgccgggttgagcccgaagagcggacagcggatgggacatgtgtccgtgaatgatgccgggggaccggtcgaatgtttcacgacatgcgaaaaaccccgtaaagtgttgattcatatcaacaactccaatccaattctgttcgaagacagccccgaacgcaaagacgtcgaacgcgccggatggacggttgcggaagacggcatgactttcagactggacacaccatgacgctcctcacacctgaccagcttgaagcacagcttcgccagatcggggccgagcggtatcacaaccggcacccgttccatcgcaagctgcatgacggcaagctggacaaggcacaggttcaggcttgggcgctgaaccgctattattatcaggcccgcatcccggcgaaggatgcgacgcttctcgcacgtctgccgacggccgaactgcgccgcgaatggcgtcgccggatcgaggaccatgacggcacggagcccggaacgggcggtgttgcgcgctggctgatgctgacggatggtctggggctggaccgggattatgtggaaagcctcgatggtctgcttccagccacgcgcttctcggtcgatgcctatgtgaacttcgtgcgggaccagtcgattctggcggccattgcgtcgtcgctgacggaactgttttcgcccacgatcatcagcgagcgcgtctcggggatgctgcggcactacgactttgtgtcggaaaagacgctggcctatttcacgccgcgcctgacgcaggccccgcgggattccgatttcgcgctggcctatgtccgcgaaaaggcccgcacgccggagcagcagaaagaagtcctgggagcgctggagttcaagtgctccgtgctgtggacgatgctggatgcgctcgactacgcctatgtggaaggccacattccgccgggggctttcgttccatgacggaggccccgcatgtcgtggcggaggggacggttctctcctttgcccgggggcatcgtctccagcacgatcgtgtgcgggacgtgtggatcgtgcaggcgcctgaaaaagcatttgtagttgagggcgccgcgccgcatattctgcggctgctggatgggaagcgcagcgtcggcgagatcatccagcagcttgcaatcgagttttccgccccgcgtgaggtcattgcgaaagatgtcctcgcgcttctttctgaactgacagaaaagaacgtcctgcacacatgacactcccttcgccgccgatgagccttctggctgaactgacgcatcgatgcccgctttcctgcccctactgctccaatccgcttgaactcgaacgcaaggcggcagaactcgacacggccacctggactgccgtactggagcaggcggccgagcttggggtgctccaggttcatttctctggcggcgagcctatggcgcggcctgatctggtcgaactggtctccgtcgcacggagactcaacctgtattccaacttgatcacgtccggcgtgttgctggacgaaccgaaactggaagctctcgacagggcggggctggatcacatccagctctctttccaagacgtgacggaggcgggagccgagcgtatcggcggtctcaagggagcgcaggcccgcaaggttgcggcggcgcggctcatccgcgcgtccggcattccgatgacgctcaattttgtggtgcacagggaaaatgtcgcccgtatccccgagatgttcgccctggcgcgggaactcggagcggggcgggtggagatcgcgcatacccagtattatggctgggggctgaaaaaccgtgaggcgcttcttcccagccgggatcagctggaggaatccacacgcgccgtggaagcggagcgcgctaagggtggtttgtccgttgattatgtgacgccggactatcatgcagaccggcccaagccctgcatggggggatggggccagcgtttcgtgaatgtcacaccttcgggccgggtcctgccgtgtcatgcagccgaaatcattccggatgtcgcattcccgaatgtgcaggatgtgaccctgtccgaaatctggaacatctcaccgctgttcaacatgttccgcgggacggactggatgccggagccctgccgctcctgcgagcgcaaggagcgtgactggggcgggtgtcgctgtcaggcgatggcgctgacggggaatgccgcgaataccgatcccgtatgcagtctctccccctatcacgatcgggtggagcaggccgtcgagaacaacatgcagccagaaagcacgttgttctacaggcgttatacgtaa

SEQ ID NO: 69 - DNA, 5474 bp, Kluyvera intermedia
atgtggaaaaaacctgcgtttattgacctgcgtctgggcttggaagtgactctgtacatctccaaccgctaagacttctgcccgccgttcgtgcgggcattttgttttctctctttggtcaacgttcatgtttattaaagtcctcggttcagcggcgggcggcggcttcccgcagtggaactgtaactgcgccaattgcagcggcctgcgtaacggcagtattcaggcgcaggcgcgcacccaatcttcaatcattgtcagcgacaacggtgaagattgggtgctgtgcaacgcctcgccggacatcagccagcaaattgcccacacgccagaactgatcaaaaagggtgtgctgcgcggcacggcgattggcagcatcattctcaccgacagccaaatcgatcacaccactggcttgttgagcctgcgcgaaggctgtccgcatcaggtgtggtgcacgccggaagtgcatggcgatctcaccagcggtttcccgattttcaccatgttgcagcactggaatggcggcttgcagcatcatgcgttaacgccgctggagccgttccgcgtcgatgtctgccacagtttgcagttcaccgcgattccgattctcagcaacgcgccgccgtattcgccgtatcgcgatcgccctgccggaccacaacgtggcgctgttcatgaaaccaccggcaatggccaaacgctgctctacgcgccgggactcggcgagccggatgcggtgatccatgccgtggctgcaaagagccgattgcctgttattgatggcacggtgtggcaggacgacgagctgctcgccaccggcgttggcaaaaatactggcaaagcgatgggccacttagcgctggccgaagaacaaggtttaatggcgttgctggctgcgctgccggccaaacgcaagattttgattcacattaacaacaccaatccgatccttaatgagcaatccccgcagcgcgcctatttgacgcagcagggcattgaagtgagttgggatggcatggcgattcatctgcaggagtttgcatcatgatcatcactgaagcattatcgcccgccgcgtttgagcaggcgctgcgcggtttgcatcatgatcatcactgaagcattatcgcccgccgcgtttgagcaggcgctgcgcgataaaggcgcgttctaccacatacatcacccgtatcacattgccatgcacaatggcgaggcgacgcgcgagcagattcagggctgggtggcgaaccgcttctattatcagaccaacattccgctgaaagatgcggcgatcatggcgaattgcccggatgcggcgacgcggcgtaaatgggtgcagcgcatcctcgatcacgacggcagcaacggtcacgaaggcgggattgaagcctggctgcagttgggtgaggcggtggggctggaacgtgacgtgttgctgtcagagaagctggtgctgccgggcgtgcgttttgcggtggacgcctacgtgaacttcgcgcgccgtgctaactggcaggaagcggcctgtagctcgctcaccgagctgtttgcgccgcagattcatcagtcgcgtctcgacagctggccgcagcactatccgtggatcaaagaggaaggctatttctacttccgcagccgtttgagccaggcgaaccgcgatgtggagcatgggctggcgctggcgcttgaggtgtttacccgcgccgaacagcagaaccgcatgctggagatcctgcagtttaaactcgatattctctggaccatgctcgatgccatgaccatggcgtatgcgctgcaacgtccgccttatcatacggtcaccgatcaggtgtcctggcacagcacaagactggtataacaacatgaaagataacggtcaccgatcaggtgtcctggcacagcacaagactggtataacaacatgaaagataatgtgattccggctttccgtcgcggctaccgcatgcagtgggaagcggcgcaggatagccatgtggtgctctatccagaaggcatggccaaactgaacgagaccgccgtggcgatcctcgaactggtggatggcaaacaggatgtggcggcgatcgttgccacgctggatgcgcgtttcccggacgcgggcggtgtcggcgacgacgtcaaagagttcctgcaatccgccattgaacaaaaatggatacagtgtcgtgaacccgagtaaaagcgtgacgccaccgctgtggctgctggcagaactgacctatcgctgcccgctgcagtgtccatattgctccaatccgctggatttttcccagcagaagaaagagctgaccaccgaacagtggattgaggtgtttcgtcaggcgcgcgccatgggcagcgtgcagctcggtttttccggcggcgaaccgctaacccgtaaagacctgccggagttgattcgcgccgcgcgcgatctcggtttctacaccaacctgatcacctccggcatcggcctgacggcgaaaaaactcgacgcctttgccgacgccggactcgatcatatccagatcagcttccaggccagcgatgaaacgctgaacgcggcgctggccgggtcaaaaaaggccttccagcagaagctggagatggcgaaagcggtgaaagcgcacggctatccgatggtgctgaatttcgtgctgcatcgccacaatatcgaccagatcgacaaaatcatcgatctctgtatcgaactggatgccgacgatgtcgagctggcgacttgccaattctatggctgggcgcaactcaatcgtgaaggattgctgccgacgcgcgagcagatcgccaacgccgaagcggtggtggccgattatcgtcagcgcatgggcgccagcggcaatctcaccaatcctgctgttcgtgacgccggattactacgaagagcggccgaaaccctgcatgggcggatggggatcgatcttcctcagcgtcacgccgactgcaccgcgttgccgtgccacagcgcgcgtcagctgccggttgcgttcccgtcggtgctggagcgcacgctggacgatatctggtacaactcgtttggttttaaccgctatcgcggctttgactggatgccggaaccctgtcgctcctgcgatgaaaaagcgaaagactttggcggctgtcgctgtcaggcctttatgctgaccggcgatgccgataacaccgatccggtgtgcagcaaatcgccgcatcacggcaagatcctcgaagcacgacgtgaagccaactgcagcgacatcaaaatccagcagctgcagttccgcaatcgcagcaactccgaactgatcttttaagcagcgcgtcacctgatgcacgcgcgccagctcacgctcgccaacggcctgcgctgccatctttatcatcagcccgacgcgcgtgaggcggcggcgctgatgcgcgtgcaggccggcagtctggatgaagcggatcgctggccgggtttggcacatctgctggaacatctgctgttttgcggcagcgaagcttttcacggcgacgatcgcctgatgccttggctgcagcagcagggcggccaggtgaacgccaccacgcagttgagccgcagcgcctatttctttcagctcccagctgcggcgttgtcagcaggggtgctgcggctgtgcaatatgttggcgtcaccgttgctgacggcgcaggccattcagcaggaaacggcggtgattgaggctgagtatcaactgctgcaaaaccatgccgacacgctgagtgaagctgccgtgctggataagtggcagggacgttttcagcgcttccgcgtcggcagtcggcaggcctttggtgacaatgtcacggagttgcagtccgcattacgcgacttccatagccgcctgtattgcgctgaaaacatggaactttggctacaaggcccgcaatcactggatgagctcgaacagctggcggcacgcttcggcggcagtttgctggcgggtggagaatgcgctgcggctgttgaagctacgctgttgcgaggcgatcgtttactgctatcaggcggtgaagaaaatttctggctgacgatgttggtcgcgggcgatgagcagactgtgcgtgacaatgtcaccttattaaaagcattctggcaggacgaagcgccctacagcttgctggcgcagctgcgcgttgaggcgttatgcgaaacctttgacgcgcattggttatggcaagatgagcaacaagcgttactggcgctgcgctttagcgccaggtgcatttccccggcgcaggcgcaacagattgagcaacgcgtttggcagcatctggctgcgttagctgaatgtacggcgcttcagctgcggcactacgcacagctcgcgcagcaggatttcgcgaccttgaccttgtcgccgctggaacagttgcgcggccgggcgttgggcttcgcgccgggttgcgcgctgccagacaactttacggattttgccgcagcgttgccagattgtccgcgcacgcgtctgctcacgcagcaacagattgacggcgcaccgcatgcacacgcagggcttcaccttgcagctggtcgagtggctgccgccgtcgttggctgcgaccaaacccgcagcgtttcacttctatcctgcttcagccgctgttccgctccctcgtctcccacccgttgcgcagccgttgccgctgattgcaccggtaaaacaggtagaaacgctgctgctgcgtccggcgttttatcacaccttgaggaggaagacgcgcagtcgcggcagcgccagttacgcccgttgctggcggaattgcgccacgcgggtggcaacggcagctggcaacagacgcatgccagctggcagctactgctcaatttgcctgcatccgccgatcgcgcgttgtttagcgtgcatcaggcattacaggcgctgaacacgccgatgctggcgcaacccgcagcgaatactccttcgattgtgattcgtcaattgctcgccgcgttgcccactcgcttgatcaagccattacccgcgccacaatggctggcggcatggtgcggcaccaataccgcattaagccagcgcgtggcgcatctgctcagcgattttacgccggatctggcgcgcgacacgccgccggcacgactgcaacacggcatcgtgcccatcgcctgcgacgggcgcgatcaggcgctgctgctatttatgccgttaccgcaggccgacgatgccagcctggccgctttacgcgtgctggcgctgatgctggaaacgcgctttttccagcggttgcgcgtggatcagcaaattggctatgtggtgtcggcgcgctatcagcgggtggcggatgtggatggcttgatgctggcgctgcaatcaccggatatctcctggcgcgcgctgctgggtcattgcaaacgctttatgcgttgagatggtagccgagatcgcggcaatctcgccgcaaaagctggcggcatggcaggcaagctt

SEQ ID NO: 70 - DNA, 5641 bp, Erwinia amylovora
atgcagtggactaaaccaacgtttatcgatatgcgtctggggctggaagtcacgttatatatatctaatcgttaacccctcgccttatcacaatgtgaataacgctggctaaaacggtgatgcctgtggttaagcccaggcattttttcattccctttcccggttaacccatgaaaatcaaagttctcggatctgcggccggcggcggttttccgcaatggaactgctattgccctaattgccagggcgtgcgtaacggtagcatccgcgccacggcccgcacccagtcttccatcgcggtcagcgataacggcagcgactgggtgctttgcaacgcctcgcccgatatctgtcatcagattgctgccaaccccgaacttcatccacagggtaagctgcgcggttcgggaattggctccatcattcttaccgacagtcagatcgatcactgcaccggcttgctgaatctgcgagaaggttgcccacatcatgtgtggtgcacagcagaggtgcacgcggatctgaccagtggttttccgatcttcaccatgctgcaacactggaacggtggactggtgcatcatgctattcagctcgcgcagcctttttccgtcgcggtgtgtccggcgttgcggtttactgctattcccatcctcagcaacgccccaccttactcaccgtatcgcgggcgtccgcttcccggacacaatatcgcgctgatgatcgaaaataccgccagcggcagcaaactgctgtatgcccccggtttgggcgaaccagatgcgcagctgttggatttgatgtcgcaggccgactgcctgctggttgacggtacgctgtggcaggacaacgagctggcaaataccggcgttgggcgcaacaccggccgcgatatgggccacctggcgctggatgaagatcgcgggctgatggcgctactgggcgatctgcccgcaccgcgcaagatcctgatccatattaataatactaatccgatccttgatgaatcctccgccgagcgtctggcgttgagcgcacgcggcatcgaggtcagctacgacggcatgagcatagaactatgacccaaccggcattgatgactccacaacagtttgaacaggcgctgcgcgctcgcggcgcttactaccatattcatcacccgtaccatatcgctatgcataacggcgaggctacgcgtgagcagatccagggctgggtggcgaaccgctattattaccagacccgtattcctttgaaagacgccgcgattatggcgaactgccgggatgcgttaacccggcgtaaatgggtacagcgtattctcgaccatgatggccagggcgacagcgagggcggtatcgaagcctggctgcggcttggtgaagccgttggcctggatcgtgatgtgctgcagtcagagcagcgggtgctgcccggcgtgcgctttgccgttgatgcctacgtttcctttgcccgccgtgccgtctggcaggaagccgcctgtagctcactgactgaactgtatgcccctgaaatccatcagtcgcgtctcaacagctggccgcagcactacccgtggatcgaggaagagggctacggctatttccgtggtcgcctgggccaggcccggcgcgatgttgaacatggtttacagctggcgcttgagtactgcaacagcgttgaaaaacagcagcgtatgctggagatcctgcaatttaagctggatattttgtggagcatgctggatgccatgaccatggcgtatacacttaaccgtgcgccttatcacaccgtgaccgatcaacccgtctggcataaaggaaatctgctgtgaaatgtgaccaacgcattcccatattccgccgtggatatcgtctgcaatgggaagagatgcagaattgccatgtcatcctctatcccgaagggatggctaaactcaacgacagcgcaacgatgatcctcgaactggtggatggtcaacgctcgctggctgatatcgcccgtacgctgaatgtgcggttcccggacgcgggtggcgttgatgatgacgtcaccgatttctttgccgccgcgcgtgaacagaagtggataattttccgtgaacccagctgaatcaccgatcaaaccgccgctctggctactggcggagctgacctaccgctgcccgttacaatgcccgtattgctccaacccgcttgactttgcacggcaggaaaaagagctgacgactgcccagtggatcgacgtctttaagcaggcgcgggcgatgggcgcggtacagattggcttttcgggtggcgaaccgctggtgcgtaacgatctgcccgagctgatccgcagcgcgcgcgatctcggtttctataccaacctgattacctccgggatcggcctgacgcagaagaagatcgacgccttcgccgaagcgggactggatcatattcagatcagcttccaggccagcgatgaaaccctgaatgcggcgctggccggatcgacaaaggcatttcaacaaaagctggagatggcgcgtgcggttaaagcgcacggctatccaatggtgctgaattttgtgctgcatcgccacaatatcgaccagcttgaccgcattatcgagctgtgcatcgaactggaagccgatgatgtcgagctggcaacctgccagttttacggctgggcgcagctcaaccgcgagggactgctgcctacccgcgaccagctggcacgcgccgaggcggtggtgcaccactaccgcgagaagatggccgccagcggtaatctggccaatctgctgttcgtcaccccggactattatgaagagcggcctaagggctgcatgggcggctggggcgcgatcttcctcagcgtcacgccggaaggcatggcgctgccctgtcacagcgcccgccagctaccgatacctttcccgtcggtgctggagcacagcctgcaggatatctggtttaactcgtttggctttaaccgttatcgcggctttgactggatgccggagccatgtcgatcctgtgatgaaaaagagaaagacttcggcggctgccgctgccaggcgtttatgctgaccggcaacgctgataacgccgacccggtatgcagcaagtcagaacatcacggcaccatccttgccgcgcgtgaacaggccaactgcagccacattcaggtgaaccagctacggttccgtaatcgcgcaaactcccagcgggttaacgcccagctgatcttcaagggctgacggatgcagccacagcgacgacggctcgacaacggtttgcgcgtggtgttgattagcgatgcgcaggcagtacaggctgcagcgcttttccaggtggataccggtagccattatgaaccggacagctggccggggctggctcatctgttggaacatttgctgtttgccggcagttgcgcttatgcggatgacgagcggctgatggcctggttaccggccaggggagggcggctgaatgccaccacccagggcagcagcacggccttttttttcgagtgcgatgccgggctgctggctccggggctggctcgtttgagcgatatgctgctggctccgctgttggcagaaaatgccatccgccaggaagtggctaccattgacgccgagtgccgcctgctggccgggcagcaggatacgttgtgcaatgccgcgcagagcatggcatttgccgcacatccgtggcagcgttttcatatcggtaacgccgcgagttttaccggggactggccggcactgcggctggcgttacagcagtttcatcaacgctattatcacgcggcgaatatcacgctgtggttgcagggaccgcagtcgcccgaagcgttatggcagctggcccgacagtacggcagtgcgttttctgctgttggtgtgccaccgccagcgcttccgttactgcattattcatcgcagcctgatatggctttgcaactggcgggatcgccgcgtttgcgtctctctttcctgctggacaggccgcgcagtaacgagctgactttgctgcgccagctcttactggatgaggccgcaggtggcctgatggcaacgctgcgtgcgcaccatctctgcgatggcgcgcggctgctggtgccttatcacagcgcgatgcaaacactggtcagcgttgaactggcgctgattgatgagcagcaggccgcagaggtggaaggttgggtgcatcactggctgcaacgactgacggcactgacggcgcggcagcgccagcactatatgcggctcgctgactttcagtttgccaggctttcgccgatggatcagttacgcgaacacgccgttggttttgctccgccgcaagtacagcaagacgactggccagggttctgtgcgcggctgtcagtagcgcgtttagcccgcttgtggatcggcgcgcagtcgctggcgcagcagtgcagcgttcagggcttcaggctgcgctgcgctgcgcaaacgcgtgcgccggtaaccgcgttaatccccgctgaagcgctgacttttttctgcgaaagccagcctgacgctcaggcagcattacccgccgggcaggtcgcgctgaaccatcagcgggcgggtaagggccgtgcggcgctgttgctaagcccgctggatgaacttcatgcgccctggggagtcatcctgcaatcgcgcctgagagcgctggcagccgactgtgcccataagggcggcgatctgagcgtcagctgccagcagggtcattggctgatccagctgtgcggcagcccggcgttaatggtgcgcacgctggcaacgctgatccgccagctgtgtgagatatcccctgtgatgatcgcgctgggcgagcggcaataccaacgccagcagcaggcgcagcgtgaggggattgcggtacgcgccctgatcgatgcactgcccgcgctgttgcgttcatcggtttaccaaccggcagggcggcggctgccgcgcctggcgtggcaggcggtcctcgatggcggggacgatgcgctccgccagtcgttgtcgcagctgcttagcgcatttcccggcaccatcaatccgccgggccggatgcagccagagccgttagcaccgcagcctgaatatcaggtggcgaccatcagccacgatgcgaccttgttacgcttctgcccgctggtggaaaacagcacacagtgcctggcggcctggcagctgctggcgctgatctatcaacctgcatttttccaacgtctgcgcgtggagcagaatattggttatgtggtcagctgccgtttttaccaggcggcggggcggtcggggctgctgtttgccctgcaatctccccatctgagcaccggcgagctttcggcgcatatcgaccgtttcttgccaggaatggacgatgaactggccgctctcagcatggaaacagtgcgtgaaaagggggcggcattgctggcgcaacaaaggctggcagcgtctgatttccagcaggagtgccggcagcgctggctggctgcgcaacagtcggtacctcagcccgatgagtacgttatccaggggctgaccccggaacgcctgtcagactaccatcagcgcctgctctccgaccgccataacgcctggacgctggttggcatccctgcaaatcgcttctga

SEQ ID NO: 71 - DNA, 25 bp, Artificial
  Primer sequence
gcgccatatgcatcgacaatccttt

SEQ ID NO: 72 - DNA, 35 bp, Artificial
  Primer sequence
gcgcgctcaggctaattgcgtgggctaactttaag

SEQ ID NO: 73 - DNA, 28 bp, Artificial
  Primer sequence
gcgccatatggctcctgcaacggtaaat

SEQ ID NO: 74 - DNA, 30 bp, Artificial
  Primer sequence
ccggcatatgactgatctgaaagcaagcag

SEQ ID NO: 75 - DNA, 31 bp, Artificial
  Primer sequence
ccgctcagctcattagtagctgctggcgctc

SEQ ID NO: 76 - DNA, 39 bp, Artificial
  Primer sequence
gcaggctgagcttaactttaagaaggagatatacatatg

SEQ ID NO: 77 - DNA, 35 bp, Artificial
  Primer sequence
gcgcgctcagcctaattgcgtgggctaactttaag

SEQ ID NO: 78 - DNA, 37 bp, Artificial
  Primer sequence
gcgcggtaccgcacatgtcgcggatgttcaggtgttc

SEQ ID NO: 79 - DNA, 37 bp, Artificial
  Primer sequence
gcgcggatccgggcggagagtttggagaacctcttca

SEQ ID NO: 80 - DNA, 30 bp, Artificial
  Primer sequence
gcgcggatccacgcagcatcgggccgttct

SEQ ID NO: 81 - DNA, 31 bp, Artificial
  Primer sequence
gcgcggtaccaagcttcgctgccgcaaaaca

SEQ ID NO: 82 - DNA, 42 bp, Artificial
  Linker sequence
gcgcaagcatgcggatccggtaccaagcttgcatgcacacta